(19) United States
(12) Patent Application Publication
(10) Pub. No.: US 2009/0137572 A1
(43) Pub. Date: May 28, 2009

(54) 2-substituted-4-heteroaryl-pyrimidines useful for the treatment of proliferative disorders

(76) Inventors: Shudong Wang, Nottingham (GB); Gavin Wood, Cupar Fife (GB); Kenneth Duncan, Waltham, MA (US); Christopher Meades, Dundee (GB); Darren Gibson, Dundee (GB); Janice McLachlan, Dundee (GB); Peter Martin Fischer, Nottingham (GB)

Correspondence Address: LAHIVE & COCKFIELD, LLP;FLOOR 30, SUITE 3000, ONE POST OFFICE SQUARE, BOSTON, MA, 02109, US

(21) Appl. No.: 11/596,740

(22) PCT Filed: May 26, 2005
(86) PCT No.: PCT/GB05/02134

§ 371 Date: Oct. 21, 2008

(30) Foreign Application Priority Data
May 26, 2004 (GB) 0411791.7

(51) Int. Cl. A61K 31/541; C07D 417/14; A61K 31/5377; C12Q 1/48; G01N 33/53; C12N 9/99; A61P 35/00

(52) U.S. Cl. 514/227.8; 544/122; 514/235.8; 544/60; 435/184; 435/15; 435/7.8

(57) ABSTRACT

The present invention relates to selected substituted pyrimidines their preparation, pharmaceutical compositions containing them and their use as inhibitors of one or more protein kinases, and hence their use in the treatment of proliferative disorders, viral disorders and/or other disorders.

The present invention relates to new 2-substituted-4-heteroaryl-pyrimidine derivatives and their use in therapy. More specifically, the invention relates to 2-substituted-4-heteroaryl-pyrimidine derivatives having improved solubility properties.

### BACKGROUND

We have previously disclosed 2-substituted-4-heteroaryl-pyrimidines and their use in the treatment of proliferative disorders (Fischer P M, Wang S. PCT Intl. Patent Appl. Publ. WO 01/072745; Cyclacel Limited, UK, 2001). These compounds inhibit cyclin-independent protein kinases (CDKs), in particular CDK4/cyclin D, CDK2/cyclin E, CDK2/cyclin A, and CDK1/cyclin B, i.e. enzyme complexes that are important in human cell cycle progression. Furthermore, 2-phenylamino-heteroaryl-pyrimidines possess selective in vitro and in vivo antiproliferative activity against a range of human tumour cells (Wang S, Blake D, Clarke R, Duff S, McClue S J, McInnes C, Melville J, Stewart K, Taylor P, Westwood R, Wood G, Wu S-Y, Zhelev N Z, Zheleva D I, Walkinshaw M, Lane D P, Fischer P M. Proc. Amer. Assoc. Cancer Res. 2002; 43: 4202).

The present invention seeks to provide further 2-substituted-4-heteroaryl-pyrimidines. More specifically, the present invention preferably seeks to provide 2-substituted-4-heteroaryl-pyrimidines which display improved aqueous solubility and/or bioavailability.

### STATEMENT OF INVENTION

A first aspect of the invention relates to a compound selected from compounds [1]-[220] as set forth in Table 1, or a pharmaceutically acceptable salt thereof.

The present compounds are equipped with solubilising functions on the phenyl and/or heteroaryl rings of the 2-phenylamino-4-heteroaryl-pyrimidine system. Modification with solubilising moieties has preserved the desired in vitro biological activity (inhibition of CDKs and cytotoxicity against transformed human cells) and in some cases has led to surprising and unexpected increases in potency. Furthermore, in vivo absorption, and oral bioavailability in particular can also be improved using the solubilising strategies presented herein.

A second aspect of the invention relates to a compound of formula I, or a pharmaceutically acceptable salt thereof,

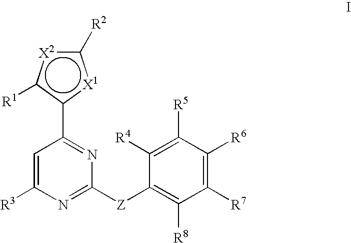

wherein:
one of X¹ and X² is S, and the other of X¹ and X² is N;

### Z is NH, NHCO, NHCOCH₂, NHSO₂, NHCH₂, CH₂, CH₂CH₂, CH═CH, O, S, SO₂, or SO;

R¹, R², R³, R⁴, R⁵, R⁶, R⁷ and R⁸ are each independently H, alkyl, alkyl-R⁹, aryl, aryl-R⁹, aralkyl, aralkyl-R⁹, halogeno, NO₂, CN, OH, O-alkyl, COR⁹, COOR⁹, O-aryl, O—R⁹, NH₂, NH-alkyl, NH-aryl, NH(aralkyl), N-(alkyl)₂, N-(aryl)₂, N-(alkyl)(aryl), NH—R⁹, N—(R⁹)(R¹⁰), N-(alkyl)(R⁹), N-(aryl)(R⁹), COOH, CONH₂, CONH-alkyl, CONH-aryl, CON-(alkyl)(R⁹), CON(aryl)(R⁹), CONH—R⁹, CON—(R¹⁰), SO₃H, SO₂-alkyl, SO₂-alkyl-R⁹, SO₂-aryl, SO₂-aryl-R⁹, SO₂NH₂, SO₂NH—R⁹, SO₂N—(R⁹)(R¹⁰), CF₃, CO-alkyl, CO-alkyl-R⁹, CO-aryl, CO-aryl-R⁹ or R¹¹, wherein alkyl, aryl, aralkyl groups may be further substituted with one or more groups selected from halogeno, NO₂, OH, O-methyl, NH₂, COOH, CONH₂ and CF₃;
or two of R⁴-R⁸ are linked to form a cyclic ether containing one or more oxygens;
R⁹ and R¹⁰ are each independently solubilising groups selected from:
(i)
   a mono-, di- or polyhydroxylated alicyclic group;
   a di- or polyhydroxylated aliphatic or aromatic group;
   a carbohydrate derivative;
   an O- and/or S-containing heterocyclic group optionally substituted by one or more hydroxyl groups;
   an aliphatic or aromatic group containing a carboxamide, sulfoxide, sulfone, or sulfonamide function; or
   a halogenated alkylcarbonyl group;
(ii) COOH, SO₃H, OSO₃H, PO₃H₂, or OPO₃H₂;
(iii) Y, where Y is selected from an alicyclic, aromatic, or heterocyclic group comprising one or more of the functions ═N—, —N—, —O—, —NH₂, —NH—, a quarternary amine salt, guanidine, and amidine, where Y is optionally substituted by one or more substituents selected from:
   halogen:
   SO₂-alkyl;
   alkyl optionally substituted by one or more OH or halogen groups;
   CO-alkyl;
   aralkyl;
   COO-alkyl; and
      an ether group optionally substituted by one or more OH groups;
(iv) a natural or unnatural amino acid, a peptide or a peptide derivative;
   each R¹¹ is a solubilising group as defined for R⁹ and R¹⁰ in (i) or (iv) above; or is selected from:
(v) OSO₃H, PO₃H₂, or OPO₃H₂;
(vi) Y as defined above, but excluding guanidine and quarternary amine salts;
(vii) NHCO(CH₂)ₘ[NHCO(CH₂)_{m′}]ₚ[NHCO(CH₂)_{m″}]_{q}Y or NHCO(CH₂)ₜNH(CH₂)_{t′}Y where p and q are each 0 or 1, and m, m′, m″, t and t′ are each independently an integer from 1 to 10; and
(viii) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are optionally substituted by one or more substituents selected from OH, NH₂, halogen and NO₂, R¹⁴ and R¹⁵ are each independently H or alkyl, and n and n′ are each independently 0, 1, 2, or 3;
(ix) an ether or polyether optionally substituted by one or more hydroxyl groups or one or more Y groups;
(x) (CH₂)ᵣNH₂; where r is 0, 1, 2, or 3;
(xi) (CH₂)_{r′}OH; where r′ is 0, 1, 2, or 3;
(xii) (CH₂)_{n″}NR¹⁷COR¹⁸ where R¹⁷ is H or alkyl, n″ is 0, 1, 2 or 3 and R¹⁸ is an aryl or heteroaryl group, each of which may be optionally substituted by one or more substituents selected from halogeno, NO₂, OH, alkoxy, NH₂, COOH, CONH₂ and CF₃;
(xiii) SO₂NR¹⁹R²⁰ where R¹⁹ and R²⁰ are each independently H, alkyl, aralkyl, CO-alkyl or aryl, with the proviso that at least one of R¹⁹ and R²⁰ is other than H, or R¹⁹ and R²⁰ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CH₂CO₂-alkyl, CONH₂ and CF₃;
(xiv) N-piperidinyl, N-pyrrolidinyl or N-thiomorpholinyl, each of which may be optionally substituted by one or more alkyl, alkoxy or CO-alkyl groups;
   with the proviso mat when Z is —NH— at least one of R⁴-R⁸ is selected from:
   (CH₂)_{n″}NR¹⁷COR¹⁸;
   SO₂NR¹⁹R²⁰; and
   N-piperidinyl, N-pyrrolidinyl and N-thiomorpholinyl, each of which may be optionally substituted by one or more alkyl, alkoxy or CO-alkyl groups;
      or two of R⁴-R⁸ are linked to form a cyclic ether containing one or more oxygens.

A third aspect of the invention relates to a compound of formula II, or a pharmaceutically acceptable salt thereof,

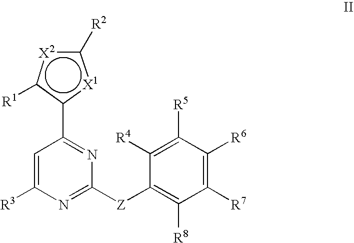

wherein:
one of X¹ and X² is S, and the other of X¹ and X² is N;

### Z is NH, NHCO, NHCOCH₂, NHSO₂, NHCH₂, CH₂, CH₂CH₂, CH═CH, O, S, SO₂, or SO;

R¹, R³, R⁴, R⁵, R⁶ and R⁷ and R⁸ are each independently H, alkyl, alkyl-R⁹, aryl, aryl-R⁹, aralkyl, aralkyl-R⁹, halogeno, NO₂, CN, OH, O-alkyl, COR⁹, COOR⁹, O-aryl, O—R⁹, NH₂, NH-alkyl, NH-aryl, NH(aralkyl), N-(alkyl)₂, N-(aryl)₂, N-(alkyl)(aryl), NH—R⁹, N—(R⁹)(R¹⁰), N-(alkyl)(R⁹), N-(aryl)(R⁹), COOH, CONH₂, CONH-alkyl, CONH-aryl, CON-(alkyl)(R⁹), CON(aryl)(R⁹), CONH—R⁹, CON—(R⁹)(R¹⁰), SO₃H, SO₂-alkyl, SO₂-alkyl-R⁹, SO₂-aryl, SO₂-aryl-R⁹, SO₂NH₂, SO₂NH—R⁹, SO₂N—(R⁹)(R¹⁰), CF₃, CO-alkyl, CO-alkyl-R⁹, CO-aryl, CO-aryl-R⁹ or R¹¹, wherein alkyl, aryl, aralkyl groups may be further substituted with one or more groups selected from halogeno, NO₂, OH, O-methyl, NH₂, COOH, CONH₂ and CF₃;
R² is selected from pyridinyl, N(alkyl)pyridinyl, NH(aralkyl) and N(alkyl)(aralkyl), wherein said alkyl, pyridinyl and aralkyl groups may be optionally substituted by one or more alkyl, CF₃ or ether groups;
R⁹ and R¹⁰ are each independently solubilising groups selected from:
(i)
   a mono-, di- or polyhydroxylated alicyclic group;
   a di- or polyhydroxylated aliphatic or aromatic group;
   a carbohydrate derivative;
   an O- and/or S-containing heterocyclic group optionally substituted by one or more hydroxyl groups;
   an aliphatic or aromatic group containing a carboxamide, sulfoxide, sulfone, or sulfonamide function; or
   a halogenated alkylcarbonyl group;
(ii) COOH, SO₃H, OSO₃H, PO₃H₂, or OPO₃H₂;
(iii) Y, where Y is selected from an alicyclic, aromatic, or heterocyclic group comprising one or more of the functions ═N—, —N—, —O—, —NH₂, —NH—, a quarternary amine salt, guanidine, and amidine, where Y is optionally substituted by one or more substituents selected from:
   halogen:
   SO₂-alkyl;
   alkyl optionally substituted by one or more OH or halogen groups;
   CO-alkyl;
   aralkyl;
   COO-alkyl; and
      an ether group optionally substituted by one or more OH groups;
(iv) a natural or unnatural amino acid, a peptide or a peptide derivative;
   each R¹¹ is a solubilising group as defined for R⁹ and R¹⁰ in (i) or (iv) above; or is selected from:
(v) OSO₃H, PO₃H₂) or OPO₃H₂;
(vi) Y as defined above, but excluding guanidine and quarternary amine salts;
(vii) NHCO(CH₂)ₘ[NHCO(CH₂)_{m′}]ₚ[NHCO(CH₂)_{m″}]_{q}Y or NHCO(CH₂)ₜNH(CH₂)_{t′}Y where p and q are each 0 or 1, and m, m′, m″, t and t′ are each independently an integer from 1 to 10; and
(viii) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are optionally substituted by one or more substituents selected from OH, NH₂, halogen and NO₂, R¹⁴ and R¹⁵ are each independently H or alkyl, and n and n′ are each independently 0, 1, 2, or 3;
(ix) an ether or polyether optionally substituted by one or more hydroxyl groups or one or more Y groups;
(x) (CH₂)ᵣNH₂; where r is 0, 1, 2, or 3;
(xi) (CH₂)ᵣ—OH; where r′ is 0, 1, 2, or 3;
(xii) (CH₂)_{n″}NR¹⁷COR¹⁸ where R¹⁷ is H or alkyl, n″ is 0, 1, 2 or 3 and R¹⁸ is an aryl or heteroaryl group, each of which may be optionally substituted by one or more substituents selected from halogeno, NO₂, OH, alkoxy, NH₂, COOH, CONH₂ and CF₃;
(xiii) SO₂NR¹⁹R²⁰ where R¹⁹ and R²⁰ are each independently H, alkyl, aralkyl, CO-alkyl or aryl, with the proviso that at least one of R¹⁹ and R²⁰ is other than H, or R¹⁹ and R²⁰ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CH₂CO₂-alkyl, CONH₂ and CF₃;
(xiv) N-piperidinyl, N-pyrrolidinyl or N-thiomorpholinyl, each of which may be optionally substituted by one or more alkyl, alkoxy or CO-alkyl groups;
   wherein at least one of R⁶ and R⁷ is a (CH₂)ₙNR¹⁴COR¹² group or an alicyclic group containing at least one —N— wherein said alicyclic group is optionally substituted by one or more alkyl, alkoxy, CO-alkyl or aralkyl groups.

A fourth aspect of the invention relates to pharmaceutical compositions comprising the above described compounds admixed with a pharmaceutically acceptable diluent, excipient or carrier.

A fifth aspect of the invention relates to the use of the above described compounds in the preparation of a medicament for treating one or more of the following: a proliferative disorder, a viral disorder, a stroke, diabetes, a CNS disorder and alopecia.

A sixth aspect of the invention relates to the use of the above described compounds for inhibiting a protein kinase.

A seventh aspect of the invention relates to the use of the above described compounds in an assay for identifying further candidate compounds capable of inhibiting a protein kinase.

### DETAILED DESCRIPTION

As used herein the term “alkyl” includes both straight chain and branched alkyl groups having from 1 to 8 carbon atoms, e.g. methyl, ethyl propyl, isopropyl, butyl, isobutyl, tert-butyl, pentyl, hexyl etc. and the term “lower alkyl” is similarly used for groups having from 1 to 4 carbon atoms.

As used herein, the term “aryl” refers to a substituted (mono- or poly-) or unsubstituted monoaromatic or polyaromatic system, wherein said polyaromatic system may be fused or unfused. Preferably, the term “aryl” is includes groups having from 6 to 10 carbon atoms, e.g. phenyl, naphthyl etc. The term “aryl” is synonymous with the term “aromatic”.

The term “aralkyl” is used as a conjunction of the terms alkyl and aryl as given above. Preferred aralkyl groups include CH₂Ph and CH₂CH₂Ph and the like.

The term “alicyclic” refers to a cyclic aliphatic group.

The term “aliphatic” takes its normal meaning in the art and includes non-aromatic groups such as alkanes, alkenes and alkynes and substituted derivatives thereof.

As used herein, the term “carbohydrate derivative” refers to a compound of general formula Cₓ(H₂O)_{y} or a derivative thereof. Preferably, the carbohydrate is a a mono-, di- or tri-saccharide. Monosaccharides can exist as either straight chain or ring-shaped molecules and are classified according to the number of carbon atoms they possess; trioses have three carbons, tetroses four, pentoses five and hexoses six. Each of these subgroups may be further divided into aldoses and ketoses, depending on whether the molecule contains an aldehyde group (—CHO) or a ketone group (C═O). Typical examples of monosaccharides include glucose, fructose, and galactose. Disaccharides consist of two linked monosaccharide molecules, and include for example, maltose and lactose. Trisaccharides consist of three linked monosaccharide molecules.

The term “derivative” as used herein includes chemical modification of an entity. Illustrative of such chemical modifications would be replacement of hydrogen by a halo group, an alkyl group, an acyl group or an amino group.

The term “heterocycle” refers to a saturated or unsaturated cyclic group containing one or more heteroatoms in the ring. The term “heteroaryl” refers to a heterocyclic group that is aromatic.

In one preferred embodiment of the invention, the compound is selected from the following:
1-(4-{3-[4-(4-Methyl-2-methylamino-thiazol-5-yl)-pyrimidin-2-ylamino]-phenyl}-piperazin-1-yl)-ethanone[3];
[4-(2,4-Dimethyl-thiazol-5-yl)-pyrimidin-2-yl]-(3-methanesulfonyl-phenyl)-amine [4];
N-{3-[4-(2-Ethylamino-4-methyl-thiazol-5-yl)-pyrimidin-2-ylamino]-benzyl}-methanesulfonamide[5];
N-{3-[4-(2-Amino-4-methyl-thiazol-5-yl)-pyrimidin-2-ylamino]-benzyl}-methanesulfonamide [6];
[4-(4-Methyl-2-methylamino-thiazol-5-yl)-pyrimidin-2-yl]-(3-piperazin-1-yl-phenyl)-amine[7];
[4-(2,4-Dimethyl-thiazol-5-yl)-pyrimidin-2-yl]-(3-piperazin-1-yl-phenyl)-amine[8];
N-{3-[4-(2-Ethylamino-4-methyl-thiazol-5-yl)-pyrimidin-2-ylamino]-benzyl}-C,C,C-trifluoro-methanesulfonamide[10];
N-{3-[4-(2,4-Dimethyl-thiazol-5-yl)-pyrimidin-2-ylamino]-benzyl}-C,C,C-trifluoro-methanesulfonamide[11];
N-{3-[4-(2-Amino-4-methyl-thiazol-5-yl)-pyrimidin-2-ylamino]-benzyl}-C,C,C-trifluoro-methanesulfonamide[12];
N-{4-[4-(2-Ethylamino-4-methyl-thiazol-5-yl)-pyrimidin-2-ylamino]-benzyl}-acetamide[13];
N-{4-[4-(4-Methyl-2-methylamino-thiazol-5-yl)-pyrimidin-2-ylamino]-benzyl}-acetamide[14];
N-{4-[4-(2,4-Dimethyl-thiazol-5-yl)-pyrimidin-2-ylamino]-benzyl}-acetamide[15];
N-{4-[4-(2-Amino-4-methyl-thiazol-5-yl)-pyrimidin-2-ylamino]-benzyl}-acetamide[16];
[4-(2-Ethylamino-4-methyl-thiazol-5-yl)-pyrimidin-2-yl]-(4-methanesulfonyl-phenyl)-amine[17];
(4-Methanesulfonyl-phenyl)-[4-(4-methyl-2-methylamino-thiazol-5-yl)-pyrimidin-2-yl]-amine[20];
(3-Methanesulfonyl-phenyl)-[4-(4-methyl-2-methylamino-thiazol-5-yl)-pyrimidin-2-yl]-amine[27];
[4-(2-Ethylamino-4-methyl-thiazol-5-yl)-pyrimidin-2-yl]-(3-methanesulfonyl-phenyl)-amine[28];
1-(4-{3-[4-(2,4-Dimethyl-thiazol-5-yl)-pyrimidin-2-ylamino]-phenyl}-piperazin-1-yl)-ethanone[46];
{3-[4-(2,4-Dimethyl-thiazol-5-yl)-pyrimidin-2-ylamino]-5-hydroxymethyl-phenyl}-methanol[47];
{3-Hydroxymethyl-5-[4-(4-methyl-2-methylamino-thiazol-5-yl)-pyrimidin-2-ylamino]-phenyl}-methanol[48];
N-{3-[4-(2,4-Dimethyl-thiazol-5-yl)-pyrimidin-2-ylamino]-benzyl}-methanesulfonamide[49];
1-(4-{4-[4-(2-Amino-4-methyl-thiazol-5-yl)-pyrimidin-2-ylamino]-phenyl}-piperazin-1-yl)-ethanone [57];
1-(4-{4-[4-(2-Ethylamino-4-methyl-thiazol-5-yl)-pyrimidin-2-ylamino]-phenyl}-piperazin-1-yl)-ethanone [58];
[4-(2-Ethylamino-4-methyl-thiazol-5-yl)-pyrimidin-2-yl]-(4-piperazin-1-yl-phenyl)-amine [59];
[4-(4-Benzyl-piperazin-1-yl)-phenyl]-[4-(2-ethylamino-4-methyl-thiazol-5-yl)-pyrimidin-2-yl]-amine [60];
[4-(2-Amino-4-methyl-thiazol-5-yl)-pyrimidin-2-yl]-(4-piperazin-1-yl-phenyl)-amine[61];
[4-(2-Benzylamino-4-methyl-thiazol-5-yl)-pyrimidin-2-yl]-(4-morpholin-4-yl-phenyl)-amine [117];
1-(4-{4-[4-(4-Methyl-2-pyridin-3-yl-thiazol-5-yl)-pyrimidin-2-ylamino]-phenyl}-piperazin-1-yl)-ethanone [119];
{4-[2-(Ethyl-methyl-amino)-4-methyl-thiazol-5-yl]-pyrimidin-2-yl}-(4-morpholin-4-yl-phenyl)-amine [120];
[4-(2,6-Dimethyl-morpholin-4-yl)-phenyl]-[4-(2,4-dimethyl-thiazol-5-yl)-pyrimidin-2-yl]-amine [121];
{5-[2-(4-Dimethylamino-phenylamino)-pyrimidin-4-yl]-4-methyl-thiazol-2-yl}-methanol [132];
{4-[4-Methyl-2-(thiophene-2-sulfonylmethyl)-thiazol-5-yl]-pyrimidin-2-yl}-(4-morpholin-4-yl-phenyl)-amine [138];
{4-[2-(2-Methoxy-ethylamino)-4-methyl-thiazol-5-yl]-pyrimidin-2-yl}-(4-morpholin-4-yl-phenyl)-amine [144];
N⁴-[4-(2,4-Dimethyl-thiazol-5-yl)-pyrimidin-2-yl]-N¹-methyl-2-trifluoromethyl-benzene-1,4-diamine [149];
[4-(2,4-Dimethyl-thiazol-5-yl)-pyrimidin-2-yl]-(3-morpholin-4-ylmethyl-phenyl)-amine [150];
4-[4-(2,4-Dimethyl-thiazol-5-yl)-pyrimidin-2-ylamino]-2-morpholin-4-ylmethyl-phenol [151];
(3-Methoxy-4-morpholin-4-yl-phenyl)-[4-(4-methyl-2-pyridin-3-yl-thiazol-5-yl)-pyrimidin-2-yl]-amine [156];
[4-(2-Ethylamino-4-methyl-thiazol-5-yl)-pyrimidin-2-yl]-(3-methoxy-4-morpholin-4-yl-phenyl)-amine [157];
[4-(2,6-Dimethyl-morpholin-4-yl)-phenyl]-[4-(2-ethylamino-4-methyl-thiazol-5-yl)-pyrimidin-2-yl]-amine [169];
[4-(2-Amino-4-methyl-thiazol-5-yl)-pyrimidin-2-yl]-(3-methoxy-4-morpholin-4-yl-phenyl)-amine [182];
[4-(2,4-Dimethyl-thiazol-5-yl)-pyrimidin-2-yl]-(4-piperidin-1-yl-phenyl)-amine [193];
[4-(2-Ethylamino-4-methyl-thiazol-5-yl)-pyrimidin-2-yl]-(4-piperidin-1-yl-phenyl)-amine [194];
[4-(2-Amino-4-methyl-thiazol-5-yl)-pyrimidin-2-yl]-(4-piperidin-1-yl-phenyl)-amine [195];
{4-Methyl-5-[2-(4-piperidin-1-yl-phenylamino)-pyrimidin-4-yl]-thiazol-2-yl}-methanol [197];
{5-[2-(3-Methoxy-4-morpholin-4-yl-phenylamino)-pyrimidin-4-yl]-4-methyl-thiazol-2-yl}-methanol [200];
[4-(2-Amino-4-methyl-thiazol-5-yl)-pyrimidin-2-yl]-(4-thiomorpholin-4-yl-phenyl)-amine [201];
[4-(2,4-Dimethyl-thiazol-5-yl)-pyrimidin-2-yl]-(4-thiomorpholin-4-yl-phenyl)-amine [202];
[4-(2-Ethylamino-4-methyl-thiazol-5-yl)-pyrimidin-2-yl]-(4-thiomorpholin-4-yl-phenyl)-amine) [203]
[4-(2,4-Dimethyl-thiazol-5-yl)-pyrimidin-2-yl]-(3-methyl-4-piperidin-1-yl-phenyl)-amine [205];
[4-(2-Amino-4-methyl-thiazol-5-yl)-pyrimidin-2-yl]-(3-methyl-4-piperidin-1-yl-phenyl)-amine [206]
[4-(2-Ethylamino-4-methyl-thiazol-5-yl)-pyrimidin-2-yl]-(3-methyl-4-piperidin-1-yl-phenyl)-amine [207];
{4-Methyl-5-[2-(3-methyl-4-piperidin-1-yl-phenylamino)-pyrimidin-4-yl]-thiazol-2-yl}-methanol [209];
Cyclopropyl-(4-{4-[4-(2,4-dimethyl-thiazol-5-yl)-pyrimidin-2-ylamino]-phenyl}-piperazin-1-yl)-methanone [213];
[4-(2,4-Dimethyl-thiazol-5-yl)-pyrimidin-2-yl]-(4-methoxy-3-morpholin-4-ylmethyl-phenyl)-amine [215];
   or a pharmaceutically acceptable salt thereof.

In one preferred embodiment of the invention, the compound is selected from the following:
1-(4-{3-[4-(4-Methyl-2-methylamino-thiazol-5-yl)-pyrimidin-2-ylamino]-phenyl}-piperazin-1-yl)-ethanone[3];
[4-(2,4-Dimethyl-thiazol-5-yl)-pyrimidin-2-yl]-(3-methanesulfonyl-phenyl)-amine [4];
N-{3-[4-(2-Ethylamino-4-methyl-thiazol-5-yl)-pyrimidin-2-ylamino]-benzyl}-methanesulfonamide[5];
N-{3-[4-(2-Amino-4-methyl-thiazol-5-yl)-pyrimidin-2-ylamino]-benzyl}-methanesulfonamide [6];
[4-(4-Methyl-2-methylamino-thiazol-5-yl)-pyrimidin-2-yl]-(3-piperazin-1-yl-phenyl)-amine[7];
[4-(2,4-Dimethyl-thiazol-5-yl)-pyrimidin-2-yl]-(3-piperazin-1-yl-phenyl)-amine[8];
N-{3-[4-(2-Ethylamino-4-methyl-thiazol-5-yl)-pyrimidin-2-ylamino]-benzyl}-C,C,C-trifluoro-methanesulfonamide[10];
N-{3-[4-(2,4-Dimethyl-thiazol-5-yl)-pyrimidin-2-ylamino]-benzyl}-C,C,C-trifluoro-methanesulfonamide[11];
N-{3-[4-(2-Amino-4-methyl-thiazol-5-yl)-pyrimidin-2-ylamino]-benzyl}-C,C,C-trifluoro-methanesulfonamide[12];
N-{4-[4-(2-Ethylamino-4-methyl-thiazol-5-yl)-pyrimidin-2-ylamino]-benzyl}-acetamide[13];
N-{4-[4-(4-Methyl-2-methylamino-thiazol-5-yl)-pyrimidin-2-ylamino]-benzyl}-acetamide[14];
N-{4-[4-(2,4-Dimethyl-thiazol-5-yl)-pyrimidin-2-ylamino]-benzyl}-acetamide[15];
N-{4-[4-(2-Amino-4-methyl-thiazol-5-yl)-pyrimidin-2-ylamino]-benzyl}-acetamide[16];
[4-(2-Ethylamino-4-methyl-thiazol-5-yl)-pyrimidin-2-yl]-(4-methanesulfonyl-phenyl)-amine[17];
(4-Methanesulfonyl-phenyl)-[4-(4-methyl-2-methylamino-thiazol-5-yl)-pyrimidin-2-yl]-amine[20];
(3-Methanesulfonyl-phenyl)-[4-(4-methyl-2-methylamino-thiazol-5-yl)-pyrimidin-2-yl]-amine[27];
[4-(2-Ethylamino-4-methyl-thiazol-5-yl)-pyrimidin-2-yl]-(3-methanesulfonyl-phenyl)-amine[28];
1-(4-{3-[4-(2,4-Dimethyl-thiazol-5-yl)-pyrimidin-2-ylamino]-phenyl}-piperazin-1-yl)-ethanone[46];
{3-[4-(2,4-Dimethyl-thiazol-5-yl)-pyrimidin-2-ylamino]-5-hydroxymethyl-phenyl}-methanol[47];
{3-Hydroxymethyl-5-[4-(4-methyl-2-methylamino-thiazol-5-yl)-pyrimidin-2-ylamino]-phenyl}-methanol[48];
N-{3-[4-(2,4-Dimethyl-thiazol-5-yl)-pyrimidin-2-ylamino]-benzyl}-methanesulfonamide [49];
   or a pharmaceutically acceptable salt thereof.

### Compounds of Formula I

As mentioned above, one aspect of the invention relates to compounds of formula I as defined above, or pharmaceutically acceptable salts thereof.

Preferably, the compounds of formula I bear a mono- or di-substituted thiazol-3-yl or thiazol-5-yl radical attached to the pyrimidine ring through one of the ring carbon atoms

Most preferably, the heterocycle is a thiazol-5-yl group. Thus, in one preferred embodiment of the invention, X¹ is S and X² is N.

Preferably, R¹ and R² are each independently selected from alkyl, NH₂ and NH-alkyl, N-(alkyl)₂ and N-(alkyl)(aryl).

More preferably, R¹ and R² are each independently selected from alkyl, NH₂ and NH-alkyl.

Even more preferably, R¹ is selected from methyl, NH₂, NHMe and NHEt, and R² is methyl. More preferably still, R¹ is Me.

In yet another preferred embodiment, at least one of R², R⁵, R⁶ or R⁷ is an R⁹ or R¹⁰-containing group, or is R¹¹.

In one particularly preferred embodiment, X¹ is S, X² is N, Z is NH, R¹ is Me, R² is alkyl or amino, R³ is H, one or two of R⁵, R⁶, and R⁷ are CF₃, OH, O-alkyl, halogeno, NO₂, NH₂, NH-alkyl or N-(alkyl)₂ and at least one of R², R⁵, R⁶ or R⁷ is an R⁹ or R¹⁰-containing group, or is R¹¹.

In another preferred embodiment, at least one of R¹, R², R³, R⁴, R⁵, R⁶, R⁷ and R⁸ is R¹¹.

In one preferred embodiment, R¹¹ is a solubilising group as defined for R⁹ and R¹⁰ in (i)-(iv) above, or (v)-(xiv) as defined above.

In another preferred embodiment, R¹¹ is a solubilising group as defined for R⁹ and R¹⁰ in (i)-(iv) above, or (v)-(vii), (ix)-(xiv) as defined above, or is selected from:
(CH₂)ₙNR¹⁴COR¹², where R¹² is an alkyl group optionally comprising one or more heteroatoms, and which is optionally substituted by one or more substituents selected from OH, NH₂ and NO₂,
(CH₂)_{n′}NR¹⁵SO₂R¹³, where R¹³ is an alkyl group optionally comprising one or more heteroatoms, and which is substituted by one or more substituents selected from OH, NH₂, halogen and NO₂,
SO₂R¹⁶, where R¹⁶ is an alkyl group optionally comprising one or more heteroatoms, and which is optionally substituted by one or more substituents selected from OH, NH₂, halogen and NO₂; and
   R¹⁴ and R¹⁵ are each independently H or alkyl, and n and n′ are each independently 0, 1, 2, or 3.

Preferably, the solubilising group is R¹¹ and is:
(a) Y as defined in above, but excluding guanidine, where Y can also be an alicyclic, aromatic, or heterocyclic group comprising one or more ═N— groups;
(b) NHCO(CH₂)ₘ[NHCO(CH₂)_{m′}]ₚ[NHCO(CH₂)_{m″}]_{q}Y or NHCO(CH₂)ₜNH(CH₂)_{t′}Y where p and q are each 0 or 1, and m, m′, m″, t and t′ are each an integer from 1 to 10; or
(c) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are substituted by one or more substituents selected from OH, NH₂, halogen and NO₂, R¹⁴ and R¹⁵ are each independently H or alkyl, and n and n′ are each independently 0, 1, 2, or 3.

Preferably, the solubilising group is R¹¹, and R¹¹ is:
(a) Y as defined above, but excluding guanidine, where Y can also be an alicyclic, aromatic, or heterocyclic group comprising one or more ═N— groups;
(b) NHCO(CH₂)ₘ[NHCO(CH₂)_{m′}]ₚ[NHCO(CH₂)_{m″}]_{q}Y where p and q are each 0 or 1, and m, m′ and m″ are each integers from 1 to 10
(c) NHCOR¹² or NHSO₂R¹³, where R¹² and R¹³ are each alkyl groups optionally comprising one or more heteroatoms, and which are optionally substituted by one or more substituents selected from OH, NH₂, halogen and NO₂.

Even more preferably, Y is an alicyclic group comprising one or more of the functions —O—, —N—, NH₂, —NH—, ═N—, a quarternary amine salt, or amidine, and wherein Y is optionally substituted by one or more substituents as defined above. Preferably, Y is other than pyridinyl.

More preferably still, Y is a morpholinyl or piperazinyl group, each of which may be optionally substituted by one or more substituents selected from SO₂-alkyl, alkyl optionally substituted by one or more OH groups, CO-alkyl, aralkyl, COO-alkyl, and an ether group optionally substituted by one or more OH groups

In one especially preferred embodiment of the invention, Y is a 2-oxo-hexahydro-thien[3,4-d]imidazole group.

In one preferred embodiment, at least one of R², R⁶ or R⁷ is R¹¹.

For this embodiment, preferably R¹¹ is selected from the following:

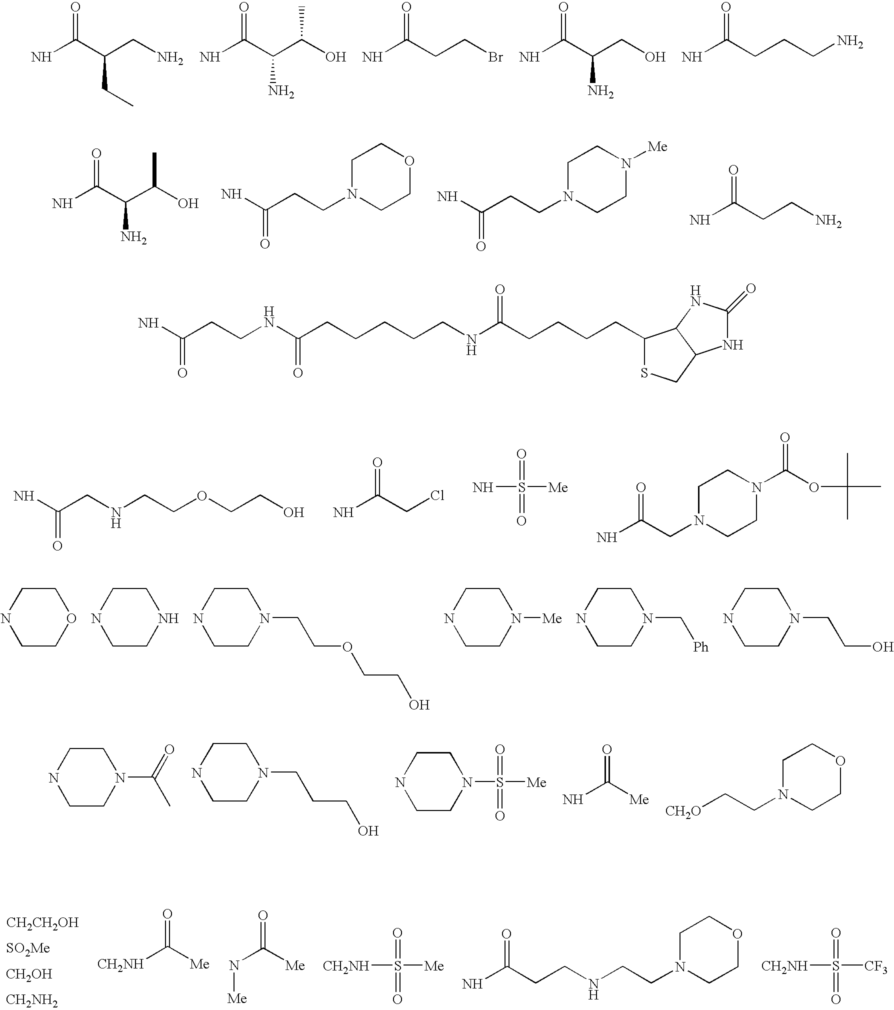

In one especially preferred embodiment, R⁶ or R⁷ is R¹¹. More preferably, R⁶ is R¹¹ and R², R⁴, R⁵, R⁷ and R⁸ are each independently selected from alkyl, H, CF₃, OH, O-alkyl, halogeno, NO₂, NH₂, NH-alkyl and N-(alkyl)₂. More preferably still, R⁶ is R¹¹ and R², R⁴, R⁵, R⁷ and R⁸ are each independently selected from alkyl, H, O-alkyl, halogeno, NO₂, NH₂ and NH-alkyl. Even more preferably, R⁶ is R¹¹ and R⁴, R⁵, R⁷ and R⁸ are all H and R² is selected from alkyl, O-alkyl, NH₂ and NH-alkyl.

Even more preferably still, for this embodiment, R¹¹ is selected from:

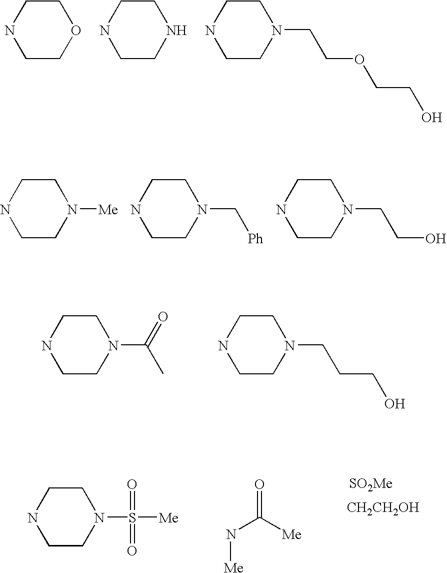

In another preferred embodiment, R⁷ is R¹¹ and R⁴, R⁵, R⁶, R⁸ are all H, and R² is selected from alkyl, O-alkyl, NH₂ and NH-alkyl. Preferably, for this embodiment, R¹¹ is selected from:

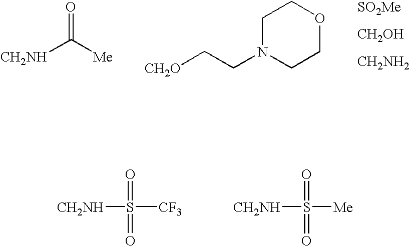

In another preferred embodiment of the invention, at least one of R² or R⁶ is R¹¹.

For this embodiment, R¹¹ is preferably selected from the following:

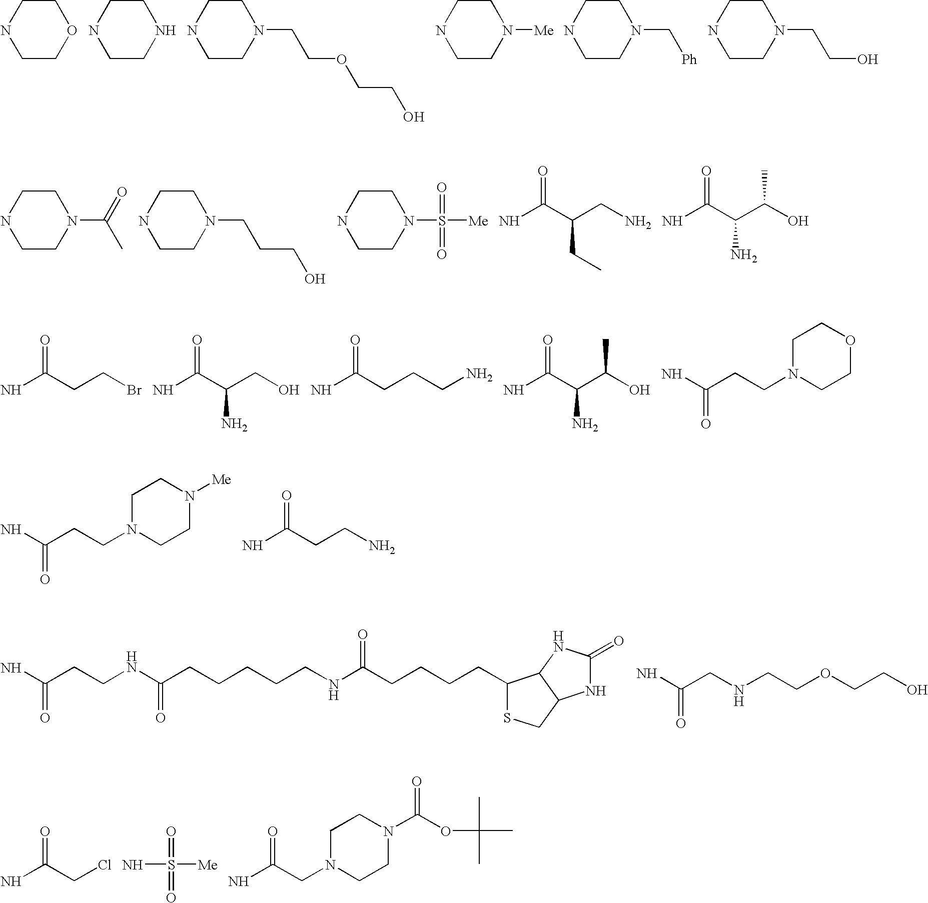

In one especially preferred embodiment, R⁶ is R¹¹.

For this embodiment, where R⁶ is R¹¹, preferably R², R⁴, R⁵, R⁷ and R⁸ are each independently selected from alkyl, H, CF₃, OH, O-alkyl, halogeno, NO₂, NH₂, NH-alkyl and N-(alkyl)₂.

Even more preferably, R², R⁴, R⁵, R⁷ and R⁸ are each independently selected from alkyl, H, O-alkyl, halogeno, NO₂, NH₂ and NH-alkyl.

More preferably still, R⁴, R⁵, R⁷ and R⁸ are all H and R² is selected from alkyl, O-alkyl, NH₂ and NH-alkyl.

More preferably still, R¹¹ is selected from:

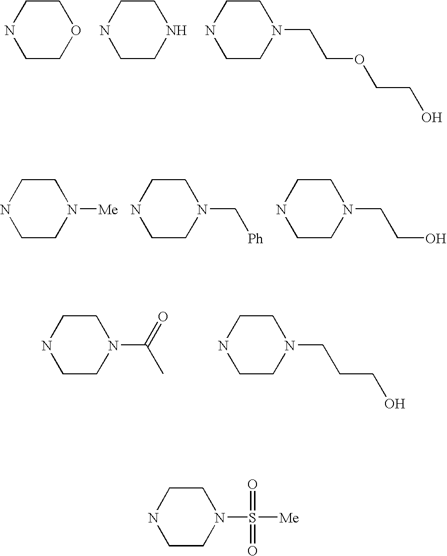

In an alternative preferred embodiment, R² is R¹¹.

For this embodiment, R² is R¹¹, preferably R⁴, R⁵, R⁶, R⁷ and R⁸ are each independently selected from alkyl, H, CF₃, OH, O-alkyl, halogeno, NO₂, NH₂, NH-alkyl and N-(alkyl)₂.

More preferably, R⁴, R⁵, R⁶, R⁷ and R⁸ are each independently selected from H, O-alkyl, halogeno, N-(alkyl)₂, NO₂.

More preferably still, one of R⁵ or R⁷ is selected from NO₂, alkoxy, halogeno and N-(alkyl)₂, and the remainder of R⁴, R⁵, R⁶, R⁷ and R⁸ are all H.

More preferably still, R¹¹ is selected from:

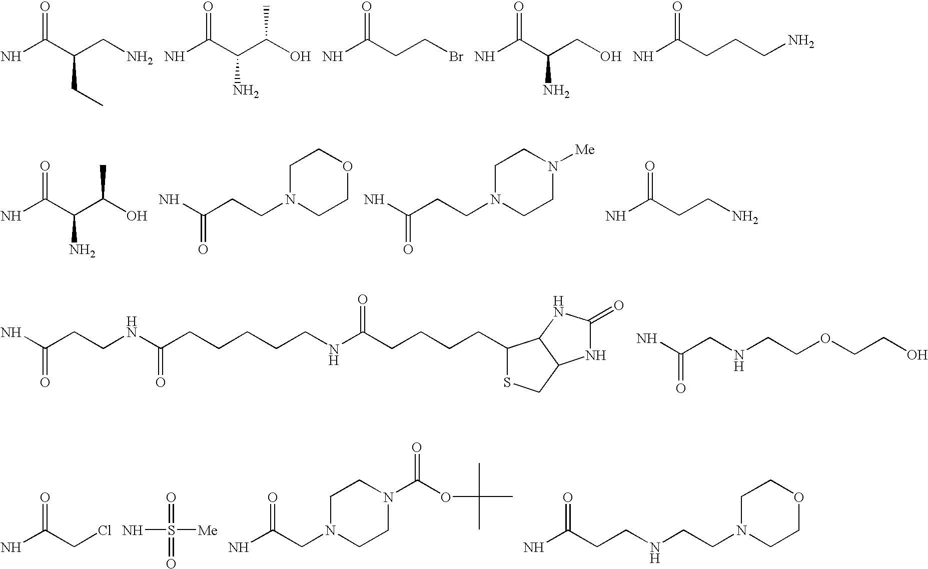

In one preferred embodiment, R³ is H.

In one preferred embodiment of the invention, R¹ is methyl, Z is NH and R³ is H.

In one preferred embodiment, Z is NH.

In another preferred embodiment, Z is NHCOCH₂. Preferably, for this embodiment, R² is N(alkyl)₂, NH-alkyl, alkyl, more preferably NMe₂, NHEt or Me. Preferably, for this embodiment, R³ is H and R¹ is alkyl, more preferably Me. Preferably, for this embodiment, R4-8 are each independently selected from H, NO₂, alkoxy and halogen, more preferably H, NO₂, chloro and OMe.

In one preferred embodiment, Z is —NH— and at least one of R⁴-R⁸ is selected from (CH₂)_{n″}NR¹⁷COR¹⁸ and SO₂NR¹⁹R²⁰.

In another preferred embodiment, Z is —NH— and at least one of R⁴-R⁸ is N-piperidinyl, N-pyrrolidinyl or N-thiomorpholinyl, each of which may be optionally substituted by one or more alkyl, alkoxy or CO-alkyl groups. Preferably, for this embodiment, R¹ is alkyl, more preferably Me, and R³ is H. Preferably, for this embodiment, R¹ is alkyl, NH₂, NH-alkyl, hydroxy-substituted alkyl or pyridinyl, more preferably, Me, NH₂, NHEt, CH₂OH or 3-pyridinyl. Preferably, for this embodiment, the remainder of R⁴-R⁸ are each independently selected from H, alkyl and alkoxy, more preferably, H, Me and OMe. More preferably still, R⁶ is N-piperidinyl, N-pyrrodinyl or N-thiomorpholinyl, R⁷ is H, Me or OMe, and R⁴, R⁵ and R⁸ are all H.

More preferably, at least one of R⁴-R⁸ is selected from

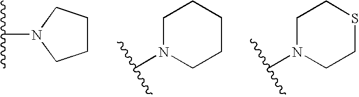

In one preferred embodiment, Z is —NH—, one of R⁶ and R⁷ is selected from:

(CH₂)_{n″}NR¹⁷COR¹⁸;

### SO₂NR¹⁹R²⁰; and

N-piperidinyl, N-pyrrolidinyl and N-thiomorpholinyl, each of which may be optionally substituted by one or more alkyl, alkoxy or CO-alkyl groups;
and the other of R⁶ and R⁷ is H, alkyl or alkoxy, preferably, H, Me or OMe.

In one preferred embodiment, Z is —NH— and two of R⁴-R⁸ are linked to form a cyclic ether containing one or more oxygens.

More preferably, R⁶ and R⁷ are linked to form a cyclic ether containing one or more oxygens. Preferably, for this embodiment, R⁴, R⁵ and R⁸ are H. Preferably, for this embodiment, R² is NH-alkyl, NH₂, pyridinyl or NH-aralkyl, more preferably NHEt, NH₂, 3-pyridinyl or NHCH₂CH₂Ph. Preferably, for this embodiment, R¹ is alkyl, more preferably Me.

Even more preferably, R⁶ and R⁷ are linked to form a cyclic ether as shown below

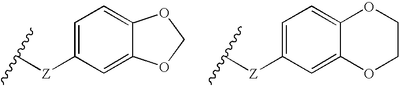

In one preferred embodiment, at least one of R⁶ and R⁷ is (CH₂)_{n″}NR¹⁷COR¹⁸ or SO₂NR¹⁹R²⁰.

In one especially preferred embodiment, at least one of R⁴-R⁸ is (CH₂)_{n″}NR¹⁷COR¹⁸. Preferably, n″ is 1, R¹⁷ is H and R¹⁸ is phenyl or pyridinyl.

In one especially preferred embodiment, at least one of R⁴-R⁸ is SO₂NR¹⁹R²⁰.

More preferably,
(i) one of R¹⁹ and R²⁰ is H and the other is an alkyl, aralkyl, aryl or heteroaryl group, each of which is optionally substituted by one or more alkoxy, alkyl, OH or CH₂CO₂-alkyl groups;
(ii) R¹⁹ and R²⁰ are each independently alkyl; or
(iii) R¹⁹ and R²⁰ together with the nitrogen to which they are attached are linked to form a morpholinyl group.

In another preferred embodiment, at least one of R⁴-R⁸ is selected from

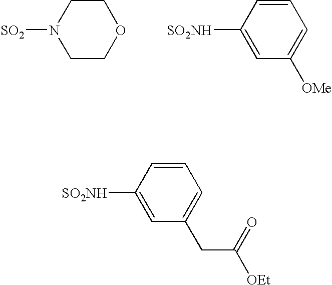

CH₂NHCOPh, CH₂NHCO-pyridinyl, SO₂NHCOMe, SO₂NHCH₂Ph, SO₂NHMe, SO₂NHC(Me)₂CH₂OH, SO₂NHⁱPr, SO₂NHEt, SO₂NEt₂, SO₂NHCH₂CH₂OH and SO₂NHCH₂CH₂OMe.

In one particularly preferred embodiment, R⁴, R⁵ and R⁸ are all H, one of R⁶ and R⁷ is selected from the following:

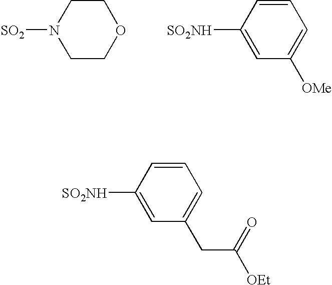

CH₂NHCOPh, CH₂NHCO-pyridinyl, SO₂NHCOMe, SO₂NHCH₂Ph, SO₂NHC(Me)₂CH₂OH, SO₂NHMe, SO₂NHⁱPr, SO₂NHEt, SO₂NEt₂, SO₂NHCH₂CH₂OH and SO₂NHCH₂CH₂OMe;
and the other of R⁶ and R⁷ is H, alkyl or alkoxy, preferably H, MeO, or Me.

In another particularly preferred embodiment, R⁴, R⁵, R⁷ and R⁸ are all H and R⁶ is SO₂NHCH₂CH₂OMe.

In one preferred embodiment, R² is selected from aryl, aryl-R⁹, NH₂, NH(alkyl), alkyl, N(alkyl)₂)₂, N(alkyl)CO-alkyl, N(alkyl)(aryl), NH(aryl), CH₂OH, wherein said alkyl and aryl groups are optionally substituted by one or more alkoxy, halo, CF₃ or R¹¹ groups.

More preferably, R² is selected from NH₂, NHMe, N(Me(Et), NHEt, NH^{t}Bu, Me, NHCH₂CH₂OMe, NMe₂, CH₂OH, NHPh,

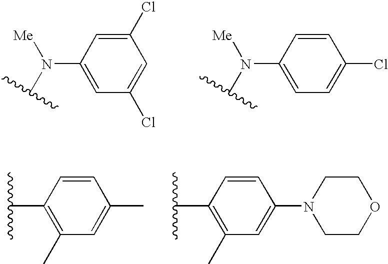

Another aspect of the invention relates to compounds of formula Ia, or pharmaceutically acceptable salts thereof,

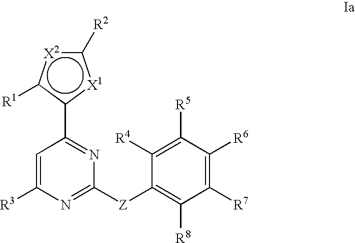

wherein:
one of X¹ and X² is S, and the other of X¹ and X² is N;
Z is NH, NHCO, CONH-alkyl, NHSO₂, NHCH₂, CH₂, CH₂CH₂, CH═CH, SO-alkyl, SO₂-alkyl, SO₂, SO, S or O;
R¹, R³, R⁴, R⁵, R⁶, R⁷ and R⁸ are each independently H, alkyl, alkyl-R⁹, aryl, aryl-R⁹, aralkyl, aralkyl-R⁹, halogeno, NO₂, CN, OH, O-alkyl, COR⁹, COOR⁹, O-aryl, O—R⁹, NH₂, NH-alkyl, NH-aryl, N-(alkyl)₂, N-(aryl)₂, N-(alkyl)(aryl), NH—R⁹, N—(R⁹)(R¹⁰), N-(alkyl)(R⁹), N-(aryl)(R⁹), COOH, CONH₂, CONH-alkyl, CONH-aryl, CON-(alkyl)(R⁹), CON(aryl)(R⁹), CONH—R⁹, CON—(R⁹)(R¹⁰), SO₃H, SO₂-alkyl, SO₂-alkyl-R⁹, SO₂-aryl, SO₂-aryl-R⁹, SO₂NH₂, SO₂NH—R⁹, SO₂N—(R⁹)(R¹⁰), CF₃, CO-alkyl, CO-alkyl-R⁹, CO-aryl, CO-aryl-R⁹ or R¹¹, wherein alkyl, aryl, aralkyl groups may be further substituted with one or more groups selected from halogeno, NO₂, OH, O-methyl, NH₂, COOH, CONH₂ and CF₃;
R⁹ and R¹⁰ are each independently solubilising groups selected from:
(i)
   a mono-, di- or polyhydroxylated alicyclic group;
   a di- or polyhydroxylated aliphatic or aromatic group;
   a carbohydrate derivative;
   an O- and/or S-containing heterocyclic group optionally substituted by one or more hydroxyl groups;
   an aliphatic or aromatic group containing a carboxamide, sulfoxide, sulfone, or sulfonamide function; or

a halogenated alkylcarbonyl group;
(ii) COOH, SO₃H, OSO₃H, PO₃H₂, or OPO₃H₂;

(iii) Y, where Y is selected from an alicyclic, aromatic, or heterocyclic group comprising one or more of the functions ═N—, —O—, —NH₂, —NH—, a quarternary amine salt, guanidine, and amidine, where Y is optionally substituted by one or more substituents selected from:
SO₂-alkyl;
alkyl optionally substituted by one or more OH groups;
CO-alkyl;
aralkyl;
COO-alkyl; and
   an ether group optionally substituted by one or more OH groups; and
where Y is other than pyridinyl;
(iv) a natural or unnatural amino acid, a peptide or a peptide derivative;
   each R¹¹ is a solubilising group as defined for R⁹ and R¹⁰ in (i) or (iv) above; or is selected from:
(v) OSO₃H, PO₃H₂, or OPO₃H₂;
(vi) Y as defined above, but excluding guanidine and quarternary amine salts;
(vii) NHCO(CH₂)ₘ[NHCO(CH₂)_{m′}]ₚ[NHCO(CH₂)_{m′}]_{q}Y or NHCO(CH₂)ₜNH(CH₂)_{t′}Y where p and q are each 0 or 1, and m, m′, m″, t and t′ are each independently an integer from 1 to 10; and
(viii) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are optionally substituted by one or more substituents selected from OH, NH₂, halogen and NO₂, R¹⁴ and R¹⁵ are each independently H or alkyl, and n and n′ are each independently 0, 1, 2, or 3;
(ix) an ether or polyether optionally substituted by one or more hydroxyl groups or one or more Y groups;
(x) (CH₂)ᵣNH₂; where r is 0, 1, 2, or 3;
(xi) (CH₂)ᵣ—OH; where r′ is 0, 1, 2, or 3;
(xii) (CH₂)_{n″}NR¹⁷COR¹⁸ where R¹⁷ is H or alkyl, n″ is 0, 1, 2 or 3 and R¹⁸ is an aryl group optionally substituted by one or more substituents selected from halogeno, NO₂, OH, alkoxy, NH₂, COOH, CONH₂ and CF₃;
(xiii) SO₂NR¹⁹R²⁰ where R¹⁹ and R²⁰ are each independently H, alkyl or aryl, with the proviso that at least one of R¹⁹ and R²⁰ is other than H, or R¹⁹ and R²⁰ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CONH₂ and CF₃;
   with the proviso that at least one of R⁴-R⁸ is selected from (CH₂)_{n″}NR¹⁷COR¹⁸ and SO₂NR¹⁹R²⁰.

In one preferred embodiment of the invention, at least one of R⁶ and R⁷ is (CH₂)_{n″}NR¹⁷COR¹⁸ or SO₂NR¹⁹R²⁰.

In another preferred embodiment of the invention, at least one of R⁴-R⁸ is (CH₂)_{n″}NR¹⁷COR¹⁸. More preferably, n″ is 1, R¹⁷ is H and R¹⁸ is phenyl.

In one preferred embodiment of the invention, at least one of R⁴-R⁸ is SO₂NR¹⁹R²⁰.

More preferably,
(i) one of R¹⁹ and R²⁰ is H and the other is an alkyl or aryl group each of which is optionally substituted by an alkoxy group;
(ii) R¹⁹ and R²⁰ are each independently alkyl; or
(iii) R¹⁹ and R²⁰ together with the nitrogen to which they are attached are linked to form a morpholine group.

In one particularly preferred embodiment, at least one of R⁴-R⁸ is selected from

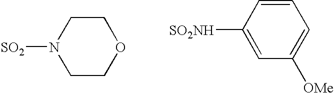

### CH₂NHCOPh, SO₂NHMe, SO₂NHEt and SO₂NHCH₂CH₂OMe.

In an even more preferred embodiment, R⁴, R⁵ and R⁸ are all H, R⁶ is H or Me and R⁷ is selected from the following:

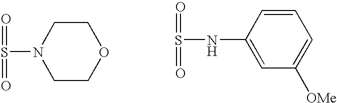

### CH₂,NHCOPh, SO₂NHMe and SO₂NHEt.

In another particularly preferred embodiment, R⁴, R⁵, R⁷ and R⁸ are all H and R⁶ is SO₂NHCH₂CH₂OMe.

In one especially preferred embodiment of the invention, the compound of formula I is selected from compounds [9], [21], [22], [26], [29], [30]-[33], [36]-[41], [43], [52]-[56], [62]-[78], [80]-[82], [84], [91]-[98], [102], [110], [177]-[181], [183], [193]-[195], [197]-[199], [201]-[209] and [216].

In another especially preferred embodiment of the invention, the compound of formula Ia is selected from the following:
N-{3-[4-(2,4-Dimethyl-thiazol-5-yl)-pyrimidin-2-ylamino]-benzyl}-benzamide[9];
N-Methyl-3-[4-(4-methyl-2-methylamino-thiazol-5-yl)-pyrimidin-2-ylamino]-benzenesulfonamide[21];
3-[4-(2-Ethylamino-4-methyl-thiazol-5-yl)-pyrimidin-2-ylamino]-N-methyl-benzenesulfonamide[22];
3-[4-(2-Amino-4-methyl-thiazol-5-yl)-pyrimidin-2-ylamino]-N-methyl-benzenesulfonamide[26];
N-Ethyl-3-[4-(2-ethylamino-4-methyl-thiazol-5-yl)-pyrimidin-2-ylamino]-benzenesulfonamide[29];
3-[4-(2-Amino-4-methyl-thiazol-5-yl)-pyrimidin-2-ylamino]-N-ethyl-benzenesulfonamide[30];
N-Ethyl-3-[4-(4-methyl-2-methylamino-thiazol-5-yl)-pyrimidin-2-ylamino]benzenesulfonamide[31];
N-(3-Methoxy-phenyl)-3-[4-(4-methyl-2-methylamino-thiazol-5-yl)-pyrimidin-2-ylamino]benzenesulfonamide[32];
3-[4-(2,4-Dimethyl-thiazol-5-yl)-pyrimidin-2-ylamino]-N-methyl-benzenesulfonamide[33];
[4-(2-Ethylamino-4-methyl-thiazol-5-yl)-pyrimidin-2-yl]-[4-methyl-3-(morpholine-4-sulfonyl)-phenyl]-amine[36];
[4-(4-Methyl-2-methylamino-thiazol-5-yl)-pyrimidin-2-yl]-[4-methyl-3-(morpholine-4-sulfonyl)-phenyl]-amine[37];
[4-(2-Amino-4-methyl-thiazol-5-yl)-pyrimidin-2-yl]-[4-methyl-3-(morpholine-4-sulfonyl)-phenyl]-amine[38];
4-[4-(2-Ethylamino-4-methyl-thiazol-5-yl)-pyrimidin-2-ylamino]-N-(2-methoxy-ethyl)-benzenesulfonamide[39];
N-(2-Methoxy-ethyl)-4-[4-(4-methyl-2-methylamino-thiazol-5-yl)-pyrimidin-2-ylamino]-benzenesulfonamide[40];
4-[4-(2-Amino-4-methyl-thiazol-5-yl)-pyrimidin-2-ylamino]-N-(2-methoxy-ethyl)-benzenesulfonamide[41];
4-[4-(2,4-Dimethyl-thiazol-5-yl)-pyrimidin-2-ylamino]-N-(2-methoxy-ethyl)-benzenesulfonamide[43];
N,N-Diethyl-4-[4-(4-methyl-2-methylamino-thiazol-5-yl)-pyrimidin-2-ylamino]-benzenesulfonamide[52];

### Compounds of Formula II

As mentioned above, another aspect of the invention relates to compounds of formula II as defined above, or pharmaceutically acceptable salts thereof.

Preferred definitions of R¹, R³⁻⁶, R⁷⁻⁸, Z, X¹, X² are as set forth above in respect of compounds of formula I.

Preferably, R² is selected from pyridinyl, N(methyl)pyridinyl, NH(aralkyl) and N(methyl)(aralkyl), wherein said pyridinyl or aralkyl groups may be optionally substituted by one or more alkyl, CF₃ or ether groups.

More preferably, R² is selected from N(Me)CH₂Ph, NHCH₂CH₂Ph, NHCH₂Ph,

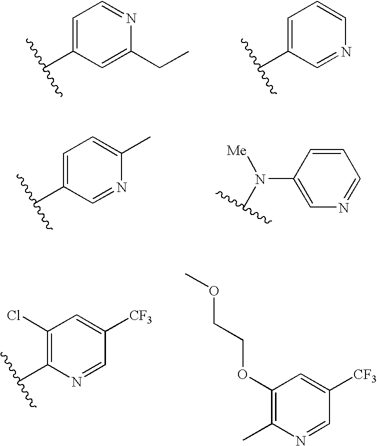

In one preferred embodiment, R⁶ is an alicyclic group selected from

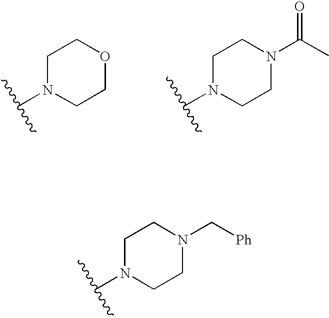

Preferably, for this embodiment, R⁴, R⁵, R⁷ and R⁸ are each independently selected from H, alkyl, alkoxy and halo. More preferably, R⁴, R⁵, R⁷ and R⁸ are all H.

In another preferred embodiment, R⁶ or R⁷ is CH₂NHCOMe. Preferably, for this embodiment, the remainder of R⁴, R⁵, R⁶, R⁷ and R⁸ are each independently selected from H, alkyl, alkoxy and halo. More preferably, the remainder of R⁴, R⁵, R⁶, R⁷ and R⁸ are all H.

In one particularly preferred embodiment, the compound of formula II is selected from compounds [99], [100], [101], [103], [104]-[109], [117]-[119], [122], [126], [127], [153], [156], [158] and [162]-[165].

### Biological Activity

In one preferred embodiment the compound of the invention is capable of exhibiting an antiproliferative effect in human cell lines, as measured by a standard 72 h MTT cytotoxicity assay. Preferably, the compound of the invention exhibits an IC₅₀ value of less than 10 μM, more preferably less than 5 μM, even more preferably less than 1 μM as measured by said MTT assay. More preferably still, the compound exhibits an IC₅₀ value of less than 0.5 less μM, more preferably still less than 0.2 μM.

In another preferred embodiment, the compound of the invention is capable of inhibiting one or more protein kinases, as measured by the assays described in the accompanying Examples section. Preferably, the compound of the invention exhibits an IC₅₀ value of less than 10 μM, more preferably less than 5 μM, even more preferably less than 1 μM or less than 0.5 less μM, more preferably still less than 0.1 μM.

More preferably still, the compound exhibits an IC₅₀ value of less than 0.01 μM. For example, preferably the compound is selected from compound numbers [5]-[7], [13], [18]-[28], [30], 31], [34], [35], [38]-[40] and [44]-[49] of Table 1.

Even more preferably still, the compound exhibits an IC₅₀ value of less than 0.005 μM. For example, preferably the compound is selected from compound numbers [5], [6], [19]-[22], [24], [26]-[28], [31], [34], [35], [39], [40] and [48] of Table 1.

More preferably still, the compound exhibits an IC₅₀ value of less than 0.002 μM. For example, preferably the compound is selected from compound numbers [19], [20], [27], [28], [35] and [40] of Table 1. More preferably still, the compound is compound [27].

In one preferred embodiment, the compound exhibits a pIC₅₀ value, where pIC₅₀=−log(IC₅₀, M), of at least 4, more preferably at least 5, more preferably still at least 6, even more preferably at least 7, and more preferably at least 8.

In one preferred embodiment, the compound of the invention is selected from compound numbers [59] and [138].

In another preferred embodiment, the compound of the invention is selected from compound numbers [19], [27], [34], [37], [38], [55] and [59].

In one preferred embodiment, the compound of the invention exhibits a selectivity for inhibiting one or more particular kinases over one or more other kinases. For example, in one particularly preferred embodiment, the compound of the invention exhibits a selectivity for inhibiting one or more protein kinases selected from a CDK, GSK, aurora and VEGFR2 over other one or more other kinases. More preferably, the compound of the invention exhibits a selectivity for a CDK, GSK, aurora kinase or VEGFR2 over one or more other kinases of at least 2-fold, more preferably at least 5-fold, more preferably still at least 10-fold, even more preferably at least 25-fold or 50-fold.

### Therapeutic Use

The compounds of the invention have been found to possess anti-proliferative activity and are therefore believed to be of use in the treatment of proliferative disorders such as cancers, leukaemias and other disorders associated with uncontrolled cellular proliferation such as psoriasis and restenosis.

Thus, one aspect of the invention relates to the use of a compound of the invention, or a pharmaceutically acceptable salt thereof, in the preparation of a medicament for treating a proliferative disorder.

As used herein the phrase “preparation of a medicament” includes the use of one or more of the above described compounds directly as the medicament in addition to its use in a screening programme for further anti-viral and/or antiproliferative agents or in any stage of the manufacture of such a medicament.

As defined herein, an anti-proliferative effect within the scope of the present invention may be demonstrated by the ability to inhibit cell proliferation in an in vitro whole cell assay, for example using any of the cell lines AGS, H1299 or SJSA-1, or by showing inhibition of the interaction between HDM2 and p53 in an appropriate assay. These assays, including methods for their performance, are described in more detail in the accompanying Examples. Using such assays it may be determined whether a compound is anti-proliferative in the context of the present invention.

One preferred embodiment therefore relates to the use of one or more compounds of the invention in the treatment of proliferative disorders. Preferably, the proliferative disorder is a cancer or leukaemia. The term proliferative disorder is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment, the compound of the invention is administered in an amount sufficient to inhibit at least one CDK enzyme. Assays for determining CDK activity are described in more detail in the accompanying examples.

A further aspect of the invention relates to a method of treating a CDK-dependent disorder, said method comprising administering to a subject in need thereof, a compound of the invention or a pharmaceutically acceptable salt thereof, as defined above in an amount sufficient to inhibit a CDK.

Another aspect relates to the use of a compound of the invention as an anti-mitotic agent.

Yet another aspect relates to the use of a compound of the invention for treating a neurodegenerative disorder.

Preferably, the neurodegenerative disorder is neuronal apoptosis.

Another aspect of the invention relates to the use of a compound of the invention as an antiviral agent.

Thus, another aspect of the invention relates to the use of a compound of the invention in the preparation of a medicament for treating a viral disorder, such as human cytomegalovirus (HCMV), herpes simplex virus type 1 (HSV-1), human immunodeficiency virus type 1 (HIV-1), and varicella zoster virus (VZV).

In a more preferred embodiment of the invention, the compound of the invention is administered in an amount sufficient to inhibit one or more of the host cell CDKs involved in viral replication, i.e. CDK2, CDK7, CDK8, and CDK9 [Wang D, De la Fuente C, Deng L, Wang L, Zilberman I, Eadie C, Healey M, Stein D, Denny T, Harrison L E, Meijer L, Kashanchi F. Inhibition of human immunodeficiency virus type 1 transcription by chemical cyclin-dependent kinase inhibitors. J. Virol. 2001; 75: 7266-7279].

As defined herein, an anti-viral effect within the scope of the present invention may be demonstrated by the ability to inhibit CDK2, CDK7, CDK8 or CDK9.

In a particularly preferred embodiment, the invention relates to the use of one or more compounds of the invention in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

Another aspect relates to the use of compounds of the invention, or pharmaceutically acceptable salts thereof, in the preparation of a medicament for treating diabetes.

In a particularly preferred embodiment, the diabetes is type II diabetes.

GSK3 is one of several protein kinases that phosphorylate glycogen synthase (GS). The stimulation of glycogen synthesis by insulin in skeletal muscle results from the dephosphorylation and activation of GS. GSK3's action on GS thus results in the latter's deactivation and thus suppression of the conversion of glucose into glycogen in muscles.

Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes [Chen, Y. H.; Hansen, L.; Chen, M. X.; Bjorbaek, C; Vestergaard, H.; Hansen, T.; Cohen, P. T.; Pedersen, O. Diabetes, 1994, 43, 1234]. Furthermore, it has been demonstrated that GSK3 is over-expressed in muscle cells of type Et diabetics and that an inverse correlation exists between skeletal muscle GSK3 activity and insulin action [Nikoulina, S. E.; Ciaraldi, T. P.; Mudaliar, S.; Mohideen, P.; Carter, L.; Henry, R. R. Diabetes, 2000, 49, 263].

GSK3 inhibition is therefore of therapeutic significance in the treatment of diabetes, particularly type H, and diabetic neuropathy.

It is notable that GSK3 is known to phosphorylate many substrates other than GS, and is thus involved in the regulation of multiple biochemical pathways. For example, GSK is highly expressed in the central and peripheral nervous systems.

Another aspect therefore relates to the use of compounds of the invention, or pharmaceutically acceptable salts thereof, in the preparation of a medicament for treating a CNS disorders, for example neurodegenerative disorders. Preferably, the CNS disorder is Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages.

Without wishing to be bound by theory, it is believed that GSK3 inhibitors may be able to prevent and/or reverse the abnormal hyperphosphorylation of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process [Goedert, M. Curr. Opin. Gen. Dev., 2001, 11, 343].

Another aspect relates to the use of compounds of the invention, or pharmaceutically acceptable salts thereof, in the preparation of a medicament for treating bipolar disorder.

Yet another aspect relates to the use of compounds of the invention, or pharmaceutically acceptable salts thereof, in the preparation of a medicament for treating a stroke.

Reducing neuronal apoptosis is an important therapeutic goal in the context of head trauma, stroke, epilepsy, and motor neuron disease [Mattson, M. P. Nat. Rev. Mol. Cell. Biol., 2000, 1, 120]. Therefore, GSK3 as a pro-apoptotic factor in neuronal cells makes this protein kinase an attractive therapeutic target for the design of inhibitory drugs to treat these diseases.

Yet another aspect relates to the use of compounds of the invention, or pharmaceutically acceptable salts thereof, in the preparation of a medicament for treating alopecia.

Hair growth is controlled by the Wnt signalling pathway, in particular Wnt-3. In tissue-culture model systems of the skin, the expression of non-degradable mutants of β-catenin leads to a dramatic increase in the population of putative stem cells, which have greater proliferative potential [Zhu, A. J.; Watt, F. M. Development, 1999, 126, 2285]. This population of stem cells expresses a higher level of non-cadherin-associated β-catenin [DasGupta, R.; Fuchs, E. Development, 1999, 126, 4557], which may contribute to their high proliferative potential. Moreover, transgenic mice overexpressing a truncated β-catenin in the skin undergo de novo hair-follicle morphogenesis, which normally is only established during embryogenesis. The ectopic application of GSK3 inhibitors may therefore be therapeutically useful in the treatment of baldness and in restoring hair growth following chemotherapy-induced alopecia.

A further aspect of the invention relates to a method of treating a GSK3-dependent disorder, said method comprising administering to a subject in need thereof, a compound of the invention or a pharmaceutically acceptable salt thereof, as defined above in an amount sufficient to inhibit GSK3.

Preferably, the compound of the invention, or pharmaceutically acceptable salt thereof, is administered in an amount sufficient to inhibit GSK3β.

In one embodiment of the invention, the compound of the invention is administered in an amount sufficient to inhibit at least one PLK enzyme.

A further aspect of the invention relates to a method of treating a PLK-dependent disorder, said method comprising administering to a subject in need thereof, a compound of the invention or a pharmaceutically acceptable salt thereof, as defined above in an amount sufficient to inhibit PLK.

The polo-like kinases (PLKs) constitute a family of serine/threonine protein kinases. Mitotic Drosophila melanogaster mutants at the polo locus display spindle abnormalities [Sunkel et al., J. Cell Sci., 1988, 89, 25] and polo was found to encode a mitotic kinase [Llamazares et al., Genes Dev., 1991, 5, 2153]. In humans, there exist three closely related PLKs [Glover et al., Genes Dev., 1998, 12, 3777]. They contain a highly homologous amino-terminal catalytic kinase domain and their carboxyl termini contain two or three conserved regions, the polo boxes. The function of the polo boxes remains incompletely understood but they are implicated in the targeting of PLKs to subcellular compartments [Lee et al., Proc. Natl. Acad. Sci. USA, 1998, 95, 9301; Leung et al., Nat. Struct. Biol, 2002, 9, 719], mediation of interactions with other proteins [Kauselmann et al., EMBO J., 1999, 18, 5528], or may constitute part of an autoregulatory domain [Nigg, Curr. Opin. Cell Biol, 1998, 10, 776]. Furthermore, the polo box-dependent PLK1 activity is required for proper metaphase/anaphase transition and cytokinesis [Yuan et al., Cancer Res., 2002, 62, 4186; Seong et al., J. Biol. Chem., 2002, 277, 32282].

Studies have shown that human PLKs regulate some fundamental aspects of mitosis [Lane et al., J. Cell. Biol, 1996, 135, 1701; Cogswell et al., Cell Growth Differ., 2000, 11, 615]. In particular, PLK1 activity is believed to be necessary for the functional maturation of centrosomes in late G2/early prophase and subsequent establishment of a bipolar spindle. Depletion of cellular PLK1 through the small interfering RNA (siRNA) technique has also confirmed that this protein is required for multiple mitotic processes and completion of cytokinesis [Liu et al., Proc. Natl. Acad. Sci. USA, 2002, 99, 8672].

In a more preferred embodiment of the invention, the compound of the invention is administered in an amount sufficient to inhibit PLK1.

Of the three human PLKs, PLK1 is the best characterized; it regulates a number of cell division cycle effects, including the onset of mitosis [Toyoshima-Morimoto et al., Nature, 2001, 410, 215; Roshak et al., Cell. Signalling, 2000, 12, 405], DNA-damage checkpoint activation [Smits et al., Nat. Cell Biol, 2000, 2, 672; van Vugt et al., J. Biol. Chem., 2001, 276, 41656], regulation of the anaphase promoting complex [Sumara et al., Mol. Cell, 2002, 9, 515; Golan et al., J. Biol. Chem., 2002, 277, 15552; Kotani et al., Mol. Cell, 1998, 1, 371], phosphorylation of the proteasome [Feng et al., Cell Growth Differ., 2001, 12, 29], and centrosome duplication and maturation [Dai et al., Oncogene, 2002, 21, 6195].

Specifically, initiation of mitosis requires activation of M-phase promoting factor (MPF), the complex between the cyclin dependent kinase CDK1 and B-type cyclins [Nurse, Nature, 1990, 344, 503]. The latter accumulate during the S and G2 phases of the cell cycle and promote the inhibitory phosphorylation of the MPF complex by WEE1, MK1, and MYT1 kinases. At the end of the G2 phase, corresponding dephosphorylation by the dual-specificity phosphatase CDC25C triggers the activation of MPF [Nigg, Nat. Rev. Mol. Cell. Biol., 2001, 2, 21]. In interphase, cyclin B localizes to the cytoplasm [Hagting et al., EMBO J., 1998, 17, 4127], it then becomes phosphorylated during prophase and this event causes nuclear translocation [Hagting et al., Curr. Biol., 1999, 9, 680; Yang et al., J. Biol. Chem., 2001, 276, 3604]. The nuclear accumulation of active MPF during prophase is thought to be important for initiating M-phase events [Takizawa et al., Curr. Opin. Cell Biol., 2000, 12, 658]. However, nuclear MPF is kept inactive by WEE1 unless counteracted by CDC25C. The phosphatase CDC25C itself, localized to the cytoplasm during interphase, accumulates in the nucleus in prophase [Seki et al., Mol. Biol. Cell, 1992, 3, 1373; Heald et al., Cell, 1993, 74, 463; Dalai et al., Mol. Cell. Biol., 1999, 19, 4465]. The nuclear entry of both cyclin B [Toyoshima-Morimoto et al., Nature, 2001, 410, 215] and CDC25C [Toyoshima-Morimoto et al., EMBO Rep., 2002, 3, 341] are promoted through phosphorylation by PLK1 [Roshak et al., Cell. Signalling, 2000, 12, 405]. This kinase is an important regulator of M-phase initiation.

In one particularly preferred embodiment, the compounds of the invention are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of the invention is administered in an amount sufficient to inhibit PLK2 and/or PLK3.

Mammalian PLK2 (also known as SNK) and PLK3 (also known as PRK and FNK) were originally shown to be immediate early gene products. PLK3 kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [Wang et al., Mol. Cell. Biol, 2002, 22, 3450]. PLK2 is the least well understood homologue of the three PLKs. Both PLK2 and PLK3 may have additional important post-mitotic functions [Kauselmann et al., EMBO J., 1999, 18, 5528].

In another preferred embodiment, the compound of the invention is administered in an amount sufficient to inhibit at least one aurora kinase.

A further aspect of the invention relates to a method of treating an aurora kinase-dependent disorder, said method comprising administering to a subject in need thereof, a compound of the invention or a pharmaceutically acceptable salt thereof, as defined above in an amount sufficient to inhibit an aurora kinase.

In another preferred embodiment, the compound of the invention is administered in an amount sufficient to inhibit at least one tyrosine kinase.

Preferably, the tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

A further aspect of the invention relates to a method of treating a tyrosine kinase-dependent disorder, said method comprising administering to a subject in need thereof, a compound of the invention or a pharmaceutically acceptable salt thereof, as defined above in an amount sufficient to inhibit a tyrosine kinase.

Another aspect relates to the use of a compound of the invention for inhibiting a protein kinase.

A further aspect of the invention relates to a method of inhibiting a protein kinase, said method comprising contacting said protein kinase with a compound of the invention.

Preferably, the protein kinase is selected from a CDK, GSK, an aurora kinase, PLK and a tyrosine kinase.

In a preferred embodiment of this aspect, the protein kinase is a cyclin dependent kinase. Preferably, the protein kinase is CDK1, CDK2, CDK3, CDK4, CDK6, CDK7, CDK8 or CDK9, more preferably CDK2.

### Pharmaceutical Compositions

A further aspect of the invention relates to a pharmaceutical composition comprising a compound of the invention admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminum hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of the compounds of the invention. The person skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes the use of solvate forms of the compounds of the present invention. The terms used in the claims encompass these forms.

### Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilizable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### Combinations

In a particularly preferred embodiment, the one or more compounds of the invention are administered in combination with one or more other anticancer agents, for example, existing anticancer drugs available on the market. In such cases, the compounds of the invention may be administered consecutively, simultaneously or sequentially with the one or more other anticancer agents.

Anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

### Natural/Unnatural Amino Acids

In one preferred embodiment of the invention, R⁹, R¹⁰ or R¹¹ may be a natural or unnatural amino acid.

As used herein, the term “unnatural amino acid” refers to a derivative of an amino acid and may for example include alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, β-alanine, L-α-amino butyric acid, L-γ-amino butyric acid, L-α-amino isobutyric acid, L-ε-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminoproprionic acid and L-Phe (4-benzyl).

### Devices

In one preferred embodiment of the invention, the R⁹, R¹⁰ or R¹¹ groups allow for the immobilisation of the 2-phenylamino-4-heteroaryl-pyrimidine compounds onto a substrate. By way of example, the R⁹, R¹⁰ or R¹¹ groups may contain chemical functions that can be used for covalent attachment to solid phases such as functionalised polymers (e.g. agarose, polyacrylamide, polystyrene etc.) as commonly found in matrices (microtitre plate wells, microbeads, membranes, etc.), or used for biochemical assays or affinity chromatography. Alternatively, the R⁹, R¹⁰ or R¹¹ groups may linked to other small molecules (e.g. biotin) or polypeptides (e.g. antigens), which can be used for non-covalent immobilisation through binding to an immobilised receptor (e.g. avidin or streptavidin in the case of biotin, or a specific antibodies in the case of antigens).

### Assays

Another aspect of the invention relates to the use of a compound of the invention as defined hereinabove in an assay for identifying further candidate compounds that influence the activity of one or more of the following: a CDK, an aurora kinase, GSK-3, PLK and/or a tyrosine kinase.

Preferably, the assay is capable of identifying candidate compounds that are capable of inhibiting one or more of a CDK enzyme, an aurora kinase, a tyrosine kinase, GSK or a PLK enzyme.

More preferably, the assay is a competitive binding assay.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drag screening assays in which neutralizing antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a CDK, an aurora kinase, GSK-3, PLK and/or a tyrosine kinase in the presence of a known substrate of said CDK enzyme and detecting any change in the interaction between said CDK enzyme and said known substrate.

A further aspect of the invention provides a method of detecting the binding of a tigand to a CDK, an aurora kinase, GSK-3, PLK or a tyrosine kinase enzyme, said method comprising the steps of:
(i) contacting a ligand with a CDK, an aurora kinase, GSK-3, PLK or a tyrosine kinase enzyme in the presence of a known substrate of said enzyme;
(ii) detecting any change in the interaction between said enzyme and said known substrate;
   and wherein said ligand is a compound of the invention.

One aspect of the invention relates to a process comprising the steps of:

(a) performing an assay method described hereinabove;
(b) identifying one or more ligands capable of binding to a ligand binding domain; and
(c) preparing a quantity of said one or more ligands.

Another aspect of the invention provides a process comprising the steps of:

(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:

(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders.

The above methods may be used to screen for a ligand useful as an inhibitor of one or more CDK enzymes.

The present invention is further described by way of example.

### EXAMPLES

### Example 1

General. Compounds were prepared according to the general methods we have outlined previously: Wang et al. J. Med. Chem. 2004, 47, 1662-1675. NMR spectra were obtained using a Varian INOVA-500 instrument. Chemical shifts are reported in parts per million relative to internal tetramethylsilane standard. Mass spectra were obtained using a Waters ZQ2000 single quadrupole mass spectrometer with electrospray ionization (ESI). Analytical and preparative RP-HPLC was performed using Vydac 218TP54 (250×4.6 mm) and 218TP1022 (250×22 mm) columns, respectively. Linear gradient elution using H₂O/MeCN systems (containing 0.1% CF₃COOH) at flow rates of 1 mL/min (analytical) and 9 mL/min (preparative) was performed. Purity was assessed by integration of chromatograms (λ=254 nm). Silica gel (E M Kieselgel 60, 0.040-0.063 mm, Merck) or ISOLUTE pre-packed columns (Jones Chromatography Ltd. UK) were used for flash chromatography.

Chemical synthesis. The covalent attachment of solubilising moieties can be achieved in a number of different ways known in the art (Wermuth C G. Preparation of water-soluble compounds by covalent attachment of solubilizing moieties. In: Practice of Medicinal Chemistry, Academic Press: London, UK, 1996; pp 755-776). For example, amino substituents in 2-phenylamino-4-heteroaryl-pyrimidine derivatives, or their synthetic precursors, can be acylated or alkylated with carbonyl functions in appropriate solubilising moiety precursors. Similarly, carbonyl groups in the 2-phenylamino-4-heteroaryl-pyrimidine derivatives can be aminated or alkylated with appropriate solubilising moiety precursors. Halogen groups on aromatic C in phenylamino-4-heteroaryl-pyrimidines or precursors can be substituted through nucleophilic groups in solubilising moiety precursors. Suitable 2-phenylamino-4-heteroaryl-pyrimidine precursors may be prepared in accordance with the teachings of Fischer et al (WO 01/072745 and WO 03/029248; Cyclacel Limited). The compounds of the invention may be prepared in accordance with the methods disclosed in WO 01/072745 and WO 03/029248.

### Example 1

{3-[4-(2-Amino-4-methyl-thiazol-5-yl)-pyrimidin-2-ylamino]-phenyl}-acetic acid 2-methoxy-ethyl ester (1). Yellow solid. Mp 182-184° C. Anal. RP-HPLC: t_{R}=13.8 min (10-70% MeCN; purity 97%). ¹H-NMR (CD₃OD) δ: 2.59 (s, 3H, CH₃), 3.35 (s, 3H, CH₃), 3.60 (m, 2H, CH₂), 3.71 (s, 2H, CH₂), 4.24 (q, 2H, J=4.5 Hz, CH₂), 7.02 (d, 1H, J=7.5 Hz, Ph-H), 7.06 (d, 1H, J=5.5 Hz, pyrimidinyl-H), 7.30 (t, 1H, J=8.0 Hz, Ph-H), 7.55 (d, 1H, J=7.5 Hz, Ph-H), 7.61 (s, 1H, Ph-H), 8.39 (d, 1H, J=5.5 Hz, pyrimidinyl-H). MS (ESI⁺) m/z 400.44 [M+H]⁺ (C₁₉H₂₁N₅O₃S requires 399.47).

[4-(2-tert-Butylamino-4-methyl-thiazol-5-yl)-pyrimidin-2-yl]-(4-methyl-3-nitro-phenyl)-amine (2). By condensation of 1-(2-tert-butylamino-4-methyl-thiazol-5-yl)-3-dimethylamino-propenone and N-(4-methyl-3-nitro-phenyl)-guanidine nitrate. Yellow solid. Anal. RP-HPLC: t_{R}=18.5 min (10-70% MeCN; purity 97%). ¹H-NMR (DMSO-d₆) δ: 1.39 (s, 9H, CH₃), 2.44 (s, 3H, CH₃), 2.50 (s, 3H, CH₃), 6.95 (d, 1H, J=5.5 Hz, pyrimidinyl-H), 7.38 (d, 1H, J=8.0 Hz, Ph-H), 7.89 (d, 1H, J=8.0 Hz, Ph-H), 7.92 (br. s, 1H, NH), 8.36 (d, 1H, J=5.5 Hz, pyrimidinyl-H), 8.56 (d, 1H, J=2.5 Hz, Ph-H), 9.81 (sbr, 1H, NH). MS (ESI⁺) m/z 399.37 [M+H]⁺ (C₁₉H₂₂N₆O₂S requires 398.48).

1-(4-{3-[4-(4-Methyl-2-methylamino-thiazol-5-yl)-pyrimidin-2-ylamino]-phenyl}-piperazin-1-yl)-ethanone (3). The precursor 1-[4-(3-nitro-phenyl)-piperazin-1-yl]-ethanone was prepared as described (Orus et al. Pharmazie 57, 515 2004) as an orange solid. 1H-NMR (DMSO-d₆) δ: 2.15 (s, 3H, CH₃), 3.26 (dd, 2H, J=5.5 Hz, CH₂), 3.30 (dd, 2H, J=5.5 Hz, CH₂), 3.66 (dd, 2H, J=5.5 Hz, CH₂), 3.80 (dd, 2H, J=5.5 Hz, CH₂), 7.20 (d, 1H, J=5.0 Hz, Ph-H), 7.40 (d, 1H, J=5.0 Hz, Ph-H), 7.70 (d, 1H, J=5.0 Hz, Ph-H) and 7.72 (s, 1H, Ph-H). Treatment of a mixture of this compound in AcOH/EtOH (1:2, v/v) with Fe (3 eq) and heating at 80° C. for 3 h afforded the corresponding aniline as a yellow oil in 90% yield. ¹H-NMR (DMSO-d₆) δ: 2.07 (s, 3H, CH₃), 3.05-3.12 (m, 4H, CH₂), 3.49-3.57 (m, 2H, CH₂), 3.70-3.73 (m, 2H, CH₂), 6.22 (d, 1H, J=8.0 Hz, Ph-H), 6.25 (s, 1H, Ph-H), 6.32 (d, 1H, J=8.0 Hz, Ph-H) and 7.00 (dd, 1H, J=8.0 Hz, Ph-H). The title compound was obtained by treatment of the corresponding N-[3-(4-acetyl-piperazin-1-yl)-phenyl]-guanidine with 3-dimethylamino-1-(4-methyl-2-methylamino-thiazol-5-yl)-propenone Yellow solid. Anal. RP-HPLC: t_{R}=10.1 min (10-70% MeCN, purity 99%). ¹H-NMR (DMSO-d₆) δ: 2.03 (s, 3H, CH₃), 2.46 (s, 3H, CH₃), 2.85 (s, 3H, CH₃), 3.09 (m, 2H, CH₂), 3.16 (m, 2H, CH₂), 3.58 (m, 4H, CH₂), 6.55 (d, 1H, J=8.0 Hz, Ph-H), 6.89 (d, 1H, J=6.0 Hz, pyrimidinyl-H), 7.12 (t, 1H, J=8.5 Hz, Ph-H), 7.22 (d, 1H, J=8.0 Hz, Ph-H), 7.46 (s, 1H, Ph-H), 8.03 (m, 1H, NH), 8.32 (d, 1H, J=5.5 Hz, pyrimidinyl-H), 9.26 (s, 1H, NH). MS (ESI⁺) m/z 446.49 [M+Na] (C₂₁H₂₅N₇OS requires 423.54).

[4-(2,4-Dimethyl-thiazol-5-yl)-pyrimidin-2-yl]-(3-methanesulfonyl-phenyl)-amine (4). This compound was obtained by treatment of 3-dimethylamino-1-(2,4-dimethyl-thiazol-5-yl)-propenone with N-(3-methanesulfonyl-phenyl)-guanidine as a yellow solid. Anal. RP-HPLC: t_{R}=12.7 min (10-70% MeCN, purity 97%). ¹H-NMR (DMSO-d₆) δ: 2.50 (s, 6H, CH₃), 3.12 (s, 3H, CH₃), 7.18 (d, 1H, J=5.0 Hz, pyrimidinyl-H), 7.50 (m, 1H, Ph-H), 7.58 (m, 1H, Ph-H), 8.01 (m, 1H, Ph-H), 8.48 (s, 1H, Ph-H), 8.58 (d, 1H, J=5.0 Hz, pyrimidinyl-H), 10.09 (s, 1H, NH). MS (ESI⁺) m/z 361.29 [M+H]⁺ (C₁₆H₁₆N₄O₂S₂ requires 360.46).

N-{3-[4-(2-Ethylamino-4-methyl-thiazol-5-yl)-pyrimidin-2-ylamino]-benzyl}-methanesulfonamide (5). By condensation between 3-dimethylamino-1-(2-aminoethyl-4-methyl-thiazol-5-yl)-propenone and N-(3-methanesulfonamide-benzyl)-guanidine nitrate. Yellow solid. Anal. RP-HPLC: t_{R}=13.6 min (0-60% MeCN, purity >98%). ¹H-NMR (DMSO-d₆) δ: 1.28 (t, 3H, J=7.5 Hz, CH₃), 2.52 (s, 3H, CH₃), 2.88 (s, 3H, CH₃), 3.36 (m, 2H, CH₂), 4.28 (s, 2H, CH₂), 6.94 (d, 1H, J=5.5 Hz, pyrimidinyl-H), 7.02 (d, 1H, J=7.5 Hz, Ph-H), 7.29 (t, 1H, J=8.0 Hz, Ph-H), 7.51 (d, 1H, J=8.0 Hz, Ph-H), 7.92 (s, 1H, Ph-H), 8.27 (d, 1H, J=5.5 Hz, pyrimidinyl-H). MS (ESI⁺) m/z 419.33 [M+H]⁺ (C₁₈H₂₂N₆O₂S₂ requires 418.54).

N-{3-[4-(2-Amino-4-methyl-thiazol-5-yl)-pyrimidin-2-ylamino]-benzyl}-methane-sulfonamide (6). By condensation between N′-[5-(3-dimethylamino-acryloyl)-4-methyl-thiazol-2-yl]-N,N-dimethyl-formamidine and N-(3-methanesulfonamide-benzyl)-guanidine nitrate. Yellow solid. Anal. RP-HPLC: t_{R}=12.4 min (0-60% MeCN, purity >98%). ¹H-NMR (DMSO-d₆) δ: 2.49 (s, 3H, CH₃), 2.90 (s, 3H, CH₃), 4.29 (s, 2H, CH₂), 6.94 (d, 1H, J= 5.5 Hz, pyrimidinyl-H), 7.01 (d, 1H, J=8.0 Hz, Ph-H), 7.28 (d, 1H, J=8.0 Hz, Ph-H), 7.49 (t, 1H, J=8.0 Hz, Ph-H), 7.96 (s, 1H, Ph-H), 8.29 (d, 1H, J=5.5 Hz, pyrimidinyl-H). MS (ESI⁺) m/z 391.06 [M+H]⁺ (C₁₆H₁₈N₆O₂S₂ requires 390.49).

[4-(4-Methyl-2-methylamino-thiazol-5-yl)-pyrimidin-2-yl]-(3-piperazin-1-yl-phenyl)-amine (7). The titled compound was obtained by hydrolysis of 1-(4-{3-[4-(4-methyl-2-methylamino-thiazol-5-yl)-pyrimidin-2-ylamino]-phenyl}-piperazin-1-yl)-ethanone (3) in 2 M aq HCl/EtOH as a yellow solid. Anal. RP-HPLC: t_{R}=8.5 min (10-70% MeCN, purity 99%). ¹H-NMR (DMSO-d₆) δ: 2.33 (s, 3H, CH₃), 2.43 (s, 3H, CH₃), 2.83 (m, 4H, CH₂), 3.06 (m, 4H, CH₂), 6.50 (d, 1H, J=8.0 Hz, Ph-H), 6.87 (d, 1H, J=5.0 Hz, pyrimidinyl-H), 7.06 (t, 1H, J=8.5 Hz, Ph-H), 7.41 (s, 1H, Ph-H), 8.03 (m, 1H, NH), 8.31 (d, 1H, J=5.5 Hz, pyrimidinyl-H), 9.22 (s, 1H, NH). MS (ESI⁺) m/z 382.47 [M+H]⁺ (C₁₉H₂₃N₇S requires 381.50).

[4-(2,4-Dimethyl-thiazol-5-yl)-pyrimidin-2-yl]-(3-piperazin-1-yl-phenyl)-amine. (8). Yellow solid. Anal. RP-HPLC: t_{R}=9.9 min (10-70% MeCN, purity 99%). ¹H-NMR (DMSO-d₆) δ: 1.98 (s, 3H, CH₃), 2.62 (s, 3H, CH₃), 2.87 (m, 4H, CH₂), 3.06 (m, 4H, CH₂), 6.52 (d, 1H, J=8.0 Hz, Ph-H), 7.07 (d, 1H, J=5.0 Hz, pyrimidinyl-H), 7.12 (t, 1H, J=8.5 Hz, Ph-H), 7.18 (d, 1H, J=8.5 Hz, Ph-H), 7.52 (s, 1H, Ph-H), 8.50 (d, 1H, J=5.0 Hz, pyrimidinyl-H), 9.48 (s, 1H, NH). MS (ESI⁺) m/z 367.40 [M+H]⁺ (C₁₉H₂₂N₆S requires 366.48).

[4-(4-Methyl-2-methylamino-thiazol-5-yl)-pyrimidin-2-yl]-(3-piperazin-1-yl-phenyl)-amine (9). 3-Nitro-benzylamine hydrochloride (1.0 g, 5.3 mmol) was dissolved in CH₂Cl₂ (5 mL) and pyridine (3 eq, 1.29 mL) was added, followed by benzyl chloride (1.2 eq, 0.74 mL). The mixture was stirred at room temperature overnight and then washed with 2 M aq HCl solution. Following drying (Mg₂SO₄), the solvent was evaporated to leave a colourless solid. Silica gel flash chromatography (2:1 petroleum ether-ethyl acetate) gave N-(3-nitro-benzyl)-benzamide as a colourless solid (80% yield). ¹H-NMR (DMSO-d₆) δ: 4.66 (d, 2H, J=5.5 Hz, CH₂), 6.75 (sbr, 1H, NH), 7.37 (m, 2H, Ph-H), 7.45 (m, 2H, Ph-H), 7.63 (d, 1H, J=7.0 Hz, Ph-H), 7.74 (d, 2H, J=7.0 Hz, Ph-H), 8.05 (d, 1H, J=7.0 Hz, Ph-H) and 8.11 (s, 1H, Ph-H). This compound was hydrogenated in the presence of Pd/C to afford N-(3-amino-benzyl)-benzamide. ¹H-NMR (DMSO-d₆) δ: 4.34 (d, 2H, J=6.0 Hz, CH₂), 5.06 (sbr, 2H, NH₂), 6.41 (d, 1H, J=8.0 Hz, Ph-H), 6.46 (d, 1H, J=8.0 Hz, Ph-H), 6.51 (s, 1H, Ph-H), 6.94 (dd, 1H, J=8.0 Hz, Ph-H), 7.46-7.48 (m, 2H, Ph-H), 7.49-7.54 (m, 1H, Ph-H), 7.88-7.90 (m, 2H, Ph-H) and 8.91 (1H, t, J=6.0 Hz, NH). The title compound was obtained by condensation of the corresponding N-(3-guanidino-benzyl)-benzamide and 3-dimethylamino-1-(2,4-dimethyl-thiazol-5-yl)-propenone as a yellow solid. RP-HPLC: t_{R}=14.5 min (10-70% MeCN, purity 98%). ¹H-NMR (DMSO-d₆) δ: 2.53 (s, 3H, CH₃), 2.60 (s, 3H, CH₃), 4.49 (d, 2H, J=5.5 Hz, CH₂), 6.93 (d, 1H, J=8.0 Hz, Ph-H), 7.06 (d, 1H, J= 5.5 Hz, pyrimidinyl-H), 7.24 (t, 1H, J=8.0 Hz, Ph-H), 7.46-7.47 (m, 2H, Ph-H), 7.50-7.54 (m, 1H, Ph-H), 7.66 (d, 1H, J=8.0 Hz, Ph-H), 7.77 (s, 1H, Ph-H), 7.88-7.90 (m, 2H, Ph-H), 8.48 (d, 1H, J=5.0 Hz, pyrimidinyl-H), 8.99 (1H, t, J=6.0 Hz, NH). 9.67 (s, 1H, NH). MS (ESI⁺) m/z 416.45 [M+H]⁺ (C₂₃H₂₁N₅ OS requires 415.51).

N-{3-[4-(2-Ethylamino-4-methyl-thiazol-5-yl)-pyrimidin-2-ylamino]-benzyl}-C,C,C-trifluoro-methanesulfonamide (10). Anal. RP-HPLC: t_{R}=17.8 min (0-60% MeCN, purity 80%). ¹H-NMR (DMSO-d₆) δ: 1.29 (t, 3H, J=7.0 Hz, CH₃), 2.02 (s, 3H, CH₃), 3.36 (m, 2H, CH₂), 4.40 (s, 2H, CH₂), 6.93 (d, 1H, J=5.5 Hz, pyrimidinyl-H), 7.01 (d, 1H, J=7.5 Hz, Ph-H), 7.31 (d, 1H, J=8.0 Hz, Ph-H), 7.59 (d, 1H, J=8.0 Hz, Ph-H), 7.82 (s, 1H, Ph-H), 8.28 (d, 1H, J=5.5 Hz, pyrimidinyl-H). MS (ESI⁺) m/z 473.29 [M+H]⁺ (C₁₈H₁₉F₃N₆O₂S₂ requires 472.51).

N-{3-[4-(2,4-Dimethyl-thiazol-5-yl)-pyrimidin-2-ylamino]-benzyl}-C,C,C-trifluoro-methanesulfonamide (11). By treatment of 3-dimethylamino-1-(2,4-dimethyl-thiazol-5-yl)-propenone with N-(3-trifluoromethanesulfonamide-benzyl)-nitrate. Yellow solid. Anal. RP-HPLC: t_{R}=20.1 min (0-60% MeCN, purity >98%). ¹H-NMR (DMSO-d₆) δ: 2.65 (s, 3H, CH₃), 2.68 (s, 3H, CH₃), 4.39 (s, 2H, CH₂), 7.01 (d, 1H, J=8.0 Hz, Ph-d₆), 7.05 (d, 1H, J=5.0 Hz, pyrimidinyl-H), 7.31 (t, 1H, J=8.0 Hz, Ph-H), 7.55 (d, 1H, J=8.0 Hz, Ph-H), 7.91 (s, 1H, Ph-H), 8.43 (d, 1H, J=5.5 Hz, pyrimidinyl-H). MS (ESI⁺) m/z 444.35 [M+H]⁺ (C₁₇H₁₆F₃N₅O₂S₂ requires 443.47).

N-{3-[4-(2-Amino-4-methyl-thiazol-5-yl)-pyrimidin-2-ylamino]-benzyl}-C,C,C-trifluoro-methanesulfonamide (12). Anal. RP-HPLC: t_{R}=16.4 min (0-60% MeCN, purity 90%). ¹NMR (DMSO-d₆) δ: 2.01 (s, 3H, CH₃), 4.39 (s, 2H, CH₂), 6.93 (d, 1H, J=5.5 Hz, pyrimidinyl-H), 7.00 (d, 1H, J=7.5 Hz, Ph-H), 7.31 (d, 1H, J=8.0 Hz, Ph-H), 7.65 (d, 1H, J=8.0 Hz, Ph-H), 7.74 (s, 1H, Ph-H), 8.29 (d, 1H, J=5.5 Hz, pyrimidinyl-H). MS (ESI⁺) m/z 445.23 [M+H]⁺ (C₁₆H₁₅F₃N₆O₂S₂ requires 444.46).

N-{4-[4-(2-Ethylamino-4-methyl-thiazol-5-yl)-pyrimidin-2-ylamino]-benzyl}-acetamide (13). By treatment of 3-dimethylamino-1-(2-ethylamino-4-methyl-1-thiazol-5-yl)-propenone with N-(4-guanidino-benzyl)-acetamide nitrate. Yellow solid. Anal. RP-HPLC: t_{R}=17.3 min (0-60% MeCN, purity >98%). ¹H-NMR (DMSO-d₆) δ: 1.28 (t, 3H, J=7.0 Hz, CH₃), 2.52 (s, 3H, CH₃), 3.32 (s, 3H, CH₃), 3.36 (m, 2H, CH₂), 4.31 (s, 2H, CH₂), 6.90 (d, 1H, J=5.5 Hz, pyrimidinyl-H), 7.22 (d, 2H, J=8.5 Hz, Ph-H), 7.66 (d, 2H, J=9.0 Hz, Ph-H), 8.25 (d, 1H, J=5.5 Hz, pyrimidinyl-H). MS (ESI⁺) m/z 383.53 [M+H]⁺ (C₁₉H₂₂N₆OS requires 382.48).

N-{4-[4-(4-Methyl-2-methylamino-thiazol-5-yl)-pyrimidin-2-ylamino]-benzyl}-acetamide (14). By condensation between 3-dimethylamino-1-(4-methyl-2-methylamino-thiazol-5-yl)-propenone and N-(4-guanidino-benzyl)-acetamide nitrate. Yellow solid. Anal. RP-HPLC: t_{R}=11.6 min (0-60% MeCN, purity >90%). ¹NMR (DMSO-d₆) δ: 2.52 (s, 3H, CH₃), 2.97 (s, 3H, CH₃), 3.35 (s, 3H, CH₃), 4.31 (s, 2H, CH₂), 6.91 (d, 1H, J=5.5 Hz, pyrimidinyl-H), 7.23 (d, 2H, J=8.5 Hz, Ph-H), 7.66 (d, 2H, J=8.5 Hz, Ph-H), 8.26 (d, 1H, J=5.5 Hz, pyrimidinyl-H). MS (ESI⁺) m/z 369.54 [M+H]⁺ (C₁₈H₂₀N₆OS requires 368.46).

N-{4-[4-(2,4-Dimethyl-thiazol-5-yl)-pyrimidin-2-ylamino]-benzyl}-acetamide (15). By treatment of 3-dimethylamino-1-(2,4-(dimethyl-thiazol-5-yl)-propenone with N-(4-guanidino-benzyl)-acetamide nitrate. Yellow solid. Anal. RP-HPLC: t_{R}=13.5 min (0-60% MeCN, purity >90%). ¹NMR (DMSO-d₆) δ: 2.68 (s, 3H, CH₃), 2.70 (s, 3H, CH₃), 3.35 (s, 3H, CH₃), 4.33 (s, 2H, CH₂), 7.05 (d, 1H, J=5.0 Hz, pyrimidinyl-H), 7.25 (d, 2H, J=8.5 Hz, Ph-H), 7.67 (d, 2H, J=8.5 Hz, Ph-H), 8.43 (d, 1H, J=5.0 Hz, pyrimidinyl-H). MS (ESI⁺) m/z 354.48 [M+H]⁺ (C₁₈H₁₉N₅OS requires 353.44).

N-{4-[4-(2-Amino-4-methyl-thiazol-5-yl)-pyrimidin-2-ylamino]-benzyl}-acetamide (16). By treatment of N′-[5-(3-dimethylamino-acryloyl)-4-methyl-thiazol-2-yl]-N,N-dimethyl-formamidine with N-(4-guanidino-benzyl)-acetamide nitrate. Yellow solid. Anal. RP-HPLC: t_{R}=10.9 min (0-60% MeCN, purity >90%). ¹H-NMR (DMSO-d₆) δ: 2.50 (s, 3H, CH₃), 3.35 (s, 3H, CH₃), 4.32 (s, 2H, CH₂), 4.51 (sbr, 2H, NH₂), 6.92 (d, 1H, J=5.5 Hz, pyrimidinyl-H), 7.23 (d, 2H, J=9.0 Hz, Ph-H), 7.68 (d, 2H, J=8.5 Hz, Ph-H), 8.28 (d, 1H, J=5.5 Hz, pyrimidinyl-H). MS (ESI⁺) m/z 355.49 [M+H]⁺ (C₁₇H₁₈N₆OS requires 354.43).

4-(2-Ethylamino-4-methyl-thiazol-5-yl)-pyrimidin-2-yl]-(4-methanesulfonyl-phenyl)-amine (17). By condensation between 3-dimethylamino-1-(2-aminoethyl-4-methylthiazol-5-yl)-propenone and N-(4-methanesulfonyl-phenyl)-guanidine. Yellow solid. Anal. RP-HPLC: t_{R}=14.3 min (0-60% MeCN, purity >98%). ¹H-NMR (DMSO-d₆) δ: 1.18 (t, 3H, J=7.0 Hz, CH₃), 2.51 (s, 3H, CH₃), 3.15 (s, 3H, CH₃), 3.28 (m, 2H, CH₂), 7.01 (d, 1H, J=5.5 Hz, pyrimidinyl-H), 7.79 (m, 2H, Ph-H), 8.02 (m, 2H, Ph-H), 8.18 (m, 1H, NH), 8.40 (d, 1H, J= 5.5 Hz, pyrimidinyl-H), 10.01 (s, 1H, NH). MS (ESI⁺) m/z 390.43 [M+H]⁺ (C₁₇H₁₉N₅O₂S₂ requires 389.50).

3-[4-(2-Ethylamino-4-methyl-thiazol-5-yl)-pyrimidin-2-ylamino]-benzenesulfonamide (18). By condensation between 3-dimethylamino-1-(2-ethylamino-4-methyl-thiazol-5-yl)-propenone and N-(3-sulfonylacetamido-phenyl)-guanidine. Light yellow solid. Anal. RP-HPLC: t_{R}=13.1 min (0-60% MeCN, purity >98%). ¹H-NMR (DMSO-d₆) δ: 1.18 (t, 3H, J=7.5 Hz, CH₃), 2.48 (s, 3H, CH₃), 3.27 (m, 2H, CH₂), 6.94 (d, 1H, J=6.0 Hz, pyrimidinyl-H), 7.39 (m, 1H, Ph-H), 7.45 (m, 1H, Ph-H), 7.94 (m, 1H, Ph-H), 8.10 (m, 2H, NH₂), 8.32 (s, 1H, Ph-H), 8.35 (d, 1H, J=5.5 Hz, pyrimidinyl-H), 9.74 (s, 1H, NH). MS (ESI⁺) m/z 391.43 [M+H]⁺ (C₁₆H₁₈N₆CO₂S₂ requires 390.49).

3-[4-(4-Methyl-2-methylamino-thiazol-5-yl)-pyrimidin-2-ylamino]-benzenesulfonamide (19). Yellow solid. Anal. RP-HPLC: t_{R}=11.83 min (0-60% MeCN, purity 84%). ¹H-NMR (DMSO-d₆) d. 2.73 (s, 3H, CH₃), 3.12 (d, 3H, J=5.0 Hz, CH₃), 7.20 (d, 1H, J=6.0 Hz, pyrimidinyl-H), 7.52 (s, 2H, NH₂), 7.65 (d, 1H, J=8.0 Hz, Ph-H), 7.70 (t, 1H, J=8.0 Hz, Ph-H), 8.18 (d, 1H, J=8.0 Hz, Ph-H), 8.29 (m, 1H, NH), 8.59 (sbr, 1H, Ph-H), 8.61 (d, 1H, J=6.0 Hz, pyrimidinyl-H). MS (ESI⁺) m/z 377.46 [M+H]⁺ (C₁₅H₁₆N₆O₂S₂ requires 376.46).

(4-Methanesulfonyl-phenyl)-[4-(4-methyl-2-methylamino-thiazol-5-yl)-pyrimidin-2-yl]amine (20). By treatment of 3-dimethylamino-1-(2-methylamino-4-methyl-thiazol-5-yl)-propenone with N-(4-methanesulfonyl-phenyl)-guanidine. Light yellow solid. Anal. RP-HPLC: t_{R}=14.9 min (0-60% MeCN, purity >98%). ¹H-NMR (DMSO-d₆) δ: 2.87 (s, 3H, CH₃), 2.89 (s, 3H, CH₃), 3.16 (s, 3H, CH₃), 7.03 (d, 1H, J=5.5 Hz, pyrimidinyl-H), 7.81 (d, 2H, J=8.5 Hz, Ph-H), 8.04 (d, 2H, J=8.5 Hz, Ph-H), 8.12 (m, 1H, NH), 8.41 (d, 1H, J=5.5 Hz, pyrimidinyl-H), 10.02 (s, 1H, NH). MS (ESI⁺) m/z 376.20 [M+H]⁺ (C₁₆H₁₇N₅O₂S₂ requires 375.47).

N-Methyl-3-[4-(4-methyl-2-methylamino-thiazol-5-yl)-pyrimidin-2-ylamino]-benzenesulfonamide sulfonamide (21). By condensation between 3-dimethylamino-1-(4-methyl-2-methylamino-thiazol-5-yl)-propenone and N-methyl-3-guanidino-benzene-sulfon-amide. Yellow solid. Anal. RP-HPLC: t_{R}=13.2 min (0-60% MeCN, purity >98%). ¹NMR (DMSO-d₆) δ: 2.53 (s, 3H, CH₃), 2.57 (d, 3H, J=5.0 Hz, CH₃), 2.99 (s, 3H, CH₃), 7.00 (d, 1H, J=5.5 Hz, pyrimidinyl-H), 7.44 (d, 1H, J=8.0 Hz, Ph-H), 7.48 (t, 1H, J=8.0 Hz, Ph-H), 7.80 (d, 1H, J=8.0 Hz, Ph-H), 8.33 (d, 1H, J=5.5 Hz, pyrimidinyl-H), 8.52 (s, 1H, Ph-H). MS (ESI⁺) m/z 391.27 (C₁₆H₁₈N₆O₂S₂ requires 390.49).

3-[4-(2-Ethylamino-4-methyl-thiazol-5-yl)-pyrimidin-2-ylamino]-N-methyl-benzene-sulfonamide (22). By condensation between 3-dimethylamino-1-(2-ethylamino-4-methyl-thiazol-5-yl)-propenone and N-methyl-3-guanidino-benzenesulfonamide. Yellow solid. Anal. RP-HPLC: t_{R}=14.0 min (0-60% MeCN, purity >98%). ¹H-NMR (DMSO-d₆) 1.29 (t, 3H, J=7.0 Hz, CH₃), 2.53 (s, 3H, CH₃), 2.58 (s, 3H, CH₃), 3.39 (m, 2H, CH₂), 6.99 (d, 1H, J=5.0 Hz, pyrimidinyl-H), 7.42 (d, 1H, J=8.0 Hz, Ph-H), 7.48 (t, 1H, J=8.0 Hz, Ph-H), 7.80 (d, 1H, J=8.0 Hz, Ph-H), 8.32 (d, 1H, J=5.5 Hz, pyrimidinyl-H), 8.52 (s, 1H, Ph-H). MS (ESI⁺) m/z 409.20 [M+H]⁺ (C₁₇N₂₀N₆O₂S₂ requires 404.51).

[4-(4-Methyl-2-methylamino-thiazol-5-yl)-pyrimidin-2-yl]-(3,4,5-trimethoxy-phenyl)-amine (23). By treatment of 3-dimethylamino-1-(4-methyl-2-methylamino-thiazol-5-yl)-propenone with N-(3,4,5-trimethoxy-phenyl)-guanidine nitrate. Yellow solid. Anal. RP-HPLC: t_{R}=11.1 min (10-70% MeCN, purity >99%). ¹H-NMR (DMSO-d₆) d. 2.46 (s, 3H, CH₃), 3.61 (s, 3H, CH₃), 3.61 (s, 3H, CH₃), 3.81 (s, 6H, 2×CH₃), 6.90 (d, 1H, J=5.5 Hz, pyrimidinyl-H), 7.17 (s, 2H, Ph-H), 8.32 (d, 1H, J=5.5 Hz, pyrimidinyl-H), 9.28 (s, 1H, NH). MS (ESI⁺) m/z 388.33 [M+H]⁺ C₁₈N₂₁N₅O₃S requires 387.46).

[4-(2-Ethylamino-4-methyl-thiazol-5-yl)-pyrimidin-2-yl]-(3,4,5-trimethoxy-phenyl)-amine (24). By treatment of 3-dimethylamino-1-(2-ethylamino-4-methyl-thiazol-5-yl)-propenone with N-(3,4,5-trimethoxy-phenyl)-guanidine nitrate. Yellow solid. Anal. RP-HPLC: t_{R}=12.1 min (10-70% MeCN, purity >99%). ¹H-NMR (DMSO-d₆) δ: 1.17 (t, 3H, J=7.5 Hz, CH₃), 2.45 (s, 3H, CH₃), 3.61 (s, 3H, CH₃), 3.80 (s, 6H, 2×CH₃), 6.90 (d, 1H, J=6.0 Hz, pyrimidinyl-H), 7.16 (s, 2H, Ph-H), 8.13 (m, 1H, NH), 8.32 (d, 1H, J=5.5 Hz, pyrimidinyl-H), 9.27 (s, 1H, NH). MS (ESI⁺) m/z 402.37 [M+H]⁺ (C₁₉H₂₃N₅O₃S requires 401.48).

[4-(2,4-Dimethyl-thiazol-5-yl)-pyrimidin-2-yl]-(3,4,5-trimethoxy-phenyl)-amine (25). By treatment of 3-dimethylamino-1-(2,4-dimethyl-thiazol-5-yl)-propenone with N-(3,4,5-trimethoxy-phenyl)-guanidine nitrate. Yellow solid. Anal. RP-HPLC: t_{R}=14.1 min (10-70% MeCN, purity >99%). ¹H-NMR (DMSO-d₆) δ: 2.07 (s, 6H, 2×CH₃), 3.62 (s, 3H, CH₃), 3.79 (s, 6H, 2×CH₃), 7.08 (d, 1H, J=5.0 Hz, pyrimidinyl-H), 7.18 (s, 2H, Ph-H), 8.51 (d, 1H, J=5.0 Hz, pyrimidinyl-H), 9.51 (s, 1H, NH). MS (ESI⁺) m/z 373.34 [M+H]⁺ (C₁₈H₂₀N₄O₃S requires 372.44).

3-[4-(2-Amino-4-methyl-thiazol-5-yl)-pyrimidin-2-ylamino]-N-methyl-benzenesulfonamide (26). By condensation between N′-[5-(3-dimethylamino-acryloyl)-4-methyl-thiazol-2-yl]-N,N-dimethyl-formamidine and N-methyl-3-guanidino-benzenesulfonamide. Yellow solid. Anal. RP-HPLC: t_{R}=15.9 min (0-60% MeCN, purity >98%). ¹NMR (DMSO-de) δ: 2.43 (s, 3H, CH₃), 2.44 (s, 3H, CH₃), 7.14 (d, 1H, J=5.5 Hz, pyrimidinyl-H), 7.35 (m, 1H, Ph-H), 7.52 (t, 1H, J=8.0 Hz, Ph-H), 7.98 (d, 1H, J=8.0 Hz, Ph-H), 8.30 (s, 1H, Ph-H), 8.58 (d, 1H, J=5.5 Hz, pyrimidinyl-H). MS (ESI⁺) m/z 376.28 (C₁₅H₁₆N₆O₂S₂ requires 376.46).

(3-Methanesulfonyl-phenyl)-[4-(4-methyl-2-methylamino-thiazol-5-yl)-pyrimidin-2-yl]-amine (27). Yellow solid. Anal. RP-HPLC: t_{R}=13.8 min (0-60% MeCN, purity 100%). ¹NMR (DMSO-d₆) δ: 2.86 (s, 3H, CH₃), 2.87 (s, 3H, CH₃), 3.20 (s, 3H, CH₃), 6.98 (d, 1H, J=5.5 Hz, pyrimidinyl-H), 7.46 (d, 1H, J=7.5 Hz, Ph-H), 7.54 (t, 1H, J=7.5 Hz, Ph-H), 7.95 (m, 1H, Ph-H), 8.08 (m, 1H, NH), 8.38 (d, 1H, J=5.0 Hz, pyrimidinyl-H), 8.54 (s, 1H, Ph-H), 9.87 (s, 1H, NH). MS (ESI⁺) m/z 376.38 [M+H]⁺ (C₁₆H₁₇N₅O₂S₂ requires 375.47).

[4-(2-Ethylamino-4-methyl-thiazol-5-yl)-pyrimidin-2-yl]-(3-methanesulfonyl-phenyl)-amine (28). Yellow solid. Anal. RP-HPLC: t_{R}=14.6 min (0-60% MeCN, purity 100%). ¹H-NMR (DMSO-d₆) δ: 1.19 (t, 3H, J=7.0 Hz, CH₃), 2.48 (s, 3H, CH₃), 2.63-3.17 (m, 5H, CH₃ and CH₂), 6.97 (d, 1H, J=5.5 Hz, pyrimidinyl-H), 7.47 (d, 1H, J=7.5 Hz, Ph-H), 7.52 (t, 1H, J=7.5 Hz, Ph-H), 7.94 (m, 1H, Ph-H), 8.16 (m, 1H, NH), 8.38 (d, 1H, J=5.0 Hz, pyrimidinyl-H), 8.51 (s, 1H, Ph-H), 9.86 (s, 1H, NH). MS (ESI⁺) m/z 390.37[M+H]⁺ (C₁₇H₁₉N₅O₂S₂ requires 389.50).

N-Ethyl-3-[4-(2-ethylamino-4-methyl-thiazol-5-yl)-pyrimidin-2-ylamino]-benzene-sulfonamide (29). By condensation between 3-dimethylamino-1-(2-ethylamino-4-methyl-thiazol-5-yl)-propenone and N-ethyl-3-guanidino-benzenesulfonamide. Yellow solid. Anal. RP-HPLC: t_{R}=14.9 min (0-60% MeCN, purity >98%). ¹H-NMR (DMSO-d₆) δ: 1.07 (t, 3H, J=7.5 Hz, CH₃), 1.29 (t, 3H, J=7.0 Hz, CH₃), 2.53 (s, 3H, CH₃), 2.95 (m, 2H, CH₂), 3.39 (m, 2H, CH₂), 6.99 (d, 1H, J=5.5 Hz, pyrimidinyl-H), 7.45 (m, 2H, Ph-H), 7.79 (d, m, J=8.0 Hz, Ph-H), 8.32 (d, 1H, J=5.5 Hz, pyrimidinyl-H), 8.51 (s, 1H, Ph-H). MS (ESI⁺) m/z 419.33 [M+H]⁺ (C₁₈H₂₂N₆O₂S₂ requires 418.54).

3-[4-(2-Amino-4-methyl-thiazol-5-yl)-pyrimidin-2-ylamino]-N-ethyl-benzenesulfon-amide (30). Yellow solid. Anal. RP-HPLC: t_{R}=13.5 min (0-60% MeCN, purity >98%). ¹H-NMR (DMSO-d₆) δ: 1.08 (t, 3H, J=7.5 Hz, CH₃), 2.51 (s, 3H, CH₃), 2.95 (m, 2H, CH₂), 6.98 (d, 1H, J=5.5 Hz, pyrimidinyl-H), 7.44-7.49 (m, 2H, Ph-H), 7.89 (d, 1H, J=7.5 Hz, Ph-H), 8.34 (d, 1H, J=5.5 Hz, pyrimidinyl-H), 8.37 (sbr, 1H, Ph-H). MS (ESI⁺) m/z 391.37 [M+H]⁺ (C₁₆H₁₈N₆O₂S₂ requires 390.49).

N-Ethyl-3-[4-(4-methyl-2-methylamino-thiazol-5-yl)-pyrimidin-2-ylamino]-benzenesulfonamide (31). By condensation between 3-dimethylamino-1-(4-methyl-2-methylamino-thiazol-5-yl)-propenone and N-ethyl-3-guanidino-benzenesulfonamide. Yellow solid. Anal. RP-HPLC: t_{R}=14.1 min (0-60% MeCN, purity >98%). ¹H-NMR (DMSO-d₆) δ: 1.07 (t, 3H, J=7.5 Hz, CH₃), 2.53 (s, 3H, CH₃), 2.96 (m, 2H, CH₂), 2.99 (s, 3H, CH₃), 6.99 (d, 1H, J=5.5 Hz, pyrimidinyl-H), 7.47 (m, 2H, Ph-H), 7.79 (d, 1H, J=7.5 Hz, Ph-H), 8.33 (d, 1H, J=5.5 Hz, pyrimidinyl-H), 8.51 (s, 1H, Ph-H). MS (ESI⁺) m/z 405.29 [M+H]⁺ (C₁₇H₂₀N₆O₂S₂ requires 404.51).

N-(3-Methoxy-phenyl)-3-[4-(4-methyl-2-methylamino-thiazol-5-yl)-pyrimidin-2-ylamino]-benzenesulfonamide (32). By treatment of 3-dimethylamino-1-(4-methyl-2-methylamino-thiazol-5-yl)-propenone with 3-Guanidino-N-(3-methoxy-phenyl)-benzenesulfonamide nitrate. Yellow solid. Anal. RP-HPLC: t_{R}=14.1 (10-70% MeCN, purity >98%). ¹H-NMR (DMSO-d₆) δ: 2.47 (s, 3H, CH₃), 2.86 (s, 3H, CH₃), 3.62 (s, 3H, CH₃), 6.54 (m, 1H, Ph-H), 6.69 (m, 2H, Ph-H), 6.97 (d, 1H, J=5.5 Hz, pyrimidinyl-H), 7.08 (t, 1H, J=8.0 Hz, Ph-H), 7.32 (d, 1H, J=8.0 Hz, Ph-H), 7.44 (t, 1H, J=8.0 Hz, Ph-H), 7.92 (d, 1H, J=8.0 Hz, Ph-H), 8.13 (s, 1H, NH), 8.36 (d, 1H, J=5.5 Hz, pyrimidinyl-H), 8.39 (s, 1H, Ph-H), 9.79 (s, 1H, NH), 10.25 (sbr, 1H, NH). MS (ESI⁺) m/z 483.38 [M+H]⁺ (C₂₂H₂₂N₆O₃S₂ requires 482.58).

3-[4-(2,4-Dimethyl-thiazol-5-yl)-pyrimidin-2-ylamino]-N-methyl-benzenesulfonamide (33). By treatment of 3-dimethylamino-1-(2,4-dimethyl-thiazol-5-yl)-propenone with N-methyl-3-guanidino-benzenesulfonamide. Yellow solid. Anal. RP-HPLC: t_{R}=12.6 min (0-60% MeCN, purity >98%). ¹H-NMR (DMSO-d₆) Sr. 2.50 (s, 3H, CH₃), 2.58 (s, 3H, CH₃), 3.31 (s, 3H, CH₃), 6.98 (d, 1H, J=5.5 Hz, pyrimidinyl-H), 7.43 (d, 1H, J=7.5 Hz, Ph-H), 7.49 (d, 1H, J=8.0 Hz, Ph-H), 7.89 (d, 1H, J=8.0 Hz, Ph-H), 8.33 (d, 1H, J=5.5 Hz, pyrimidinyl-H), 8.37 (s, 1H, Ph-H). MS (ESI⁺) m/z 377.03 [M+H]⁺ (C₁₆H₁₇N₅O₂S₂ requires 375.47).

4-[4-(4-Methyl-2-methylamino-thiazol-5-yl)-pyrimidin-2-ylamino]-benzenesulfon-amide (34). By treatment of 3-dimethylamino-1-(4-methyl-2-methylamino-thiazol-5-yl)-propenone and N-methyl-3-guanidino-benzenesulfonamide. Yellow solid. Anal. RP-HPLC: t_{R}=13.2 min (0-60% MeCN, purity >98%). ¹H-NMR (DMSO-d₆) δ: 2.62 (s, 3H, CH₃), 3.01 (d, 3H, J=5.0 Hz, CH₃), 7.13 (d, 1H, J=5.5 Hz, pyrimidinyl-H), 7.30 (sbr, 2H, NH₂), 7.85 (d, 2H, J=9.0 Hz, Ph-H), 8.07 (d, 2H, J=9.0 Hz, Ph-H), 8.23 (m, 1H, NH), 8.52 (d, 1H, J=5.5 Hz, pyrimidinyl-H). MS (ESI⁺) m/z 377.39 [M+H]⁺ (C₁₅H₁₆N₆O₂S₂ requires 376.46).

4-[4-(2-Ethylamino-4-methyl-thiazol-5-yl)-pyrimidin-2-ylamino]-benzenesulfonamide (35). By treatment of 3-dimethylamino-1-(2-ethylamino-4-methyl-thiazol-5-yl)-propenone and N-methyl-3-guanidino-benzenesulfonamide. Yellow solid. Anal. RP-HPLC: t_{R}=14.1 min (0-60% MeCN, purity >98%). ¹NMR (DMSO-d₆) δ: 1.29 (t, 3H, J=7.0 Hz, CH₃), 2.13 (s, 3H, CH₃), 3.40 (m, 2H, CH₂), 7.13 (d, 1H, J=5.5 Hz, pyrimidinyl-H), 7.30 (sbr, 2H, NH₂), 7.85 (d, 2H, J=8.5 Hz, Ph-H), 8.07 (d, 2H, J=8.5 Hz, Ph-H), 8.29 (m, 1H, NH), 8.52 (d, 1H, J=5.5 Hz, pyrimidinyl-H). MS (ESI⁺) m/z 391.31 [M+H]⁺ (C₁₆H₁₈N₆O₂S₂ requires 390.49).

[4-(2-Ethylamino-4-methyl-thiazol-5-yl)-pyrimidin-2-yl]-[4-methyl-3-(morpholine-4-sulfonyl)-phenyl]-amine (36). By treatment of 3-(dimethylamino-1-(2-ethylamino-4-methyl-thiazol-5-yl)-propenone with N-[4-methyl-3-(morpholine-4-sulfonyl)-phenyl]-guanidine nitrate. Yellow solid. Anal. RP-HPLC: t_{R}=16.7 min (0-60% MeCN, purity 99%). ¹NMR (DMSO-d₆) δ: 1.48 (t, 3H, J=7.5 Hz, CH₃), 2.39 (s, 3H, CH₃), 2.77 (s, 3H, CH₃), 2.81 (m, 2H, CH₂), 3.36 (m, 4H, CH₂), 3.93 (m, 4H, CH₂), 7.24 (d, 1H, J=5.5 Hz, pyrimidinyl-H), 7.65 (d, 1H, J=9.0 Hz, Ph-H), 8.32 (d, 1H, J=8.5 Hz, Ph-H), 8.42 (t, 1H, 5.5 Hz, Ph-H), 8.52 (s, 1H, NH), 8.65 (d, 1H, J=5.0 Hz, pyrimidinyl-H). MS (ESI⁺) m/z 475.37 [M+H]⁺ (C₂₁H₂₆N₆O₃S₂ requires 474.60).

[4-(4-Methyl-2-methylamino-thiazol-5-yl)-pyrimidin-2-yl]-[4-methyl-3-(morpholine-4-sulfonyl)-phenyl]-amine (37). By treatment of 3-dimethylamino-1-(4-methyl-2-methylamino-thiazol-5-yl)-propenone with N-[4-methyl-3-(morpholine-4-sulfonyl)-phenyl]-guanidine nitrate. Yellow solid. Anal. RP-HPLC: t_{R}=15.8 min (0-60% MeCN, purity >99%). ¹H-NMR (DMSO-d₆) δ: 2.53 (s, 3H, CH₃), 2.59 (s, 3H, CH₃), 2.99 (s, 3H, CH₃), 3.16 (m, 4H, CH₂), 3.70 (m, 4H, CH₂), 6.97 (d, 1H, J=5.5 Hz, pyrimidinyl-H), 7.34 (d, 1H, J=8.0 Hz, Ph-H), 7.80 (d, 1H, J=8.0 Hz, Ph-H), 8.31 (d, 1H, J=5.0 Hz, pyrimidinyl-H), 8.41 (s, 1H, Ph-H). MS (ESI⁺) m/z 461.45 [M+H]⁺ (C₂₀H₂₄N₆O₃S₂ requires 460.58).

[4-(2-Amino-4-methyl-thiazol-5-yl)-pyrimidin-2-yl]-[4-methyl-3-(morpholine-4-sulfonyl)-phenyl]-amine (38). By treatment of N′-[5-(3-dimethylamino-acryloyl)-4-methyl-thiazol-2-yl]-N,N-dimethyl-methyl-formamidine with N-[4-methyl-3-(morpholine-4-sulfonyl)-phenyl]-guanidine nitrate. Yellow solid. Anal. RP-HPLC: t_{R}=15.5 min (0-60% MeCN, purity 99%). ¹H-NMR (DMSO-d₆) δ: 2.76 (s, 3H, CH₃), 2.82 (s, 3H, CH₃), 3.38 (m, 4H, CH₂), 3.95 (m, 4H, CH₂), 7.23 (d, 1H, J=5.5 Hz, pyrimidinyl-H), 7.66 (d, 1H, J=9.0 Hz, Ph-H), 7.83 (s, 2H, NH₂), 8.41 (m, 1H, Ph-H), 8.66 (d, 1H, J=5.0 Hz, pyrimidinyl-H). MS (ESI⁺) m/z 447.29 [M+H]⁺ (C₁₉H₂₂N₆O₃S₂ requires 446.55).

4-[4-(2-Ethylamino-4-methyl-thiazol-5-yl)-pyrimidin-2-ylamino]-N-(2-methoxy-ethyl)-benzenesulfonamide (39). To a solution of 4-nitro-benzenesulfonyl chloride (5.9 g, 0.027 mol) in CH₂Cl₂ (15 mL) at 0° C. was added 2-methoxy-ethylamine (3.46 mL, 0.04 mol). A precipitate formed almost immediately. After stirring for a further 1-2 h the reaction mixture was evaporated under reduced pressure. The resulting residue was purified by silica gel flash chromatography with the product being eluted by 2:1 EtOAc:petroleum ether to yield N-(2-methoxy-ethyl)-4-nitro-benzenesulfonamide as a white powder (4.99 g, 72%). ¹H-NMR (CD₃OD) δ: 3.11 (t, 2H, J=5.5 Hz, CH₂), 3.21 (s, 3H, CH₃), 3.37 (t, 2H, J=5.5 Hz, CH₂), 8.08 (d, 2H, J=9.0 Hz, Ph-H), 8.40 (d, 2H, J=9.0 Hz, Ph-H); MS (ESI⁺) m/z 259.16 (C₉H₁₂N₂O₅S requires 260.27). A solution of this compound (4.95 g, 0.019 mol) in EtOH (20 mL) was reduced by hydrogenation in the presence of Pd/C. After stirring at room temperature overnight the reaction mixture was filtered through a pad of Celite. The filtrate was evaporated under reduced pressure to afford 4-amino-N-(2-methoxy-ethyl)-benzenesulfonamide (3.6 g, 82%) as a yellow oil. ¹NMR (CD₃OD) δ: 2.96 (t, 2H, J=5.5 Hz, CH₂), 3.25 (s, 3H, CH₃), 3.36 (t, 2H, J=5.5 Hz, CH₂), 6.70 (d, 2H, J=9.0 Hz, Ph-H), 7.52 (d, 2H, J=9.0 Hz, CH₂₎). MS (ESI⁺) m/z 231.23 (C₉H₁₄N₂O₃S requires 230.29). The title compound was prepared by treatment of 3-dimethylamino-1-(2-ethylamino-4-methyl-thiazol-5-yl)-propenone with 4-guanidino-N-(2-methoxy-ethyl)-benzenesulfonamide nitrate. Yellow solid. Anal. RP-HPLC: t_{R}=14.7 min (0-60% MeCN, purity >98%). ¹H-NMR (CD₃OD) δ: 1.17 (t, 3H, J=7.0 Hz, CH₃), 2.54 (s, 3H, CH₃), 3.03 (t, 2H, J=6.0 Hz, CH₂), 3.27 (s, 3H, CH₃), 3.37 (m, 2H, CH₂), 3.48 (m, 2H, CH₂), 7.01 (d, 1H, J=5.5 Hz, pyrimidinyl-H), 7.77 (d, 2H, J=8.5 Hz, Ph-H), 7.94 (d, 2H, J=8.5 Hz, Ph-H), 8.34 (d, 1H, J=5.5 Hz, pyrimidinyl-H). MS (ESI⁺) m/z 449.35 [M+H]⁺ (C₁₉H₂₄N₆O₃S₂ requires 448.56).

N-(2-Methoxy-ethyl)-4-[4-(4-methyl-2-methylamino-thiazol-5-yl)-pyrimidin-2-ylamino]-benzenesulfonamide (40). By treatment of 3-dimethylamino-1-(4-methyl-2-methylamino-thiazol-5-yl)-propenone with 4-guanidino-N-(2-methoxy-ethyl)-benzenesulfonamide nitrate. Yellow solid. Anal. RP-HPLC: t_{R}=13.1 min (0-60% MeCN, purity >98%). ¹H-NMR (DMSO-d₆) δ: 2.57 (s, 3H, CH₃), 2.97 (m, 5H, CH₃ and CH₂), 3.26 (s, 3H, CH₃), 3.39 (m, 2H, CH₂), 7.09 (d, 1H, J=5.5 Hz, pyrimidinyl-H), 7.57 (m, 1H, NH), 7.77 (d, 2H, J=8.5 Hz, Ph-H), 8.05 (d, 2H, J=8.5 Hz, Ph-H), 8.19 (m, 1H, NH), 8.49 (d, 1H, J=5.5 Hz, pyrimidinyl-H). MS (ESI⁺) m/z 435.39 [M+H]⁺ (C₁₈H₂₂N₆O₃S₂ requires 434.54).

4-[4-(2-Amino-4-methyl-thiazol-5-yl)-pyrimidin-2-ylamino]-N-(2-methoxy-ethyl)-benzenesulfonamide (41). By treatment of N′-[5-(3-dimethylamino-acryloyl)-4-methyl-thiazol-2-yl]-N,N-dimethyl-formamidine with 4-guanidino-N-(2-methoxy-ethyl)-benzenesulfonamide nitrate. Yellow solid. Anal. RP-HPLC: t_{R}=14.4 min (0-60% MeCN, purity >98%). ¹NMR (DMSO-d₆) δ: 2.54 (s, 3H, CH₃), 2.96 (m, 2H, CH₂), 3.25 (s, 3H, CH₃), 3.37 (m, 2H, CH₂), 7.07 (d, 1H, J=5.0 Hz, pyrimidinyl-H), 7.57 (m, 1H, NH), 7.65 (sbr, 2H, NH₂), 7.76 (d, 2H, J=8.5 Hz, Ph-H), 8.05 (d, 2H, J=8.5 Hz, Ph-H), 8.48 (d, 1H, J=5.5 Hz, pyrimidinyl-H).

(3-Bromo-4-methyl-phenyl)-[4-(4-methyl-2-methylamino-thiazol-5-yl)-pyrimidin-2-yl]-amine (42). By treatment of 3-dimethylamino-1-(2-methylamino-4-methyl-thiazol-5-yl)-propenone with 3-bromo-4-methyl-phenyl guanidine nitrate. Yellow solid. Anal. RP-HPLC: t_{R}=15.1 min (10-70% MeCN, purity 98%). ¹NMR (DMSO-d₆) δ: 2.28 (s, 3H, CH₃), 2.47 (s, 3H, CH₃), 2.86 (d, 3H, J=4.5 Hz, CH₃), 6.91 (d, 1H, J=5.5 Hz, pyrimidinyl-H), 7.23 (d, 1H, J=8.5 Hz, Ph-H), 7.52 (d, 1H, J=8.5 Hz, Ph-H), 8.07 (sbr, 1H, NH), 8.29 (s, 1H, Ph-H), 8.34 (d, 1H, J=5.5 Hz, pyrimidinyl-H), 9.54 (sbr, 1H, NH). MS (ESI⁺) m/z 390.30 (C₁₆H₁₆BrN₅S requires 390.30).

4-[4-(2,4-Dimethyl-thiazol-5-yl)-pyrimidin-2-ylamino]-N-(2-methoxy-ethyl)-benzene-sulfonamide (43). Yellow solid. Anal. RP-HPLC: t_{R}=19.3 min (0-60% MeCN, purity 100%). ¹H-NMR (CD₃OD) δ: 2.51 (s, 3H, CH₃), 2.53 (s, 3H, CH₃), 2.76 (m, 2H, CH₂), 3.03 (s, 3H, CH₃), 3.19 (m, 2H, CH₂), 7.06 (d, 1H, J=5.5 Hz, pyrimidinyl-H), 7.36 (sbr, 1H, NH), 7.58 (d, 2H, J=8.5 Hz, Ph-H), 7.83 (d, 2H, J=8.5 Hz, Ph-H), 8.46 (d, 1H, J=5.5 Hz, pyrimidinyl-H). MS (ESI⁺) m/z 420.47 [M+H]⁺ (C₁₈H₂₁N₅O₃S₂ requires 419.52).

{3-[4-(4-Methyl-2-methylamino-thiazol-5-yl)-pyrimidin-2-ylamino]-phenyl}-acetic acid 2-methoxy-ethyl ester (44). Yellow solid. Mp. 193-195° C. Anal. RP-HPLC: t_{R}=11.3 min (10-70% MeCN, purity 97%). ¹H-NMR (CD₃OD) δ: 2.52 (s, 3H, CH₃), 2.97 (s, 3H, CH₃), 3.33 (s, 3H, CH₃), 3.58 (q, 2H, J=4.5 Hz, CH₂), 3.68 (s, 2H, CH₂), 4.23 (q, 2H, J=4.5 Hz, CH₂), 6.91 (d, 1H, J=5.5 Hz, pyrimidinyl-H), 6.94 (m, 1H, Ph-H), 7.24 (t, 1H, J=8.0 Hz, Ph-H), 7.51 (d, 1H, J=8.0 Hz, Ph-H), 7.79 (s, 1H, Ph-H), 8.27 (d, 1H, J=5.5 Hz, Pyrimidinyl-H). MS (ESI⁺) m/z 414.34 [M+H]⁺ (C₂₀H₂₃N₅O₃S requires 413.49).

{3-[4-(2-Ethylamino-4-methyl-thiazol-5-yl)-pyrimidin-2-ylamino]-phenyl}-acetic acid 2-methoxy-ethyl ester (45). Yellow solid. Anal. RP-HPLC: t_{R}=12.3 min (10-70% MeCN, purity 100%). ¹H-NMR (CD₃OD) δ: 1.35 (t, 3H, J=7.0 Hz, CH₃), 2.61 (s, 3H, CH₃), 3.33 (s, 3H, CH₃), 3.49 (m, 2H, CH₂), 3.59 (q, 2H, J=4.5 Hz, CH₂), 3.71 (s, 2H, CH₂), 4.24 (t, 2H, J=4.5 Hz, CH₂), 7.04-7.07 (m, 2H, Ph-H and Pyrimidinyl-H), 7.31 (t, 1H, J=8.0 Hz, Ph-H), 7.48 (d, 1H, J=8.0 Hz, Ph-H), 7.66 (s, 1H, Ph-H), 8.32 (d, 1H, J=5.5 Hz, Pyrimidinyl-H). MS (ESI⁺) m/z 429.37 (C₂₁H₂₅N₅O₃S requires 427.52).

1-(4-{3-[4-(2,4-Dimethyl-thiazol-5-yl)-pyrimidin-2-ylamino]-phenyl}-piperazin-1-yl)-ethanone (46). This compound was obtained by treatment of N-[3-(4-acetyl-piperazin-1-yl)-phenyl]-guanidine with 3-dimethylamino-1-(2,4-dimethyl-thiazol-5-yl)-propenone. Yellow solid. Anal. RP-HPLC: t_{R}=12.9 min (10-70% MeCN, purity 99%). ¹H-NMR (DMSO-d₆) δ: 2.62 (s, 3H, CH₃), 2.64 (s, 3H, CH₃), 2.85 (s, 3H, CH₃), 3.09 (m, 2H, CH₂), 3.16 (m, 2H, CH₂), 3.59 (m, 4H, CH₂), 6.58 (d, 1H, J=8.0 Hz, Ph-H), 7.08 (d, 1H, J=5.5 Hz, pyrimidinyl-H), 7.12 (t, 1H, J=8.5 Hz, Ph-H), 7.20 (d, 1H, J=8.0 Hz, Ph-H), 7.56 (s, 1H, Ph-H), 8.51 (d, 1H, J=5.0 Hz, pyridinyl-H), 9.53 (s, 1H, NH). MS (ESI⁺) m/z 431.44 [M+Na] (C₂₁H₂₄N₆OS requires 408.52).

{3-[4-(2,4-Dimethyl-thiazol-5-yl)-pyrimidin-2-ylamino]-5-hydroxymethyl-phenyl}-methanol (47). By treatment of 3-dimethylamino-1-(2,4-dimethyl-thiazol-5-yl)-propenone with N-(3,5-bis-hydroxymethyl-phenyl)-guanidine nitrate. Yellow solid. Anal. RP-HPLC: t_{R}=12.0 min (10-70% MeCN, purity >98%). ¹NMR (DMSO-d₆) δ: 2.63 (s, 3H, CH₃), 2.64 (s, 3H, CH₃), 4.48 (d, 4H, J=6.0 Hz, CH₂), 5.13 (t, 2H, J=5.5 Hz, OH), 6.92 (s, 1H, Ph-H), 7.05 (d, 1H, J=5.5 Hz, pyrimidinyl-H), 7.61 (s, 2H, Ph-H), 8.49 (d, 1H, J=5.5 Hz, pyrimidinyl-H), 9.60 (s, 1H, NH). MS (ESI⁺) m/z 343.37 [M+H]⁺ (C₁₇H₁₈N₄O₃S requires 342.42).

{3-Hydroxymethyl-5-[4-(4-methyl-2-methylamino-thiazol-5-yl)-pyrimidin-2-ylamino]-phenyl}-methanol (48). By treatment of 3-dimethylamino-1-(4-methyl-2-methylamino-thiazol-5-yl)-propenone with N-(3,5-bis-hydroxymethyl-phenyl)-guanidine nitrate. Yellow solid. Anal. RP-HPLC: t_{R}=11.2 min (10-70% MeCN, purity >98%). ¹H-NMR (DMSO-d₆) δ: 2.85 (s, 3H, CH₃), 3.29 (s, 3H, CH₃), 4.47 (d, 4H, J=6.0 Hz, CH₂), 5.09 (t, 2H, J=5.5 Hz, OH), 6.86 (d, 1H, 5.5 Hz, pyrimidinyl-H), 6.91 (s, 1H, Ph-H), 7.60 (s, 2H, Ph-H), 8.04 (s, 1H, NH), 8.31 (d, 1H, J=5.5 Hz, pyrimidinyl-H), 9.37 (s, 1H, NH). MS (ESI⁺) m/z 358.43 [M+H]⁺ (C₁₇H₁₉N₅O₂S requires 357.43).

N-{3-[4-(2,4-Dimethyl-thiazol-5-yl)-pyrimidin-2-ylamino]-benzyl}-methanesulfon-amide (49). By condensation between 3-dimethylamino-1-(2,4-dimethyl-thiazol-5-yl)-propenone and N-(3-methanesulfonamide-benzyl)-guanidine nitrate. Yellow solid. Anal. RP-HPLC: t_{R}=14.8 min (0-60% MeCN, purity >98%). ¹H-NMR (DMSO-d₆) δ: 2.68 (s, 3H, CH₃), 2.70 (s, 3H, CH₃), 2.88 (s, 3H, CH₃), 4.28 (s, 2H, CH₂), 7.04 (d, 1H, J=8.0 Hz, Ph-H), 7.08 (d, 1H, J=5.5 Hz, pyrimidinyl-H), 7.30 (d, 1H, J=8.0 Hz, Ph-H), 7.53 (t, 1H, J=8.0 Hz, Ph-H), 7.94 (s, 1H, Ph-H), 8.45 (d, 1H, J=5.5 Hz, pyrimidinyl-H). MS (ESI⁺) m/z 390.34 [M+H]⁺ (C₁₇H₁₉N₅O₂S₂ requires 389.50).

(3-Bromo-phenyl)-[4-(2-ethylamino-4-methyl-thiazol-5-yl)-pyrimidin-2-yl]-amine (50). By treatment of 3-dimethylamino-1-(2-ethylamino-4-methyl-thiazol-5-yl)-propenone with 3-bromo-phenyl guanidine nitrate. Yellow solid. Anal. RP-HPLC: t_{R}=15.0 min (10-70% MeCN, purity 98%). ¹H-NMR (DMSO-d₆) δ: 1.18 (t, 3H, J=6.5 Hz, CH₃), 2.47 (s, 3H, CH₃), 3.25 (m, 2H, CH₂), 6.94 (d, 1H, J=5.5 Hz, Pyrimidinyl-H), 7.10 (d, 1H, J=8.0 Hz, Ph-H), 7.19 (t, 1H, J=8.0 Hz, Ph-H), 7.61 (d, 1H, J=8.0 Hz, Ph-H), 8.17 (m, 1H, NH), 8.28 (m, 1H, Ph-H), 8.36 (d, 1H, J=5.5 Hz, Pyrimidinyl-H), 9.65 (sbr, 1H, NH). MS (ESI⁺) m/z 390.37 (C_{≠}H₁₆BrN₅S requires 390.30).

[4-(2-tert-Butylamino-4-methyl-thiazol-5-yl)-pyrimidin-2-yl]-(3-nitro-phenyl)-amine (51). By treatment of 1-(2-tert-butylamino-4-methyl-thiazol-5-yl)-3-dimethylamino-propenone with N-(3-nitro-phenyl)-guanidine nitrate. Yellow solid. Anal. RP-HPLC: t_{R}=17.2 min (10-70% MeCN, purity 97%). ¹H-NMR (DMSO-d₆) δ: 1.39 (s, 9H, 3×CH₃), 2.47 (s, 3H, CH₃), 6.98 (d, 1H, J=6.0 Hz, Pyrimidinyl-H), 7.54 (t, 1H, J=8.5 Hz, Ph-H), 7.78 (d, 1H, J=7.5 Hz, Ph-H), 7.94 (sbr, 1H, NH), 8.12 (d, 1H, J=8.5 Hz, Ph-H), 8.40 (d, 1H, J=6.0 Hz, Pyrimidinyl-H), 8.82 (s, 1H, Ph-H), 9.95 (sbr, 1H, NH). MS (ESI⁺) m/z 385.35[M+H]⁺ (C₁₈H₂₀N₆O₂S₂ requires 384.46).

N,N-Diethyl-4-[4-(4-methyl-2-methylamino-thiazol-5-yl)-pyrimidin-2-ylamino]-benzenesulfonamide (52). By treatment of 3-dimethylamino-1-(4-methyl-2-methylamino-thiazol-5-yl)-propenone with N,N-diethyl-4-guanidino-benzene-sulfonamide. Yellow solid. JH-NMR (DMSO-d₆) δ: 1.05 (m, 6H, 2×CH₃), 2.89 (m, 6H, CH₃), 3.14 (m, 4H, CH₂), 7.00 (m, 1H, pyrimidinyl-H), 7.68 (m, 2H, Ph-H), 7.98 (m, 2H, Ph-H), 8.10 (m, 1H, NH), 8.40 (m, 1H, pyrimidinyl-H), 9.95 (s, 1H, NH). MS (ESI⁺) m/z 433.44 [M+H]⁺ (C₁₉H₂₄N₆O₂S₂ requires 432.57).

3-[4-(2-Ethylamino-4-methyl-thiazol-5-yl)-pyrimidin-2-ylamino]N-(2-methoxy-ethyl)-benzenesulfonamide (53). By reaction between 3-dimethylamino-1-(2-ethylamino-4-methyl-thiazol-5-yl)-propenone and 3-guanidino-N-(2-methoxy-ethyl)-benzenesulfon-amide. Light yellow solid. Anal. RP-HPLC: t_{R}=13.9 min (0-60% MeCN, purity 98%). ¹H-NMR (DMSO-d₆) δ: 1.29 (t, 3H, J=7.5 Hz, CH₃), 2.53 (s, 3H, CH₃), 3.09 (t, 2H, J=5.5 Hz, CH₂), 3.24 (s, 3H, CH₃), 3.36-3.41 (m, 4H, CH₂), 6.99 (d, 1H, J=5.5 Hz, pyrimidinyl-H), 7.46 (m, 2H, Ph-H), 7.77 (m, 1H, Ph-H), 8.32 (d, 1H, J=5.0 Hz, pyrimidinyl-H), 8.53 (s, 1H, NH). MS (ESI⁺) m/z 449.37 (C₁₉H₂₄N₆O₃S₂ requires 448.56).

N-(2-Methoxy-ethyl)-3-[4-(4-methyl-2-methylamino-thiazol-5-yl)-pyrimidin-2-ylamino]-benzenesulfonamide (54). By reaction between 3-dimethylamino-1-(4-methyl-2-methylamino-thiazol-5-yl)-propenone and 3-guanidino-N-(2-methoxy-ethyl)-benzene-sulfonamide. Light yellow solid. Mp. 205-206° C. Anal. RP-HPLC: t_{R}=13.1 min (0-60% MeCN; purity 96%). ¹NMR (DMSO-d₆) δ: 2.71 (s, 3H, CH₃), 3.10 (d, 3H, J=4.5 Hz, CH₃), 3.17 (q, 2H, J=6 Hz, CH₂), 3.39 (s, 3H, CH₃), 3.52-3.54 (m, 2H, CH₂), 7.19 (d, 1H, J=5.5 Hz, pyrimidinyl-H), 7.58 (d, 1H, J=8.0 Hz, Ph-H), 7.70 (t, 1H, J=8.0 Hz, Ph-H), 7.83 (t, 1H, J=6.0 Hz, NH), 8.19 (s, 1H, Ph-H), 8.29 (d, 1H, J=5.0 Hz, NH), 8.57 (s, 1H, Ph-H), and 8.60 (d, 1H, J=5.5 Hz, pyrimidinyl-H). MS (ESI⁺) m/z 435.33 (C₁₈H₂₂N₆O₃S₂ requires 434.54).

3-[4-(2-Amino-4-methyl-thiazol-5-yl)-pyrimidin-2-ylamino]-N-(2-methoxy-ethyl)-benzenesulfonamide (55). By reaction between N′-[5-(3-dimethylamino-acryloyl)-4-methyl-thiazol-2-yl]-N,N-dimethyl-formamidine and 3-guanidino-N-(2-methoxy-ethyl)-benzenesulfonamide. Light yellow solid. Mp. 179-180° C. Anal. RP-HPLC: t_{R}=12.6 min (0-60% MeCN; purity 100%). ¹NMR (DMSO-d₆) δ: 2.71 (s, 3H, CH₃), 3.20 (q, 2H, J=6.0 Hz, CH₂), 3.42 (s, 3H, CH₃), 3.56-3.57 (m, 2H, CH₂), 7.18 (d, 1H, J=5.5 Hz, pyrimidinyl-H), 7.60 (d, 1H, J=7.5 Hz, Ph-H), 7.73 (t, 1H, J=7.5 Hz, Ph-H), 7.78 (s, 1H, Ph-H), 7.88 (t, 1H, J=6.0 Hz, NH), 8.35 (d, 1H, J=8.0 Hz, Ph-H), 8.42 (s, 1H, NH), 8.62 (d, 1H, J=5.5 Hz, pyrimidinyl-H). MS (ESI⁺) m/z 421.47 (C₁₇H₂₀N₆O₃S₂ requires 420.51).

3-[4-(2,4-Dimethyl-thiazol-5-yl)-pyrimidin-2-ylamino]-N-(2-methoxy-ethyl)-benzenesulfonamide (56). By reaction between 3-dimethylamino-1-(2,4-dimethyl-thiazol-5-yl)-propenone and 3-guanidino-N-(2-methoxy-ethyl)-benzenesulfonamide. Light yellow solid. Mp. 197-198° C. Anal. RP-HPLC: t_{R}=16.1 min (0-60% MeCN; purity 100%). ¹NMR (DMSO-d₆) δ: 2.64 (s, 3H, CH₃), 2.65 (s, 3H, CH₃), 2.95 (q, 2H, J=6.0 Hz, CH₂), 3.16 (s, 3H, CH₃), 7.15 (d, 1H, J=5.5 Hz, pyrimidinyl-H), 7.38 (d, 1H, J=7.5 Hz, Ph-H), 7.51 (t, 1H, J=6.0 Hz, NH), 7.96 (d, 1H, J=8.0 Hz, Ph-H), 8.32 (s, 1H, Ph-H), 8.56 (d, 1H, J=5.0 Hz, pyrimidinyl-H). MS (ESI⁺) m/z 420.28 (C₁₈H₂₁N₅O₃S₂ requires 419.52).

1-(4-{4-[4-(2-Amino-4-methyl-thiazol-5-yl)-pyrimidin-2-ylamino]-phenyl}-piperazin-1-yl)-ethanone (57). By reaction between N′-[5-(3-dimethylamino-acryloyl)-4-methyl-thiazol-2-yl]-N,N-dimethyl-formamidine and N-[4-(4-acetyl-piperazin-1-yl)-phenyl]-guanidine. Light yellow solid. Mp 267-269° C. Anal. RP-HPLC: t_{R}=7.2 min (10-70% MeCN; purity 100%). ¹H-NMR (DMSO-d₆) δ: 2.42 (s, 3H, CH₃), 3.00 (m, 2H, CH₂), 3.07 (m, 2H, CH₂), 3.29 (s, 3H, CH₃), 3.58 (m, 4H, CH₂), 6.81 (d, 1H, J=5.5 Hz, pyrimidinyl-H), 6.89 (d, 2H, 9.0 Hz, Ph-H), 7.46 (s, 2H, NH₂), 7.62 (d, 2H, J=8.0 Hz, Ph-H), 8.26 (d, 1H, J= 5.5 Hz, pyrimidinyl-H), 9.19 (br. s, 1H, NH). ¹³C-NMR (DMSO-d₆) δ: 19.10, 21.90, 41.49, 46.31, 49.97, 50.40, 107.01, 117.19, 118.90, 120.69, 134.11, 146.38, 152.43, 158.26, 159.33, 160.38, 168.94, 169.42. MS (ESI⁺) m/z 410.52 (C₂₀H₂₃N₇OS requires 409.51).

1-(4-{4-[4-(2-Ethylamino-4-methyl-thiazol-5-yl)-pyrimidin-2-ylamino]-phenyl}-piperazin-1-yl)-ethanone (58). By reaction between 3-dimethylamino-1-(2-ethylamino-4-methyl-thiazol-5-yl)-propenone and N-[4-(4-acetyl-piperazin-1-yl)-phenyl]-guanidine. Light yellow solid. Mp. 208-209° C. Anal. RP-HPLC: t_{R}=9.11 min (10-70% MeCN; purity 100%). ¹H-NMR (DMSO-d₆) δ: 1.17 (t, 3H, J=4.5 Hz, CH₃), 2.45 (s, 3H, CH₃), 2.99 (m, 2H, CH₂), 3.06 (m, 2H, CH₂), 3.28 (m, 2H, CH₂), 3.32 (s, 3H, CH₃), 3.57 (m, 4H, CH₂), 6.82 (d, 1H, J=6.0 Hz, pyrimidinyl-H), 6.89 (d, 2H, J=9.0 Hz, Ph-H), 7.62 (d, 2H, J=8.0 Hz, Ph-H), 8.05 (m, 1H, NH), 8.26 (d, 1H, J=5.5 Hz, pyrimidinyl —H), 9.18 (br. s, 1H, NH). ¹³C-NMR (DMSO-d₆) δ: 14.99, 19.33, 21.91, 41.48, 46.31, 50.00, 50.42, 106.94, 117.22, 118.39, 120.70, 134.13, 146.40, 152.65, 158.22, 159.29, 160.28, 168.91. MS (ESI⁺) m/z 438.48 (C₂₂H₂₇N₇OS requires 437.56).

[4-(2-Ethylamino-4-methyl-thiazol-5-yl)-pyrimidin-2-yl]-(4-piperazin-1-yl-phenyl)-amine (59). By hydrolysis of 1-(4-{4-[4-(2-ethylamino-4-methyl-thiazol-5-yl)-pyrimidin-2-ylamino]-phenyl}-piperazin-1-yl)-ethanone (58). Light yellow solid. Mp. 245-247° C. Anal. RP-HPLC: t_{R}=7.8 min (10-70% MeCN; purity 100%). ¹H-NMR (DMSO-d₆) δ: 1.16 (m, 3H, CH₃), 2.45 (s, 3H, CH₃), 2.82 (m, 4H, CH₂), 2.96 (m, 4H, CH₂), 3.25 (m, 2H, CH₂), 6.80 (d, 1H, J=5.5 Hz, pyrimidinyl-H), 6.85 (d, 2H, J=6.5 Hz, Ph-H), 7.58 (d, 2H, J=6.5 Hz, Ph-H), 8.05 (m, 1H, NH), 8.26 (d, 1H, J=5.0 Hz, pyrimidinyl-H), 9.13 (s, 1H, NH). MS (ESI⁺) m/z 396.35 (C₂₀H₂₅N₇S requires 395.53).

[4-(4-Benzyl-piperazin-1-yl)-phenyl]-[4-(2-ethylamino-4-methyl-thiazol-5-yl)-pyrimidin-2-yl]-amine (60). By reaction between 3-dimethylamino-1-(2-ethylamino-4-methyl-thiazol-5-yl)-propenone and N-[4-(4-benzyl-piperazin-1-yl)-phenyl]-guanidine. Yellow solid. Anal. RP-HPLC: t_{R}=10.1 min (10-70% MeCN; purity 100%). ¹H-NMR (DMSO-d₆) δ: 1.16 (m, 3H, CH₃), 2.44 (s, 3H, CH₃), 3.07 (m, 4H, CH₂), 3.23-3.35 (m, 6H, CH₂), 3.52 (s, 2H, CH₂), 6.80 (d, 1H, J=5.5 Hz, pyrimidinyl-H), 6.85 (d, 2H, J=9.5 Hz, Ph-H), 7.27 (m, 1H, NH), 7.34 (m, 5H, Ph-H), 7.58 (m, 2H, Ph-H), 8.04 (t, 1H, J=5.5 Hz, NH), 8.26 (d, 1H, J=5.0 Hz, pyrimidinyl-H), 9.14 (s, 1H, NH). MS (ESI⁺) m/z 486.45 (C₂₇H₃₁N₇S requires 485.65).

[4-(2-Amino-4-methyl-thiazol-5-yl)-pyrimidin-2-yl]-(4-piperazin-1-yl-phenyl)-amine (61). By hydrolysis of 1-(4-{4-[4-(2-amino-4-methyl-thiazol-5-yl)-pyrimidin-2-ylamino]-phenyl}-piperazin-1-yl)-ethanone (57). Light yellow solid. Anal. RP-HPLC: t_{R}=7.4 min (10-70% MeCN; purity 100%). ¹NMR (DMSO-d₆) δ: 2.42 (s, 3H, CH₃), 3.86 (m, 4H, CH₂), 2.98 (m, 4H, CH₂), 6.79 (d, 1H, J=5.0 Hz, pyrimidinyl-H), 6.85 (d, 2H, J=7.0 Hz, Ph-H), 7.45 (s, 2H, NH₂), 7.59 (d, 2H, J=7.0 Hz, Ph-H), 8.26 (d, 1H, J=5.5 Hz, pyrimidinyl-H), 9.14 (br. s, 1H, NH). MS (ESI⁺) m/z 368.55 (C₁₈H₂₁N₇S requires 367.47).

(3-{4-[4-(4-Methyl-2-methylamino-thiazol-5-yl)-pyrimidin-2-ylamino]-benzenesulfon-ylamino}-phenyl)-acetic acid ethyl ester (62). By reaction between 3-dimethylamino-1-(4-methyl-2-methylamino-thiazol-5-yl)-propenone and [3-(4-guanidino-benzenesulfonyl-amino)-phenyl]-acetic acid ethyl ester. Yellow solid. Anal. RP-HPLC: t_{R}=17.1 min (0-60% MeCN; purity 100%). ¹H-NMR (DMSO-d₆) δ: 1.17 (t, 3H, J=7.0 Hz, CH₃), 2.56 (s, 3H, CH₃), 2.94 (d, 3H, J=4.0 Hz, CH₃), 3.59 (s, 2H, CH₂), 4.07 (q, 2H, J=7.0 Hz, CH₂), 6.94 (d, 1H, J=7.5 Hz, Ph-H), 7.02 (d, 1H, J=7.0 Hz, Ph-H), 7.08 (m, 2H, Ph-H and pyrimidinyl-H), 7.20 (t, 1H, J=8.0 Hz, Ph-H), 7.69 (d, 1H, J=9.0 Hz, Ph-H), 7.94 (d, 2H, J=9.0 Hz, Ph-H), 8.45 (d, 1H, J=6.0 Hz, pyrimidinyl-H). MS (ESI⁺) m/z 539.36 (C₂₅H₂₆N₆O₄S₂ requires 538.64).

N-Acetyl-3-[4-(4-methyl-2-methylamino-thiazol-5-yl)-pyrimidin-2-ylamino]-benzene-sulfonamide (63). By reaction between 3-dimethylamino-1-(4-methyl-2-methylamino-thiazol-5-yl)-propenone and N-acetyl-3-guanidino-benzenesulfonamide. Yellow solid. Anal. RP-HPLC: t_{R}=12.5 min (0-60% MeCN; purity 97%). ¹H-NMR (DMSO-d₆) δ: 2.56 (s, 3H, CH₃), 2.57 (s, 3H, CH₃), 2.98 (d, 3H, J=2.5 Hz, CH₃), 7.08 (d, 1H, J=5.5 Hz, pyrimidinyl-H), 7.53 (d, 1H, J=8.0 Hz, Ph-H), 7.57 (t, 1H, J=8.0 Hz, Ph-H), 8.08 (d, 1H, J=8.0 Hz, Ph-H), 8.47 (s, 1H, Ph-H), 8.48 (d, 1H, J=5.5 Hz, pyrimidinyl-H). MS (ESI⁺) m/z 419.46 (C₁₇H₁₈N₆O₃S₂ requires 418.50).

N-Acetyl-3-[4-(2-amino-4-methyl-thiazol-5-yl)-pyrimidin-2-ylamino]-benzenesulfonamide (64). By reaction between N1-[5-(3-dimethylamino-acryloyl)-4-methyl-thiazol-2-yl]-N,N-dimethyl-formamidine and N-acetyl-3-guanidino-benzenesulfonamide. Yellow solid. Anal. RP-HPLC: t_{R}=11.9 min (0-60% MeCN; purity 96%). ¹NMR (DMSO-d₆) δ: 2.11 (s, 3H, CH₃), 2.63 (s, 3H, CH₃), 7.11 (d, 1H, J=5.5 Hz, pyrimidinyl-H), 7.62-7.00 (m, 3H, Ph-H and NH), 8.31 (d, 1H, J=8.0 Hz, Ph-H), 8.46 (s, 1H, Ph-H), 8.54 (d, 1H, J=5.5 Hz, pyrimidinyl-H). MS (ESI⁺) m/z 405.42 (C_{≠}H₁₆N₆O₃S₂ requires 404.47).

4-[4-(2-Ethylamino-4-methyl-thiazol-5-yl)-pyrimidin-2-ylamino]-N-(2-hydroxy-ethyl)-benzenesulfonamide (65). By reaction between 3-dimethylamino-1-(2-ethylamino-4-methyl-thiazol-5-yl)-propenone and 4-guanidino-N-(2-hydroxy-ethyl)-benzenesulfon-amide. Light yellow solid. Anal. RP-HPLC: t_{R}=12.2 min (0-60% MeCN; purity 100%). ¹H-NMR (DMSO-d₆) δ: 1.27 (t, 3H, J=7.5 Hz, CH₃), 2.57 (s, 3H, CH₃), 2.86 (q, 2H, J=6.0 Hz, CH₂), 3.38 (m, 2H, CH₂), 4.75 (t, 2H, J=5.5 Hz, CH₂), 7.09 (d, 1H, J=5.5 Hz, pyrimidinyl-H), 7.45 (t, 1H, J=6.0 Hz, OH), 7.77 (d, 2H, J=9.0 Hz, Ph-H), 8.06 (d, 2H, J= 9.0 Hz, Ph-H), 8.24 (t, 1H, J=5.5 Hz, NH), 8.48 (d, 1H, J=5.5 Hz, pyrimidinyl-H). MS (ESI⁺) m/z 435.39 (C₁₈H₂₂N₆O₃S₂ requires 434.54).

4-[4-(2,4-Dimethyl-thiazol-5-yl)-pyrimidin-2-ylamino]-N-ethyl-benzenesulfonamide (66). By reaction between 3-dimethylamino-1-(2,4-dimethyl-thiazol-5-yl)-propenone and N-ethyl-4-guanidino-benzenesulfonamide. Light yellow solid. Anal. RP-HPLC: t_{R}= min (0-60% MeCN; purity 97%). ¹H-NMR (DMSO-d₆) Sr. 0.95 (m, 3H, CH₃), 2.65 (s, 3H, CH₃), 2.44 (m, 2H, CH₂), 2.67 (s, 3H, CH₃), 7.19 (d, 1H, J=5.5 Hz, pyrimidinyl-H), 7.34 (m, 1H, NH), 7.71 (d, 2H, J=8.0 Hz, Ph-H), 7.99 (d, 2H, J=8.0 Hz, Ph-H), 8.59 (d, 1H, 5.5 Hz, pyrimidinyl-H), 10.14 (s, 1H, NH). MS (ESI⁺) m/z 390.37 (C₁₇H₁₉N₅S₂ requires 389.50).

N-(2-Hydroxy-ethyl)-4-[4-(4-methyl-2-methylamino-thiazol-5-yl)-pyrimidin-2-ylamino]-benzenesulfonamide (67). By reaction between 3-dimethylamino-1-(4-methyl-2-methylamino-thiazol-5-yl)-propenone and 4-guanidino-N-(2-hydroxy-ethyl)-benzene-sulfonamide. Light yellow solid. Anal. RP-HPLC: t_{R}=11.6 min (0-60% MeCN; purity 97%). ¹H-NMR (DMSO-d₆) δ: 2.58 (s, 3H, CH₃), 2.86 (q, 2H, J=6.0 Hz, CH₂), 2.97 (d, 3H, J=5.0 Hz, CH₃), 4.75 (t, 2H, J=5.5 Hz, CH₂), 7.09 (d, 1H, J=5.5 Hz, pyrimidinyl-H), 7.45 (t, 1H, J=6.0 Hz, OH), 7.77 (d, 2H, J=9.0 Hz, Ph-H), 8.06 (d, 2H, J=9.0 Hz, Ph-H), 8.19 (m, 1H, NH), 8.48 (d, 1H, J=5.5 Hz, pyrimidinyl-H). MS (ESI⁺) m/z 421.35 (C₁₇H₂₀N₆O₃S₂ requires 420.51).

4-[4-(2-Amino-4-methyl-thiazol-5-yl)-pyrimidin-2-ylamino]-N-(2-hydroxy-ethyl)-benzenesulfonamide (68). By reaction between N′-[5-(3-dimethylamino-acryloyl)-4-methyl-thiazol-2-yl]-N,N-dimethyl-formamidine and 4-guanidino-N-(2-hydroxy-ethyl)-benzenesulfonamide. Light yellow solid. Anal. RP-HPLC: t_{R}=11.3 min (0-60% MeCN; purity 97%). ¹H-NMR (DMSO-d₆) δ: 2.54 (s, 3H, CH₃), 2.85 (q, 2H, J=6.5 Hz, CH₂), 4.75 (t, 2H, J=5.5 Hz, CH₂), 7.07 (d, 1H, J=5.5 Hz, pyrimidinyl-H), 7.45 (t, 1H, J=6.0 Hz, OH), 7.65 (s, 2H, NH₂), 7.76 (d, 2H, J=9.0 Hz, Ph-H), 8.05 (d, 2H, 9.0 Hz, Ph-H), 8.47 (d, 1H, J=5.5 Hz, pyrimidinyl-H). MS (ESI⁺) m/z 407.31 (C₁₆H₁₈N₆O₃S₂ requires 406.48).

4-[4-(2,4-Dimethyl-thiazol-5-yl)-pyrimidin-2-ylamino]-N-(2-hydroxy-ethyl)-benzenesulfon-amide (69). By reaction between 3-dimethylamino-1-(2,4-dimethyl-thiazol-5-yl)-propenone and 4-guanidino-N-(2-hydroxy-ethyl)-benzenesulfonamide. Light yellow solid. Anal. RP-HPLC: t_{R}=10.6 min (0-60% MeCN; purity 99%). ¹H-NMR (DMSO-d₆) δ: 2.52 (s, 3H, CH₃), 2.65 (q, 2H, J=6.5 Hz, CH₂), 4.54 (t, 2H, J=5.5 Hz, CH₂), 7.06 (d, 1H, J=5.0 Hz, pyrimidinyl-H), 7.26 (t, 1H, J=6.0 Hz, OH), 7.59 (d, 2H, J=9.0 Hz, Ph-H), 7.84 (d, 2H, J=9.0 Hz, Ph-H), 8.46 (d, 1H, J=5.0 Hz, pyrimidinyl-H). MS (ESI⁺) m/z 406.36 (C₁₇H₁₉N₆O₃S₂ requires 405.50).

3-[4-(2-Ethylamino-4-methyl-thiazol-5-yl)-pyrimidin-2-ylamino]-N-isopropyl-benzene-sulfonamide (70). By reaction between 3-dimethylamino-1-(2-ethylamino-4-methyl-thiazol-5-yl)-propenone and 3-guanidino-N-isopropyl-benzenesulfonamide. Yellow solid. Anal. RP-HPLC: t_{R}=12.6 min (0-60% MeCN; purity 100%). ¹H-NMR (CDCl₃) δ: 1.06 (d, 6H, J=6.5 Hz, CH₃), 1.29 (m, 3H, CH₃), 2.54 (s, 3H, CH₃), 3.39 (m, 2H, CH₂), 6.90 (d, 1H, J=6.0 Hz, pyrimidinyl-H), 7.42 (d, 1H, J=7.5 Hz, Ph-H), 7.49 (d, 1H, J=9.0 Hz, Ph-H), 7.54 (d, 1H, J=9.0 Hz, Ph-H), 8.27 (d, 1H, J=5.5 Hz, pyrimidinyl-H), 8.59 (s, 1H, Ph-H). MS (ESI⁺) m/z 433.38 (C₁₉H₂₄N₆O₂S₂ requires 432.57).

N-Benzyl-4-[4-(2-ethylamino-4-methyl-thiazol-5-yl)-pyrimidin-2-ylamino]-benzenesulfon-amide (71). By reaction between 3-dimethylamino-1-(2-ethylamino-4-methyl-thiazol-5-yl)-propenone and N-benzyl-4-guanidino-benzenesulfonamide. Yellow solid. Anal. RP-HPLC: t_{R}=17.4 min (0-60% MeCN; purity 99%). ¹NMR (DMSO-d₆) δ: 1.26 (t, 3H, J=7.0 Hz, CH₃), 2.57 (s, 3H, CH₃), 3.38 (m, 2H, CH₂), 4.04 (d, 2H, J=6.5 Hz, CH₂), 7.09 (d, 1H, J=5.5 Hz, pyrimidinyl-H), 7.34 (m, 5H, Ph-H), 7.79 (d, 2H, J=9.0 Hz, Ph-H), 8.01 (t, 1H, J=6.5 Hz, NH), 8.05 (d, 1H, J=9.0 Hz, Ph-H), 8.24 (t, 1H, J=5.5 Hz, NH), 8.48 (d, 1H, J=5.5 Hz, pyrimidinyl-H). MS (ESI⁺) m/z 481.35 (C₂₃H₂₄N₆O₂S₂ requires 480.61).

N-Benzyl-4-[4-(4-methyl-2-methylamino-thiazol-5-yl)-pyrimidin-2-ylamino]-benzene-sulfonamide (72). By reaction between 3-dimethylamino-1-(4-methyl-2-methylamino-thiazol-5-yl)-propenone and N-benzyl-4-guanidino-benzenesulfonamide. Yellow solid. Anal. RP-HPLC: t_{R}=16.6 min (0-60% MeCN; purity 99%). ¹H-NMR (DMSO-d₆)<S 2.57 (s, 3H, CH₃), 2.95 (d, 3H, J=4.5 Hz, CH₃), 4.02 (d, 2H, J=6.0 Hz, CH₂), 7.08 (d, 1H, J=5.5 Hz, pyrimidinyl-H), 7.35 (m, 5H, Ph-H), 7.78 (d, 2H, J=9.0 Hz, Ph-H), 8.00 (t, 1H, J=6.5 Hz, NH), 8.04 (d, 1H, J=9.0 Hz, Ph-H), 8.18 (m, 1H, NH), 8.47 (d, 1H, J=5.5 Hz, pyrimidinyl-H). MS (ESI⁺) m/z 467.54 (C₂₂H₂₂N₆O₂S₂ requires 466.58).

4-[4-(2-Amino-4-methyl-thiazol-5-yl)-pyrimidin-2-ylamino]-benzene-(73). By reaction between N′-[5-(3-dimethylamino-acryloyl)-4-methyl-thiazol-2-yl]-N,N-dimethyl-formamidine and N-benzyl-4-guanidino-benzenesulfonamide. Yellow solid. Anal. RP-HPLC: t_{R}=16.2 min (0-60% MeCN; purity 100%). ¹H-NMR (DMSO-d₆) δ: 2.53 (s, 3H, CH₃), 4.03 (d, 2H, J=6.5 Hz, CH₂), 7.07 (d, 1H, J=5.5 Hz, pyrimidinyl-H), 7.36 (m, 5H, Ph-H), 7.64 (s, 2H, NH₂), 7.78 (d, 2H, J=8.5 Hz, Ph-H), 8.00 (t, 1H, J=6.5 Hz, NH), 8.04 (d, 2H, J=9.0 Hz, Ph-H), 8.47 (d, 1H, J=5.5 Hz, pyrimidinyl-H). MS (ESI⁺) m/z 453.33 (C₂₁H₂₀N₆O₂S₂ requires 452.55).

N-Benzyl-4-[4-(2,4-dimethyl-thiazol-5-yl)-pyrimidin-2-ylamino]-benzenesulfonamide (74). By reaction between 3-dimethylamino-1-(2,4-dimethyl-thiazol-5-yl)-propenone and N-benzyl-4-guanidino-benzenesulfonamide. Yellow solid. Anal. RP-HPLC: t_{R}=20.7 min (0-60% MeCN; purity 100%). ¹H-NMR (DMSO-d₆) δ: 2.51 (s, 3H, CH₃), 2.53 (s, 3H, CH₃), 3.82 (d, 2H, J=6.5 Hz, CH₂), 7.07 (d, 1H, J=5.5 Hz, pyrimidinyl-H), 7.60 (d, 2H, J=9.0 Hz, Ph-H), 7.80 (t, 1H, J=6.5 Hz, NH), 7.83 (d, 2H, J=9.0 Hz, Ph-H), 8.47 (d, 1H, J=5.0 Hz, pyrimidinyl-H). MS (ESI⁺) m/z 452.26 (C₂₂H₂₁N₅O₂S₂ requires 451.57).

3-[4-(2-Ethylamino-4-methyl-thiazol-5-yl)-pyrimidin-2-ylamino]-N-(2-hydroxy-ethyl)-benzenesulfonamide (75). By reaction between 3-dimethylamino-1-(2-ethylamino-4-methyl-thiazol-5-yl)-propenone and 3-guanidino-N-(2-hydroxy-ethyl)-benzenesulfon-amide. Yellow solid. Mp. 124-125° C. Anal. RP-HPLC: t_{R}=12.5 min (0-60% MeCN; purity 100%). ¹H-NMR (CDCl₃) δ: 1.41 (t, 3H, J=7.0 Hz, CH₃), 2.71 (s, 3H, CH₃), 3.07 (q, 2H, J=6.5 Hz, CH₂), 3.48-3.53 (m, 2H, CH₂), 3.61 (q, 2H, J=6.5 Hz, CH₂), 7.19 (d, 1H, J=5.5 Hz, pyrimidinyl-H), 7.58 (d, 1H, J=7.5 Hz, Ph-H), 7.69-7.73 (m, 2H, Ph-H and NH), 8.21 (t, 1H, J=7.5 Hz, Ph-H), 8.35 (t, 1H, J=5.5 Hz, Ph-H), 8.55 (s, 1H, Ph-H), and 8.60 (d, 1H, J=5.5 Hz, pyrimidinyl-H). MS (ESI⁺) m/z 435.37 (C₁₈H₂₂N₆O₃S₂ requires 434.54).

N-(2-Hydroxy-ethyl)-3-[4-(4-methyl-2-methylamino-thiazol-5-yl)-pyrimidin-2-ylamino]-benzenesulfonamide (76). By reaction between 3-dimethylamino-1-(4-methyl-2-methylamino-thiazol-5-yl)-propenone and 3-guanidino-N-(2-hydroxy-ethyl)-benzene-sulfonamide. Yellow solid. Mp. 189-190° C. Anal. RP-HPLC: t_{R}=11.8 min (0-60% MeCN; purity 100%). ¹H-NMR (CDCl₃) δ: 2.71 (s, 3H, CH₃), 3.06 (q, 2H, J=6.5, 12.5 Hz, CH₂), 3.09 (d, 3H, J=5.0 Hz, CH₃), 3.58-3.62 (m, 2H, CH₂), 7.18 (d, 1H, 5.5 Hz, pyrimidinyl-H), 7.58 (d, 1H, J=7.5 Hz, Ph-H), 7.69-7.72 (m, 2H, Ph-H and NH), 8.19 (d, 1H, J=8.0 Hz, Ph-H), 8.28 (q, 1H, J=4.5 Hz, OH), 8.56 (s, 1H, Ph-H), 8.59 (d, 1H, J=5.5 Hz, pyrimidinyl-H). MS (ESI⁺) m/z 421.39 (C₁₇H₂₀N₆O₃S₂ requires 420.51).

3-[4-(2-Amino-4-methyl-thiazol-5-yl)-pyrimidin-2-ylamino]-N-(2_hydroxy-ethyl)-benzene-sulfonamide (77). By reaction between N′-[5-(3-dimethylamino-acryloyl)-4-methyl-thiazol-2-yl]-N,N-dimethyl-formamidine and 3-guanidino-N-(2-hydroxy-ethyl)-benzene-sulfonamide. Yellow solid. Mp. 150-151° C. Anal. RP-HPLC: t_{R}: 11.4 min (0-60% MeCN; purity 100%). ¹H-NMR (CDCl₃) δ: 2.71 (s, 3H, CH₃), 3.09 (q, 2H, J=6.5, 12.5 Hz, CH₂), 3.63 (q, 2H, J=6.5, 12.0 Hz, CH₂), 7.18 (d, 1H, J=5.5 Hz, pyrimidinyl-H), 7.60 (d, 1H, J=8.0 Hz, Ph-H), 7.73 (t, 1H, J=8.0 Hz, Ph-H), 7.75-7.77 (m, 2H, Ph-H and NH), 8.42 (s, 1H, Ph-H), and 8.61 (d, 1H, J=5.5 Hz, pyrimidinyl-H). MS (ESI⁺) m/z 407.35 (C₁₆H₁₈N₆O₃S₂ requires 406.48).

3-[4-(2,4-Dimethyl-thiazol-5-yl)-pyrimidin-2-ylamino]-N-(2-hydroxy-ethyl)-benzenesulfon-amide (78). By reaction between 3-dimethylamino-1-(2,4-dimethyl-thiazol-5-yl)-propenone and 3-guanidino-N-(2-hydroxy-ethyl)-benzene-sulfonamide. Yellow solid. Mp. 184-186° C. Anal. RP-HPLC: t_{R}=13.6 min (0-60% MeCN; purity 100%). ¹H-NMR (CDCl₃) δ: 2.64 (s, 3H, CH₃), 2.84 (q, 2H, J=6.5, 12.5 Hz, CH₂), 3.31 (s, 3H, CH₃), 3.38 (q, 2H, J=6.5, 12.0 Hz, CH₂), 4.66 (t, 1H, J=5.5 Hz, NH), 7.15 (d, 1H, J=5.0 Hz, pyrimidinyl-H), 7.5 (d, 1H, J=7.5 Hz, Ph-H), 7.50-7.53 (m, 2H, Ph-H), and NH), 7.97 (d, 1H, J=8.0 Hz, Ph-H), 8.32 (s, 1H, Ph-H), and 8.56 (d, 1H, J=5.0 Hz, pyrimidinyl-H). MS (ESI⁺) m/z 406.42 (C₁₇H₁₉N₅O₃S₂ requires 405.50).

[4-(2-Ethylamino-4-methyl-thiazol-5-yl)-pyrimidin-2-yl]pyridin-3-ylmethyl-amine (79). By reaction between 3-dimethylamino-1-(2-ethylamino-4-methyl-thiazol-5-yl)-propenone and N-pyridin-3-ylmethyl-guanidine. Yellow solid. Anal. RP-HPLC: t_{R}=5.48 min (10-70% MeCN; purity 98%). ¹NMR (CDCl₃) d. 1.15 (t, 3H, J=7.0 Hz, CH₃), 2.38 (s, 3H, CH₃), 3.23 (m, 2H, CH₂), 4.47 (d, 2H, J=6.5 Hz, CH₂), 6.65 (d, 1H, J=5.0 Hz, pyrimidinyl-H), 7.31 (m, 1H, pyridyl-H), 7.72 (d, 1H, J=7.5 Hz, pyridyl-H), 7.98 (t, 1H, J= 5.5 Hz, pyridyl-H), 8.14 (d, 1H, J=5.5 Hz, pyrimidinyl-H), 8.40 (m, 1H, pyridyl-H), 8.55 (s, 1H, NH). MS (ESI⁺) m/z 327.43 (C₁₆H₁₈N₆S requires 326.42).

N-Benzyl-3-[4-(2-ethylamino-4-methyl-thiazol-5-yl)-pyrimidin-2-ylamino]-benzenesulfon-amide (80). By reaction between 3-dimethylamino-1-(2-ethylamino-4-methyl-thiazol-5-yl)-propenone and N-benzyl-3-guanidino-benzenesulfonamide. Yellow solid. Mp. 204-205° C. Anal. RP-HPLC: t_{R}=17.3 min (10-70% MeCN; purity 100%). ¹H-NMR (DMSO-ds) d. 1.39 (t, 3H, J=7.0 Hz, CH₃), 2.71 (s, 3H, CH₃), 3.45-3.51 (m, 2H, CH₂), 4.25 (s, 2H, CH₂), 7.19 (d, 1H, J=5.5 Hz, pyrimidinyl-H), 7.45-7.61 (m, 5H, Ph-H), 7.60 (d, 1H, J=7.5 Hz, Ph-H), 7.7 (t, 1H, J=8.0 Hz, Ph-H), 8.2 (d, 1H, J=8.0 Hz, Ph-H), 8.29 (bs, 1H, NH), 8.34 (t, 1H, J=5.0 Hz, Ph-H), and 8.6 (d, 1H, J=5.0 Hz, pyrimidinyl-H). MS (ESI⁺) m/z 480.83 (C₂₃H₂₄N₆O₂S₂ requires 480.61).

[4-(2-Amino-4-methyl-thiazol-5-yl)-pyrimidin-2-yl]-[3-(morpholine-4-sulfonyl)-phenyl]-amine (81). By reaction between N′-[5-(3-dimethylamino-acryloyl)-4-methyl-thiazol-2-yl]-N,N-dimethyl-formamidine and N-[3-(morpholine-4-sulfonyl)-phenyl]-guanidine. Yellow solid. Mp. 215-216° C. Anal. RP-HPLC: t_{R}=17.4 min (10-70% MeCN; purity 100%). ¹H-NMR (DMSO-d₆) δ: 2.57 (s, 3H, CH₃), 2.89 (m, 4H, CH₂), 3.63 (m, 4H, CH₂), 7.04 (d, 1H, J=5.0 Hz, pyrimidinyl-H), 7.29 (d, 1H, J=7.5 Hz, Ph-H), 7.58 (t, 1H, J=8.0 Hz, Ph-H), 8.06 (d, 1H, J=8.0 Hz, Ph-H), 8.23 (s, 1H, Ph-H), 8.49 (d, 1H, J=5.0 Hz, pyrimidinyl-H). MS (ESI⁺) m/z 433.48 (C₁₈H₂₀N₆O₃S₂ requires 432.52).

[4-(2,4-Dimethyl-thiazol-5-yl)-pyrimidin-2-yl]-[4-methyl-3-(morpholine-4-sulfonyl)-phenyl]-amine (82). By reaction between 3-dimethylamino-1-(2,4-dimethyl-thiazol-5-yl)-propenone and N-[4-methyl-3-(morpholine-4-sulfonyl)-phenyl]-guanidine. Yellow solid. Mp. 81-83° C. Anal. RP-HPLC: t_{R}=18.9 min (10-70% MeCN; purity 100%). ¹H-NMR (DMSO-d₆) δ: 2.58 (s, 3H, CH₃), 2.71 (d, 6H, J=6.0 Hz, CH₃), 3.13 (t, 4H, J=4.5 Hz, CH₂), 3.7 (t, 4H, J=4.5 Hz, CH₂), 7.2 (d, 1H, J=5.5 Hz, pyrimidinyl-H), 7.46 (d, 1H, J=8.5 Hz, Ph-H), 8.08 (d, 1H, J=8.0 Hz, Ph-H), 8.29 (s, 1H, Ph-H), and 8.61 (d, 1H, J=5.0 Hz, pyrimidinyl-H). MS (ESI⁺) m/z 446.41 (C₂₀H₂₃N₅O₃S₂ requires 445.56).

3-{4-[2-(2-Methoxy-ethylamino)-4-methyl-thiazol-5-yl]-pyrimidin-2-ylamino}-benzene-sulfonamide (83). By reaction between 3-dimethylamino-1-[2-(2-methoxy-ethylamino)-4-methyl-thiazol-5-yl]-propenone and 3-guanidino-benzenesulfonamide. Yellow solid. Anal. RP-HPLC: t_{R}=9.77 min (10-70% MeCN; purity 100%). ¹H-NMR (DMSO-d₆) δ: 2.49 (s, 3H, CH₃), 3.27 (s, 3H, CH₃), 3.44 (t, 2H, J=6.0 Hz, CH₂), 3.50 (t, 2H, J=6.0 Hz, CH₂), 6.94 (d, 1H, J=5.5 Hz, pyrimidinyl-H), 7.27 (s, 2H, NH₂), 7.38 (m, 1H, Ph-H), 7.45 (t, 1H, J=8.0 Hz, Ph-H), 7.95 (m, 1H, Ph-H), 8.18 (m, 1H, Ph-H), 8.30 (s, 1H, NH), 8.36 (d, 1H, J=5.5 Hz, pyrimidinyl-H), 9.75 (s, 1H, NH). MS (ESI⁺) m/z 421.35 (C₁₇H₂₀N₆O₃S₂ requires 420.51).

3-[4-(2-Ethylamino-4-methyl-thiazol-5-yl)-pyrimidin-2-ylamino]-N-(2-hydroxy-1,1-dimethyl-ethyl)-benzenesulfonamide (84). By reaction between 3-dimethylamino-1-(2-ethylamino-4-methyl-thiazol-5-yl)-propenone and 3-guanidino-N-(2-hydroxy-1,1-dimethyl-ethyl)-benzenesulfonamide. Yellow solid. Anal. RP-HPLC: t_{R}=11.1 min (10-70% MeCN; purity 100%). ¹H-NMR (DMSO-d₆) δ: 1.17 (m, 9H, CH₃), 2.48 (s, 3H, CH₃), 3.22 (m, 2H, CH₂), 3.26 (m, 2H, CH₂), 3.50 (t, 2H, J=6.0 Hz, CH₂), 6.94 (d, 1H, J=5.5 Hz, pyrimidinyl-H), 7.17 (s, 1H, NH), 7.38-7.46 (m, 2H, Ph-H), 7.94 (m, 1H, Ph-H), 8.11 (m, 1H, Ph-H), 8.32 (br. s, 1H, OH), 8.35 (d, 1H, J=5.5 Hz, pyrimidinyl-H), 9.74 (s, 1H, NH). MS (ESI⁺) m/z 463.47 (C₂₀H₂₆N₆O₃S₂ requires 462.59).

4-(4-Methyl-2-methylamino-thiazol-5-yl)-pyrimidin-2-ylamine (85). By reaction between 3-dimethylamino-1-(4-methyl-2-methylamino-thiazol-5-yl)-propenone and guanidine. Yellow solid. Anal. RP-HPLC: t_{R}=20.7 min (0-60% MeCN; purity 100%). ¹H-NMR (CDCl₃) δ: 2.42 (s, 3H, CH₃), 2.81 (d, 3H, J=4.5 Hz, CH₃), 6.41 (s, 2H, NH₂), 6.64 (d, 1H, J=5.5 Hz, pyrimidinyl-H), 7.89 (m, 1H, NH), 8.10 (d, 1H, J=5.5 Hz, pyrimidinyl-H). MS (ESI⁺) m/z 222.32 (C₉H₁₁N₅S₂ requires 221.28).

4-(2-Ethylamino-4-methyl-thiazol-5-yl)-pyrimidin-2-ylamine (86). By reaction between 3-dimethylamino-1-(2-ethylamino-4-methyl-thiazol-5-yl)-propenone and guanidine. Yellow solid. Anal. RP-HPLC: t_{R}=6.0 min (10-70% MeCN; purity 100%). ¹H-NMR (CDCl₃) δ: 1.15 (t, 3H, J=7.5 Hz, CH₃), 2.41 (s, 3H, CH₃), 3.22 (m, 2H, CH₂), 6.40 (s, 2H, NH₂), 6.63 (d, 1H, J=5.5 Hz, pyrimidinyl-H), 8.10 (d, 1H, J=5.5 Hz, pyrimidinyl-H). MS (ESI⁺) m/z 234.24 (C₁₀H₁₃N₅S requires 235.31).

N-[5-(2-Amino-pyrimidin-4-yl)-4-methyl-thiazol-2-yl]-N-ethyl-acetamide (87). By reaction between N-[5-(3-dimethylamino-acryloyl)-4-methyl-thiazol-2-yl]-N-ethyl-aceta and guanidine. Yellow solid. Anal. RP-HPLC: t_{R}=10.3 min (10-70% MeCN; purity 100%). ¹NMR (CDCl₃) δ: 1.28 (t, 3H, J=7.0 Hz, CH₃), 2.41 (s, 3H, CH₃), 2.57 (s, 3H, CH₃), 4.18 (m, 2H, CH₂), 6.64 (s, 2H, NH₂), 6.80 (d, 1H, J=5.5 Hz, pyrimidinyl-H), 8.23 (d, 1H, J=5.5 Hz, pyrimidinyl-H). MS (ESI⁺) m/z 278.46 (C₁₂H₁₅N₅OS requires 277.35).

4-(2-Dimethylamino-4-methyl-thiazol-5-yl)-pyrimidine-2-ylamine (88). By reaction between 3-dimethylamino-1-(2-dimethylamino-4-methyl-thiazol-5-yl)-propenone and guanidine. Yellow solid. Anal. RP-HPLC: t_{R}=6.3 min (10-70% MeCN; purity 100%). ¹H-NMR (CDCl₃) δ: 2.44 (s, 3H, CH₃), 3.06 (s, 6H, CH₃), 6.43 (s, 2H, NH₂), 6.66 (d, 1H, J=5.5 Hz, pyrimidinyl-H), 8.12 (d, 1H, J=5.5 Hz, pyrimidinyl-H). MS (ESI⁺) m/z 234.67 (C₁₀H₁₃N₅S requires 235.31).

4-Chloromethyl-N-[4-(2-dimethylamino-4-methyl-thiazol-5-yl)-pyrimidin-2-yl]-benzamide (89). By reaction between 3-dimethylamino-1-(2-dimethylamino-4-methyl-thiazol-5-yl)-propenone and N-(4-chloromethyl-benzoyl)-guanidine. Yellow solid. Anal. RP-HPLC: t_{R}=13.3 min (10-70% MeCN; purity 100%). ¹NMR (CHCl₃) δ: 2.63 (s, 3H, CH₃), 3.16 (s, 6H, CH₃), 4.63 (s, 2H, CH₂), 7.06 (d, 1H, J=5.5 Hz, pyrimidinyl-H), 7.51 (d, 2H, J=8.0 Hz, Ph-H), 7.93 (d, 1H, J=8.0 Hz, Ph-H), 8.46 (d, 1H, J=5.5 Hz, pyrimidinyl-H). MS (ESI⁺) m/z 387.90 (C₁₈H₁₈ClN₅OS requires 387.89).

(3-Aminomethyl-phenyl)-[4-(2,4-dimethyl-thiazol-5-yl)-pyrimidin-2-yl]-amine (90). By reaction between 3-dimethylamino-1-(2,4-dimethyl-thiazol-5-yl)-propenone and N-(3-guanidino-benzyl)-acetamide. Yellow solid. Mp. 183-184° C. Anal. RP-HPLC: t_{R}=12.0 min (0-60% MeCN; purity 100%). ¹H-NMR (CHCl₃) δ: 2.82 (s, 3H, CH₃), 2.84 (s, 3H, CH₃), 4.17 (q, 2H, J=6.0, 11.5 Hz, CH₂), 7.29 (m, 2H, Ph-H and pyrimidinyl-H), 7.54 (t, 1H, J=8.0 Hz, Ph-H), 7.92 (d, 1H, J=7.5 Hz, Ph-H), 8.01 (s, 1H, Ph-H), 8.5 (br. s, 2H, NH₂), and 8.71 (d, 1H, J=5.0 Hz, pyrimidinyl-H). MS (ESI⁺) m/z 312.31 (C₁₆H₁₇N₅S requires 311.41).

Pyridine-2-carboxylic acid 3-[4-(2,4-dimethyl-thiazol-5-yl)-pyrimidin-2-ylamino]-benzyl-amide (91). By reaction between 3-dimethylamino-1-(2,4-dimethyl-thiazol-5-yl)-propenone and pyridine-2-carboxylic acid 3-guanidino-benzylamide. Yellow solid. Anal. RP-HPLC: t_{R}=17.8 min (0-60% MeCN; purity 95%). ¹H-NMR (CHCl₃) δ: 2.62 (s, 3H, CH₃), 2.63 (s, 3H, CH₃), 4.52 (d, 2H, J=6.5 Hz, CH₂), 6.62 (d, 1H, J=8.5 Hz, Ar—H), 6.95 (d, 1H, J=7.5 Hz, Ph-H), 7.06 (d, 1H, J=5.5 Hz, pyrimidinyl-H), 7.25 (t, 1H, J=8.0 Hz, Ph-H), 7.41 (d, 1H, J=8.5 Hz, Ar—H), 7.61 (m, 1H, Ph-H), 7.75 (s, 1H, Ph-H), 8.01 (t, 1H, J=7.5 Hz, Ar—H), 8.07 (m, 1H, Ar—H), 8.48 (d, 1H, J=5.0 Hz, pyrimidinyl-H), 8.64 (d, 1H, J=9.0 Hz, NH), 9.22 (d, 1H, J=6.0 Hz, NH). MS (ESI⁺) m/z 417.42 (C₂₂H₂₀N₆OS requires 416.50).

2-(4-Chloro-phenyl)-N-[4-(2-dimethylamino-4-methyl-thiazol-5-yl)-pyrimidin-2-yl]-acetamide (92). By reaction between 3-dimethylamino-1-(2-dimethylamino-4-methyl-thiazol-5-yl)-propenone and N-[2-(4-chloro-phenyl)-acetyl]-guanidine. Yellow solid. Anal. RP-HPLC: t=13.8 min (10-70% MeCN; purity 100%). ¹H-NMR (CDCl₃) δ: 2.54 (s, 3H, CH₃), 3.08 (s, 6H, CH₃), 3.85 (s, 2H, CH₂), 7.14 (d, 1H, J=6.0 Hz, pyrimidinyl-H), 7.31 (m, 4H, Ph-H), 8.46 (d, 1H, J=5.5 Hz, pyrimidinyl-H), 10.60 (s, 1H, NH). MS (ESI⁺) m/z 388.25 (C₁₈H₁₈ClN₅OS requires 387.89).

N-[4-(2-Dimethylamino-4-methyl-thiazol-5-yl)-pyrimidin-2-yl]-2-(4-nitro-phenyl)-acetamide (93). By reaction between 3-dimethylamino-1-(2-dimethylamino-4-methyl-thiazol-5-yl)-propenone and N-[2-(4-nitro-phenyl)-acetyl]-guanidine. Yellow solid. 1H-NMR (CDCl₃) δ: 2.59 (s, 3H, CH₃), 3.16 (s, 6H, CH₃), 3.23 (s, 2H, CH₂), 7.06 (d, 1H, J=6.0 Hz, pyrimidinyl-H), 7.53 (d, 2H, J=9.0 Hz, Ph-H), 8.20 (d, 2H, J=9.0 Hz, Ph-H), 8.38 (d, 1H, J=5.5 Hz, pyrimidinyl-H). MS (ESI⁺) m/z 399.22 (C₁₈H₁₈N₆O₃S requires 398.44).

N-[4-(2-Dimethylamino-4-methyl-thiazol-5-yl)-pyrimidin-2-yl]-2-(4-methoxy-phenyl)-acetamide (94). By reaction between 3-dimethylamino-1-(2-dimethylamino-4-methyl-thiazol-5-yl)-propenone and N-[2-(4-methoxy-phenyl)-acetyl]-guanidine. Yellow solid. Anal. RP-HPLC: t_{R}=12.5 min (10-70% MeCN; purity 100%). ¹H-NMR (DMSO-d₆) δ: 2.59 (s, 3H, CH₃), 3.16 (s, 6H, CH₃), 3.81 (s, 3H, CH₃), 4.05 (s, 2H, CH₂), 6.90 (d, 2H, J=9.0 Hz, Ph-H), 7.01 (d, 1H, J=6.0 Hz, pyrimidinyl-H), 7.27 (d, 2H, J=9.0 Hz, Ph-H), 7.88 (s, 1H, NH), 8.38 (d, 1H, J=5.0 Hz, pyrimidinyl-H).

N-[4-(2-Ethylamino-4-methyl-thiazol-5-yl)-pyrimidin-2-yl]-2-(4-methoxy-phenyl)-acetamide (95). By reaction between 3-dimethylamino-1-(2-ethylamino-4-methyl-thiazol-5-yl)-propenone and N-[2-(4-methoxy-phenyl)-acetyl]-guanidine. Yellow solid. Anal. RP-HPLC: t_{R}=12.2 min (10-70% MeCN; purity 100%). ¹NMR (DMSO-d₆) δ: (m, 3H, CH₃), 2.54 (s, 3H, CH₃), 3.32 (m, 2H, CH₂), 3.81 (s, 3H, CH₃), 4.06 (s, 2H, CH₂), 6.90 (d, 2H, J=9.0 Hz, Ph-H), 7.02 (d, 1H, J=5.5 Hz, pyrimidinyl-H), 7.27 (d, 2H, J=9.0 Hz, Ph-H), 8.00 (s, 1H, NH), 8.41 (d, 1H, J=6.0 Hz, pyrimidinyl-H). MS (ESI⁺) m/z 384.19 (C₁₉H₂₁N₅O₂S requires 383.47).

N-[4-(2,4-Dimethyl-thiazol-5-yl)-pyrimidin-2-yl]-2-(4-methoxy-phenyl)acetamide (96). By reaction between 3-dimethylamino-1-(2,4-dimethyl-thiazol-5-yl)-propenone and N-[2-(4-methoxy-phenyl)-acetyl]-guanidine. Yellow solid. Anal. RP-HPLC: t_{R}=12.7 min (10-70% MeCN; purity 100%). ¹H-NMR (DMSO-d₆) δ: 2.70 (s, 3H, CH₃), 2.73 (s, 3H, CH₃), 3.81 (s, 3H, CH₃), 4.05 (s, 2H, CH₂), 6.91 (d, 2H, J=8.0 Hz, Ph-H), 7.16 (d, 1H, J=5.5 Hz, pyrimidinyl-H), 7.27 (d, 2H, J=9.0 Hz, Ph-H), 7.95 (s, 1H, NH), 8.56 (d, 1H, 5.5 Hz, pyrimidinyl-H). MS (ESI⁺) m/z 354.91 (C₁₈H₁₈N₄O₂S requires 354.43).

2-(4-Chloro-phenyl)-N-[4-(2,4-dimethyl-thiazol-5-yl)-pyrimidin-2-yl]-acetamide (97). By reaction between 3-dimethylamino-1-(2,4-dimethyl-thiazol-5-yl)-propenone and N-[2-(4-chloro-phenyl)-acetyl]-guanidine. Yellow solid. Anal. RP-HPLC: t_{R}=15.2 min (10-70% MeCN; purity 100%). ¹NMR (DMSO-d₆) δ: 2.70 (s, 3H, CH₃), 2.73 (s, 3H, CH₃), 4.19 (s, 2H, CH₂), 7.19 (d, 1H, J=5.5 Hz, pyrimidinyl-H), 7.27 (m, 4H, Ph-H), 8.15 (s, 1H, NH), 8.58 (d, 1H, J=5.5 Hz, pyrimidinyl-H). MS (ESI⁺) m/z 357.02 (C₁₇H₁₅ClN₄OS requires 358.85).

N-[4-(2,4-Dimethyl-thiazol-5-yl)-pyrimidin-2-yl]-2-(4-nitro-phenyl)-acetamide (98). By reaction between 3-dimethylamino-1-(2,4-dimethyl-thiazol-5-yl)-propenone and N-[2-(4-nitro-phenyl)-acetyl]-guanidine. Yellow solid. Anal. RP-HPLC: to =13.8 min (10-70% MeCN; purity 100%). ¹NMR (DMSO-d₆) δ: 2.71 (s, 3H, CH₃), 2.73 (s, 3H, CH₃), 4.42 (s, 2H, CH₂), 7.21 (d, 1H, J=5.5 Hz, pyrimidinyl-H), 7.53 (d, 2H, J=9.0 Hz, Ph-H), 8.17 (s, 1H, NH), 8.22 (d, 2H, J=9.0 Hz, Ph-H), 8.59 (d, 1H, J=5.5 Hz, pyrimidinyl-H). MS (ESI⁺) m/z 367.76 (C₁₇H₁₅N₅O₃S requires 369.40).

{4-[2-(2-Ethyl-pyridin-4-yl)-4-methyl-thiazol-5-yl]-pyrimidin-2-yl}-(4-morpholin-4-yl-phenyl)-amine (99). By reaction between 3-dimethylamino-1-[2-(2-ethyl-pyridin-4-yl)-4-methyl-thiazol-5-yl]-propenone and N-(4-morpholin-4-yl-phenyl)-guanidine. Yellow solid. Mp. 214-215° C. Anal. RP-HPLC: t_{R}=11.2 min (20-70% MeCN; purity 100%). ¹H-NMR (DMSO-d₆) δ: 1.77 (t, 3H, J=7.5 Hz, CH₃), 3.25 (s, 3H, CH₃), 3.35 (q, 2H, J=7.5, 15.0 Hz, CH₂), 3.54 (t, 4H, J=5.0 Hz, CH₂), 4.23 (t, 4H, J=5.0 Hz, CH₂), 7.44 (d, 2H, J=9.0 Hz, Ph-H), 7.61 (d, 1H, J=5.5 Hz, pyrimidinyl-H), 8.12 (d, 2H, J=9.0 Hz, Ph-H), 8.21 (m, 1H, Ar—H), 8.27 (s, 1H, Ar—H), 9.02 (d, 1H, J=5.0 Hz, pyrimidinyl-H), 9.12 (d, 1H, J=5.0 Hz, Ar—H). MS (ESI⁺) m/z 458.89 (C₂₅H₂₆N₆OS requires 458.58).

[4-(4-Methyl-2-pyridin-3-yl-thiazol-5-yl)-pyrimidin-2-yl]-(4-morpholin-4-yl-phenyl)-amine (100). By reaction between 3-dimethylamino-1-(4-methyl-2-pyridin-3-yl-thiazol-5-yl)-propenone and N-(4-morpholin-4-yl-phenyl)-guanidine. Yellow solid. Mp. 240-242° C. Anal. RP-HPLC: t_{R}=13.5 min (0-60% MeCN; purity 100%). ¹H-NMR (DMSO-d₆) δ: 2.75 (s, 3H, CH₃), 3.05 (m, 2H, CH₂), 3.74 (m, 4H, CH₂), 6.95 (d, 2H, J=9.0 Hz, Ph-H), 7.11 (d, 1H, J=5.5 Hz, pyrimidinyl-H), 7.57 (dd, 2H, J=5.0, 8.0 Hz, Ar—H), 7.64 (d, 1H, J= 9.0 Hz, Ph-H), 8.34 (d, 1H, J=8.0 Hz, Ar—H), 8.52 (d, 1H, J=5.0 Hz, pyrimidinyl-H), 8.71 (d, 1H, J=5.0 Hz, Ar—H), 9.17 (s, 1H, Ar—H). MS (ESI⁺) m/z 431.07 (C₂₃H₂₂N₆OS requires 430.53). Ar—H), 8.38 (m, 1H, Ar—H), 8.58 (d, 1H, J=5.5 Hz, pyrimidinyl-H), 8.70 (d, 1H, J=5.0 Hz, Ar—H), 9.19 (d, 1H, J=5.0 Hz, NH). MS (ESI⁺) m/z 416.93 (C₂₂H₂₀N₆OS requires 416.50).

4-{4-[2-(2-Ethyl-pyridin-4-yl)-4-methyl-thiazol-5-yl]-pyrimidin-2-ylamino}-N-(2-hydroxy-ethyl)-benzenesulfonamide (102). By reaction between 3-dimethylamino-1-[2-(2-ethyl-pyridin-4-yl)-4-methyl-thiazol-5-yl]-propenone and 4-guanidino-N-(2-hydroxy-ethyl)-benzenesulfonamide. Yellow solid. Mp. 233-234° C. Anal. RP-HPLC: t_{R}=14.6 min (0-60% MeCN; purity 100%). ¹H-NMR (DMSO-d₆) δ: 1.06 (t, 3H, J=7.5 Hz, CH₃), 2.57 (s, 3H, CH₃), 2.61-2.68 (m, 2H, CH₂), 3.05 (m, 2H, CH₂), 3.14 (m, 2H, CH₂), 4.43 (t, 1H, J=5.5 Hz, OH), 7.11 (d, 1H, J=5.5 Hz, pyrimidinyl-H), 7.16 (t, 1H, 6.0 Hz, NH), 7.54 (m, 2H, Ar—H), 7.59 (s, 1H, Ar—H), 7.77 (d, 2H, J=9.0 Hz, Ph-H), 8.42 (t, 1H, J=5.5 Hz, pyrimidinyl-H), 8.45 (d, 1H, J=5.0 Hz, Ar—H). MS (ESI⁺) m/z 497.01 (C₂₃H₂₄N₆O₃S₂ requires 496.61).

N-{4-[4-(4-Methyl-2-pyridin-3-yl-thiazol-5-yl)-pyrimidin-2-ylamino]-benzyl}-acetamide (103). By reaction between 3-dimethylamino-1-(4-methyl-2-pyridin-3-yl-thiazol-5-yl)-propenone and N-(4-guanidino-benzyl)-acetamide. Yellow solid. Mp. 199-201° C. Anal. RP-HPLC: t_{R}=14.1 min (0-60% MeCN; purity 100%). ¹NMR (DMSO-d₆) δ: 1.87 (s, 3H, CH₃), 2.76 (s, 3H, CH₃), 4.21 (d, 2H, J=6.0 Hz, CH₂), 7.18 (d, 1H, J=5.0 Hz, Py-H), 7.23 (d, 2H, J=9.0 Hz, Ph-H), 7.57 (dd, 1H, J=5.0, 8.0 Hz, Ar—H), 7.74 (d, 2H, J=9.0 Hz, Ph-H), 8.27 (t, 1H, J=6.0 Hz, NH), 8.34 (d, 1H, J=5.0 Hz, pyrimidinyl-H), 8.56 (d, 1H, 5.0 Hz, Ar—H), 8.71 (d 1H, J=4.5 Hz, Ar—H), 9.17 (s, 1H, NH). MS (ESI⁺) m/z 416.80 (C₂₂H₂₀N₆OS requires 416.50).

N-(4-{4-[2-(2-Ethyl-pyridin-4-yl)-4-methyl-thiazol-5-yl]-pyrimidin-2-ylamino}-benzyl)-acetamide (104). By reaction between 3-dimethylamino-1-[2-(2-ethyl-pyridin-4-yl)-4-methyl-thiazol-5-yl]-propenone and N-(4-guanidino-benzyl)-acetamide. Yellow solid. Mp. 224-225° C. Anal. RP-HPLC: t_{R}=14.3 min (0-60% MeCN; purity 100%). ¹H-NMR (DMSO-d₆) δ: 1.56 (t, 3H, J=7.5 Hz, CH₃), 2.14 (s, 3H, CH₃), 3.05 (s, 3H, CH₃), 3.14 (q, 2H, J=8.5, 11.5 Hz, CH₂), 4.48 (d, 1H, J=6.0 Hz, CH₂), 7.48 (m, 3H, pyrimidinyl-H and Ph-H), 7.99 (m, 3H, Ar—H and Ph-H), 8.06 (s, 1H, Ar—H), 8.54 (t, 1H, J=6.0 Hz, NH), 8.86 (d, 1H, J=4.5 Hz, pyrimidinyl-H), 8.92 (d, 1H, J=5.0 Hz, Ar—H).

N-(3-{4-[2-(2-Ethyl-pyridin-4-yl)-4-methyl-thiazol-5-yl]-pyrimidin-2-ylamino}benzyl)-acetamide (105). By reaction between 3-dimethylamino-1-[2-(2-ethyl-pyridin-4-yl)-4-methyl-thiazol-5-yl]-propenone and N-(3-guanidino-benzyl)-acetamide. Yellow solid. Mp. 180-182° C. Anal. RP-HPLC: t_{R}=10.8 min (10-70% MeCN; purity 100%). ¹H-NMR (DMSO-d₆) δ: 1.51 (t, 3H, J=7.5 Hz, CH₃), 2.06 (s, 3H, CH₃), 3.00 (s, 3H, CH₃), 3.14 (dd, 2H, J=7.5 Hz, CH₂), 4.51 (d, 1H, J=6.0 Hz, CH₂), 7.12 (d, 1H, J=7.0 Hz, Ph-H), 7.45 (d, 1H, J=5.0 Hz, pyrimidinyl-H), 7.50 (t, 1H, J=8.0 Hz, Ph-H), 7.79 (d, 1H, J=8.0 Hz, Ph-H), 8.02 (s, 1H, Ph-H), 8.16 (d, 1H, J=5.0 Hz, Ar—H), 8.24 (s, 1H, Ar—H), 8.56 (d, 1H, J=4.5 Hz, Ar—H), 8.82 (d, 1H, J=5.0 Hz, pyrimidinyl-H), 8.92 (d, 1H, J=5.5 Hz, NH).

{4-[4-Methyl-2-(6-methyl-pyridin-3-yl)-thiazol-5-yl]-pyrimidin-2-yl}-(4-morpholin-4-yl-phenyl)-amine (106). By reaction between 3-dimethylamino-1-[4-methyl-2-(6-methyl-pyridin-3-yl)-thiazol-5-yl]-propenone and N-(4-morpholin-4-yl-phenyl)-guanidine. Yellow solid. Mp. 244-245° C. Anal. RP-HPLC: t_{R}=9.8 min (10-70% MeCN; purity 100%). ¹NMR (DMSO-d₆) δ: 2.55 (s, 3H, CH₃), 2.74 (s, 3H, CH₃), 3.05 (m, 4H, CH₂), 3.74 (m, 4H, CH₂), 6.95 (d, 2H, J=9.0 Hz, Ph-H), 7.10 (d, 1H, J=5.5 Hz, pyrimidinyl-H), 7.42 (d, 2H, J=8.0 Hz, Ph-H), 7.64 (d, 1H, J=9.0 Hz, Ar—H), 8.23 (d, 1H, J=8.0 Hz, Ar—H), 8.51 (d, 1H, J=5.0 Hz, pyrimidinyl-H), 9.04 (s, 1H, Ar—H), 9.49 (s, 1H, NH).

(4-{2-[3-(2-Methoxy-ethoxy)-5-trifluoromethyl-pyridin-2-yl]-4-methyl-thiazol-5-yl}-pyrimidin-2-yl)-(4-morpholin-4-yl-phenyl)-amine (107). By reaction between 3-dimethyl-ammo-1-{2-[3-(2-methoxy-ethoxy)-5-trifluoromethyl-pyridin-2-yl]-4-methyl-thiazol-5-yl}-propenone and N-(4-morpholin-4-yl-phenyl)-guanidine. Yellow solid. Mp. 175-178° C. Anal. RP-HPLC: t_{R}=13.8 min (10-70% MeCN; purity 100%). ¹NMR (DMSO-d₆) δ: 2.77 (s, 3H, CH₃), 3.06 (m, 4H, CH₂), 3.75 (m, 4H, CH₂), 3.84 (t, 2H, J=4.0 Hz, CH₂), 4.53 (t, 2H, J=4.0 Hz, CH₂), 6.93 (d, 2H, J=8.5 Hz, Ph-H), 7.13 (d, 1H, J=5.0 Hz, pyrimidinyl-H), 7.64 (d, 2H, J=8.5 Hz, Ph-H), 8.10 (s, 1H, Ar—H), 8.52 (d, 1H, J=5.0 Hz, pyrimidinyl-H), 8.69 (s, 1H, Ar—H), 9.49 (s, 1H, NH). MS (ESI⁺) m/z 572.84 (C₂₇H₂₇F₃N₆O₃S requires 572.60).

N-(3-{4-[4-Methyl-2-(6-methyl-pyridin-3-yl)-thiazol-5-yl]-pyrimidin-2-ylamino}-benzyl)-acetamide (108). By reaction between 3-dimethylamino-1-[4-methyl-2-(6-methyl-pyridin-3-yl)-thiazol-5-yl]-propenone and N-(3-guanidino-benzyl)-acetamide. Yellow solid. Mp. 227-229° C. Anal. RP-HPLC: t_{R}=9.6 min (10-70% MeCN; purity 100%). ¹H-NMR (DMSO-d₆) δ: 1.85 (s, 3H, CH₃), 2.54 (s, 3H, CH₃), 2.75 (s, 3H, CH₃), 4.27 (d, 2H, J=6.0 Hz, CH₂), 6.89 (d, 1H, J=7.5 Hz, Ph-H), 7.18 (d, 1H, J=5.0 Hz, pyrimidinyl-H), 7.28 (t, 1H, J=8.0 Hz, Ph-H), 7.41 (d, 1H, J=8.0 Hz, Ph-H), 7.62 (d, 1H, J=8.0 Hz, Ar—H), 7.76 (s, 1H, Ph-H), 8.25 (d, 1H, J=8.0 Hz, Ar—H), 8.32 (t, 1H, J=6.0 Hz, NH), 8.57 (d, 1H, J= 5.0 Hz, pyrimidinyl-H), 9.05 (s, 1H, Ar—H). MS (ESI⁺) m/z 430.96 (C₂₃H₂₂N₆OS requires 430.53).

N-(3-{4-[2-(3-Chloro-5-trifluoromethyl-pyridin-2-yl)-4-methyl-thiazol-5-yl]-pyrimidin-2-ylamino}-benzyl)-acetamide (109). By reaction between 1-[2-(3-chloro-5-trifluoromethyl-pyridin-2-yl)-4-methyl-thiazol-5-yl]-3-dimethylamino-propenone and N-(3-guanidino-benzyl)-acetamide. Yellow solid. Mp 217-218° C. Anal. RP-HPLC: t_{R}=19.8 min (10-70% MeCN; purity 100%). ¹H-NMR (DMSO-d₆) δ: 1.86 (s, 3H, CH₃), 2.80 (s, 3H, CH₃), 4.25 (d, 2H, J=6.0 Hz, CH₂), 6.89 (d, 2H, J=7.5 Hz, Ph-H), 7.25 (m, 2H, pyrimidinyl-H and Ph-H), 7.69 (m, 2H, Ph-H and Ar—H), 8.30 (t, 1H, NH), 8.61 (d, 1H, J=5.0 Hz, pyrimidinyl-H), 8.65 (s, 1H, Ar—H), 9.07 (s, 1H, NH). MS (ESI⁺) m/z 540.88 [M+Na](C₂₃H₁₈ClF₃N₆OS requires 518.94).

N-(2-Methoxy-ethyl)-4-[4-(4-methyl-2-pyridin-3-yl-thiazol-5-yl)-pyrimidin-2-ylamino]-benzenesulfonamide (110). By reaction between 3-dimethylamino-1-(4-methyl-2-pyridin-3-yl-thiazol-5-yl)-propenone and 4-guanidino-N-(2-methoxy-ethyl)-benzenesulfonamide. Yellow solid. Mp 252-254° C. Anal. RP-HPLC: t_{R}=16.5 min (0-60% MeCN; purity 100%). ¹H-NMR (DMSO-d₆) δ: 2.79 (s, 3H, CH₃), 2.90 (q, 2H, J=6.0, 11.5 Hz, CH₃), 3.17 (s, 3H, CH₃), 3.30 (m, 2H, CH₂), 7.32 (d, 1H, J=5.5 Hz, pyrimidinyl-H), 7.51 (t, 1H, J=6.0 Hz, NH), 7.59 (s, 1H, Ar—H), 7.78 (d, 2H, J=9.0 Hz, Ph-H), 8.01 (d, 2H, J=9.0 Hz, Ph-H), 8.38 (d, 1H, J=8.0 Hz, Ar—H), 8.67 (d, 1H, J=5.0 Hz, pyrimidinyl-H), 8.72 (d, 1H, J= 5.0 Hz, Ar—H), 8.21 (s, 1H, Ar—H). MS (ESI⁺) m/z 482.82 (C₂₂H₂₂N₆O₃S₂ requires 482.58).

[4-(2-Ethylamino-4-methyl-thiazol-5-yl)-pyrimidin-2-yl]-(4-methoxy-2-methyl-phenyl)-amine (111). By reaction between 3-dimethylamino-1-(2-ethylamino-4-methyl-thiazol-5-yl)-propenone and N-(4-methoxy-2-methyl-phenyl)-guanidine. Yellow solid. ¹H-NMR (CD₃OD) δ: 1.24 (t, 3H, J=7.5 Hz, CH₃), 2.23 (s, 3H, CH₃), 2.42 (s, 3H, CH₃), 3.31 (m, 2H, CH₂), 3.75 (s, 3H, CH₃), 6.74 (m, 3H, Ph-H and pyrimidinyl-H), 6.80 (s, 1H, Ph-H), 7.33 (d, 2H, J=9.0 Hz, Ph-H), 8.10 (d, 1H, J=6.0 Hz, pyrimidinyl-H). MS (ESI⁺) m/z 355.98 (C₁₈H₂₁N₅OS requires 355.46).

[4-(2,4-Dimethyl-thiazol-5-yl)-pyrimidin-2-yl]-(4-methoxy-2-methyl-phenyl)-amine (112). By reaction between 3-dimethylamino-1-(2,4-dimethyl-thiazol-5-yl)-propenone and N-(4-methoxy-2-methyl-phenyl)-guanidine. Yellow solid. Anal. RP-HPLC: t_{R}=13.7 min (10-70% MeCN; purity 100%). ¹NMR (CD₃OD) δ: 2.21 (s, 3H, CH₃), 2.54 (s, 3H, CH₃), 2.61 (s, 3H, CH₃), 3.77 (s, 3H, CH₃), 6.74 (d, 2H, J=9.0 Hz, Ph-H), 6.80 (s, 1H, Ph-H), 6.85 (d, 1H, J=5.5 Hz, pyrimidinyl-H), 7.30 (d, 2H, J=9.0 Hz, Ph-H), 8.24 (d, 1H, J=5.5 Hz, pyrimidinyl-H). MS (ESI⁺) m/z 323.02 (C₁₇H₁₈N₄OS requires 326.42).

[4-(2,4-Dimethyl-thiazol-5-yl)-pyrimidin-2-yl]-(5-methoxy-2-methyl-phenyl)-amine (113). By reaction between 3-dimethylamino-1-(2,4-dimethyl-thiazol-5-yl)-propenone and N-(5-methoxy-2-methyl-phenyl)-guanidine. Yellow solid. Anal. RP-HPLC: t_{R}=15.6 min (10-70% MeCN; purity 100%). ¹H-NMR (CD₃OD) δ: 2.18 (s, 3H, CH₃), 2.53 (s, 3H, CH₃), 2.58 (s, 3H, CH₃), 3.75 (s, 3H, CH₃), 6.59 (d, 2H, J=9.0 Hz, Ph-H), 6.86 (d, 1H, J=5.5 Hz, pyrimidinyl-H), 7.06 (d, 2H, J=9.0 Hz, Ph-H), 7.37 (d, 1H, J=2.5 Hz, Ph-H), 8.26 (d, 1H, J=5.5 Hz, pyrimidinyl-H). MS (ESI⁺) m/z 326.92 (C₁₇H₁₈N₄OS requires 326.42).

[4-(4-Benzyl-piperazin-1-yl)-phenyl]-[4-(2,4-dimethyl-thiazol-5-yl)-pyrimidin-2-yl]-amine (114). By reaction between 3-dimethylamino-1-(2,4-dimethyl-thiazol-5-yl)-propenone and N-[4-(4-benzyl-piperazin-1-yl)-phenyl]-guanidine. Yellow solid. Anal. RP-HPLC: t_{R}=12.3 min (10-70% MeCN; purity 100%). ¹H-NMR (DMSO-d₆) δ: 2.61 (s, 3H, CH₃), 2.64 (s, 3H, CH₃), 2.52 (m, 4H, CH₂), 3.07 (m, 4H, CH₂), 3.52 (s, 2H, CH₂), 6.88 (d, 2H, J= 7.0 Hz, Ph-H), 6.99 (d, 1H, J=5.5 Hz, pyrimidinyl-H), 7.27 (m, 1H, NH), 7.34 (m, 5H, Ph-H), 7.58 (d, 2H, J=9.0 Hz, Ph-H), 8.44 (d, 1H, J=5.0 Hz, pyrimidinyl-H), 9.38 (s, 1H, NH). MS (ESI⁺) m/z 456.96 (C₂₆H₂₈N₆S requires 456.61).

[4-(2-Ethylamino-4-methyl-thiazol-5-yl)-pyrimidin-2-yl]-(5-methoxy-2-methyl-phenyl)-amine (115). By reaction between 3-dimethylamino-1-(2-ethylamino-4-methyl-thiazol-5-yl)-propenone and N-[4-(4-benzyl-piperazin-1-yl)-phenyl]-guanidine. Yellow solid. Anal. RP-HPLC: t_{R}=13.2 min (10-70% MeCN; purity 98%). ¹H-NMR (DMSO-D₆) δ: 1.15 (t, 3H, J=7.0 Hz, CH₃), 2.16 (s, 3H, CH₃), 2.41 (s, 3H, CH₃), 3.23 (m, 2H, CH₂), 3.73 (s, 3H, CH₃), 6.59 (d, 2H, J=9.0 Hz, Ph-H), 6.82 (d, 1H, J=5.5 Hz, pyrimidinyl-H), 7.07 (d, 2H, J=9.0 Hz, Ph-H), 7.25 (d, 1H, J=2.5 Hz, Ph-H), 8.05 (m, 1H, NH), 8.25 (d, 1H, J=5.5 Hz, pyrimidinyl-H), 8.41 (s, 1H, NH). MS (ESI⁺) m/z 355.98 (C₁₈H₂₁N₅OS requires 355.46).

(3-Aminomethyl-phenyl)-[4-(4-methyl-2-pyridin-3-yl-thiazol-5-yl)pyrimidin-2-yl]-amine (116). By hydrolysis of N-{3-[4-(4-methyl-2-pyridin-3-yl-thiazol-5-yl)-pyrimidin-2-ylamino]-benzyl}-acetamide. Yellow solid. Mp 211-213° C. Anal. RP-HPLC: t_{R}=8.1 min (10-70% MeCN; purity 100%). ¹NMR (DMSO-d₆) δ: 2.79 (s, 3H, CH₃), 4.02 (q, 2H, J=6.0, 11.5 Hz, CH₂), 7.16 (d, 1H, J=7.5 Hz, Ph-H), 7.26 (d, 1H, J=5.0 Hz, pyrimidinyl-H), 7.40 (t, 1H, J=8.0 Hz, Ph-H), 7.73 (d, 1H, J=8.0 Hz, Ph-H), 7.79 (m, 1H, Ar—H), 7.93 (s, 1H, Ph-H), 8.45 (br. s, 2H, NH₂), 8.59 (d, 1H, J=8.0 Hz, Ar—H), 8.62 (d, 1H, J=5.0 Hz, pyrimidinyl-H), 8.81 (t, 1H, J=6.0 Hz, Ar—H), 9.05 (s, 1H, Ar—H). MS (ESI⁺) m/z 375.05 (C₂₀H₁₈N₆S requires 374.46).

[4-(2-Benzylamino-4-methyl-thiazol-5-yl)-pyrimidin-2-yl]-(4-morpholin-4-yl-phenyl)-amine (117). By reaction between 1-(2-benzylamino-4-methyl-thiazol-5-yl)-3-dimethylamino-propenone and N-(4-morpholin-4-yl-phenyl)-guanidine. Yellow solid. Mp 180-183° C. Anal. RP-HPLC: t_{R}=17.1 min (0-60% MeCN; purity 100%). ¹H-NMR (DMSO-d₆) δ: 2.51 (s, 3H, CH₃), 3.09 (m, 4H, CH₂), 3.75 (m, 2H, CH₂), 4.77 (s, 2H, CH₂), 6.90 (d, 1H, J=6.0 Hz, pyrimidinyl-H), 6.97 (d, 2H, J=8.5 Hz, Ph-H), 7.30 (m, 3H, Ph-H), 7.37 (m, 2H, Ph-H and NH), 8.26 (d, 1H, J=5.5 Hz, pyrimidinyl-H). MS (ESI⁺) m/z 458.96 (C₂₅H₂₆N₆OS requires 458.58).

N-{3-[4-(2-Benzylamino-4-methyl-thiazol-5-yl)-pyrimidin-2-ylamino]-benzyl}-acetamide (118). By reaction between 1-(2-benzylamino-4-methyl-thiazol-5-yl)-3-dimethylamino-propenone and N-(3-guanidino-benzyl)-acetamide. Yellow solid. Mp 181-183° C. Anal. RP-HPLC: t_{R}=12.6 min (10-70% MeCN; purity 100%). ¹NMR (DMSO-d₆) δ: 2.37 (s, 3H, CH₃), 3.02 (s, 3H, CH₃), 4.00 (s, 2H, CH₂), 4.57 (d, 2H, J=6.0 Hz, CH₂), 7.28 (d, 1H, J=7.5 Hz, Ph-H), 7.44 (d, 1H, J=5.5 Hz, pyrimidinyl-H), 7.63 (t, 1H, J==7.5 Hz, Ph-H), 7.85 (m, 2H, Ph-H), 7.96 (d, 1H, J=9.5 Hz, Ph-H), 7.99 (m, 3H, Ph-H and NH), 8.18 (s, 1H, Ph-H), 8.73 (t, 1H, J=6.0 Hz, NH), 8.83 (d, 1H, J=5.5 Hz, pyrimidinyl-H). MS (ESI⁺) m/z 444.94 (C₂₄H₂₄N₆OS requires 444.55).

1-(4-{4-[4-(4-Methyl-2-pyridin-3-yl-thiazol-5-yl)-pyrimidin-2-ylamino]-phenyl}-piperazin-1-yl)-ethanone (119). By reaction between 3-dimethylamino-1-(4-methyl-2-pyridin-3-yl-thiazol-5-yl)-propenone and N-[4-(4-acetyl-piperazin-1-yl)-phenyl]-guanidine. Yellow solid. Mp 123-125° C. Anal. RP-HPLC: t_{R}=9.3 min (0-60% MeCN; purity 100%). 1H-NMR (DMSO-d₆) δ: 2.04 (s, 3H, CH₃), 2.76 (s, 3H, CH₃), 3.03 (t, 2H, J=5.0 Hz, CH₂), 3.09 (t, 2H, J=5.0 Hz, CH₂), 3.58 (q, 4H, J=5.5, 10.0 Hz, CH₂), 6.98 (d, 2H, J=9.0 Hz, Ph-H), 7.13 (d, 1H, J=5.0 Hz, pyrimidinyl-H), 7.58 (m, 1H, Ar—H), 7.65 (d, 2H, J=9.0 Hz, Ph-H), 8.35 (d, 1H, J=8.0 Hz, Ar—H), 8.53 (d, 1H, J=5.0 Hz, pyrimidinyl-H), 8.71 (d, 1H, J=5.0 Hz, Ar—H), 9.18 (s, 1H, Ar—H). MS (ESI⁺) m/z 493.99 [M+Na](C₂₅H₂₅N₇OS requires 471.58).

{4-[2-(Ethyl-methyl-amino)-4-methyl-thiazol-5-yl]-pyrimidin-2-yl}-(4-morpholin-4-yl-phenyl)-amine (120). By reaction between 3-dimethylamino-1-[2-(ethyl-methyl-amino)-4-methyl-thiazol-5-yl]-propenone and N-(4-morpholin-4-yl-phenyl)-guanidine. Yellow solid. Anal. RP-HPLC: t_{R}=10.5 min (10-70% MeCN; purity 100%). ¹NMR (DMSO-d₆) δ: 1.26 (t, 3H, J=6.5 Hz, CH₃), 2.57 (s, 3H, CH₃), 3.13 (m, 7H, CH₂ and CH₃), 3.58 (m, 2H, CH₂), 3.88 (m, 4H, CH₂), 6.77 (d, 1H, J=6.0 Hz, pyrimidinyl-H), 6.92 (d, 2H, J=9.0 Hz, Ph-H), 7.52 (d, 2H, J=9.0 Hz, Ph-H), 8.18 (d, 1H, J=6.0 Hz, pyrimidinyl-H). MS (ESI⁺) m/z 409.25 (C₂₁H₂₆N₆OS requires 410.54).

[4-(2,6-Dimethyl-morpholin-4-yl)-phenyl]-[4-(2,4-dimethyl-thiazol-5-yl)-pyrimidin-2-yl]-amine (121). By reaction between 3-dimethylamino-1-(2,4-dimethyl-thiazol-5-yl)-propenone and N-[4-(2,6-dimethyl-morpholin-4-yl)-phenyl]-guanidine. Yellow solid. Anal. RP-HPLC: t_{R}=12.7 min (10-70% MeCN; purity 100%). ¹H-NMR (DMSO-d₆) δ: 2.21 (m, 2H, CH₂), 2.63 (s, 3H, CH₃), 2.65 (s, 3H, CH₃), 3.51 (d, 2H, J=1.0 Hz, CH), 3.70 (m, 2H, CH₂), 6.90 (d, 2H, J=9.5 Hz, Ph-H), 7.00 (d, 1H, J=5.5 Hz, pyrimidinyl-H), 7.60 (d, 2H, J=9.5 Hz, Ph-H), 8.44 (d, 1H, J=5.5 Hz, pyrimidinyl-H), 9.40 9s, 1H, NH).

1-[4-(4-{4-[2-(Benzyl-methyl-amino)-4-methyl-thiazol-5-yl]-pyrimidin-2-ylamino}-phenyl)-piperazin-1-yl]-ethanone (122). By reaction between 1-[2-(benzyl-methyl-amino)-4-methyl-thiazol-5-yl]-3-dimethylamino-propenone and N-[4-(4-acetyl-piperazin-1-yl)-phenyl]-guanidine. Yellow solid. Anal. RP-HPLC: t_{R}=16.9 min (0-60% MeCN; purity 100%). ¹NMR (DMSO-d₆) δ: 2.04 (s, 3H, CH₃), 2.51 (s, 3H, CH₃), 3.06 (m, 2H, CH₂), 3.13 (m, 5H, CH₃ and CH₂), 4.60 (m, 4H, CH₂), 4.77 (s, 2H, CH₂), 6.91 (d, 1H, J=6.0 Hz, pyrimidinyl-H), 6.98 (d, 2H, J=8.5 Hz, Ph-H), 7.30 (m, 3H, Ph-H), 7.37 (m, 2H, Ph-H), 7.59 (d, 2H, J=9.0 Hz, Ph-H), 8.26 (d, 1H, J=5.5 Hz, pyrimidinyl-H). MS (ESI⁺) m/z 514.05 (C₂₈H₃₁N₇OS requires 513.66).

(4-{2-[(3,5-Dichloro-phenyl)-methyl-amino]-4-methyl-thiazol-5-yl}-pyrimidin-2-yl)-(4-morpholin-4-yl-phenyl)-amine (123). By reaction between 1-{2-[(3,5-dichloro-phenyl)-methyl-amino]-4-methyl-thiazol-5-yl}-3-dimethylamino-propenone and N-(4-morpholin-4-yl-phenyl)-guanidine. Yellow solid. Mp 216-218° C. Anal. RP-HPLC: t_{R}=18.8 min (20-70% MeCN; purity 100%). ¹H-NMR (DMSO-d₆) δ: 2.52 (s, 3H, CH₃), 3.02 (t, 4H, J=5.0 Hz, CH₂), 3.51 (s, 3H, CH₃), 3.74 (t, 4H, J=5.0 Hz, CH₂), 6.84 (d, 2H, J=9.0 Hz, Ph-H), 6.91 (d, 1H, J=5.0 Hz, pyrimidinyl-H), 7.57 (m, 3H, Ph-H), 7.72 (s, 2H, Ph-H), 8.34 (d, 1H, J=5.0 Hz, pyrimidinyl-H), 9.26 (s, 1H, NH). MS (ESI⁺) m/z 528.85 (C₂₅H₂₄Cl₂N₆OS requires 527.47).

(4-{2-[(4-Chloro-phenyl)-methyl-amino]-4-methyl-thiazol-5-yl}-pyrimidin-2-yl)-(4-morpholin-4-yl-phenyl)-amine (124). By reaction between 1-{2-[(4-chloro-phenyl)-methyl-amino]-4-methyl-thiazol-5-yl}-3-dimethylamino-propenone and N-(4-morpholin-4-yl-phenyl)-guanidine. Yellow solid. Mp 245-246° C. Anal. RP-HPLC: t_{R}=16.8 min (20-70% MeCN; purity 100%). ¹H-NMR (DMSO-d₆) 2.51 (s, 3H, CH₃), 3.02 (t, 4H, J=5.0 Hz, CH₂), 3.48 (s, 3H, CH₃), 3.75 (t, 4H, J=5.0 Hz, CH₂), 6.79 (d, 2H, J=9.0 Hz, Ph-H), 6.82 (d, 1H, J=5.5 Hz, pyrimidinyl-H), 7.53 (d, 2H, J=9.0 Hz, Ph-H), 7.58 (s, 4H, Ph-H), 8.30 (d, 1H, J=5.0 Hz, pyrimidinyl-H), 9.20 (s, 1H, NH). MS (ESI⁺) m/z 492.83 (C₂₅H₂₅ClN₆OS requires 493.02).

N-[3-(4-{2-[(3,5-Dichloro-phenyl)-methyl-amino]-4-methyl-thiazol-5-yl}-pyrimidin-2-ylamino)-benzyl]-acetamide (125). By reaction between 1-{2-[(3,5-dichloro-phenyl)-methyl-amino]-4-methyl-thiazol-5-yl}-3-dimethylamino-propenone and N-(3-guanidino-benzyl)-acetamide. Yellow solid. Mp 213-214° C. Anal. RP-HPLC: t_{R}=17.8 min (20-70% MeCN; purity 100%). ¹NMR (DMSO-d₆) δ: 2.36 (s, 3H, CH₃), 4.00 (s, 3H, CH₃), 4.64 (d, 2H, J=6.0 Hz, CH₂), 7.31 (d, 1H, J=7.5 Hz, Ph-H), 7.48 (d, 1H, 5.5 Hz, pyrimidinyl-H), 7.68 (t, 1H, J=8.0 Hz, Ph-H), 8.02 (s, 1H, Ph-H), 8.06 (d, 1H, J=8.0 Hz, Ph-H), 8.17 (s, 1H, Ph-H), 8.21 (s, 1H, Ph-H), 8.76 (t, 1H, J=6.0 Hz, NH), 8.89 (d, 1H, J=5.0 Hz, pyrimidinyl-H). MS (ESI⁺) m/z 514.94 (C₂₄H₂₂Cl₂N₆OS requires 513.44).

(3,5-Dichloro-4-morpholin-4-yl-phenyl)-[4-(4-methyl-2-pyridin-3-yl-thiazol-5-yl)-pyrimidin-2-yl]-amine (126). By reaction between 3-dimethylamino-1-(4-methyl-2-pyridin-3-yl-thiazol-5-yl)-propenone and N-(3,5-dichloro-4-morpholin-4-yl-phenyl)-guanidine. Yellow solid. Mp 276-278° C. Anal. RP-HPLC: t_{R}=24.5 min (20-70% MeCN; purity 100%). ¹H-NMR (DMSO-d₆) δ: 2.77 (s, 3H, CH₃), 3.11 (t, 4H, J=4.5 Hz, CH₂), 3.7 (t, 4H, J=4.5 Hz, CH₂), 7.27 (d, 1H, J=5.0 Hz, pyrimidinyl-H), 7.59 (dd, 1H, J=5.0, 8.0 Hz, Ar—H), 7.95 (s, 2H, Ph-H), 8.28 (d, 1H, J=8.0 Hz, Ar—H), 8.63 (d, 1H, 7=5.0 Hz, pyrimidinyl-H), 8.71 (d, 1H, J=5.0 Hz, Ar—H), and 9.12 (s, 1H, Ar—H). MS (ESI⁺) m/z 500.83 (C₂₃H₂₀Cl₂N₆OS requires 499.42).

(3-Chloro-4-morpholin-4-yl-phenyl)-[4-(4-methyl-2-pyridin-3-yl-thiazol-5-yl)-pyrimidin-2-yl]-amine (127). By reaction between 3-dimethylamino-1-(4-methyl-2-pyridin-3-yl-thiazol-5-yl)-propenone and N-(3-chloro-4-morpholin-4-yl-phenyl)-guanidine. Yellow solid. Mp 230-231° C. Anal. RP-HPLC: t_{R}=18.7 min (20-70% MeCN; purity 100%). ¹H-NMR (DMSO-d₆) δ: 2.77 (s, 3H, CH₃), 2.94 (t, 4H, J= 4.5 Hz, CH₂), 3.74 (t, 4H, 7=4.5 Hz, CH₂), 7.18 (d, 2H, J=9.0 Hz, Ph-H), 7.21 (d, 1H, J=5.5 Hz, pyrimidinyl-H), 7.59 (dd, 1H, J=5.0, 8.0 Hz, Ar—H), 7.64 (d, 1H, J=8.5 Hz, Ph-H), 8.08 (s, 1H, Ph-H), 8.32 (d, 1H, J=5.0 Hz, pyrimidinyl-H), 8.59 (s, 1H, Ar—H), 8.71 (d, 1H, J=5.0 Hz, Ar—H), 9.16 (s, 1H, Ar—H). MS (ESI⁺) m/z 465.00 (C₂₃H₂₁ClN₆OS requires 464.97).

(3-Chloro-4-morpholin-4-yl-phenyl)-(4-{2-[(3,5-dichloro-phenyl)-methyl-amino]-4-methyl-thiazol-5-yl}-pyrimidin-2-yl)-amine (128). By reaction between 1-{2-[(3,5-dichloro-phenyl)-methyl-amino]-4-methyl-thiazol-5-yl}-3-dimethylamino-propenone and N-(3-chloro-4-morpholin-4-yl-phenyl)-guanidine. Yellow solid. Mp 224-225° C. Anal. RP-HPLC: t_{R}=22.7 min (20-70% MeCN; purity 100%). ¹H-NMR (DMSO-d₆) d 2.95 (s, 3H, CH₃), 3.31 (t, 4H, J=4.5 Hz, CH₂), 3.92 (s, 3H, CH₃), 4.15 (t, 4H, J=4.5 Hz, CH₂), 7.42 (d, 1H, J=5.5 Hz, pyrimidinyl-H), 7.48 (d, 2H, J=9.0 Hz, Ph-H), 7.9 (d, 1H, J=9.0 Hz, Ph-H), 7.98 (t, 1H, J=2.0 Hz, Ph-H), 8.13 (d, 2H, J=2.0 Hz, Ph-H), 8.46 (d, 1H, J=3.0 Hz, Ph-H), and 8.81 (d, 1H, J= 5.5 Hz, pyrimidinyl-H). MS (ESI⁺) m/z 562.68 (C₂₅H₂₃Cl₃N₆OS requires 561.91).

[4-(4-Methyl-2-thiophen-2-yl-thiazol-5-yl)-pyrimidin-2-yl]-(4-morpholin-4-yl-phenyl-amine (129). By reaction between 3-dimethylamino-1-(4-methyl-2-thiophen-2-yl-thiazol-5-yl)-propenone and N-(4-morpholin-4-yl-phenyl)-guanidine. Yellow solid. Mp 247-249° C. Anal. RP-HPLC: t_{R}=16.7 min (20-70% MeCN; purity 100%). ¹NMR (DMSO-d₆) δ: 2.68 (s, 3H, CH₃), 3.05 (t, 4H, J=4.5 Hz, CH₂), 3.74 (t, 4H, J=4.5 Hz, CH₂), 6.94 (d, 2H, J=9.0 Hz, Ph-H), 7.08 (d, 1H, J=5.5 Hz, pyrimidinyl-H), 7.21 (t, 1H, J=4.0 Hz, Ar—H), 7.63 (d, 2H, J=9.0 Hz, Ph-H), 7.76 (d, 1H, J=4.0 Hz, Ar—H), 7.79 (d, 1H, J=5.0 Hz, Ar—H), and 8.49 (d, 1H, J=5.0 Hz, Ph-H). MS (ESI⁺) m/z 435.86 (C₂₂H₂₁N₅OS₂ requires 435.57).

N-{3-[4-(4-Methyl-2-thiophen-2-yl-thiazol-5-yl)-pyrimidin-2-ylamino]-benzyl}-acetamide (130). By reaction between 3-dimethylamino-1-(4-methyl-2-thiophen-2-yl-thiazol-5-yl)-propenone and N-(3-guanidino-benzyl)-acetamide. Yellow solid. Mp 223-225° C. Anal. RP-HPLC: t_{R}=17.5 min (20-70% MeCN; purity 97%). ¹H-NMR (DMSO-d₆) δ: 2.15 (s, 3H, CH₃), 2.99 (s, 3H, CH₃), 4.56 (d, 2H, J=6.0 Hz, CH₂), 7.18 (d, 1H, J=7.5 Hz, Ph-H), 7.45 (d, 1H, J=5.5 Hz, pyrimidinyl-H), 7.5 (t, 1H, J=3.5 Hz, Ar—H), 7.56 (t, 1H, J=8.0 Hz, Ph-H), 7.9 (d, 1H, J=8.0 Hz, Ph-H), 8.04 (s, 1H, Ph-H), 8.07-8.1 (m, 2H, Ar—H), 8.61 (t, 1H, J=6.0 Hz, NH), 8.84 (d, 1H, J=5.0 Hz, pyrimidinyl-H). MS (ESI⁺) m/z 421.90 (C₂₁H₁₉N₅OS₂ requires 421.54).

1-(4-{4-[4-(4-Methyl-2-thiophen-2-yl-thiazol-5-yl)-pyrimidin-2-ylamino]-phenyl}-piperazin-1-yl)-ethanone (131). By reaction between 3-dimethylamino-1-(4-methyl-2-thiophen-2-yl-thiazol-5-yl)-propenone and N-[4-(4-acetyl-piperazin-1-yl)-phenyl]-guanidine. Yellow solid. Mp 134-136° C. Anal. RP-HPLC: t_{R}=14.9 min (20-70% MeCN; purity 97%). ¹H-NMR (DMSO-d₆) δ: 2.04 (s, 3H, CH₃), 2.68 (s, 3H, CH₃), 3.02 (t, 4H, J=5.0 Hz, CH₂), 3.09 (t, 4H, J=5.0 Hz, CH₂), 6.97 (d, 2H, J=9.0 Hz, Ph-H), 7.09 (d, 1H, J=5.5 Hz, pyrimidinyl-H), 7.21 (t, 1H, J=4.5 Hz, Ar—H), 7.64 (d, 2H, J=9.0 Hz, Ph-H), 7.76 (d, 1H, J=4.0 Hz, Ar—H), 7.79 (d, 1H, J=5.0 Hz, Ar—H), 8.49 (d, 1H, J=5.0 Hz, pyrimidinyl-H). MS (ESI⁺) m/z 476.9 (C₂₄H₂₄N₆OS₂ requires 476.62).

{5-[2-(4-Dimethylamino-phenylamino)-pyrimidin-4-yl]-4-methyl-thiazol-2-yl}-methanol (132). Into a solution of hydroxyl-acetonitrile (0.30 mol), pyridine (0.37 mol) and Et₃N (0.14 mol) H₂S was bubbled at such a rate that the reaction temperature reached 63° C. over 20 min. The addition of H₂S was continued for 1.5 h. After stirring at room temperature for a further 1.5 h, the mixture was evaporated to dryness. The residue of 2-hydroxy-thioacetamide was treated with 3-chloro-pentane-2,4-dione (0.30 mol) in EtOH (0.5 mL) and H₂SO₄ (5.0 mL) was added drop-wise. The reaction mixture was heated under reflux for 1 h. After cooling, the mixture was concentrated and the residue was treated with H₂O (400 mL). After neutralisation with solid Na₂CO₃, the mixture was extracted with EtOAc (3×350 mL). The combined organic fractions were washed with brine, dried, filtered, and evaporated to afford 1-(2-hydroxymethyl-4-methyl-thiazol-5-yl)-ethanone (29.5 g, 57%) as an orange solid. ¹NMR (CDCl₃) δ: 2.53 (s, 3H, CH₃), 2.70 (s, 3H, CH₃), 3.31 (br. s, 1H, OH), 4.92 (s, 2H, CH₂). MS (ESI⁺) m/z 172.61 (C₇H₉NO₂S requires 171.22). From this material 3-dimethylamino-1-(2-hydroxymethyl-4-methyl-thiazol-5-yl)-propenone was prepared in the usual manner. ¹H-NMR (CDCl₃) δ: 2.71 (s, 3H, CH₃), 2.77 (br. s, 1H, OH), 2.90 (s, 3H, CH₃), 3.15 (s, 6H, CH₃), 4.90 (s, 2H, CH₂), 5.41 (d, 1H, J=12.2 Hz, CH), 7.74 (d, 1H, J=12.2 Hz, CH). MS (ESI⁺) m/z 227.14 (C₁₀H₁₄N₂O₂S requires 226.30). The title compound was prepared by the condensation of 3-dimethylamino-1-(2-hydroxymethyl-4-methyl-thiazol-5-yl)-propenone with N-(4-dimethylamino-phenyl)-guanidine under the usual conditions. The title compound was obtained as a yellow solid. Anal. RP-HPLC: t_{R}=10.9 min (0-60% MeCN; purity 100%). ¹NMR (DMSO-d₆) δ: 2.05 (s, 3H, CH₃), 2.71 (s, 6H, CH₃), 2.90 (s, 3H, CH₃), 4.94 (s, 2H, CH₂), 6.79 (m, 2H, Ph-H), 6.88 (d, 1H, J=5.0 Hz, pyrimidinyl-H), 6.94 (br. s, 1H, OH), 7.46 (d, 2H, J=8.3 Hz, Ph-H), 8.38 (d, 1H, J=5.5 Hz, pyrimidinyl-H). MS (ESI⁺) m/z 341.43 (C₁₇H₁₉N₅OS requires 341.43).

(3,5-Dichloro-4-morpholin-4-yl-phenyl)-[4-(2-ethylamino-4-methyl-thiazol-5-yl)-pyrimidin-2-yl]-amine (133). By reaction between 3-dimethylamino-1-(2-ethylamino-4-methyl-thiazol-5-yl)-propenone and N-(3,5-dichloro-4-morpholin-4-yl-phenyl)-guanidine. Yellow solid. Mp>300° C. Anal. RP-HPLC: t_{R}=17.1 min (10-70% MeCN; purity 100%). ¹H-NMR (DMSO-d₆) δ: 1.45 (t, 3H, J=7.0 Hz, CH₃), 2.74 (s, 3H, CH₃), 3.36 (t, 4H, J= 4.5 Hz, CH₂), 3.52 (m, 2H, CH₂), 3.96 (t, 4H, J=4.5 Hz, CH₂), 7.23 (d, 1H, J=5.5 Hz, pyrimidinyl-H), 8.2 (s, 2H, Ph-H), 8.47 (t, 1H, J=5.0 Hz, NH), 8.63 (d, 1H, J=5.5 Hz, pyrimidinyl-H). MS (ESI⁺) m/z 466.86 (C₂₀H₂₂Cl₂N₆OS requires 465.40).

(3-Chloro-4-morpholin-4-yl-phenyl)-[4-(2-ethylamino-4-methyl-thiazol-5-yl)pyrimidin-2-yl]-amine (134). By reaction between 3-dimethylamino-1-(2-ethylamino-4-methyl-thiazol-5-yl)-propenone and N-(3-chloro-4-morpholin-4-yl-phenyl)-guanidine. Yellow solid. Mp 273-274° C. Anal. RP-HPLC: t_{R}=13.4 min (10-70% MeCN; purity 100%). ¹H-NMR (DMSO-d₆) δ: 1.18 (t, 3H, J=7 Hz, CH₃), 2.46 (s, 3H, CH₃), 2.91 (t, 2H, J=4.5 Hz, CH₂), 3.26 (m, 2H, CH₂), 3.73 (t, 4H, J=4.5 Hz, CH₂), 6.90 (d, 1H, J=5.5 Hz, pyrimidinyl-H), 7.1 (d, 1H, J=9.0 Hz, PhH), 7.58 (d, 1H, J=9.0 Hz, Ph-H), 8.09 (s, 1H, Ph-H), 8.14 (d, 1H, J=5.5 Hz, NH), 8.33 (d, 1H, J=5.5 Hz, pyrimidinyl-H), 9.50 (s, 1H, NH). MS (ESI⁺) m/z 431.01 (C₂₀H₂₃ClN₆OS requires 430.01).

[4-(4,2′-Dimethyl-[2,4′]bithiazolyl-5-yl)-pyrimidin-2-yl]-(4-morpholin-4-yl-phenyl)-amine (135). By reaction between 3-dimethylamino-1-(4,2′-dimethyl-[2,4′]-bithiazolyl-5-yl)-propenone and N-(4-morpholin-4-yl-phenyl)-guanidine. Yellow solid. Mp 262-263° C. Anal. RP-HPLC: t_{R}=13.8 min (10-70% MeCN; purity 100%). ¹H-NMR (DMSO-d₆) δ: 2.72 (s, 3H, CH₃), 2.75 (s, 3H, CH₃), 3.06 (t, 4H, J=4.5 Hz, CH₂), 3.74 (t, 4H, J=4.5 Hz, CH₂), 6.92 (d, 2H, J=9.0 Hz, Ph-H), 7.09 (d, 1H, J=5.0 Hz, pyrimidinyl-H), 7.62 (d, 2H, J=9.0 Hz, Ph-H), 8.17 (s, 1H, Ar—H), 8.49 (d, 1H, J=5.0 Hz, pyrimidinyl-H), 9.44 (s, 1H, NH). MS (ESI⁺) m/z 450.96 (C₂₂H₂₂N₆OS₂ requires 450.58).

(3-Chloro-4-morpholin-4-yl-phenyl)-[4-(4,2′-dimethyl-[2,4′-bithiazolyl-5-yl)-pyrimidin-2-yl]-amine (136). By reaction between 3-dimethylamino-1-(4,2′-dimethyl-[2,4′]bithiazolyl-5-yl)-propenone and N-(3-chloro-4-morpholin-4-yl-phenyl)-guanidine. Yellow solid. Mp 228-229° C. Anal. RP-HPLC: t_{R}=22.7 min (10-70% MeCN; purity 98%). ¹NMR (DMSO-d₆) δ: 2.98 (s, 3H, CH₃), 2.99 (s, 3H, CH₃), 3.18 (t, 4H, J=4.5 Hz, CH₂), 3.99 (t, 4H, J=4.5 Hz, CH₂), 7.39 (d, 1H, J=9.0 Hz, Ph-H), 7.42 (d, 1H, J=5.0 Hz, pyrimidinyl-H), 7.88 (d, 1H, J=9.0 Hz, Ph-H), 8.30 (s, 1H, Ph-H), 8.42 (s, 1H, Ar—H), 8.80 (d, 1H, J=5.0 Hz, pyrimidinyl-H). MS (ESI⁺) m/z 506.87 [M+Na] (C₂₂H₂₁ClN₆OS₂ requires 485.03).

(3,5-Dichloro-4-morpholin-4-yl-phenyl)-[4-(4,2′-dimethyl-[2,4′]bithiazolyl-5-yl)-pyrimidin-2-yl]-amine (137). By reaction between 3-dimethylamino-1-(4,2′-dimethyl-[2,4′]bithiazolyl-5-yl)-propenone and N-(3,5-dichloro-4-morpholin-4-yl-phenyl)-guanidine. Yellow solid. Mp. >300° C. ¹H-NMR (DMSO-d₆) δ: 2.75 (s, 3H, CH₃), 2.76 (s, 3H, CH₃), 3.12 (t, 4H, J=4.5 Hz, CH₂), 3.71 (t, 4H, J=4.5 Hz, CH₂), 7.27 (d, 1H, J=5.0 Hz, pyrimidinyl-H), 7.96 (s, 1H, Ph-H), 8.2 (s, 1H, Ar—H), 8.62 (d, 1H, J=5.0 Hz, pyrimidinyl-H). MS (ESI⁺) m/z 520.79 (C₂₂H₂₀Cl₂N₆OS₂ requires 519.47).

{4-[4-Methyl-2-(thiophene-2-sulfonylmethyl)-thiazol-5-yl]-pyrimidin-2-yl}-(4-morpholin-4-yl-phenyl)-amine (138). By reaction between 3-dimethylamino-1-[4-methyl-2-(thiophene-2-sulfonylmethyl)-thiazol-5-yl]-propenone and N-(4-morpholin-4-yl-phenyl)-guanidine. Yellow solid. Mp 187-189° C. Anal. RP-HPLC: t_{R}=15.5 min (10-70% MeCN; purity 100%). ¹NMR (DMSO-d₆) δ: 2.60 (s, 3H, CH₃), 3.05 (t, 4H, J=4.5 Hz, CH₂), 3.75 (t, 4H, J=4.5 Hz, CH₂), 5.3 (s, 2H, CH₂), 6.9 (d, 2H, J=9.0 Hz, Ph-H), 7.05 (d, 1H, J=5.0 Hz, pyrimidinyl-H), 7.28 (d, 1H, J=4.5 Hz, Ar—H), 7.59 (d, 2H, J=9.0 Hz, Ph-H), 7.76 (d, 1H, J=4.0 Hz, Ar—H), 8.12 (d, 1H, J=5.0 Hz, Ar—H), 8.49 (d, 1H, J=5.0 Hz, pyrimidinyl-H), 9.48 (s, 1H, NH).). MS (ESI⁺) m/z 520.79 (C₂₃H₂₃N₅O₃S₃ requires 513.66).

[4-(2,4-Dimethyl-thiazol-5-yl)-pyrimidin-2-yl]-(2-methyl-4-morpholin-4-yl-phenyl)-amine (139). By reaction between 3-dimethylamino-1-(2,4-dimethyl-thiazol-5-yl)-propenone and N-(2-methyl-4-morpholin-4-yl-phenyl)-guanidine. Yellow solid. Anal. RP-HPLC: t_{R}=9.8 minutes (10-70% MeCN; purity 100%). ¹NMR (DMSO-d₆)<2.24 (s, 3H, CH₃), 2.59 (s, 3H, CH₃), 2.65 (s, 3H, CH₃), 3.13 (m, 4H, CH₂), 3.85 (m, 4H, CH₂), 6.84 (m, 1H, Ph-H), 6.89 (d, 1H, Ph-H), 6.92 (d, 1H, J=5.0 Hz, pyrimidinyl-H), 7.33 (d, 1H, J=9.0 Hz, Ph-H), 8.28 (d, 1H, J=5.0 Hz, pyrimidinyl-H). MS (ESI⁺) m/z 381.90 (C₂₀H₂₃N₅OS requires 381.50).

{4-[2-(2,4-Dimethyl-phenyl)-4-methyl-thiazol-5-yl]-pyrimidin-2-yl}-(4-morpholin-4-yl-phenyl)-amine (140). By reaction between 3-dimethylamino-1-[2-(2,4-dimethyl-phenyl)-4-methyl-thiazol-5-yl]-propenone and N-(4-morpholin-4-yl-phenyl)-guanidine. Yellow solid. Mp 191-192° C. Anal. RP-HPLC: t_{R}=18.4 minutes (10-70% MeCN; purity 100%). ¹H-NMR (DMSO-d₆) δ: 2.17 (s, 3H, CH₃), 2.34 (s, 3H, CH₃), 2.99 (t, 4H, J=4.5 Hz, CH₂), 3.37 (s, 3H, CH₃), 3.74 (t, 4H, J=4.5 Hz, CH₂), 6.75 (d, 2H, J=9.0 Hz, Ph-H), 6.82 (d, 1H, J=5.5 Hz, pyrimidinyl-H), 7.18 (d, 1H, J=7.5 Hz, Ph-H), 7.19-7.26 (m, 2H, Ph-H), 7.5 (d, 2H, J=9.0 Hz, Ph-H), 8.26 (d, 1H, J=5.5 Hz, pyrimidinyl-H), 9.11 (s, 1H, NH). MS (ESI⁺) m/z 381.90 (C₂₆H₂₇N₅OS requires 457.59).

(3-Chloro-4-morpholin-4-yl-phenyl)-[4-(2,4-dimethyl-thiazol-5-yl)-pyrimidin-2-yl]-amine (141). By reaction between 3-methylamino-1-(2,4-dimethyl-thiazol-5-yl)-propenone and N-(3-chloro-4-morpholin-4-yl-phenyl)-guanidine. Yellow solid. Anal. RP-HPLC: t_{R}=18.4 min (10-70% MeCN; purity 100%). ¹NMR (DMSO-d₆) δ: 2.64 (s, 3H, CH₃), 2.65 (s, 3H, CH₃), 2.92 (t, 4H, J=4.5 Hz, CH₂), 3.85 (t, 4H, J=4.5 Hz, CH₂), 7.09 (d, 1H, J=5.5 Hz, pyrimidinyl-H), 7.13 (d, 1H, J=9.0 Hz, Ph-H), 7.64 (m, 1H, Ph-H), 7.99 (d, 1H, J=2.5 Hz, Ph-H), 8.52 (d, 1H, J=5.5 Hz, pyrimidinyl-H), 9.73 (s, 1H, NH). MS (ESI⁺) m/z 401.95 (C₁₉H₂₀ClN₅OS requires 401.91).

(3,5-Dichloro-4-morpholin-4-yl-phenyl)-[4-(2,4-dimethyl-thiazol-5-yl)-pyrimidin-2-yl]-amine (142). By reaction between 3-dimethylamino-1-(2,4-dimethyl-thiazol-5-yl)-propenone and N-(3,5-dichloro-4-morpholin-4-yl-phenyl)-guanidine. Yellow solid. Anal. RP-HPLC: t_{R}=14.31 minutes (20-80% MeCN; purity 100%). ¹H-NMR (DMSO-d₆) δ: 2.65 (s, 3H, CH₃), 2.66 (s, 3H, CH₃), 3.10 (t, 4H, J=4.5 Hz, CH₂), 3.69 (t, 4H, J=4.5 Hz, CH₂), 7.16 (d, 1H, J=5.5 Hz, pyrimidinyl-H), 7.92 (s, 2H, Ph-H), 8.57 (d, 1H, J=5.5 Hz, pyrimidinyl-H), 9.96 (s, 1H, NH). MS (ESI⁺) m/z 437.89 (C₁₉H₁₉Cl₂N₅OS requires 436.36).

[4-(2-tert-Butylamino-4-methyl-thiazol-5-yl)-pyrimidin-2-yl]-(4-morpholin-4-yl-phenyl)-amine (143). By reaction between 1-(2-tert-butylamino-4-methyl-thiazol-5-yl)-3-dimethylamino-propenone and N-(4-morpholin-4-yl-phenyl)-guanidine. Yellow solid. Anal. RP-HPLC: t_{R}=11.5 minutes (10-70% MeCN; purity 98%). ¹NMR (DMSO-d₆) δ: 1.39 (s, 9H, CH₃), 2.46 (s, 3H, CH₃), 3.02 (t, 4H, J=4.5 Hz, CH₂), 3.74 (t, 4H, J=4.5 Hz, CH₂), 6.80 (d, 1H, J=5.5 Hz, pyrimidinyl-H), 7.87 (d, 2H, J=9.0 Hz, Ph-H), 7.61 (d, 2H, J=9.0 Hz, Ph-H), 8.26 (d, 1H, J=5.5 Hz, pyrimidinyl-H), 9.17 (s, 1H, NH). MS (ESI⁺) m/z 425.05 (C₂₂H₂₈N₆OS requires 424.56).

{4-[2-(2-Methoxy-ethylamino)-4-methyl-thiazol-5-yl]-pyrimidin-2-yl}-(4-morpholin-4-yl-phenyl)-amine (144). By reaction between 3-dimethylamino-1-[2-(2-methoxy-ethylamino)-4-methyl-thiazol-5-yl]-propenone and N-(4-morpholin-4-yl-phenyl)-guanidine. Yellow solid. Anal. RP-HPLC: t_{R}=9.4 min (10-70% MeCN; purity 98%). ¹NMR (DMSO-d₆) δ: 2.45 (s, 3H, CH₃), 3.03 (t, 4H, J=4.5 Hz, CH₂), 3.44 (q, 2H, J=5.5 Hz, CH₂), 3.49 (q, 2H, J=5.5 Hz, CH₂), 3.73 (t, 4H, J=4.5 Hz, CH₂), 6.81 (d, 1H, J=5.5 Hz, pyrimidinyl-H), 6.87 (d, 2H, J=8.5 Hz, Ph-H), 7.61 (d, 2H, J=8.5 Hz, Ph-H), 8.13 (t, 1H, J=5.5 Hz, NH), 8.26 (d, 1H, J=5.5 Hz, pyrimidinyl-H), 9.18 (s, 1H, NH). MS (ESI⁺) m/z 427.00 (C₂₁H₂₆N₆O₂S requires 426.54).

[4-(4-Methyl-2-methylamino-thiazol-5-yl)-pyrimidin-2-yl]-(2-methyl-4-morpholin-4-yl-phenyl)-amine (145). By reaction between 3-dimethylamino-1-(4-methyl-2-methylamino-thiazol-5-yl)-propenone and N-(2-methyl-4-morpholin-4-yl-phenyl)-guanidine. Yellow solid. Anal. RP-HPLC: t_{R}=9.0 min (10-70% MeCN; purity 99%). ¹H-NMR (DMSO-d₆) δ: 2.15 (s, 3H, CH₃), 2.39 (s, 3H, CH₃), 2.81 (d, 3H, J=5.0 Hz, CH₃), 3.05 (m, 4H, CH₂), 3.73 (m, 4H, CH₂), 6.72 (m, 2H, Ph-H and pyrimidinyl-H), 6.79 (m, 1H, Ph-H), 7.23 (d, 1H, J=9.0 Hz, Ph-H), 7.91 (m, 1H, NH), 8.15 (d, 1H, J=5.5 Hz, pyrimidinyl-H), 8.36 (s, 1H, NH). MS (ESI⁺) m/z 396.98 (C₂₀H₂₄N₆OS requires 396.51).

[4-(2-Ethylamino-4-methyl-thiazol-5-yl)-pyrimidin-2-yl]-(2-methyl-4-morpholin-4-yl-phenyl)-amine (146). By reaction between 3-dimethylamino-1-(2-ethylamino-4-methyl-thiazol-5-yl)-propenone and N-(4-morpholin-4-yl-phenyl)-guanidine. Yellow solid. Anal. RP-HPLC: t_{R}=9.8 min (10-70% MeCN; purity 99%). ¹H-NMR (DMSO-d₆) δ: 1.14 (t, 3H, J=6.5 Hz, CH₃), 2.16 (s, 3H, CH₃), 2.49 (s, 3H, CH₃), 3.06 (m, 4H, CH₂), 3.24 (m, 2H, CH₂), 3.73 (m, 4H, CH₂), 6.72 (m, 2H, Ph-H and pyrimidinyl-H), 6.22 (m, 1H, Ph-H), 7.97 (t, 1H, J=5.0 Hz, NH), 8.16 (d, 1H, J=5.5 Hz, pyrimidinyl-H), 8.37 (s, 1H, NH). MS (ESI⁺) m/z 409.00 (C₂₁H₂₆N₆OS requires 410.54).

{4-[4-Methyl-2-(4-morpholin-4-yl-phenyl)-thiazol-5-yl]-pyrimidin-2-yl}-(4-morpholin-4-yl-phenyl)-amine (147). By reaction between 3-dimethylamino-1-[4-methyl-2-(4-morpholin-4-yl-phenyl)-thiazol-5-yl]-propenone and N-(4-morpholin-4-yl-phenyl)-guanidine. Yellow solid. Mp. 273-274° C. Anal. RP-HPLC: t_{R}=16.5 minutes (0-60% MeCN; purity 99%). ¹H-NMR (DMSO-d₆) δ: 2.7 (s, 3H, CH₃), 3.05 (t, 4H, J=4.5 Hz, CH₂), 3.25 (t, 4H, J=4.5 Hz, CH₂), 3.75 (m, 8H, CH₂), 6.94 (d, 2H, J=9.0 Hz, Ph-H), 7.05 (m, 3H, Ph-H and pyrimidinyl-H), 7.65 (d, 2H, J=9.0 Hz, Ph-H), 7.84 (d, 2H, J=8.5 Hz, Ph-H), 8.46 (d, 1H, J=5.0 Hz, pyrimidinyl-H), 9.42 (s, 1H, NH). MS (ESI⁺) m/z 515.00 (C₂₈H₃₀N₆O₂S requires 514.64).

1-[4-(4-{4-[4-Methyl-2-(4-morpholin-4-yl-phenyl)-thiazol-5-yl]-pyrimidin-2-ylamino}-phenyl)-piperazin-1-yl]-ethanone (148). By reaction between 3-dimethylamino-1-[4-methyl-2-(4-morpholin-4-yl-phenyl)-thiazol-5-yl]-propenone and N-[4-(4-acetyl-piperazin-1-yl)-phenyl]-guanidine. Yellow solid. Mp 250-252° C. Anal. RP-HPLC: t_{R}=15.9 min (0-60% MeCN; purity 99%). ¹H-NMR (DMSO-d₆) δ: 2.16 (s, 3H, CH₃), 2.77 (s, 3H, CH₃), 3.15 (m, 4H, CH₂), 3.29 (t, 4H, J=5.0 Hz, CH₂), 3.65 (t, 2H, J=5.0 Hz, CH₂), 3.81 (t, 2H, J=5.0 Hz, CH₂), 3.89 (t, 4H, J=5.0 Hz, CH₂), 6.93-6.99 (m, 5H, Ph-H and pyrimidinyl-H), 7.36 (m, 1H, NH), 7.57 (d, 2H, J=9.0 Hz, Ph-H), 7.91 (d, 2H, J=9.0 Hz, Ph-H), 8.36 (d, 1H, J=5.5 Hz, pyrimidinyl-H). MS (ESI⁺) m/z 555.94 (C₃₀H₃₃N₇O₂S requires 555.70).

N⁴-[4-(2,4-Dimethyl-thiazol-5-yl)-pyrimidin-2-yl]-N¹-methyl-2-trifluoromethyl-benzene-1,4-diamine (149). By reaction between 3-dimethylamino-1-(2,4-dimethyl-thiazol-5-yl)-propenone and N-(4-methylamino-3-trifluoromethyl-phenyl)-guanidine. Yellow solid. Anal. RP-HPLC: t_{R}=17.4 min (10-70% MeCN; purity 95%). ¹NMR (DMSO-d₆) δ: 2.61 (s, 3H, CH₃), 2.63 (s, 3H, CH₃), 2.75 (d, 3H, J=4.9 Hz, CH₃), 5.33 (m, 1H, NH), 6.74 (d, 1H, J=9.3 Hz, Ph-H), 7.01 (d, 1H, J=4.9 Hz, pyrimidinyl-H). 7.70 (d, 1H, J=9.0 Hz, Ph-H), 7.93 (s, 1H, Ph-H), 8.46 (d, 1H, J=5.0 Hz, pyrimidinyl-H), 9.45 (s, 1H, NH). MS (ESI⁺) m/z 379.01 (C₁₇H₁₆F₃N₅S requires 379.40).

[4-(2,4-Dimethyl-thiazol-5-yl)-pyrimidin-2-yl]-(3-morpholin-4-ylmethyl-4-ylmethyl-phenyl)amine (150). By reaction between 3-dimethylamino-1-(2,4-dimethyl-thiazol-5-yl)-propenone and N-(3-morpholin-4-ylmethyl-phenyl)-guanidine. Yellow solid. Anal. RP-HPLC: t_{R}=10.2 min (10-70% MeCN; purity 96%). ¹NMR (DMSO-d₆) δ: 2.51 (m, 4H, CH₂), 2.71 (s, 6H, CH₃), 3.55 (s, 2H, CH₂), 3.74 (t, 4H, J=4.9 Hz, CH₂), 6.94 (d, 1H, J=4.5 Hz, pyrimidinyl-H), 7.04 (d, 1H, J=7.0 Hz, Ph-H), 7.30 (m, 2H, Ph-H), 7.60 (m, 2H, Ph-H and NH), 8.42 (d, 1H, J=4.5 Hz, pyrimidinyl-H). MS (ESI⁺) m/z 381.90 (C₂₀H₂₃N₅OS requires 381.50).

4-[4-(2,4-Dimethyl-thiazol-5-yl)-pyrimidin-2-ylamino]-2-morpholin-4-ylmethyl-phenol (151). By reaction between 3-dimethylamino-1-(2,4-dimethyl-thiazol-5-yl)-propenone and N-(4-hydroxy-3-morpholin-4-ylmethyl-phenyl)-guanidine. Yellow solid. Anal. RP-HPLC: t_{R}=8.69 min (10-70% MeCN; purity 100%). ¹H-NMR (DMSO-d₆) δ: 2.45 (m, 4H, CH₂), 2.61 (s, 3H, CH₂), 2.64 (s, 3H, CH₃), 3.59 (s, 2H, CH₂), 3.60 (t, 4H, 4.5 Hz, CH₂), 6.70 (d, 1H, J=8.5 Hz, Ph-H), 6.97 (d, 1H, J=5.5 Hz, pyrimidinyl-H), 7.40 (dd, 1H, J=2.5, 9.0 Hz, Ph-H), 7.50 (d, 1H, J=2.5 Hz, Ph-H), 8.43 (d, 1H, J=5.5 Hz, pyrimidinyl-H), 9.32 (s, 1H, NH). MS (ESI⁺) m/z 398.05 (C₂₀H₂₃N₅O₂S requires 397.50).

[4-(2,4-Dimethyl-thiazol-5-yl)-pyrimidin-2-yl]-(3-morpholin-4-yl-phenyl)-amine (152). By reaction between 3-dimethylamino-1-(2,4-dimethyl-thiazol-5-yl)-propenone and N-(3-morpholin-4-yl-phenyl)-guanidine. Yellow solid. Anal. RP-HPLC: t_{R}=12.8 min (10-70% MeCN; purity 100%). ¹H-NMR (DMSO-d₆) δ: 2.62 (s, 3H, CH₂), 2.65 (s, 3H, CH₃), 3.12 (t, 4H, J=4.5 Hz, CH₂), 3.76 (t, 4H, J=4.5 Hz, CH₂), 6.56 (dd, 1H, J=2.0, 8.0 Hz, Ph-H), 7.07 (d, 1H, 5.0 Hz, pyrimidinyl-H), 7.14 (t, 1H, J=8.0 Hz, Ph-H), 7.22 (d, 1H, J=9.0 Hz, Ph-H), 7.51 (s, 1H, Ph-H), 8.50 (d, 1H, J=4.5 Hz, pyrimidinyl-H), 9.51 (s, 1H, NH). MS (ESI⁺) m/z 367.93 (C₁₉H₂₁N₅OS requires 367.47).

{4-[4-Methyl-2-(methyl-pyridin-3-yl-amino)-thiazol-5-yl]-pyrimidin-2-yl}(4-morpholin-4-yl-phenyl)-amine (153). By reaction between 3-dimethylamino-1-[4-methyl-2-(methyl-pyridin-3-yl-amino)-thiazol-5-yl]-propenone and N-(4-morpholin-4-yl-phenyl)-guanidine. Yellow solid. Mp 211-212° C. Anal. RP-HPLC: t_{R}=15.8 min (0-60% MeCN; purity 100%). ¹NMR (DMSO-d₆) δ: 2.61 (s, 3H, CH₃), 3.11 (m, 4H, CH₂), 3.78 (t, 4H, J=4.5 Hz, CH₂), 3.80 (s, 3H, CH₃), 6.97 (m, 3H, Ph-H and pyrimidinyl-H), 7.16 (t, 1H, J=6.0 Hz, Ar—H), 7.38 (d, 1H, J=8.0 Hz, Ar—H), 7.67 (m, 2H, Ph-H and Ar—H), 7.92 (m, 1H, Ar—H), 8.35 (d, 1H, J=5.5 Hz, pyrimidinyl-H), 8.46 (d, 1H, J=5.5 Hz, Ar—H), 9.42 (br. s, 1H, NH). MS (ESI⁺) m/z 459.92 (C₂₄H₂₅N₇OS requires 459.57).

[4-(4-Methyl-2-pyridin-3-yl-thiazol-5-yl)-pyrimidin-2-yl]-(3,4,5-trimethoxy-phenyl)-amine (154). By reaction between 3-dimethylamino-1-(4-methyl-2-pyridin-3-yl-thiazol-5-yl)-propenone and N-(3,4,5-trimethoxy-phenyl)-guanidine. Yellow solid. Mp 210-211° C. Anal. RP-HPLC: t_{R}=12.9 min (0-60% MeCN; purity 100%). ¹H-NMR (DMSO-d₆) δ: 2.76 (s, 3H, CH₃), 3.64 (s, 3H, CH₃), 3.83 (s, 9H, CH₃), 7.20 (d, J=5.5 Hz, 1H, pyrimidinyl-H), 7.22 (s, 2H, Ph-H), 7.59 (q, J=4.5 Hz, 1H, Ar—H), 8.28 (8.58 (d, J=8.0 Hz, 1H, Ar—H), 8.58 (d, 1H, J=5.0 Hz, pyrimidinyl-H), 8.71 (d, 1H, J=5.0 Hz, Ar—H), 9.11 (d, 1H, J=2.0 Hz, Ar—H). MS (ESI⁺) m/z 435.67 (C₂₂H₂₁N₅O₃S requires 435.50).

(3,5-Dimethoxy-phenyl)-[4-(4-methyl-2-pyridin-3-yl-thiazol-5-yl)-pyrimidin-2-yl]-amine (155). By reaction between 3-dimethylamino-1-(4-methyl-2-pyridin-3-yl-thiazol-5-yl)-propenone and N-(3,5-dimethoxy-phenyl)-guanidine. Yellow solid. Mp 229-230° C. Anal. RP-HPLC: t_{R}=17.8 min (0-60% MeCN; purity 100%). ¹H-NMR (DMSO-d₆) δ: 2.77 (s, 3H, CH₃), 3.77 (s, 6H, CH₃), 6.16 (t, 1H, J=1.89 Hz, Ar—H), 7.12 (m, 2H, Ph-H), 7.22 (d, 1H, J=5.0 Hz, pyrimidinyl-H), 7.59 (q, 1H, J=4.5 Hz, Ar—H), 8.29 (d, 1H, J=8.0 Hz, Ar—H), 8.60 (d, 1H, J=5.5 Hz, pyrimidinyl-H), 8.71 (d, 1H, J=4.5 Hz, Ar—H), 9.12 (d, 1H, J=2.0 Hz, Ph-H). MS (ESI⁺) m/z 406.12 (C₂₁H₁₉N₅O₂S requires 405.47).

(3-Methoxy-4-morpholin-4-yl-phenyl)-[4-(4-methyl-2-pyrimidin-3-yl-thiazol-5-yl)-pyrimidin-2-yl]-amine (156). By reaction between 3-dimethylamino-1-(4-methyl-2-pyridin-3-yl-thiazol-5-yl)-propenone and N-(3-methoxy-4-morpholin-4-yl-phenyl)-guanidine. Yellow solid. Mp. 212-214° C. Anal. RP-HPLC: t_{R}=12.3 min (0-60% MeCN; purity 100%). ¹H-NMR (DMSO-d₆) δ: 2.77 (s, 3H, CH₃), 2.92 (t, 4H, J=4.5 Hz, CH₂), 3.72 (t, 4H, J=4.5 Hz, CH₂), 3.84 (s, 3H, CH₃), 6.88 (d, 1H, J=8.5 Hz, Ph-H), 7.17 (d, 1H, J=5.0 Hz, pyrimidinyl-H), 7.31 (d, 1H, J=8.5 Hz, Ph-H), 7.52 (s, 1H, Ph-H), 7.59 (m, 1H, Ar—H), 8.32 (d, 1H, J=8.0 Hz, Ar—H), 8.56 (d, 1H, J=5.0 Hz, pyrimidinyl-H), 8.71 (d, 1H, J=5.0 Hz, Ar—H), 9.15 (s, 1H, Ar—H). MS (ESI⁺) m/z 461.79 (C₂₄H₂₄N₆O₂S requires 460.55).

[4-(2-Ethylamino-4-methyl-thiazol-5-yl)-pyrimidin-2-yl]-(3-methoxy-4-morpholin-4-yl-phenyl)-amine (157). By reaction between 3-dimethylamino-1-(2-ethylamino-4-methyl-thiazol-5-yl)-propenone and N-(3-methoxy-4-morpholin-4-yl-phenyl)-guanidine. Yellow solid. Mp 224-226° C. Anal. RP-HPLC: t_{R}=10.6 min (0-60% MeCN; purity 100%). ¹H-NMR (DMSO-d₆) δ: 1.17 (t, 3H, J=7.0 Hz, CH₃), 2.45 (s, 3H, CH₃), 2.90 (t, 4H, J=4.5 Hz, CH₂), 3.24 (m, 2H, CH₂), 3.71 (t, 4H, J=4.5 Hz, CH₂), 3.82 (s, 3H, CH₃), 6.8 (d, 1H, J=8.5 Hz, Ph-H), 7.26 (d, 1H, J=5.5 Hz, pyrimidinyl-H), 7.5 (s, 1H, Ph-H), 8.09 (t, 1H, J=5.0 Hz, NH), 8.29 (d, 1H, J=5.5 Hz, pyrimidinyl-H), 9.24 (s, 1H, NH). MS (ESI⁺) m/z 426.97 (C₂₁H₂₆N₆O₂S requires 426.54).

[4-(4-Methyl-2-phenethylamino-thiazol-5-yl)-pyrimidin-2-yl]-(4-morpholin-4-yl-phenyl)-amine (158). By reaction between 3-dimethylamino-1-(4-methyl-2-phenethylamino-thiazol-5-yl)-propenone and N-(4-morpholin-4-yl-phenyl)-guanidine. Yellow solid. Mp 226-228° C. Anal. RP-HPLC: t_{R}=14.3 min (0-60% MeCN; purity 100%). ¹H-NMR (DMSO-d₆) d. 2.46 (s, 3H, CH₃), 2.88 (t, 2H, J=7.5 Hz, CH₂), 3.02 (t, 4H, J=4.5 Hz, CH₂), 3.50 (m, 2H, CH₂), 3.73 (t, 4H, J=4.5 Hz, CH₂), 6.82 (d, 1H, J=5.5 Hz, pyrimidinyl-H), 6.87 (d, 2H, J=9.0 Hz, Ph-H), 7.20-7.33 (m, 5H, Ph-H), 7.61 (d, 2H, J= 9.0 Hz, Ph-H), 8.18 (t, 1H, J=5.0 Hz, NH), 8.27 (d, 1H, J=5.5 Hz, pyrimidinyl-H), 9.18 (s, 1H, NH). MS (ESI⁺) m/z 473.00 (C₂₆H₂₈N₆OS requires 472.61).

(3,5-Dimethoxy-phenyl)-[4-(4-methyl-2-phenethylamino-thiazol-5-yl)-pyrimidin-2-yl]-amine (159). By reaction between 3-dimethylamino-1-(4-methyl-2-phenethylamino-thiazol-5-yl)-propenone and N-(3,5-dimethoxy-phenyl)-guanidine. Yellow solid. Mp 201-202° C. Anal. RP-HPLC: to =18.4 min (0-60% MeCN; purity 100%). ¹H-NMR (DMSO-d₆) δ: 2.47 (s, 3H, CH₃), 2.89 (t, 2H, J=7.5 Hz, CH₂), 3.46 (q, 2H, J=6.5, 13.0 Hz, CH₂), 3.73 (s, 6H, CH₃), 6.1 (s, 1H, Ph-H), 6.92 (d, 1H, J=5.0 Hz, pyrimidinyl-H), 7.06 (s, 2H, Ph-H), 7.20-7.33 (m, 5H, Ph-H), 8.26 (t, 1H, J=5.0 Hz, NH), 8.33 (d, 1H, J= 5.5 Hz, pyrimidinyl-H), 9.36 (s, 1H, NH). MS (ESI⁺) m/z 447.69 (C₂₄H₂₅N₅O₂S requires 447.55).

(3,5-Dimethoxy-phenyl)-[4-(2-ethylamino-4-methyl-thiazol-5-yl)-pyrimidin-2-yl]-amine (160). By reaction between 3-dimethylamino-1-(2-ethylamino-4-methyl-thiazol-5-yl)-propenone and N-(3,5-dimethoxy-phenyl)-guanidine. Yellow solid. Mp 235-237° C. Anal. RP-HPLC: to =15.0 min (0-60% MeCN; purity 100%). ¹H-NMR (DMSO-D₆) δ: 1.17 (t, 3H, J=7.0 Hz, CH₃), 2.46 (s, 3H, CH₃), 3.22-3.26 (m, 2H, CH₂), 3.75 (s, 3H, CH₃), 6.1 (s, 1H, Ph-H), 6.91 (d, 1H, J=5.5 Hz, pyrimidinyl-H), 7.07 (s, 2H, Ph-H), 8.14 (t, 1H, J=5.0 Hz, NH), 8.33 (d, 1H, J=5.5 Hz, pyrimidinyl-H), 9.36 (s, 1H, NH). MS (ESI⁺) m/z 372.11 (C₁₈H₂₁N₅O₂S requires 371.46).

[4-(2-Amino-4-methyl-thiazol-5-yl)-pyrimidin-2-yl]-(3,5-dimethoxy-phenyl)-amine (161). By reaction between N′-[5-(3-dimethylamino-acryloyl)-4-methyl-thiazol-2-yl]-N,N-dimethyl-formamidine and N-(3,5-dimethoxy-phenyl)-guanidine. Yellow solid. Mp 269-271° C. Anal. RP-HPLC: to =13.5 min (0-60% MeCN; purity 100%). ¹H-NMR (DMSO-d₆) δ: 2.43 (s, 3H, CH₃), 3.74 (s, 6H, CH₃), 6.1 (s, 1H, Ph-H), 6.9 (d, 1H, J=5.0 Hz, pyrimidinyl-H), 7.05 (s, 2H, Ph-H), 7.51 (br. s, 2H, NH₂), 8.33 (d, 1H, J=5.5 Hz, pyrimidinyl-H), 9.35 (s, 1H, NH). MS (ESI⁺) m/z 344.09 (C₁₆H₁₇N₅O₂S requires 343.40).

{4-[4-Methyl-2-(methyl-pyridin-3-yl-amino)-thiazol-5-yl]-pyrimidin-2-yl}-(4-morpholin-4-yl-phenyl)-amine (162). By reaction between 3-dimethylamino-1-[4-methyl-2-(methyl-pyridin-3-yl-amino)-thiazol-5-yl]-propenone and N-(4-morpholin-4-yl-phenyl)-guanidine. Yellow solid. Mp 194-195° C. Anal. RP-HPLC: to =11.1 min (0-60% MeCN; purity 100%). ¹H-NMR (DMSO-d₆) δ: 2.51 (s, 3H, CH₃), 3.01 (t, 4H, J=4.5 Hz, CH₂), 3.52 (s, 3H, CH₃), 3.74 (t, 4H, J=4.5 Hz, CH₂), 6.82 (d, 2H, J=9.0 Hz, Ph-H), 6.89 (d, 1H, J=5.5 Hz, pyrimidinyl-H), 7.55 (m, 3H, Ph-H and Ar—H), 8.01 (d, 1H, J=8.0 Hz, Ar—H), 8.32 (d, 1H, J=5.5 Hz, pyrimidinyl-H), 8.53 (d, 1H, J=5.0 Hz, Ar—H), 8.80 (d, 1H, J=2.5 Hz, Ar—H), 9.21 (br. s, 1H, NH). MS (ESI⁺) m/z 459.92 (C₂₄H₂₅N₇OS requires 459.57).

1-(4-{4-[4-(4-Methyl-2-phenethylamino-thiazol-5-yl)-pyrimidin-2-ylamino]-phenyl}-piperazin-1-yl)-ethanone (163). By reaction between 3-dimethylamino-1-(4-methyl-2-phenethylamino-thiazol-5-yl)-propenone and N-[4-(4-acetyl-piperazin-1-yl)-phenyl]-guanidine. Yellow solid. Mp 212-213° C. Anal. RP-HPLC: t_{R}=14.0 min (0-60% MeCN; purity 100%). ¹H-NMR (DMSO-d₆) δ: 2.04 (s, 3H, CH₃), 2.46 (s, 3H, CH₃), 2.89 (t, 2H, J= 7.0 Hz, CH₂), 2.99 (t, 2H, J=4.5 Hz, CH₂), 3.06 (t, 2H, J=4.5 Hz, CH₂), 3.49 (q, 2H, J= 6.5, 13.0 Hz, CH₂), 3.58 (m, 4H, CH₂), 6.83 (d, 1H, J=5.0 Hz, pyrimidinyl-H), 6.9 (d, 2H, J=9.0 Hz, Ph-H), 7.20-7.33 (m, 5H, Ph-H), 7.62 (d, 2H, J=9.0 Hz, Ph-H), 8.19 (t, 1H, J=5.0 Hz, NH), 8.27 (d, 1H, J=5.5 Hz, pyrimidinyl-H), 9.2 (brs, 1H, NH). MS (ESI⁺) m/z 514.94 (C₂₈H₃₁N₇OS requires 513.66).

1-[4-(4-{4-[4-Methyl-2-(methyl-pyridin-3-yl-amino)-thiazol-5-yl]pyrimidin-2-ylamino}-phenyl)-piperazin-1-yl]-ethanone (164). By reaction between 3-dimethylamino-1-[4-methyl-2-(methyl-pyridin-3-yl-ammo)-thiazol-5-yl]-propenone and N-[4-(4-acetyl-piperazin-1-yl)-phenyl]-guanidine. Yellow solid. Mp 205-206° C. Anal. RP-HPLC: t_{R}=11.0 min (0-60% MeCN; purity 100%). ¹NMR (DMSO-d₆) δ: 2.05 (s, 3H, CH₃), 2.51 (s, 3H, CH₃), 2.99 (t, 2H, J=5.0 Hz, CH₂), 3.05 (t, 2H, J=5.0 Hz, CH₂), 3.52 (s, 3H, CH₃), 3.58 (q, 4H, J=5.0 10.0 Hz, CH₂), 6.84 (d, 2H, J=9.0 Hz, Ph-H), 6.89 (d, 1H, J=5.5 Hz, pyrimidinyl-H), 7.55 (m 3H, Ph-H and Ar—H), 8.01 (d, 1H, J=7.0 Hz, Ar—H), 8.32 (d, 1H, J=5.0 Hz, pyrimidinyl-H), 8.53 (d, 1H, J=4.5 Hz, Ar—H), 8.80 (s, 1H, Ar—H), 9.22 (s, 1H, NH). MS (ESI⁺) m/z 502.03 (C₂₆H₂₈N₈OS requires 500.62).

[4-(4-Methyl-2-phenethylamino-thiazol-5-yl)-pyrimidin-2-yl]-(3,4,5-trimethoxy-phenyl)-amine (165). By reaction between 3-dimethylamino-1-(4-methyl-2-phenethylamino-thiazol-5-yl)-propenone and N-(3,4,5-trimethoxy-phenyl)-guanidine. Yellow solid. Mp 184-186° C. Anal. RP-HPLC: t_{R}=17.1 min (0-60% MeCN; purity 100%). ¹NMR (DMSO-d₆) δ: 2.46 (s, 3H, CH₃), 2.89 (t, 2H, J=7.5 Hz, CH₂), 3.45 (q, 2H, J=7.0 Hz, CH₂), 3.61 (s, 3H, CH₃), 3.78 (s, 6H, CH₃), 6.91 (d, 1H, J=5.5 Hz, pyrimidinyl-H), 7.16 (s, 2H, Ph-H), 7.21-7.26 (m, 4H, Ph-H), 7.31 (t, 1H, J=7.5 Hz, Ph-H), 8.26 (t, 1H, J=5.5 Hz, NH), 8.32 (d, 1H, J=5.5 Hz, pyrimidinyl-H), 9.28 (s, 1H, NH). MS (ESI⁺) m/z 477.91 (C₂₅H₂₇N₅O₃S requires 477.58).

[4-(4-Benzyl-piperazin-1-yl)-phenyl]-[4-(4-methyl-2-phenethylamino-thiazol-5-yl)-pyrimidin-2-yl]-amine (166). By reaction between 3-dimethylamino-1-(4-methyl-2-phenylamino-thiazol-5-yl)-propenone and N-[4-(4-benzyl-piperazin-1-yl)-phenyl]-guanidine. Yellow solid. Mp 191-192° C. Anal. RP-HPLC: t_{R}=14.9 min (0-60% MeCN; purity 100%). ¹H-NMR (DMSO-d₆) δ: 2.45 (s, 3H, CH₃), 2.88 (t, 2H, J=7.5 Hz, CH₂), 3.05 (t, 4H, J=5.0 Hz, CH₂), 3.48 (q, 2H, J=7.5, 13.0 Hz, CH₂), 3.52 (s, 2H, CH₂), 6.81 (d, 1H, J=6.0 Hz, pyrimidinyl-H), 6.85 (d, 2H, J=9.0 Hz, Ph-H), 7.20 (t, 1H, J=7.0 Hz, Ph-H), 7.27-7.34 (m, 1H, Ph-H), 7.58 (d, 2H, J=9.0 Hz, Ph-H), 8.19 (t, 1H, J=5.5 Hz, NH), 8.26 (d, 1H, J=5.0 Hz, pyrimidinyl-H), 9.15 (s, 1H, NH). MS (ESI⁺) m/z 561.98 (C₃₃H₃₅N₇S requires 561.74).

[4-(4-Methyl-2-phenylamino-thiazol-5-yl)-pyrimidin-2-yl]-(4-morpholin-4-yl-phenyl)-amine (167). By reaction between 3-dimethylamino-1-(4-methyl-2-phenylamino-thiazol-5-yl)-propenone and N-(4-morpholin-4-yl-phenyl)-guanidine. Yellow solid. Mp 290-291° C. Anal. RP-HPLC: t_{R}=13.6 min (0-60% MeCN; purity 100%). ¹H-NMR (DMSO-d₆) δ: 2.57 (s, 3H, CH₃), 3.04 (t, 4H, J=4.5 Hz, CH₂), 3.74 (t, 4H, J=4.5 Hz, CH₂), 6.89 (d, 2H, J=9.0 Hz, Ph-H), 6.93 (d, 1H, J=5.5 Hz, pyrimidinyl-H), 7.01 (d, 1H, J=7.5 Hz, Ph-H), 7.35 (d, 2H, J=8.0 Hz, Ph-H), 7.61-7.65 (m, 4H, Ph-H), 8.35 (d, 1H, J=5.0 Hz, pyrimidinyl-H), 9.28 (br. s, 1H, NH). MS (ESI⁺) m/z 443.09 (C₂₄H₂₄N₆OS requires 444.55).

[4-(2-Amino-4-methyl-thiazol-5-yl)-pyrimidin-2-yl]-(3,4,5-trimethoxy-phenyl)-amine (168). By reaction between N′-[5-(3-dimethylamino-acryloyl)-4-methyl-thiazol-2-yl]-N,N-dimethyl-formamidine and N-(3,4,5-trimethoxy-phenyl)-guanidine. Yellow solid. Anal. RP-HPLC: t_{R}=10.9 min (10-70% MeCN; purity 97%). ¹H-NMR (DMSO-d₆) δ: 2.42 (s, 3H, CH₃), 3.61 (s, 3H, CH₃), 3.79 (s, 6H, CH₃), 6.88 (d, 1H, J=5.0 Hz, pyrimidinyl-H), 7.15 (s, 2H, Ph-H), 7.50 (s, 2H, NH₂), 8.31 (d, 1H, J=5.0 Hz, pyrimidinyl-H), 9.26 (s, 1H, NH). MS (ESI⁺) m/z 373.96 (C₁₇H₁₉N₅O₃S requires 373.43).

[4-(2,6-Dimethyl-morpholin-4-yl)-phenyl]-[4-(2-ethylamino-4-methyl-thiazol-5-yl)-pyrimidin-2-yl]-amine (169). By reaction between 3-dimethylamino-1-(2-ethylamino-4-methyl-thiazol-5-yl)-propenone and N-[4-(2,6-dimethyl-morpholin-4-yl)-phenyl]-guanidine. Yellow solid. Anal. RP-HPLC: t_{R}=11.2 min (10-70% MeCN; purity 98%). ¹H-NMR (DMSO-d₆) δ: 1.16 (m, 3H, CH₃), 2.19 (t, 3H, J=10.5 Hz, CH₃), 2.45 (s, 3H, CH₃), 3.26 (m, 4H, CH₂), 3.46 (d, 2H, J=10.5 Hz, CH₂), 3.69 (m, 1H, CH₂), 6.81 (d, 1H, J=5.5 Hz, pyrimidinyl-H), 6.87 (d, 2H, J=9.5 Hz, Ph-H), 7.60 (d, 2H, J=9.0 Hz, Ph-H), 8.04 (t, 1H, J=5.0 Hz, NH), 8.26 (d, 1H, J=5.0 Hz, pyrimidinyl-H), 9.15 (br. s, 1H, NH). MS (ESI⁺) m/z 424.99 (C₂₂H₂₈N₆OS requires 424.56).

(3,5-Dimethoxy-phenyl)-[4-(4-methyl-2-methylamino-thiazol-5-yl)-pyrimidin-2-yl]-amine (170). By reaction between 3-dimethylamino-1-(4-methyl-2-methylamino-thiazol-5-yl)-propenone and N-(3,5-dimethoxy-phenyl)-guanidine. Yellow solid. Anal. RP-HPLC: t_{R}=12.8 min (10-70% MeCN; purity 98%). ¹H-NMR (CDCl₃) δ: 2.46 (s, 3H, CH₃), 2.84 (d, 2H, 4.5 Hz, CH₂), 3.74 (s, 6H, CH₃), 6.10 (m 1H, NH), 6.92 (d, 1H, J=4.5 Hz, Ph-H), 7.07 (d, 2H, J=2.0 Hz, Ph-H), 8.07 (m, 1H, NH), 8.32 (d, 1H, J=5.5 Hz, pyrimidinyl-H), 9.37 (s, 1 h, Ph-H). MS (ESI⁺) m/z 357.92 (C₁₇H₁₉N₅O₂S requires 357.43).

(3,5-Dimethoxy-phenyl)-[4-(4-methyl-2-phenylamino-thiazol-5-yl)-pyrimidin-2-yl]-amine (171). By reaction between 3-dimethylamino-1-(4-methyl-2-phenylamino-thiazol-5-yl)-propenone and N-(3,5-dimethoxy-phenyl)-guanidine. Yellow solid. Mp 191-194° C. Anal. RP-HPLC: t_{R}=17.0 min (0-60%; =MeCN; purity 98%). ¹NMR (CDCl₃) δ: 2.61 (s, 3H, CH₃), 3.78 (s, 6H, CH₃), 6.19 (m 1H, NH), 6.89 (m, 2H, Ph-H), 6.92 (d, 1H, J=5.5 Hz, pyrimidinyl-H), 7.19 (t, 1H, J=7.0 Hz, Ph-H), 7.27 (s, 2H, Ph-H), 7.36 (m, 2H, Ph-H), 7.42 (m, 1H, Ph-H), 8.32 (d, 1H, J=5.5 Hz, pyrimidinyl-H). MS (ESI⁺) m/z 419.87 (C₂₂H₂₁N₅O₂S requires 419.50).

1-(4-{4-[4-(4-Methyl-2-phenylamino-thiazol-5-yl)-pyrimidin-2-ylamino]-phenyl}-piperazin-1-yl)-ethanone (172). By reaction between 3-dimethylamino-1-(4-methyl-2-phenylamino-thiazol-5-yl)-propenone and N-[4-(4-acetyl-piperazin-1-yl)-phenyl]-guanidine. Yellow solid. Mp 245-246° C. Anal. RP-HPLC: t_{R}=14.5 min (0-60% MeCN; purity 100%). ¹H-NMR (CDCl₃) δ: 2.16 (s, 3H, CH₃), 2.62 (s, 3H, CH₃), 3.11-3.16 (m, 4H, CH₂), 3.64 (t, 2H, J=5.0 Hz, CH₂), 3.80 (t, 2H, J=5.0 Hz, CH₂), 6.86 (d, 1H, J=5.5 Hz, pyrimidinyl-H), 6.94 (d, 2H, J=9.0 Hz, Ph-H), 7.18 (t, 1H, J=6.5 Hz, Ph-H), 7.42 (m, 4H, Ph-H), 7.52 (d, 2H, J=9.0 Hz, Ph-H), 8.27 (d, 1H, J=5.5 Hz, pyrimidinyl-H). MS (ESI⁺) m/z 486.03 (C₂₆H₂₇N₇OS requires 485.61).

[4-(2,4-Dimethyl-thiazol-5-yl)-pyrimidin-2-yl]-(3-methoxy-4-morpholin-4-yl-phenyl)-amine (173). By reaction between 3-dimethylamino-1-(2,4-dimethyl-thiazol-5-yl)-propenone and N-(3-methoxy-4-morpholin-4-yl-phenyl)-guanidine. Yellow solid. Anal. RP-HPLC: t_{R}=16.7 min (10-70% MeCN; purity 100%). ¹H-NMR (DMSO-d₆) δ: 2.62 (s, 3H, CH₃), 2.64 (s, 3H, CH₃), 2.91 (t, 4H, J=4.0 Hz, CH₂), 3.71 (t, 4H, J=4.0 Hz, CH₂), 3.80 (s, 3H, CH₃), 6.83 (d, 1H, J=8.0 Hz, Ph-H), 7.04 (d, 1H, J=5.0 Hz, pyrimidinyl-H), 7.30 (d, 1H, J=2.5, 9.0 Hz, Ph-H), 7.43 (d, 1H, J=2.5 Hz, Ph-H), 8.48 (d, 1H, J=5.5 Hz, pyrimidinyl-H), 9.47 (s, 1H, NH). MS (ESI⁺) m/z 397.94 (C₂₀H₂₃N₂OS requires 397.50).

[4-(2,4-Dimethyl-thiazol-5-yl)-pyrimidin-2-yl]-(4-morpholin-4-ylmethyl-phenyl)-amine (174). By reaction between 3-dimethylamino-1-(2,4-dimethyl-thiazol-5-yl)-propenone and N-(4-morpholin-4-ylmethyl-phenyl)-guanidine. Yellow solid. Anal. RP-HPLC: t_{R}=17.3 min (10-70% MeCN; purity 100%). ¹H-NMR (DMSO-d₆) δ: 2.34 (m, 2H, CH₂), (2.62 (s, 3H, CH₃), 2.65 (s, 3H, CH₃), 3.28 (s, 2H, CH₂), 3.40 (br. s, 2H, CH₂), 3.57 (m, 4H, J=4.0 Hz, CH₂), 7.07 (d, 1H, J=5.0 Hz, pyrimidinyl-H), 7.22 (d, 1H, J=8.5 Hz, Ph-H), 7.73 (d, 1H, J=3.5, 8.5 Hz, Ph-H), 8.51 (d, 1H, J=5.0 Hz, pyrimidinyl-H), 9.64 (s, 1H, NH). MS (ESI⁺) m/z 381.96 (C₂₀H₂₃N₅OS requires 381.50).

(3,5-Dimethoxy-phenyl)-[4-(2,4-dimethyl-thiazol-5-yl)-pyrimidin-2-yl]-amine (175). By reaction between 3-dimethylamino-1-(2,4-dimethyl-thiazol-5-yl)-propenone and N-(3,5-dimethoxy-phenyl)-guanidine. Yellow solid. Anal. RP-HPLC: t_{R}=17.3 min (10-70% MeCN; purity 100%). ¹NMR (CDCl₃) δ: 2.63 (s, 3H, CH₃), 2.65 (s, 3H, CH₃), 3.74 (s, 6H, CH₃), 6.14 (m 1H, Ph-H), 7.07 (m, 2H, Ph-H), 7.10 (d, 1H, J=5.0 Hz, pyrimidinyl-H), 8.52 (d, 1H, J=5.0 Hz, pyrimidinyl-H), 9.60 (s, 1H, NH). MS (ESI⁺) m/z 342.98 (C₁₇H₁₈N₄O₂S requires 342.42).

[4-(4-Benzyl-piperazin-1-yl)-phenyl]-[4-(4-methyl-2-phenylamino-thiazol-5-yl)-pyrimidin-2-yl]-amine (176). By reaction between 3-dimethylamino-1-(4-methyl-2-phenylamino-thiazol-5-yl)-propenone and N-[4-(4-benzyl-piperazin-1-yl)-phenyl]-guanidine. Yellow solid. Mp 227-229° C. Anal. RP-HPLC: t_{R}=15.2 min (0-60% MeCN; purity 100%). ¹H-NMR (DMSO-d₆) δ: 2.57 (s, 3H, CH₃), 3.08 (m, 4H, CH₂), 3.33 (m, 4H, CH₂), 3.53 (s, 4H, CH₂), 6.88 (d, 2H, J=8.0 Hz, Ph-H), 6.93 (d, 1H, J=5.0 Hz, pyrimidinyl-H), 7.02 (t, 1H, J=7.5 Hz, Ph-H), 7.26-7.35 (m, 7H, Ph-H), 7.50 (dd, 4H, J=3.36, 6.71 Hz, Ph-H), 8.34 (d, 1H, J=5.0 Hz, pyrimidinyl-H), 9.26 (br. s, 1H, NH). MS (ESI⁺) m/z 533.96 (C₃₁H₃₁N₇S requires 533.69).

Benzo[1,3]dioxol-5-yl-[4-(4-methyl-2-pyridin-3-yl-thiazol-5-yl)-pyrimidin-2-yl]-amine (177). By reaction between 3-dimethylamino-1-(4-methyl-2-pyridin-3-yl-thiazol-5-yl)-propenone and N-benzo[1,3]dioxol-5-yl-guanidine. Yellow solid. Mp 187-188° C. Anal. RP-HPLC: t_{R}=16.0 min (0-60% MeCN; purity 100%). ¹H-NMR (DMSO-D₆) δ: 2.75 (s, 3H, CH₃), 5.98 (s, 2H, CH₂), 6.90 (d, 1H, J=8.5 Hz, Ph-H), 7.15 (d, 1H, J=5.0 Hz, pyrimidinyl-H), 7.21 (d, 1H, J=8.5 Hz, Ph-H), 7.46 (s, 1H, Ph-H), 7.57 (m, 1H, Ar—H), 8.32 (d, 1H, J=8.0 Hz, Ar—H), 8.54 (d, 1H, J=5.0 Hz, pyrimidinyl-H), 8.70 (d, 1H, J=4.5 Hz, Ar—H), 9.14 (s, 1H, Ar—H). MS (ESI⁺) m/z 389.88 (C₂₀H₁₅N₅O₂S requires 389.43).

Benzo[1,3]dioxol-5-yl-[4-(2-ethylamino-4-methyl-thiazol-5-yl)-pyrimidin-2-yl]-amine (178). By reaction between 3-dimethylamino-1-(2-ethylamino-4-methyl-thiazol-5-yl)-propenone and N-benzo[1,3]dioxol-5-yl-guanidine. Yellow solid. Mp 194-195° C. Anal. RP-HPLC: t_{R}=13.9 min (0-60% MeCN; purity 100%). ¹H-NMR (DMSO-d₆) δ: 1.17 (t, 3H, J=7.5 Hz, CH₃), 2.45 (s, 3H, CH₃), 3.26 (m, 2H, CH₂), 5.96 (s, 2H, CH₂), 6.81 (d, 1H, J=8.5 Hz, Ph-H), 6.85 (d, 1H, J=5.0 Hz, pyrimidinyl-H), 7.19 (d, 1H, J=8.5 Hz, Ph-H), 7.54 (s, 1H, Ph-H), 8.10 (t, 1H, J=5.0 Hz, NH), 8.29 (d, 1H, J=5.0 Hz, pyrimidinyl-H), 9.30 (s, 1H, NH). MS (ESI⁺) m/z 355.87 (C₁₇H₁₇N₅O₂S requires 355.42).

[4-(2-Amino-4-methyl-thiazol-5-yl)-pyrimidin-2-yl]-benzo[1,3]dioxol-5-yl-amine (179). By reaction between N′-[5-(3-dimethylamino-acryloyl)-4-methyl-thiazol-2-yl]-N,N-dimethyl-formamidine and N-benzo[1,3]dioxol-5-yl-guanidine. Yellow solid. Mp 211-213° C. Anal. RP-HPLC: t_{R}=12.1 min (0-60% MeCN; purity 98%). ¹H-NMR (DMSO-d₆) δ: 2.45 (s, 3H, CH₃), 5.96 (s, 2H, CH₂), 6.81 (d, 1H, J=8.5 Hz, Ph-H), 6.84 (d, 1H, J=5.0 Hz, pyrimidinyl-H), 7.09 (d, 1H, J=8.0 Hz, Ph-H), 7.50 (s, 2H, NH₂), 7.54 (s, 1H, Ph-H), 8.29 (d, 1H, J=5.5 Hz, pyrimidinyl-H), 9.31 (s, 1H, NH). MS (ESI⁺) m/z 327.92 (C₁₅H₁₃N₅O₂S requires 327.36).

(2,3-Dihydro-benzo[1,4]dioxin-6-yl)-[4-(4-methyl-2-pyridin-3-yl-thiazol-5-yl)-pyrimidin-2-yl]-amine (180). By reaction between 3-dimethylamino-1-(4-methyl-2-pyridin-3-yl-thiazol-5-yl)-propenone and N-(2,3-dihydro-benzo[1,4]dioxin-6-yl)-guanidine. Yellow solid. Anal. RP-HPLC: t_{R}=16.2 min (0-60% MeCN; purity 100%). ¹H-NMR (CDCl₃) δ: 2.81 (s, 3H, CH₃), 4.30 (m, 4H, CH₂), 6.81 (d, 1H, J=8.5 Hz, Ph-H), 6.99 (m, 2H, pyrimidinyl-H and Ph-H), 7.36 (d, 1H, J=2.5 Hz, Ph-H), 7.43 (m, 2H, Ar—H), 8.30 (d, 1H, J=5.5 Hz, Ar—H), 8.41 (d, 1H, J=5.0 Hz, pyrimidinyl-H), 8.70 (d, 1H, J=5.0 Hz, Ar—H), 9.22 (s, 1H, NH). MS (ESI⁺) m/z 402.93 (C₂₁H₁₇N₅O₂S requires 403.46).

(2,3-Dihydro-benzo[1,4]dioxin-6-yl)-[4-(2-ethylamino-4-methyl-thiazol-5-yl)-pyrimidin-2-yl]-amine (181). By reaction between 3-dimethylamino-1-(2-ethylamino-4-methyl-thiazol-5-yl)-propenone and N-(2,3-dihydro-benzo[1,4]dioxin-6-yl)-guanidine. Yellow solid. Anal. RP-HPLC: t_{R}=16.2 min (0-60% MeCN; purity 100%). ¹H-NMR (CDCl₃) 1.17 (t, 3H, J=7.0 Hz, CH₃), 2.45 (s, 3H, CH₃), 3.27 (m, 2H, CH₂), 4.20 (dd, 4H, J=5.0, 14.5 Hz, CH₂), 6.74 (d, 1H, J=9.0 Hz, Ph-H), 6.84 (d, 1H, J=5.5 Hz, pyrimidinyl-H), 7.15 (dd, 1H, J=2.4, 8.8 Hz, Ph-H), 7.43 (d, 1H, J=2.4 Hz, Ph-H), 8.09 (t, 1H, J=5.0 Hz, NH), 8.28 (d, 1H, J=5.5 Hz, pyrimidinyl-H), 9.21 (s, 1H, NH). MS (ESI⁺) m/z 369.93 (C₁₈H₁₉N₅O₂S requires 369.44).

[4-(2-Amino-4-methyl-thiazol-5-yl)-pyrimidin-2-yl]-(3-methoxy-4-morpholin-4-yl-phenyl)-amine (182). By reaction between N′-[5-(3-dimethylamino-acryloyl)-4-methyl-thiazol-2-yl]-N,N-dimethyl-formamidine and N-(3-methoxy-4-morpholin-4-yl-phenyl)-guanidine. Yellow solid. Anal. RP-HPLC: t_{R}=9.9 min (0-60% MeCN; purity 98%). ¹H-NMR (DMSO-d₆) δ: 2.42 (s, 3H, CH₃), 2.90 (t, 4H, J=4.5 Hz, CH₂), 3.71 (t, 4H, J=4.5 Hz, CH₂), 3.81 (s, 3H, CH₃), 6.79 (d, 1H, J=8.5 Hz, Ph-H), 6.85 (d, 1H, J=5.0 Hz, pyrimidinyl-H), 7.27 (dd, 1H, J=2.0, 8.0 Hz, Ph-H), 7.43 (d, 1H, J=2.0 Hz, Ph-H), 7.48 (s, 2H, NH₂), 8.29 (d, 1H, J=5.0 Hz, pyrimidinyl-H), 9.23 (s, 1H, NH). MS (ESI⁺) m/z 398.96 (C₁₉H₂₂N₆O₂S requires 398.48).

(2,3-Dihydro-benzo[1,4]dioxin-6-yl)-[4-(4-methyl-2-phenethylamino-thiazol-5-yl)-pyrimidin-2-yl]-amine (183). By reaction between 3-dimethylamino-1-(4-methyl-2-phenylamino-thiazol-5-yl)-propenone and N-(2,3-dihydro-benzo[1,4]dioxin-6-yl)-guanidine. Yellow solid. Anal. RP-HPLC: t_{R}=17.3 min (0-60% MeCN; purity 100%). ¹H-NMR (DMSO-d₆) δ: 2.46 (s, 3H, CH₃), 2.89 (t, 2H, J=7.5 Hz, CH₂), 3.48 (t, 2H, J=6.5 Hz, CH₂), 4.19 (m, 4H, CH₂), 6.74 (d, 1H, J=9.0 Hz, Ph-H), 6.84 (d, 1H, J=5.5 Hz, pyrimidinyl-H), 7.13 (dd, 1H, J=2.5, 9.0 Hz, Ph-H), 7.20-7.33 (m, 5H, Ph-H), 7.42 (d, 4H, J=2.5 Hz, Ph-H), 8.22 (t, 1H, J=5.5 Hz, NH), 8.28 (d, 1H, J=5.0 Hz, pyrimidinyl-H), 9.22 (s, 1H, NH). MS (ESI⁺) m/z 445.88 (C₂₄H₂₃N₇O₂S requires 445.54).

(4-Methoxy-3-methyl-phenyl)-[4-(4-methyl-2-methylamino-thiazol-5-yl)-pyrimidin-2-yl]-amine (184). By reaction between 3-dimethylamino-1-(4-methyl-2-methylamino-thiazol-5-yl)-propenone and N-(4-methoxy-3-methyl-phenyl)-guanidine. Yellow solid. Anal. RP-HPLC: t_{R}=14.2 min (0-60% MeCN; purity 100%). ¹H-NMR (DMSO-d₆) δ: 2.16 (s, 3H, CH₃), 2.46 (s, 3H, CH₃), 2.85 (d, 3H, J=5.0 Hz, CH₃), 3.74 (s, 3H, CH₃), 6.83 (m, 2H, Ph-H and pyrimidinyl-H), 7.45 (d, 1H, J=2.5, 8.5 Hz, Ph-H), 7.62 (d, 1H, J=2.0 Hz, Ph-H), 8.02 (m, 1H, NH), 8.27 (d, 1H, J=5.5 Hz, pyrimidinyl-H), 9.16 (s, 1H, NH). MS (ESI⁺) m/z 341.94 (C₁₇H₁₉N₅OS requires 341.43).

[4-(2-Amino-4-methyl-thiazol-5-yl)-pyrimidin-2-yl]-(4-methoxy-3-methyl-phenyl)-amine (185). By reaction between N′-[5-(3-dimethylamino-acryloyl)-4-methyl-thiazol-2-yl]-N,N-dimethyl-formamidine and N-(4-methoxy-3-methyl-phenyl)-guanidine. Yellow solid. Anal. RP-HPLC: t_{R}=13.3 min (0-60% MeCN; purity 97%). ¹H-NMR (DMSO-d₆) δ: 2.16 (s, 3H, CH₃), 2.42 (s, 3H, CH₃), 3.74 (s, 3H, CH₃), 6.83 (d, 1H, J=5.5 Hz, pyrimidinyl-H), 6.83 (d, 1H, J=8.5 Hz, Ph-H), 7.46 (br. s, 1H, NH₂), 7.51 (m, 1H, Ph-H), 7.56 (d, 1H, J=2.5 Hz, Ph-H), 8.27 (d, 1H, J=5.5 Hz, pyrimidinyl-H), 9.14 (s, 1H, NH). MS (ESI⁺) m/z 327.94 (C_{≠}H₁₇N₅OS requires 327.41).

4-Methoxy-3-methyl-phenyl)-[4-(4-methyl-2-pyridin-3-yl-thiazol-5-yl)-pyrimidin-2-yl]-amine (186). By reaction between 3-dimethylamino-1-(4-methyl-2-pyridin-3-yl-thiazol-5-yl)-propenone and N-(4-methoxy-3-methyl-phenyl)-guanidine. Yellow solid. Anal. RP-HPLC: t_{R}=17.5 min (0-60% MeCN; purity 100%). ¹H-NMR (DMSO-d₆) δ: 2.18 (s, 3H, CH₃), 2.75 (s, 3H, CH₃), 3.77 (s, 3H, CH₃), 6.91 (d, 1H, J=9.0 Hz, Ph-H), 7.11 (d, 1H, J=5.0 Hz, pyrimidinyl-H), 7.52-7.58 (m, 4H, Ph-H and Ar—H), 8.32 (d, 1H, J=8.0 Hz, Ph-H), 8.52 (d, 1H, J=5.0 Hz, pyrimidinyl-H), 8.70 (d, 1H, J=4.5 Hz, Ar—H), 9.15 (m, 1H, Ar—H), 9.47 (s, 1H, NH). MS (ESI⁺) m/z 389.88 (C₂₁H₁₉N₅OS requires 389.47).

[4-(2-Ethylamino-4-methyl-thiazol-5-yl)-pyrimidin-2-yl]-(4-methoxy-3-methyl-phenyl)-amine (187). By reaction between 3-dimethylamino-1-(2-ethylamino-4-methyl-thiazol-5-yl)-propenone and N-(4-methoxy-3-methyl-phenyl)-guanidine. Yellow solid. Anal. RP-HPLC: t_{R}=15.0 min (0-60% MeCN; purity 100%). ¹H-NMR (DMSO-d₆) δ: 1.17 (t, 3H, J=7.0 Hz, CH₃), 2.16 (s, 3H, CH₃), 2.45 (s, 3H, CH₃), 3.24-3.29 (m, 2H, CH₂), 3.74 (s, 3H, CH₃), 6.83 (m, 2H, Ph-H and pyrimidinyl-H), 7.45 (d, 1H, J=9.0 Hz, Ph-H), 7.62 (br. s, 1H, Ph-H), 8.08 (t, 1H, J=5.5 Hz, NH), 8.27 (d, 1H, J=5.0 Hz, pyrimidinyl-H), 9.15 (s, 1H, NH). MS (ESI⁺) m/z 355.94 (C₁₈H₂₁N₅OS requires 355.46).

{4-Methyl-5-[2-(4-morpholin-4-yl-phenylamino)-pyrimidin-4-yl]-thiazol-2-yl}-methanol (188). By reaction between 3-dimethylamino-1-(2-hydroxymethyl-4-methyl-thiazol-5-yl)-propenone and N-(4-morpholin-4-ylmethyl-phenyl)-guanidine. Yellow solid. Anal. RP-HPLC: t_{R}=11.1 min (0-60% MeCN; purity 97%). ¹H-NMR (DMSO-d₆) δ: 2.63 (s, 3H, CH₃), 3.04 (t, 4H, J=4.5 Hz, CH₂), 3.73 (t, 4H, J=4.5 Hz, CH₂), 4.70 (d, 2H, J=6.0 Hz, CH₂), 6.13 (t, 1H, J=6.0 Hz, OH), 6.90 (d, 2H, J=9.0 Hz, Ph-H), 7.02 (d, 1H, J=5.0 Hz, pyrimidinyl-H), 7.62 (d, 2H, J=9.0 Hz, Ph-H), 8.46 (d, 1H, J=5.0 Hz, pyrimidinyl-H), 9.41 (s, 1H, NH). MS (ESI⁺) m/z 384.06 (C₁₉H₂₁N₅O₂S requires 383.47).

4-{4-[4-(2,4-Dimethyl-thiazol-5-yl)-pyrimidin-2-ylamino]-phenyl}-piperazine-1-carboxylic acid ethyl ester (189). By reaction between 3-dimethylamino-1-(2,4-dimethyl-thiazol-5-yl)-propenone and 4-(4-guanidino-phenyl)-piperazine-1-carboxylic acid ethyl ester. Yellow solid. Anal. RP-HPLC: t_{R}=13.2 min (10-70% MeCN; purity 100%). ¹H-NMR (DMSO-d₆) δ: 1.20 (t, 3H, J=7.0 Hz, CH₃), 2.62 (s, 3H, CH₃), 2.64 (s, 3H, CH₃), 3.04 (t, 4H, J=5.0 Hz, CH₂), 3.51 (t, 4H, J=5.0 Hz, CH₂), 4.07 (q, 2H, J=7.0 Hz, CH₂), 6.93 (t, 2H, J=8.5 Hz, Ph-H), 6.99 (d, 1H, J=5.0 Hz, pyrimidinyl-H), 7.62 (d, 2H, J=8.5 Hz, Ph-H), 8.45 (d, 1H, J=5.0 Hz, pyrimidinyl-H), 9.43 (s, 1H, NH). MS (ESI⁺) m/z 438.79 (C₂₂H₂₆N₆O₂S requires 438.55).

2-(4-{4-[4-(2,4-Dimethyl-thiazol-5-yl)-pyrimidin-2-ylamino]-phenyl}-piperazin-1-yl)-N-isopropyl-acetamide (190). By reaction between 3-dimethylamino-1-(2,4-dimethyl-thiazol-5-yl)-propenone and 2-[4-(4-guanidino-phenyl)-piperazin-1-yl]-N-isopropyl-acetamide. Yellow solid. Anal. RP-HPLC: t_{R}=10.6 min (10-70% MeCN; purity 100%). ¹H-NMR (DMSO-d₆) δ: 1.07 (d, 6H, J=7.0 Hz, CH₂), 2.58 (m, 4H, CH₂), 2.62 (s, 3H, CH₃), 2.64 (s, 3H, CH₃), 2.93 (s, 2H, CH₂), 3.11 (m, 4H, CH₂), 6.90 (t, 2H, J=9.0 Hz, Ph-H), 6.99 (d, 1H, J=5.5 Hz, pyrimidinyl-H), 7.49 (d, 1H, J=3.5 Hz, NH), 7.60 (d, 2H, J=8.5 Hz, Ph-H), 8.44 (d, 1H, J=5.0 Hz, pyrimidinyl-H), 9.40 (s, 1H, NH). MS (ESI⁺) m/z 465.80 (C₂₄H₃₁N₇OS requires 465.62).

[4-(4-Methyl-2-methylamino-thiazol-5-yl)-pyrimidin-2-yl]-[4-(4-methyl-piperazin-1-yl)-phenyl]-amine (191). By reaction between 3-dimethylamino-1-(4-methyl-2-methylamino-thiazol-5-yl)-propenone and N-[4-(4-methyl-piperazin-1-yl)-phenyl]-guanidine. Yellow solid. Anal. RP-HPLC: t_{R}=8.1 min (10-70% MeCN; purity 100%). ¹H-NMR (DMSO-de) δ: 2.25 (s, 3H, CH₃), 2.46 (m, 4H, CH₂), 2.85 (d, 2H, J=4.5 Hz, CH₂), 3.05 (m, 4H, CH₂), 6.81 (d, 1H, J=5.5 Hz, pyrimidinyl-H), 6.87 (d, 2H, J=9.5 Hz, Ph-H), 7.60 (d, 1H, J=9.5 Hz, Ph-H), 8.00 (m, 1H, NH), 8.26 (d, 1H, J=5.5 Hz, pyrimidinyl-H), 9.15 (s, 1H, NH). MS (ESI⁺) m/z 396.02 (C₂₀H₂₅N₇S requires 395.53).

[4-(4-Methyl-2-methylamino-thiazol-5-yl)-pyrimidin-2-yl]-[4-(4-methyl-piperazin-1-yl)-phenyl]-amine (192). By reaction between 3-dimethylamino-1-(4-methyl-2-methylamino-thiazol-5-yl)-propenone and N-[4-(4-methyl-piperazin-1-yl)-phenyl]-guanidine. Yellow solid. Anal. RP-HPLC: t_{R}=12.8 min (0-60% MeCN; purity 100%). ¹H-NMR (DMSO-de) δ: 1.51 (m, 2H, CH₂), 1.63 (m, 4H, CH₂), 2.75 (s, 3H, CH₃), 3.07 (t, 4H, J=5.0 Hz, CH₂), 6.93 (d, 2H, J=9.0 Hz, Ph-H), 7.10 (d, 1H, J=5.0 Hz, pyrimidinyl-H), 7.56 (q, 1H, J=4.5 Hz, Ar—H), 8.34 (m, 1H, Ar—H), 8.51 (d, 1H, J=5.0 Hz, pyrimidinyl-H), 8.70 (m, 1H, Ar—H), 9.17 (d, 1H, J=2.5 Hz, Ar—H), 9.46 (s, 1H, NH). MS (ESI⁺) m/z 428.96 (C₂₄H₂₄N₆S requires 428.55).

[4-(2,4-Dimethyl-thiazol-5-yl)-pyrimidin-2-yl]-(4-piperidin-1-yl-phenyl)-amine (193). By reaction between 3-dimethylamino-1-(2,4-dimethyl-thiazol-5-yl)-propenone and N-(4-piperidin-1-yl-phenyl)-guanidine. Yellow solid. Anal. RP-HPLC: t_{R}=12.4 min (0-60% MeCN; purity 100%). ¹NMR (DMSO-d₆) δ: 1.51 (m, 2H, CH₂), 1.62 (m, 4H, CH₂), 2.61 (s, 3H, CH₃), 2.64 (s, 3H, CH₃), 3.04 (t, 4H, 5.5 Hz, CH₂), 6.88 (d, 2H, 9.0 Hz, Ph-H), 6.98 (d, 1H, J=5.0 Hz, pyrimidinyl-H), 7.57 (d, 1H, J=9.0 Hz, Ph-H), 8.44 (d, 1H, J=5.0 Hz, pyrimidinyl-H), 9.37 (s, 1H, NH). MS (ESI⁺) m/z 366.96 (C₂₀H₂₃N₅S requires 365.50).

[4-(2-Ethylamino-4-methyl-thiazol-5-yl)-pyrimidin-2-yl]-(4-piperidin-1-yl-phenyl)-amine (194). By reaction between 3-dimethylamino-1-(2-ethylamino-4-methyl-thiazol-5-yl)-propenone and N-(4-piperidin-1-yl-phenyl)-guanidine. Yellow solid. Anal. RP-HPLC: t_{R}=11.0 min (0-60% MeCN; purity 100%). ¹H-NMR (DMSO-d₆) δ: 1.17 (t, 3H, J=7.0 Hz, CH₃), 1.51 (m, 2H, CH₂), 1.62 (m, 4H, CH₂), 2.45 (s, 3H, CH₃), 3.03 (t, 4H, J=5.5 Hz, CH₂), 3.27 (m, 2H, CH₂), 6.80 (d, 1H, J=5.5 Hz, pyrimidinyl-H), 6.85 (d, 2H, J=9.0 Hz, Ph-H), 7.57 (d, 1H, J=9.0 Hz, Ph-H), 8.04 (t, 1H, J=5.5 Hz, NH), 8.26 (d, 1H, J=5.5 Hz, pyrimidinyl-H), 9.13 (s, 1H, NH). MS (ESI⁺) m/z 395.00 (C₂₁H₂₆N₆S requires 394.54).

[4-(2-Amino-4-methyl-thiazol-5-yl)-pyrimidin-2-yl]-(4-piperidin-1-yl-phenyl)-amine (195). By reaction between N′-[5-(3-dimethylamino-acryloyl)-4-methyl-thiazol-2-yl]-N,N-dimethyl-formamidine and N-(4-piperidin-1-yl-phenyl)-guanidine. Yellow solid. Anal. RP-HPLC: t_{R}=11.0 min (0-60% MeCN; purity 100%). ¹H-NMR (DMSO-d₆) δ: 1.51 (m, 2H, CH₂), 1.62 (m, 4H, CH₂), 2.45 (s, 3H, CH₃), 3.03 (t, 4H, J=5.5 Hz, CH₂), 6.79 (d, 1H, J=5.0 Hz, pyrimidinyl-H), 6.85 (d, 2H, J=9.0 Hz, Ph-H), 7.44 (s, 2H, NH₂), 7.57 (d, 1H, J=9.0 Hz, Ph-H), 8.26 (d, 1H, J=5.0 Hz, pyrimidinyl-H), 9.13 (s, 1H, NH).

[4-(2-Ethylamino-4-methyl-thiazol-5-yl)-pyrimidin-2-yl]-[4-(4-methyl-piperazin-1-yl)-phenyl]-amine (196). By reaction between 3-dimethylamino-1-(2-ethylamino-4-methyl-thiazol-5-yl)-propenone and N-[4-(4-methyl-piperazin-1-yl)-phenyl]-guanidine. Yellow solid. Anal. RP-HPLC: t_{R}=8.34 min (10-70% MeCN; purity 97%). ¹H-NMR (DMSO-d₆) δ: 1.17 (m, 3H, CH₃), 2.21 (m, 7H, CH₃ and CH₂), 2.42 (s, 3H, CH₃), 3.04 (m, 4H, CH₂), 6.81 (d, 1H, 5.5 Hz, pyrimidinyl-H), 6.86 (d, 2H, J=9.5 Hz, Ph-H), 7.59 (d, 1H, J=9.5 Hz, Ph-H), 8.04 (m, 1H, NH), 8.26 (d, 1H, J=5.5 Hz, pyrimidinyl-H), 9.14 (s, 1H, NH). MS (ESI⁺) m/z 410.02 (C₂₁H₂₇N₇S requires 409.55).

{4-Methyl-5-[2-(4-piperidin-1-yl-phenylamino)-pyrimidin-4-yl]-thiazol-2-yl}-methanol (197). By reaction between 3-dimethylamino-1-(2-hydroxymethyl-4-methyl-thiazol-5-yl)-propenone and N-(4-piperidin-1-yl-phenyl)-guanidine. Yellow solid. Mp 194-195° C.; Anal. RP-HPLC: t_{R}=11.5 min (0-60% MeCN; purity 100%). ¹H-NMR (DMSO-d₆) δ: 1.48-1.53 (m, 2H, CH₂), 1.60-1.65 (m, 4H, CH₂) 2.63 (s, 3H, CH₃), 3.05 (t, 4H, J=5.5 Hz, CH₂), 4.70 (d, 2H, J=6.0 Hz, CH₂), 6.12 (t, 1H, J=6.0 Hz, OH), 6.88 (d, 2H, J=9.0 Hz, Ph-H), 7.01 (d, 1H, J=5.0 Hz, pyrimidinyl; —H), 7.58 (d, 2H, J=9.0 Hz, Ph-H), 8.45 (d, 1H, J=5.0 Hz, pyrimidinyl-H), 9.37 (s, 1H, NH). MS (ESI⁺) m/z 382.02 (C₂₀H₂₃N₅OS requires 381.50).

[4-(4-Methyl-2-pyridin-3-yl-thiazol-5-yl)-pyrimidin-2-yl]-(4-pyrrolidin-1-yl-phenyl)-amine (198). By reaction between 3-dimethylamino-1-(4-methyl-2-pyridin-3-yl-thiazol-5-yl)-propenone and N-(4-pyrrolidin-1-yl-phenyl)-guanidine. Yellow solid. Mp 212-214° C.; Anal. RP-HPLC: t_{R}=12.9 min (0-60% MeCN; purity 100%). ¹H (DMSO-d₆) δ: 1.93 (m, 4H, CH₂), 2.74 (s, 3H, CH₃), 3.20 (t, 4H, J=6.5 Hz, CH₂), 6.54 (d, 2H, J=9.0 Hz, Ph-H), 7.05 (d, 1H, J=5.0 Hz, pyrimidinyl-H), 7.53 (d, 2H, J=8.5 Hz, Ph-H), 7.57 (m, 1H, Ar—H), 8.33 (d, 1H, J=8.0 Hz, Ar—H), 8.47 (d, 1H, J=5.0 Hz, pyrimidinyl-H), 8.70 (d, 1H, J=4.5 Hz, Ar—H), 9.16 (s, 1H, Ar—H), 9.30 (br. s, 1H, NH). MS (ESI⁺) m/z 414.95 (C₂₃H₂₂N₆S requires 414.53).

[4-(2,4-Dimethyl-thiazol-5-yl)-pyrimidin-2-yl]-(4-pyrrolidin-1-yl-phenyl)-amine (199). By reaction between 3-dimethylamino-1-(2,4-dimethyl-thiazol-5-yl)-propenone and N-(4-pyrrolidin-1-yl-phenyl)-guanidine. Yellow solid. Mp 192-193° C.; Anal. RP-HPLC: t_{R}=12.5 min (0-60% MeCN; purity 100%). ¹H (DMSO-d₆) δ: 1.93-1.96 (m, 4H, CH₂), 2.61 (s, 3H, CH₃), 2.64 (s, 3H, CH₃), 3.20 (t, 4H, J=6.5 Hz, CH₂), 6.51 (d, 2H, J=9.0 Hz, Ph-H), 6.94 (d, 1H, J=5.0 Hz, pyrimidinyl-H), 7.51 (d, 2H, J=9.0 Hz, Ph-H), 8.40 (d, 1H, J=5.0 Hz, pyrimidinyl-H), 9.22 (s, 1H, NH). ¹³C-NMR (DMSO-d₆) δ: 18.56, 19.67, 25.60, 48.28, 107.92, 112.21, 121.94, 129.70, 131.70, 144.45, 152.32, 158.53, 159.60, 160.70, 166.80. MS (ESI⁺) m/z 350.95 (C₁₉H₂₁N₅S requires 351.47).

{5-[2-(3-Methoxy-4-morpholin-4-yl-phenylamino)-pyrimidin-4-yl]-4-methyl-thiazol-2-yl}-methanol (200). By reaction between 3-dimethylamino-1-(2-hydroxymethyl-4-methyl-thiazol-5-yl)-propenone and N-(3-methoxy-4-morpholin-4-yl-phenyl)-guanidine. Yellow solid. Mp 180-181° C.; Anal. RP-HPLC: t_{R}=11.4 min (0-60% MeCN; purity 100%). ¹H (DMSO-d₆) δ: 2.63 (s, 3H, CH₃), 2.91 (t, 4H, J=4.5 Hz, CH₂), 3.71 (t, 4H, J=4.5 Hz, CH₂), 3.82 (s, 3H, CH₃), 4.70 (d, 2H, J=6.0 Hz, CH₂), 6.14 (t, 1H, J=6.0 Hz, OH), 6.82 (d, 1H, J=8.5 Hz, Ph-H), 7.06 (d, 1H, J=5.0 Hz, pyrimidinyl-H), 7.26 (d, 1H, J=8.5 Hz, Ph-H), 7.53 (s, 1H, Ph-H), 8.49 (d, 1H, J=5.0 Hz, pyrimidinyl-H), 9.49 (br. s, 1H, NH). MS (ESI⁺) m/z 413.93 (C₂₀H₂₃N₅O₃S requires 413.49).

[4-(2-Amino-4-methyl-thiazol-5-yl)-pyrimidin-2-yl]-(4-thiomorpholin-4-yl-phenyl)-amine (201). By reaction between N′-[5-(3-dimethylamino-acryloyl)-4-methyl-thiazol-2-yl]-N,N-dimethyl-formamidine and N-(4-thiomorpholin-4-yl-phenyl)-guanidine. Yellow solid. Mp 180-182° C.; Anal. RP-HPLC: t_{R}=10.4 min (0-60% MeCN; purity 100%). ₁H (DMSO-d₆) δ: 2.08 (s, 3H, CH₃), 2.69 (t, 4H, J=5.0 Hz, CH₂), 3.38 (m, 4H, CH₂), 6.80 (d, 1H, J=5.5 Hz, pyrimidine-H), 6.86 (d, 2H, J=9.0 Hz, Ph-H), 7.45 (s, 2H, NH₂), 7.61 (d, 2H, J=9.0 Hz, Ph-H), 8.26 (d, 1H, J=5.5 Hz, pyrimidinyl-H), 9.18 (s, 1H, NH). MS (ESI⁺) m/z 385.43 (C₁₈H₂₀N₆S₂ requires 384.52).

[4-(2,4-Dimethyl-thiazol-5-yl)-pyrimidin-2-yl]-(4-thiomorpholin-4-yl-phenyl)-amine (202). By reaction between 3-dimethylamino-1-(2,4-dimethyl-thiazol-5-yl)-propenone and N-(4-thiomorpholin-4-yl-phenyl)-guanidine. Yellow solid. Mp 173-174° C.; Anal. RP-HPLC: t_{R}=13.0 min (0-60% MeCN; purity 100%). ¹H (DMSO-d₆) δ: 2.62 (s, 3H, CH₃), 2.64 (s, 3H, CH₃), 2.69 (t, 4H, J=5.0 Hz, CH₂), 3.40 (t, 4H, J=5.0 Hz, CH₂), 6.90 (d, 2H, J=9.0 Hz, Ph-H), 7.00 (d, 1H, J=5.0 Hz, pyrimidinyl-H), 7.61 (d, 2H, J=9.0 Hz, Ph-H), 8.45 (d, 1H, J=5.0 Hz, pyrimidinyl-H), 9.42 (s, 1H, NH). MS (ESI⁺) m/z 384.31 (C₁₉H₂₁N₅S₂ requires 383.54).

[4-(2-Ethylamino-4-methyl-thiazol-5-yl)-pyrimidin-2-yl]-(4-thiomorpholin-4-yl-phenyl)-amine (203). By reaction between 3-dimethylamino-1-(2-ethylamino-4-methyl-thiazol-5-yl)-propenone and N-(4-thiomorpholin-4-yl-phenyl)-guanidine. Yellow solid. Mp 207-209° C. Anal. RP-HPLC: t_{R}=11.6 min (0-60% MeCN; purity 100%). ¹H (DMSO-d₆) δ: 1.17 (t, 3H, J=7.5 Hz, CH₃), 2.45 (s, 3H, CH₃), 2.69 (t, 4H, J=5.0 Hz, CH₂), 3.24-3.30 (m, 2H, CH₂), 3.38 (t, 4H, J=5.0 Hz, CH₂), 6.82 (d, 1H, J=5.5 Hz, pyrimidinyl-H), 6.87 (d, 2H, J=9.0 Hz, Ph-H), 7.61 (d, 1H, J=5.0 Hz, Ph-H), 8.05 (t, 1H, J=5.0 Hz, NH), 8.27 (d, 1H, J= 5.0 Hz, pyrimidinyl-H), 9.17 (bs, 1H, NH). MS (ESI⁺) m/z 413.37 (C₂₀H₂₄N₆S₂ requires 412.58).

[4-(4-Methyl-2-pyridin-3-yl-thiazol-5-yl)-pyrimidin-2-yl]-(4-thiomorpholin-4-yl-phenyl)-amine (204). By reaction between 3-dimethylamino-1-(4-methyl-2-pyridin-3-yl-thiazol-5-yl)-propenone and N-(4-thiomorpholin-4-yl-phenyl)-guanidine. Yellow solid. Mp 191-193° C. Anal. RP-HPLC: t_{R}=13.4 min (0-60% MeCN; purity 100%). ¹H (DMSO-d₆) δ: 2.70 (t, 4H, J=5.0 Hz, CH₂), 2.76 (s, 3H, CH₃), 3.42 (t. 4H, J=5.0 Hz, CH₂), 6.94 (d, 2H, J=9.0 Hz, Ph-H), 7.12 (d, 1H, J=5.0 Hz, pyrimidinyl-H), 7.56-7.59 (m, 1H, Ar—H), 7.64 (d, 2H, J=9.0 Hz, Ph-H), 8.35 (d, J=8.0 Hz, 1H, Ar—H), 8.52 (d, 1H, J=5.0 Hz, pyrimidinyl-H), 8.71 (d, 1H, J=4.5 Hz, Ar—H), 9.18 (s, 1H, Ar—H). MS (ESI⁺) m/z 447.36 (C₂₃H₂₂N₆S₂ requires 446.59).

[4-(2,4-Dimethyl-thiazol-5-yl)-pyrimidin-2-yl]-(3-methyl-4-piperidin-1-yl-phenyl)-amine (205). By reaction between 3-dimethylamino-1-(2,4-dimethyl-thiazol-5-yl)-propenone and N-(3-methyl-4-piperidin-1-yl-phenyl)-guanidine. Yellow solid. Mp 159-160° C. Anal. RP-HPLC: t_{R}=12.8 min (0-60% MeCN; purity 100%). ₁H (DMSO-d₆) δ: 1.52 (m, 2H, CH₂), 1.64 (m, 4H, CH₂), 2.24 (s, 3H, CH₃), 2.64 (s, 3H, CH₃), 2.74 (t, 4H, J=4.5 Hz, CH₂), 6.95 (d, 1H, J=8.5 Hz, Ph-H), 7.01 (d, 1H, J=5.0 Hz, pyrimidinyl-H), 7.51 (d, 1H, J=8.5 Hz, Ph-H), 7.56 (s, 1H, Ph-H), 8.46 (d, 1H, J=5.0 Hz, pyrimidinyl-H), 9.42 (s, 1H, NH). MS (ESI⁺) m/z 380.34 (C₂₁H₂₅N₅S requires 379.52).

[4-(2-Amino-4-methyl-thiazol-5-yl)-pyrimidin-2-yl]-(3-methyl-4-piperidin-1-yl-phenyl)-amine (206). By reaction between N′-[5-(3-dimethylamino-acryloyl)-4-methyl-thiazol-2-yl]-N,N-dimethyl-formamidine and N-(3-methyl-4-piperidin-1-yl-phenyl)-guanidine. Yellow solid. Mp 221-223° C. Anal. RP-HPLC: t_{R}=10.4 min (0-60% MeCN; purity 100%). ₁H (DMSO-d₆) δ: 1.51 (m, 2H, CH₂), 1.65 (m, 4H, CH₂), 2.39 (s, 3H, CH₃), 2.43 (s, 3H, CH₃), 2.74 (t, 4H, J=5.0 Hz, CH₂), 6.82 (d, 1H, J=5.5 Hz, pyrimidinyl-H), 6.91 (d, 1H, J=8.5 Hz, Ph-H), 7.47 (br. s, 2H, NH₂), 7.51 (d, 1H, J=8.5 Hz, Ph-H), 7.56 (s, 1H, Ph-H), 8.28 (d, 1H, J=5.0 Hz, pyrimidinyl-H), 9.18 (s, 1H, NH). MS (ESI⁺) m/z 381.37 (C₂₀H₂₄N₆S requires 380.51).

[4-(2-Ethylamino-4-methyl-thiazol-5-yl)-pyrimidin-2-yl]-(3-methyl-4-piperidin-1-yl-phenyl)-amine (207). By reaction between By reaction between 3-dimethylamino-1-(2-ethylamino-4-methyl-thiazol-5-yl)-propenone and 5-guanidino-2-morpholin-4-yl-benzamide. and N-(3-methyl-4-piperidin-1-yl-phenyl)-guanidine. Yellow solid. Mp 213-214° C. Anal. RP-HPLC: t_{R}=11.2 min (0-60% MeCN; purity 100%). ¹H (DMSO-d₆) δ: 1.18 (t, 3H, J=7.0 Hz, CH₃), 1.52 (m, 2H, CH₂), 1.64 (m, 4H, CH₂), 2.24 (s, 3H, CH₃), 2.45 (s, 3H, CH₃), 2.74 (t, 4H, J=5.0 Hz, CH₂), 3.24-3.29 (m, 2H, CH₂), 6.83 (d, 1H, J=5.5 Hz, pyrimidinyl-H), 6.92 (d, 1H, J=9.0 Hz, Ph-H), 7.45 (d, 1H, J=8.5 Hz, Ph-H), 7.65 (s, 1H, Ph-H), 8.08 (t, 1H, J=5.0 Hz, NH), 8.28 (d, 1H, J=5.5 Hz, pyrimidinyl-H), 9.19 (br. s, 1H, NH). MS (ESI⁺) m/z 408.56 (C₂₂H₂₈N₆S requires 408.56).

(3-Methyl-4-piperidin-1-yl-phenyl)-[4-(4-methyl-2-pyridin-3-yl-thiazol-5-yl)-pyrimidin-2-yl]-amine (208). By reaction between 3-dimethylamino-1-(4-methyl-2-pyridin-3-yl-thiazol-5-yl)-propenone and N-(3-methyl-4-piperidin-1-yl-phenyl)-guanidine. Yellow solid. Mp 200-201° C. Anal. RP-HPLC: t_{R}=13.3 min (0-60% MeCN; purity 100%). ¹H (DMSO-d₆) δ: 1.51 (m, 2H, CH₂), 1.64 (m, 4H, CH₂), 2.27 (s, 3H, CH₃), 2.50 (s, 3H, CH₃), 2.75 (t, 4H, J=4.5 Hz, CH₂), 6.98 (d, 1H, J=8.5 Hz, Ph-H), 7.12 (d, 1H, J=5.0 Hz, pyrimidinyl-H), 7.50 (d, 1H, J=8.5 Hz, Ph-H), 7.57 (m, 1H, Ar—H), 7.63 (s, 1H, Ph-H), 8.32 (d, 1H, J=8.0 Hz, Ar—H), 8.70 (d, 1H, J=5.0 Hz, Ar—H), and 9.15 (s, 1H, Ar—H). MS (ESI⁺) m/z 443.39 (C₂₅H₂₆N₆S requires 442.58).

{4-Methyl-5-[2-(3-methyl-4-piperidin-1-yl-phenylamino)-pyrimidin-4-yl]-thiazol-2-yl}-methanol (209). By reaction between 3-dimethylamino-1-(2-hydroxymethyl-4-methyl-thiazol-5-yl)-propenone and N-(3-methyl-4-piperidin-1-yl-phenyl)-guanidine. Yellow solid. Mp 142-144° C. Anal. RP-HPLC: t_{R}=11.9 min (0-60% MeCN; purity 100%). ¹H (DMSO-d₆) δ: 1.51 (m, 2H, CH₂), 1.65 (m, 4H, CH₂), 2.24 (s, 3H, CH₃), 2.64 (s, 3H, CH₃), 2.75 (t, 4H, J=5.0 Hz, CH₂), 4.71 (d, 2H, J=6.0 Hz, CH₂), 6.13 (t, 1H, J=6.0 Hz, OH), 6.94 (d, 1H, J=8.5 Hz, Ph-H), 7.04 (d, 1H, J=5.0 Hz, pyrimidinyl-H), 7.49 (d, 1H, J=8.5 Hz, Ph-H), 7.61 (s, 1H, Ph-H), 8.47 (d, 1H, J=5.0 Hz, pyrimidinyl-H), 9.43 (br. s, 1H, NH).

5-[4-(2,4-Dimethyl-thiazol-5-yl)-pyrimidin-2-ylamino]-2-morpholin-4-yl-benzamide (210). By reaction between 3-dimethylamino-1-(2,4-dimethyl-thiazol-5-yl)-propenone and 5-guanidino-2-morpholin-4-yl-benzamide. Yellow solid. Anal. RP-HPLC: t_{R}=13.8 min (0-60% MeCN; purity >95%). ¹H (DMSO-d₆) δ: 2.59 (s, 3H, CH₃), 2.61 (s, 3H, CH₃), 2.86 (m, 4H, CH₂), 3.70 (m, 4H, CH₂), 7.03 (d, 1H, J=5.4 Hz, pyrimidinyl-H), 7.18, (d, 1H, J= 8.8 Hz, Ph-H), 7.45 (s, 1H, Ph-H), 7.87 (dd, 1H, J=8.8, 2.9 Hz, Ph-H), 8.07 (d, 1H, J=2.9 Hz, Ph-H), 8.46 (d, 1H, J=5.4 Hz, pyrimidinyl-H), 8.66 (s, 1H, NH), 9.64 (1H, s, NH). MS (ESI⁺) m/z 411.27 (C₂₀H₂₂N₆O₂S requires 410.49).

5-[4-(2-Amino-4-methyl-thiazol-5-yl)-pyrimidin-2-ylamino]-2-morpholin-4-yl-benzamide (211). By reaction between N′-[5-(3-dimethylamino-acryloyl)-4-methyl-thiazol-2-yl]-N,N-dimethyl-formamidine and 5-guanidino-2-morpholin-4-yl-benzamide. Yellow solid. Anal. RP-HPLC: t_{R}=12.1 min (0-60% MeCN; purity >95%). ¹H (DMSO-d₆) δ: 2.40 (s, 3H, CH₃), 2.84 (m, 4H, CH₂), 3.70 (m, 4H, CH₂), 6.80 (d, 1H, J=5.4 Hz, pyrimidinyl-H,), 7.11 (d, 1H, J=8.8 Hz, Ph-H,), 7.42 (s, 1H, NH), 7.43 (s, 2H, NH₂), 7.94 (dd, 1H, J=8.8, 2.9 Hz, Ph-H,), 7.96 (d, 1H, J=2.9 Hz, Ph-H,), 8.26 (d, 1H, J=5.4 Hz, pyrimidinyl-H,), 8.67 (s, 1H, NH), 9.40 (s, 1H, NH). MS (ESI⁺) m/z 412.22 (C₁₉H₂₁N₇O₂S requires 411.48).

5-[4-(2-Ethylamino-4-methyl-thiazol-5-yl)-pyrimidin-2-ylamino]-2-morpholin-4-yl-benzamide (212). By reaction between 3-dimethylamino-1-(2-ethylamino-4-methyl-thiazol-5-yl)-propenone and 5-guanidino-2-morpholin-4-yl-benzamide. Yellow solid. Anal. RP-HPLC: t_{R}=12.1 min (0-60% MeCN; purity >95%). ¹H (DMSO-d₆) δ: 1.13 (t, 3H, J=7.3 Hz, CH₃), 2.42 (s, 3H, CH₃), 2.85 (m, 4H, CH₂), 3.22 (q, 2H, J=7.3 Hz, CH₂), 3.69 (m, 4H, CH₂), 6.82 (d, 1H, J=5.4 Hz, pyrimidinyl-H), 7.12 (d, 1H, J=8.8 Hz, Ph-H), 7.41 (s, 1H, NH), 7.87 (dd, 1H, J=8.8, 2.9 Hz, Ph-H,), 8.02 (d, 1H, J=2.9, Ph-H), 8.04 (s, 1H, NH), 8.27 (d, 1H, J=5.4 Hz, pyrimidinyl-H), 8.64 (s, 1H, NH), 9.39 (s, 1H, NH). MS (ESI⁺) m/z 440.31 (C₂₁H₂₅N₇O₂S requires 439.54).

Cyclopropyl-(4-{4-[4-(2,4-dimethyl-thiazol-5-yl)-pyrimidin-2-ylamino]-phenyl}piperazin-1-yl)-methanone (213). By reaction between 3-dimethylamino-1-(2,4-dimethyl-thiazol-5-yl)-propenone and N-[4-(4-cyclopropanecarbonyl-piperazin-1-yl)-phenyl]-guanidine. Yellow solid. Anal. RP-HPLC: t_{R}=11.6 min (10-70% MeCN; purity 99%). ¹H-NMR (DMSO-d₆) 5-0.70 (m, 4H, CH₂), 1.99 (m, 1H, CH), 2.58 (s, 3H, CH₃), 2.61 (s, 3H, CH₃), 2.99 (m, 2H, CH₂), 3.08 (m, 2H, CH₂), 3.58 (m, 2H, CH₂), 3.78 (m, 2H, CH₂), 6.90 (t, 2H, J=9.0 Hz, Ph-H), 6.96 (d, 1H, J=5.0 Hz, pyrimidinyl-H), 7.58 (d, 2H, J=8.5 Hz, Ph-H), 8.41 (d, 1H, J=5.5 Hz, pyrimidinyl-H), 9.39 (s, 1H, NH). MS (ESI⁺) m/z 435.36 (C₂₃H₂₆N₆OS requires 434.56).

[4-(2,4-Dimethyl-thiazol-5-yl)-pyrimidin-2-yl]-(4-methyl-3-morpholin-4-yl-phenyl)-amine (214). By reaction between 3-dimethylamino-1-(2,4-dimethyl-thiazol-5-yl)-propenone and N-(4-methyl-3-morpholin-4-yl-phenyl)-guanidine. Yellow solid. Anal. RP-HPLC: t_{R}=15.7 min (10-70% MeCN; purity 94%). ¹H-NMR (DMSO-d₆) δ: 2.16 (s, 3H, CH₃), 2.59 (s, 3H, CH₃), 2.61 (s, 3H, CH₃), 2.82 (t, 4H, J=4.0 Hz, CH₂), 3.71 (t, 4H, J=4.0 Hz, CH₂), 7.01 (d, 1H, J=5.5 Hz, pyrimidinyl-H), 7.04 (d, 1H, J=8.0 Hz, Ph-H), 7.34 (dd, 1H, J=2.0, 8.5 Hz, Ph-H), 7.47 (s, 1H, NH), 8.46 (d, 1H, J=5.5 Hz, pyrimidinyl-H), 9.47 (s, 1H, NH). MS (ESI⁺) m/z 382.35 (C₂₀H₂₃N₅OS requires 381.50).

[4-(2,4-Dimethyl-thiazol-5-yl)-pyrimidin-2-yl]-(4-methoxy-3-morpholin-4-ylmethyl-phenyl)-amine (215). By reaction between 3-dimethylamino-1-(2,4-dimethyl-thiazol-5-yl)-propenone and N-(4-methoxy-3-morpholin-4-ylmethyl-phenyl)-guanidine. Yellow solid. Anal. RP-HPLC: t_{R}=15.9 min (10-70% MeCN; purity 94%). ¹H-NMR (DMSO-d₆) δ: 2.36 (m, 4H, CH₂), 2.59 (s, 3H, CH₃), 2.60 (s, 3H, CH₃), 3.41 (s, 2H, CH₂), 3.53 (t, 4H, J=4.0 Hz, CH₂), 3.72 (s, 3H, CH₃), 6.89 (d, 1H, J=9.0 Hz, Ph-H), 6.96 (d, 1H, J=5.5 Hz, pyrimidinyl-H), 7.58 (dd, 1H, J=2.5, 9.0 Hz, Ph-H), 7.65 (d, 1H, J=2.5 Hz, Ph-H), 8.42 (d, 1H, J=5.0 Hz, pyrimidinyl-H), 9.40 (s, 1H, NH). MS (ESI⁺) m/z 412.24 (C₂₁H₂₅N₅O₂S requires 411.52).

{5-[2-(3-Methoxy-4-piperidin-1-yl-phenylamino)-pyrimidin-4-yl]-4-methyl-thiazol-2-yl}-methanol (216). By reaction between 3-dimethylamino-1-(2-hydroxymethyl-4-methyl-thiazol-5-yl)-propenone and N-(3-methoxy-4-piperidin-1-yl-phenyl)-guanidine. Yellow solid. Anal. RP-HPLC: t_{R}=12.3 min (0-60% MeCN; purity 100%). ¹NMR (DMSO-d₆) δ: 1.57 (m, 2H, CH₂), 1.74 (m, 4H, CH₂), 2.68 (s, 3H, CH₃), 2.92 (m, 4H, CH₂), 3.94 (s, 3H, CH₃), 4.80 (s, 2H, CH₂), 6.96 (d, J=8.5 Hz, 1H, Ph-H), 7.05 (d, 1H, J=5.0 Hz, pyrimidinyl-H), 7.14 (d, 1H, J=8.5 Hz, Ph-H), 7.54 (s, 1H, Ph-H), 8.42 (d, 1H, J=5.0 Hz, pyrimidinyl-H). MS (ESI⁺) m/z 412.43 (C₂₁H₂₅N₅O₂S requires 411.52).

{4-Methyl-5-[2-(3-methyl-4-morpholin-4-yl-phenylamino)-pyrimidin-4-yl]-thiazol-2-yl}-methanol (217). By reaction between 3-dimethylamino-1-(2-hydroxymethyl-4-methyl-thiazol-5-yl)-propenone and N-(3-methyl-4-morpholin-4-yl-phenyl)-guanidine. Yellow solid. Mp 93-94° C. Anal. RP-HPLC: t_{R}=13.0 min (0-60% MeCN; purity 100%). 1H-NMR (DMSO-d₆) δ: 2.23 (s, 3H, CH₃), 2.60 (s, 3H, CH₃), 2.75 (t, 4H, J=4.5 Hz, CH₂), 3.68 (t, 4H, J=4.5 Hz, CH₂), 4.67 (d, 2H, J=6.0 Hz, CH₂), 6.10 (t, 1H, J=6.0 Hz, OH), 6.94 (d, 1H, J=8.5 Hz, Ph-H), 7.01 (d, 1H, J=5.0 Hz, pyrimidinyl-H), 7.49 (d, 1H, J=9.0 Hz, Ph-H), 7.60 (s, 1H, Ph-H), 8.44 (d, 1H, J=5.0 Hz, pyrimidinyl-H), 9.43 (br. s, 1H, NH). ¹³C-NMR (DMSO-d₆) δ: 18.34, 18.71, 52.89, 61.73, 67.35, 108.68, 118.03, 119.55, 122.50, 131.40, 132.66, 136.34, 146.18, 152.64, 158.63, 159.68, 160.39, 175.36. MS (ESI⁺) m/z 398.38 (C₂₀H₂₃N₅O₂S requires 397.50).

[4-(2-Amino-4-methyl-thiazol-5-yl)-pyrimidin-2-yl]-(3-methyl-4-morpholin-4-yl-phenyl)-amine (218). By reaction between N′-[5-(3-dimethylamino-acryloyl)-4-methyl-thiazol-2-yl]-N,N-dimethyl-formamidine and N-(3-methyl-4-morpholin-4-yl-phenyl)-guanidine. Yellow solid. Mp 256-257° C. Anal. RP-HPLC: t_{R}=11.4 min (0-60% MeCN; purity 100%). ¹H-NMR (DMSO-d₆) δ: 2.23 (s, 3H, CH₃), 2.39 (s, 3H, CH₃), 2.75 (t, 4H, J=4.5 Hz, CH₂), 3.69 (t, 4H, J=4.5 Hz, CH₂), 6.79 (d, 1H, J=5.0 Hz, pyrimidinyl-H), 6.91 (d, 1H, J=8.5 Hz, Ph-H), 7.44 (br. s, 2H, NH₂), 7.51 (d, 1H, J=9.0 Hz, Ph-H), 8.24 (d, 1H, J=5.5 Hz, pyrimidinyl-H), 9.18 (br. s, 1H, NH). ¹³C-NMR (DMSO-d₆) δ: 18.35, 19.07, 49.26, 52.94, 67.37, 107.14, 112.50, 117.71, 118.88, 119.46, 122.21, 132.61, 136.74, 145.81, 152.49, 158.25, 159.24, 160.18, 169.43. MS (ESI⁺) m/z 383.44 (C₁₉H₂₂N₆OS requires 382.48).

[4-(2-Ethylamino-4-methyl-thiazol-5-yl)-pyrimidin-2-yl]-(3-methyl-4-morpholin-4-yl-phenyl)-amine (219). By reaction between 3-dimethylamino-1-(2-ethylamino-4-methyl-thiazol-5-yl)-propenone and N-(3-methyl-4-morpholin-4-yl-phenyl)-guanidine. Yellow solid. Mp 213-214° C. Anal. RP-HPLC: t_{R}=13.0 min (0-60% MeCN; purity 100%). ¹H-NMR (DMSO-d₆) δ: 1.13 (m, 3H, CH₃), 2.23 (s, 3H, CH₃), 2.74 (t, 4H, J=4.5 Hz, CH₂), 3.24 (m, 2H, CH₂), 3.68 (t, 4H, J=4.5 Hz, CH₂), 6.80 (d, 1H, J=5.0 Hz, pyrimidinyl-H), 6.91 (d, 1H, J=8.5 Hz, Ph-H), 7.44 (d, 1H, J=8.5 Hz, Ph-H), 7.64 (s, 1H, Ph-H), 8.05 (t, 1H, J=5.0 Hz, NH), 8.24 (d, 1H, J=5.0 Hz, pyrimidinyl-H), 9.19 (br. s, 1H, NH). ¹³C-NMR (DMSO-d₆) δ; 14.90, 18.33, 19.28, 52.93, 60.41, 67.37, 106.96, 117.74, 118.48, 119.48, 122.17, 132.55, 136.75, 145.79, 152.73, 158.23, 160.15, 169.01, 170.99. MS (ESI⁺) m/z 411.47 (C₂₁H₂₆N₆OS requires 410.54).

[4-(2,4-Dimethyl-thiazol-5-yl)-pyrimidin-2-yl]-(3-methyl-4-morpholin-4-yl-phenyl)-amine (220). By reaction between 3-dimethylamino-1-(2,4-dimethyl-thiazol-5-yl)-propenone and N-(3-methyl-4-morpholin-4-yl-phenyl)-guanidine. Yellow solid. Mp. 164-166° C. Anal. RP-HPLC: t_{R}=15.1 min (0-60% MeCN; purity 100%). ¹H-NMR (DMSO-d₆) δ: 2.23 (s, 3H, CH₃), 2.59 (s, 3H, CH₃), 2.61 (s, 3H, CH₃), 2.76 (t, 4H, J=4.5 Hz, CH₂), 3.69 (t, 4H, J=4.5 Hz, CH₂), 6.95 (d, 1H, J=9.0 Hz, Ph-H), 6.99 (d, 1H, J=5.0 Hz, pyrimidinyl-H), 7.51 (d, 1H, J=8.5 Hz, Ph-H), 7.56 (s, 1H, Ph-H), 8.43 (d, 1H, J=5.0 Hz, pyrimidinyl-H), 9.43 (br. s, 1H, NH). ¹³C-NMR (DMSO-d₆) δ: 18.34, 18.53, 19.63, 52.89, 67.31, 108.60, 118.01, 119.58, 122.45, 131.55, 132.61, 136.31, 146.17, 152.53, 158.46, 159.65, 160.37, 166.95. MS (ESI⁺) m/z 382.41 (C₂₀H₂₃N₅OS requires 381.50).

### Example 3

Kinase assays. The compounds of the invention above were investigated for their ability to inhibit the enzymatic activity of various protein kinases (Table 2). This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 mM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C. Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%).

CDK 7 and 9 assays. CTD peptide substrate (biotinyl-Abx-(Tyr-Ser-Pro-Thr-Ser-Pro-Ser)₄-NH₂; 1-2 mg/mL) and recombinant human CDK7/cyclin H, CDK9/cyclin T1, or CDK9/cyclin K (0.5-2 μg) were incubated for 45 min at 30° C. in the presence of varying amounts of test compound in 20 mM MOPS pH 7.2, 25 mM β-glycerophosphate, 5 mM EGTA, 1 mM DTT, 1 mM sodium vanadate, 15 mM MgCl₂, and 100 μM ATP (containing a trace amount of ³²PγATP) in a total volume of 25 μL in a 96-well microtiter plate. The reaction was stopped by placing the plate on ice for 2 min. Avidin (50 μg) was added to each well, and the plate was incubated at room temp for 30 min. The samples were transferred to a 96-well P81 filter plate, and washed (4×200 μL per well) with 75 mM phosphoric acid. Microscint 40 scintillation liquid (50 μL) was added to each well, and the amount of ³²P incorporation for each sample was measured using a Packard Topcount microplate scintillation counter.

Aurora-A (human) kinase assay. This was achieved by measurement of incorporation of radioactive phosphate from ATP into Kemptide substrate (LRRASLG), upon phosphorylation by commercially obtained aurora-A kinase. Assays were performed using 96-well plates and appropriate assay buffers (8 mM MOPS, 0.2 mM EDTA, pH 7.0), into which were added 5-10 ng of active enzyme with 200 μM substrate (Kemptide). The reactions were initiated by addition of Mg/ATP mix (10 mM MgAcetate+15 μM ATP with 30-50 kBq per well of [γ-³³P]-ATP) and mixtures incubated for 40 min at room temperature. Reactions were stopped by addition of 3% phosphoric acid, followed by filtration through p81 filterplates (Whatman Polyfiltronics, Kent, UK). After washing 5 times with 75 mM aq orthophosphoric acid and once in methanol, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 2.0.9, IDBS, Guildford, Surrey, UK) to determine IC₅₀ values (concentration of test compound which inhibits kinase activity by 50%).

### Example 4

MTT cytotoxicity assay. The compounds of the invention were subjected to a standard cellular proliferation assay using human tumour cell lines obtained from the ATCC (American Type Culture Collection, 10801 University Boulevard, Manessas, Va. 20110-2209, USA). Standard 72-h MTT (thiazolyl blue; 3-[4,5-dimethylthiazol-2-yl]-2,5-diphenyltetrazolium bromide) assays were performed (Haselsberger, K.; Peterson, D. C.; Thomas, D. G.; Darling, J. L. Anti Cancer Drugs 1996, 7, 331-8; Loveland, B. E.; Johns, T. G.; Mackay, I. R.; Vaillant, R; Wang, Z. X.; Hertzog, P. J. Biochemistry International 1992, 27, 501-10). In short: cells were seeded into 96-well plates according to doubling time and incubated overnight at 37° C. Test compounds were made up in DMSO and a ⅓ dilution series prepared in 100 μL cell media, added to cells (in triplicates) and incubated for 72 ho at 37° C. MTT was made up as a stock of 5 mg/mL in cell media and filter-sterilised. Media was removed from cells followed by a wash with 200 μL PBS. MTT solution was then added at 20 μL per well and incubated in the dark at 37° C. for 4 h. MTT solution was removed and cells again washed with 200 μL PBS. MTT dye was solubilised with 200 μL per well of DMSO with agitation. Absorbance was read at 540 nm and data analysed using curve-fitting software (GraphPad Prism version 3.00 for Windows, GraphPad Software, San Diego Calif. USA) to determine IC₅₀ values (concentration of test compound which inhibits cell growth by 50%).

### Example 5

### Anti-HIV Efficacy Evaluation in Fresh Human PBMCs

Representative compounds of the present invention were tested for antiviral activity against HIV-1 in human peripheral blood mononuclear cells (PBMCs) using the clinical pediatric HIV strain RoJo or WeJo. PBMCs were cultured under conditions which promote cell survival and HIV replication. Antiviral activity was tested for from 6-9 log₁₀ serial dilutions of a 100 μM compound stock solution in DMSO. The following parameters were derived: IC₅₀ and IC₉₀ (concentrations inhibiting virus replication by 50 and 90%, respectively, TC₅₀ (concentration decreasing cell viability by 50%), and TI (therapeutic index: TC₅₀/IC₅₀).

Fresh PBMCs, seronegative for HIV and HBV, were isolated from screened donors (Interstate Blood Bank, Inc. Memphis, Tenn.). Cells were pelleted/washed 2-3 times by low speed centrifugation and re-suspension in PBS to remove contaminating platelets. The Leukophoresed blood was then diluted with Dulbecco's Phosphate Buffered Saline (DPBS) and layered over Lymphocyte Separation Medium (LSM; Cellgro® by Mediatech, lac.; density 1.078±0.002 g/mL; Cat. #85-072-CL) in a 50 mL centrifuge tube and then centrifuged. Banded PBMCs were gently aspirated from the resulting interface and subsequently washed with PBS by low speed centrifugation. After the final wash, cells were enumerated by trypan blue exclusion and re-suspended in RPMI 1640 supplemented with fetal bovine serum (FBS), and L-glutamine, Phytohemagglutinin (PHA-P, Sigma). The cells were allowed to incubate at 37° C. After incubation, PBMCs were centrifuged and resuspended in RPMI 1640 with FBS, L-glutamine, penicillin, streptomycin, gentamycin, and recombinant human IL-2 (R&D Systems, Inc). IL-2 is included in the culture medium to maintain the cell division initiated by the PHA mitogemc stimulation. PBMCs were maintained in this with bi-weekly medium changes until used in the assay protocol. Cells were kept in culture for a maximum of two weeks before being deemed too old for use in assays and discarded. Monocytes were depleted from the culture as the result of adherence to the tissue culture flask.

For the standard PBMC assay, PHA-P stimulated cells from at least two normal donors were pooled, diluted and plated in the interior wells of a 96-well round bottom microplate. Pooling of mononuclear cells from more than one donor was used to minimise the variability observed between individual donors, which results from quantitative and qualitative differences in HIV infection and overall response to the PHA and IL-2 of primary lymphocyte populations. Each plate contained virus/cell control wells (cells plus virus), experimental wells (drug plus cells plus virus) and compound control wells (drug plus media without cells, necessary for MTS monitoring of cytotoxicity). Since HIV-1 is not cytopathic to PBMCs, this allows the use of the same assay plate for both antiviral activity and cytotoxicity measurements. Test drug dilutions were prepared in microtiter tubes and each concentration was placed in appropriate wells using the standard format. A predetermined dilution of virus stock was placed in each test well (final MOI≅0.1). The PBMC cultures were maintained for seven days following infection at 37° C., 5% CO₂. After this period, cell-free supernatant samples were collected for analysis of reverse transcriptase activity and/or HIV p24 content. Following removal of supernatant samples, compound cytotoxicity was measured by addition of MTS to the plates for determination of cell viability. Wells were also examined microscopically and any abnormalities were noted.

Reverse transcriptase activity assay: A microtiter plate-based reverse transcriptase (RT) reaction was utilised (Buckheit et al., ADDS Research and Human Retroviruses 7:295-302, 1991). Tritiated thymidine triphosphate (³H-TTP, 80 Ci/mmol, NEN) was received in 1:1 dH₂/Ethanol at 1 mCi/mL. Poly rA:oligo dT template:primer (Pharmacia) was prepared as a stock solution, followed by aliquoting and storage at −20° C. The RT reaction buffer was prepared fresh on a daily basis. The final reaction mixture was prepared by combining ³H-TTP, dH₂O, poly rA:oligo dT stock and reaction buffer. This reaction mixture was placed in a round bottom microtiter plate and supernatant containing virus was added and mixed. The plate was incubated at 37° C. for 60 minutes. Following incubation, the reaction volume was spotted onto DE81 filter-mats (Wallac), in a sodium phosphate buffer or 2×SSC (Life Technologies). Next they were washed in distilled water, in 70% ethanol, and then dried. Incorporated radioactivity (counts per minute, CPM) was quantified using standard liquid scintillation techniques.

### Example 6

The kinase selectivity profiles of selected example compounds were determined, essentially as described (Bain, J.; McLauchlan, H.; Elliott, M.; Cohen, P. Biochemical Journal, 2003, 371, 199.); the results are shown in Tables 3 & 4. For the assays shown in Table 3, the kinases were assayed at the following ATP concentrations: SAPK4, PKBΔph, GSK3b, SAPK3, CK2, MKK1, PIM2, IKKB, ERK8, and PRK2 at 5 μM; JNK, PRAK, ROCK-II, SAPK2b, CDK2, CHK1, MSK1, CSK, P70S6K, PKA, CK1, MAPKAP-K2, SGK, PKCa, PDK1, NEK 7, and MAPKAP-K3 at 20 μM; SAPK2a, LCK, AMPK, MAPK2, DYRK1a, MAPKAP-K1a, NEK-6, NEK2a, PBK, CAMK-1, SRPK-1, JNK3, MNK2, RSK2, MNK1, PKBB, and SmMLCK at 50 μM. For the assays in Table 4 the ATP concentration was 100 μM throughout.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Chemical structures of selected compounds of the invention

| No. | Structure | Name |
|---|---|---|
| 1 | 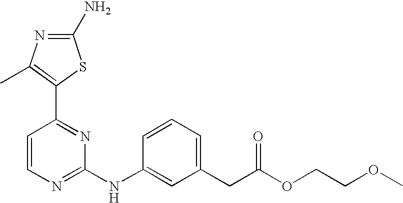 | {3-[4-(2-Amino-4-methyl-thiazol-5- yl)-pyrimidin-2-ylamino]-phenyl}- acetic acid 2-methoxy-ethyl ester |
| 2 | 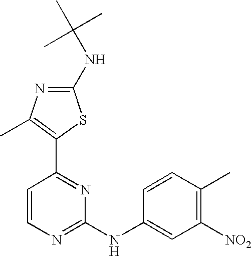 | [4-(2-tert-Butylamino-4-methyl- thiazol-5-yl)-pyrimidin-2-yl]-(4- methyl-3-nitro-phenyl)-amine |
| 3 | 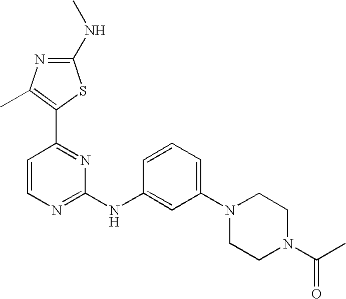 | 1-(4-{3-[4-(4-Methyl-2-methylamino- thiazol-5-yl)-pyrimidin-2-ylamino]- phenyl}-piperazin-1-yl)-ethanone |
| 4 | 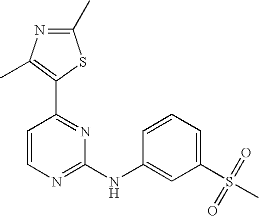 | [4-(2,4-Dimethyl-thiazol-5-yl)- pyrimidin-2-yl]-(3-methanesulfonyl- phenyl)-amine |
| 5 | 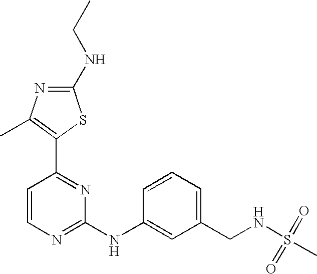 | N-{3-[4-(2-Ethylamino-4-methyl- thiazol-5-yl)-pyrimidin-2-ylamino]- benzyl}-methauesulfonamide |
| 6 | 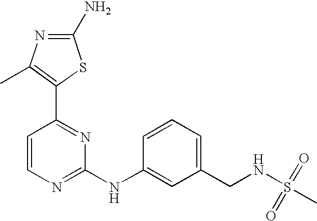 | N-{3-[4-(2-Amino-4-methyl-thiazol- 5-yl)-pyrimidin-2-ylamino]-benzyl}- methanesulfonamide |
| 7 | 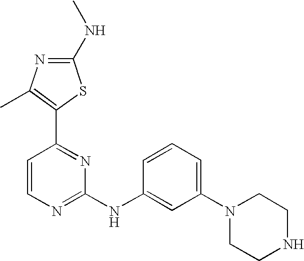 | [4-(4-Methyl-2-methylamino-thiazol- 5-yl)-pyrimidin-2-yl]-(3-piperazin-1- yl-phenyl)-amine |
| 8 | 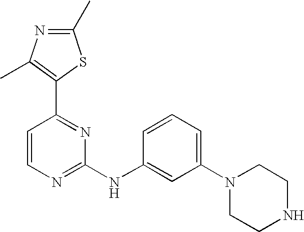 | [4-(2,4-Dimethyl-thiazol-5-yl)- pyrimidin-2-yl]-(3-piperazin-1-yl- phenyl)-amine |
| 9 | 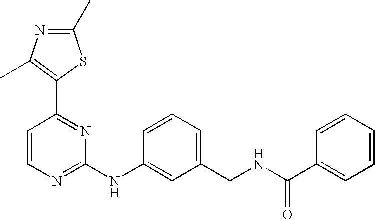 | N-{3-[4-(2,4-Dimethyl-thiazol-5-yl)- pyrimidin-2-ylamino]-benzyl}- benzamide |
| 10 | 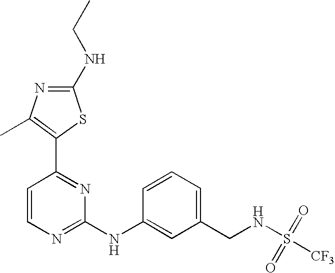 | N-{3-[4-(2-Ethylamino-4-methyl- thiazol-5-yl)-pyrimidin-2-ylamino]- benzyl}-C,C,C-trifluoro- methanesulfonamide |
| 11 | 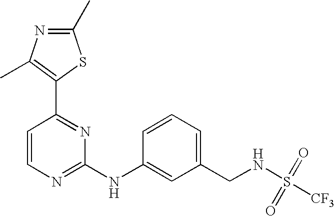 | N-{3-{4-(2,4-Dimethyl-thiazol-5-yl)- pyrimidin-2-ylamino]-benzyl}-C,C,C- trifluoro-methanesulfonamide |
| 12 | 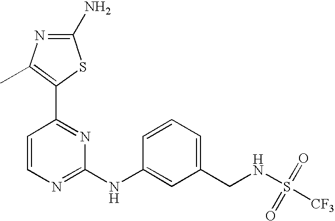 | N-{3-[4-(2-Amino-4-methyl-thiazol- 5-yl)-pyrimidin-2-ylamino]-benzyl}- C,C,C-trifluoro-methanesulfonamide |
| 13 | 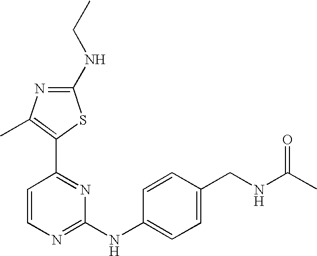 | N-{4-[4-(2-Ethylamino-4-methyl- thiazol-5-yl)-pyrimidin-2-ylamino]- benzyl}-acetamide |
| 14 | 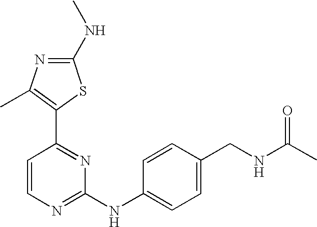 | N-{4-[4-(4-Methyl-2-methylamino- thiazol-5-yl)-pyrimidin-2-ylamino]- benzyl}-acetamide |
| 15 | 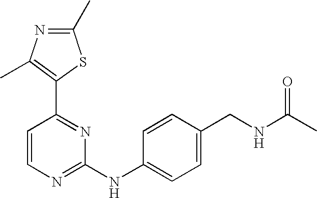 | N-{4-[4-(2,4-Dimethyl-thiazol-5-yl)- pyrimidin-2-ylamino]-benzyl}- acetamide |
| 16 | 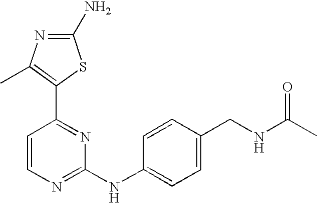 | N-{4-[4-(2-Amino-4-methyl-thiazol- 5-yl)-pyrimidin-2-ylamino]-benzyl}- acetamide |
| 17 | 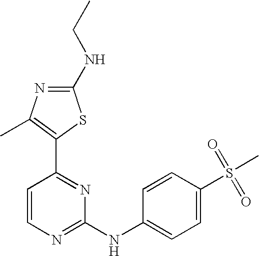 | [4-(2-Ethylamino-4-methyl-thiazol-5- yl)-pyrimidin-2-yl]-(4- methanesulfonyl-phenyl)-amine |
| 18 | 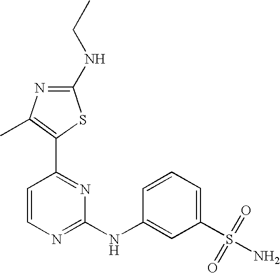 | 3-[4-(2-Ethylamino-4-methyl-thiazol- 5-yl)-pyrimidin-2-ylamino]- benzenesulfonamide |
| 19 | 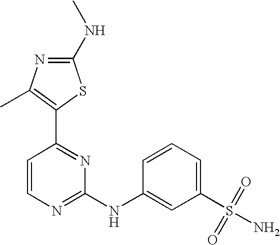 | 3-[4-(4-Methyl-2-methylamino- thiazol-5-yl)-pyrimidin-2-ylamino]- benzenesulfonamide |
| 20 | 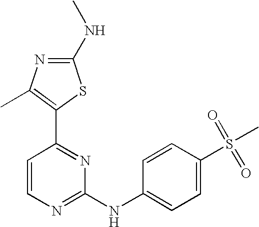 | (4-Methanesulfonyl-phenyl)-[4-(4- methyl-2-methylamino-thiazol-5-yl)- pyrimidin-2-yl]-amine |
| 21 | 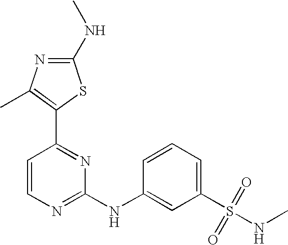 | N-Methyl-3-[4-(4-methyl-2- methylamino-thiazol-5-yl)-pyrimidin- 2-ylamino]-benzenesulfonamide |
| 22 | 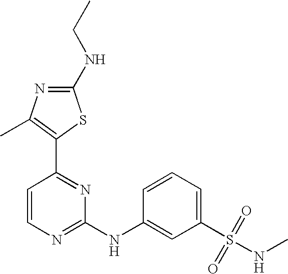 | 3-[4-(2-Ethylamino-4-methyl-thiazol- 5-yl)-pyrimidin-2-ylamino]-N- methyl-benzenesulfonamide |
| 23 | 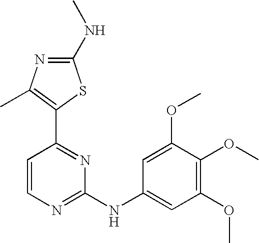 | [4-(4-Methyl-2-methylamino-thiazol- 5-yl)-pyrimidin-2-yl]-(3,4,5- trimethoxy-phenyl)-amine |
| 24 | 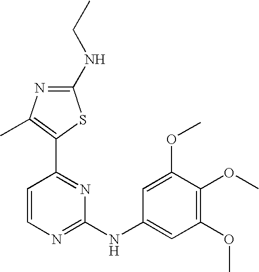 | [4-(2-Ethylamino-4-methyl-thiazol-5- yl)-pyrimidin-2-yl]-(3,4,5-trimethoxy- phenyl)-amine |
| 25 | 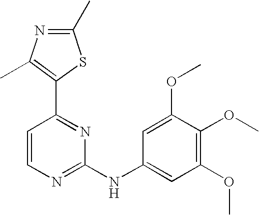 | [4-(2,4-Dimethyl-thiazol-5-yl)- pyrimidin-2-yl]-(3,4,5-trimethoxy- phenyl)-amine |
| 26 | 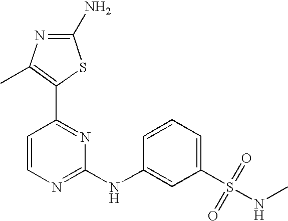 | 3-[4-(2-Amino-4-methyl-thiazol-5- yl)-pyrimidin-2-ylamino]-N-methyl- benzenesulfonamide |
| 27 | 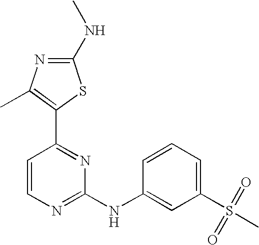 | (3-Methanesulfouyl-phenyl)-[4-(4- methyl-2-methylamino-thiazol-5-yl)- pyrimidin-2-yl]-amine |
| 28 | 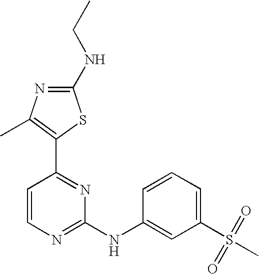 | [4-(2-Ethylamino-4-methyl-thiazol-5- yl)-pyrimidin-2-yl]-(3- methanesulfonyl-phenyl)-amine |
| 29 | 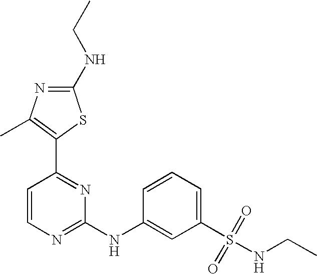 | N-Ethyl-3-{4-(2-ethylamino-4- methyl-thiazol-5-yl)-pyrimidin-2- ylamino]-benzenesulfonamide |
| 30 | 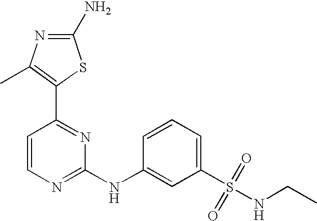 | 3-[4-(2-Amino-4-methyl-thiazol-5- yl)-pyrimidin-2-ylamino]-N-ethyl- benzenesulfonamide |
| 31 | 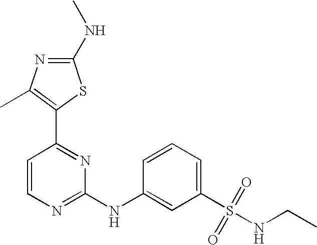 | N-Ethyl-3-[4-(4-methyl-2- methylamino-thiazol-5-yl)-pyrimidin- 2-ylamino]-benzenesulfonamide |
| 32 | 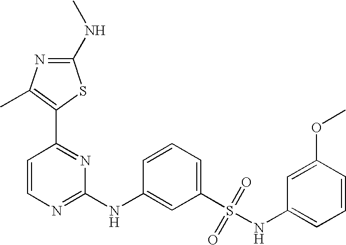 | N-(3-Methoxy-phenyl)-3-[4-(4- methyl-2-methylamino-thiazol-5-yl)- pyrimidin-2-ylamino]- benzenesulfonamide |
| 33 | 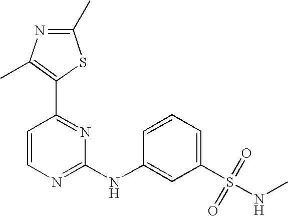 | 3-[4-(2,4-Dimethyl-thiazol-5-yl)- pyrimidin-2-ylamino]-N-methyl- benzenesulfonamide |
| 34 | 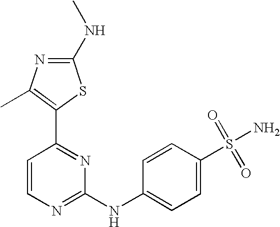 | 4-[4-(4-Methyl-2-methylamino- thiazol-5-yl)-pyrimidin-2-ylamino]- benzenesulfonamide |
| 35 | 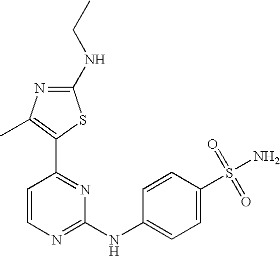 | 4-[4-(2-Ethylamino-4-methyl-thiazol- 5-yl)-pyrimidin-2-ylamino]- benzenesulfonamide |
| 36 | 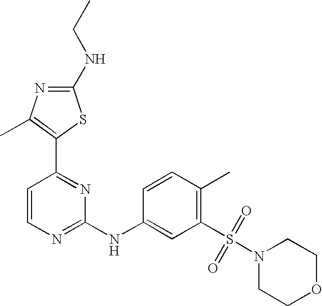 | [4-(2-Ethylamino-4-methyl-thiazol-5- yl)-pyrimidin-2-yl]-[4-methyl-3- (morpholine-4-sulfonyl)-phenyl]- amine |
| 37 | 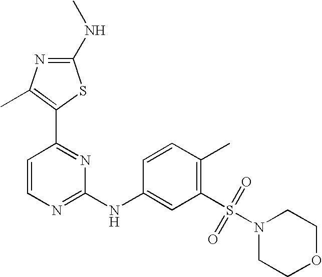 | [4-(4-Methyl-2-methylamino-thiazol- 5-yl)-pyrimidin-2-yl]-[4-methyl-3- (morpholine-4-sulfonyl)-phenyl]- amine |
| 38 | 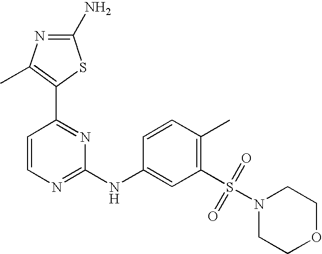 | [4-(2-Amino-4-methyl-thiazol-5-yl)- pyrimidin-2-yl]-[4-methyl-3- (morpholine-4-sulfonyl)-phenyl]- amine |
| 39 | 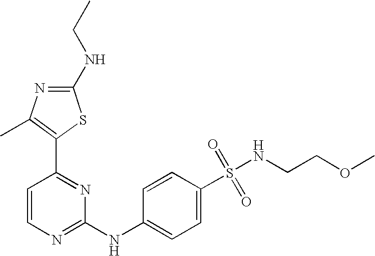 | 4-[4-(2-Ethylamino-4-methyl-thiazol- 5-yl)-pyrimidin-2-ylamino]-N-(2- methoxy-ethyl)-benzenesulfonamide |
| 40 | 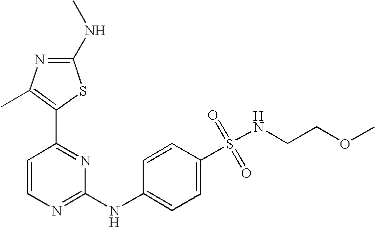 | N-(2-Methoxy-ethyl)-4{4-(4-methyl- 2-methylamino-thiazol-5-yl)- pyrimidin-2-ylamino]- benzenesulfonamide |
| 41 | 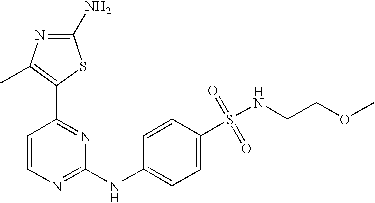 | 4-[4-(2-Amino-4-methyl-thiazol-5- yl)-pyrimidin-2-ylamino]-N-(2- methoxy-ethyl)-benzenesulfonamide |
| 42 | 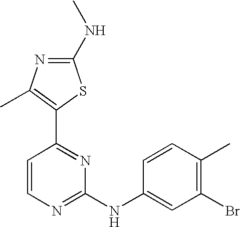 | (3-Bromo-4-methyl-phenyl)-[4-(4- methyl-2-methylamino-thiazol-5-yl)- pyrimidin-2-yl]-amine |
| 43 | 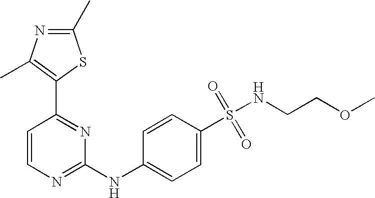 | 4-[4-(2,4-Dimethyl-thiazol-5-yl)- pyrimidin-2-ylamino]-N-(2-methoxy- ethyl)-benzenesulfonamide |
| 44 | 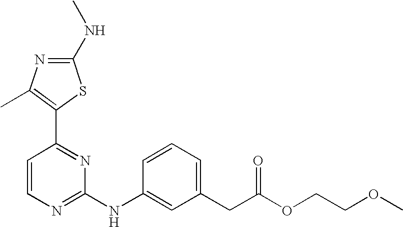 | {3-[4-(4-Methyl-2-methylamino- thiazol-5-yl)-pyrimidin-2-ylamino]- pheuyl}-acetic acid 2-methoxy-ethyl ester |
| 45 | 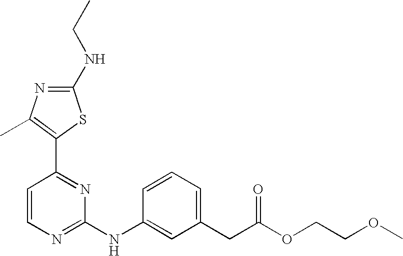 | {3-[4-(2-Ethylamino-4-methyl- thiazol-5-yl)-pyrimidin-2-ylamino]- phenyl}-acetic acid 2-methoxy-ethyl ester |
| 46 | 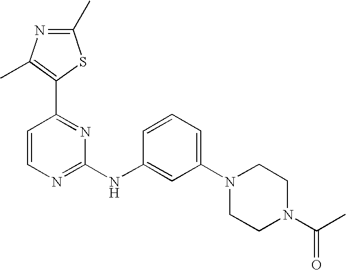 | 1-(4-{3-[4-(2,4-Dimethyl-thiazol-5- yl)-pyrimidin-2-ylamino]-phenyl}- piperazin-1-yl)-ethanone |
| 47 | 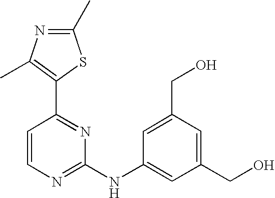 | {3-{4-(2,4-Dimethyl-thiazol-5-yl)- pyrimidin-2-ylamino]-5- hydroxymethyl-phenyl}-methanol |
| 48 | 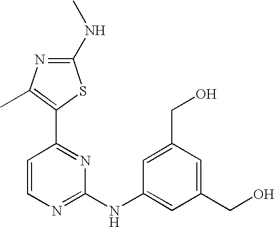 | {3-Hydroxymethyl-5-[4-(4-methyl-2- methylamino-thiazol-5-yl)-pyrimidin- 2-ylamino]-phenyl}-methanol |
| 49 | 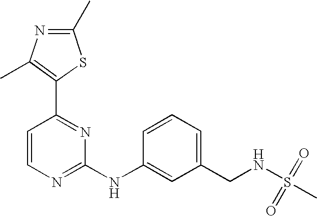 | N-{3-[4-(2,4-Dimethyl-thiazol-5-yl)- pyrimidin-2-ylamino]-benzyl}- methanesulfonamide |
| 50 | 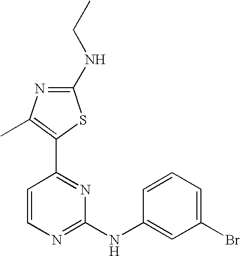 | (3-Bromo-phenyl)-[4-(2-ethylamino- 4-methyl-thiazol-5-yl)-pyrimidin-2- yl]-amine |
| 51 | 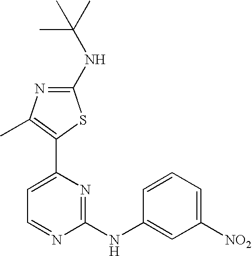 | [4-(2-tert-Butylamino-4-methyl- thiazol-5-yl)-pyrimidin-2-yl]-(3-nitro- phenyl)-amine |
| 52 | 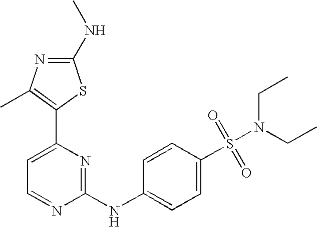 | N,N-Diethyl-4-{4-(4-methyl-2- methylamino-thiazol-5-yl)-pyrimidin- 2-ylamino]-benzenesulfonamide |
| 53 | 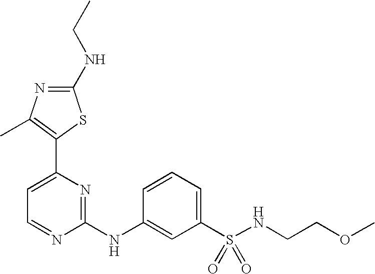 | 3-[4-(2-Ethylamino-4-methyl-thiazol- 5-yl)-pyrimidin-2-ylamino]-N-(2- methoxy-ethyl)-benzenesulfonamide |
| 54 | 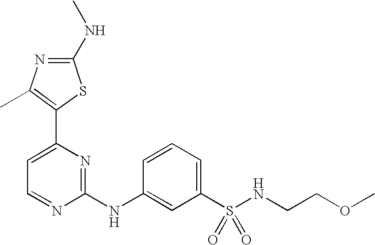 | N-(2-Methoxy-ethyl)-3-[4-(4-methyl- 2-methylamino-thiazol-5-yl)- pyrimidin-2-ylamino]- beuzenesulfonamide |
| 55 | 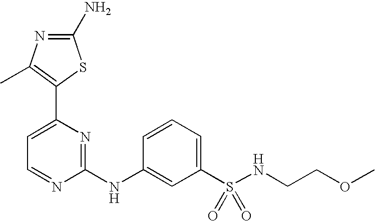 | 3-[4-(2-Amino-4-methyl-thiazol-5- yl)-pyrimidin-2-ylamino]-N-(2- methoxy-ethyl)-benzenesulfonamide |
| 56 | 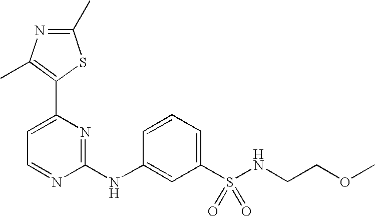 | 3-[4-(2,4-Dimethyl-thiazol-5-yl)- pyrimidin-2-ylamino]-N-(2-methoxy- ethyl)-benzenesulfonamide |
| 57 | 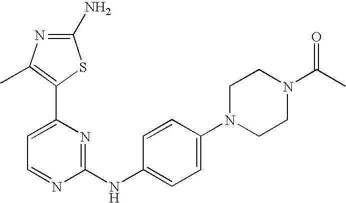 | 1-(4-{4-[4-(2-Amino-4-methyl- thiazol-5-yl)-pyrimidin-2-amino]- phenyl}-piperazin-1-yl)-ethanone |
| 58 | 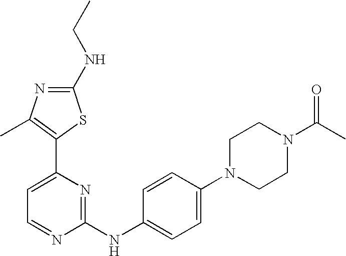 | 1-(4-{4-[4-(2-Ethylamino-4-methyl- thiazol-5-yl)-pyrimidin-2-ylamino]- phenyl}-piperazin-1-yl)-ethanone |
| 59 | 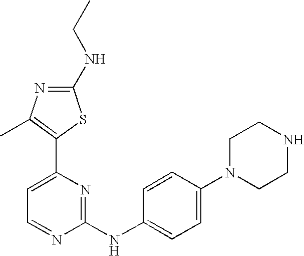 | [4-(2-Ethylamino-4-methyl-thiazol-5- yl)-pyrimidin-2-yl]-(4-piperazin-1-yl- phenyl)-amine |
| 60 | 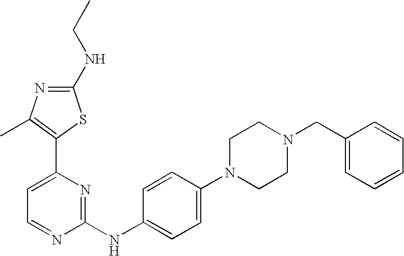 | [4-(4-Benzyl-piperazin-1-yl)-phenyl]- 4-(2-ethylamino-4-methyl-thiazol-5- yl)-pyrimidin-2-yl]-amine |
| 61 | 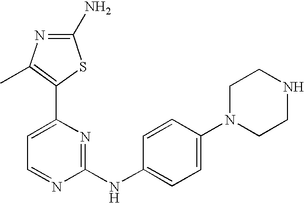 | [4-(2-Amino-4-methyl-thiazol-5-yl)- pyrimidin-2-yl]-(4-piperazin-1-yl- phenyl)-amine |
| 62 | 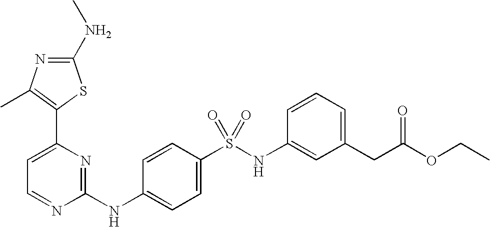 | (3-{4-[4-(4-Methyl-2-methylamino- thiazol-5-yl)-pyrimidin-2-ylamino]- benzenesulfonylamino}-phenyl)- acetic acid ethyl ester |
| 63 | 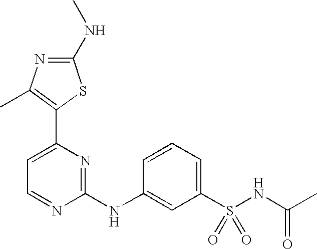 | N-Acetyl-3-[4-(4-methyl-2- methylamino-thiazol-5-yl)-pyrimidin- 2-ylamino]-benzenesulfonamide |
| 64 | 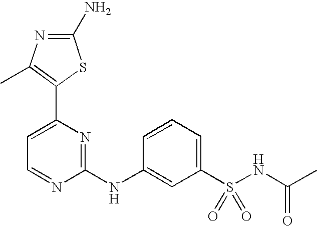 | N-Acetyl-3-[4-(2-amino-4-methyl- thiazol-5-yl)-pyrimidin-2-ylamino]- benzenesulfonamide |
| 65 | 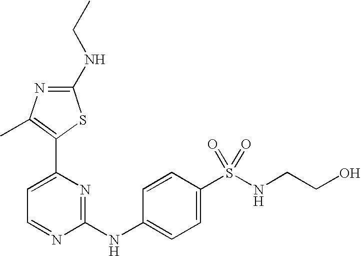 | 4-[4-(2-Ethylamino-4-methyl-thiazol- 5-yl)-pyrimidin-2-ylamino]-N-(2- hydroxy-ethyl)-benzenesulfonamide |
| 66 | 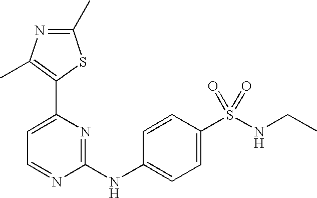 | 4-[4-(2,4-Dimethyl-thiazol-5-yl)- pyrimidin-2-ylamino]-N-ethyl- benzenesulfonamide |
| 67 | 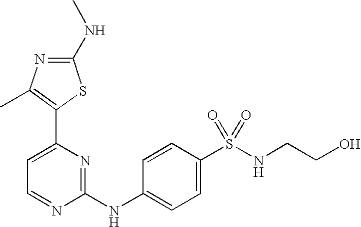 | N-(2-Hydroxy-ethyl)-4-[4-(4-methyl- 2-methylamino-thiazol-5-yl)- pyrimidin-2-ylamino]- benzenesulfonamide |
| 68 | 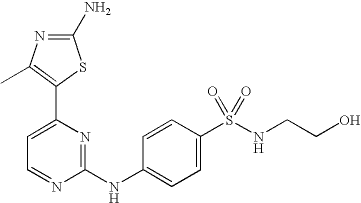 | 4-[4-(2-Amino-4-methyl-thiazol-5- yl)-pyrimidin-2-ylamino]-N-(2- hydroxy-ethyl)-benzenesulfonamide |
| 69 | 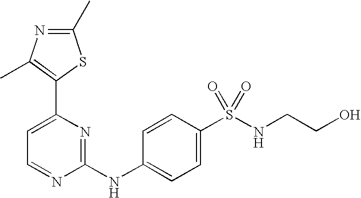 | 4-[4-(2,4-Dimethyl-thiazol-5-yl)- pyrimidin-2-ylamino]-N-(2-hydroxy- ethyl)-benzenesulfonamide |
| 70 | 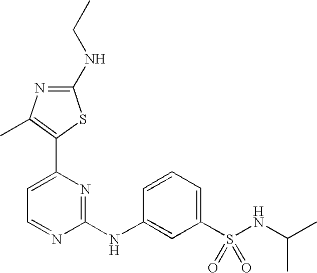 | 3-[4-(2-Ethylamino-4-methyl-thiazol- 5-yl)-pyrimidin-2-ylamino]-N- isopropyl-benzenesulfonamide |
| 71 | 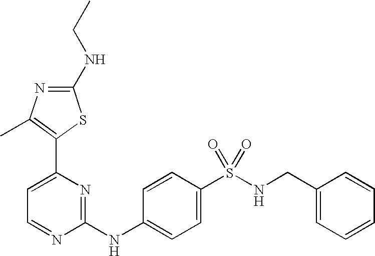 | N-Benzyl-4-[4-(2-ethylamino-4- methyl-thiazol-5-yl)-pyrimidin-2- ylamino]-benzenesulfonamide |
| 72 | 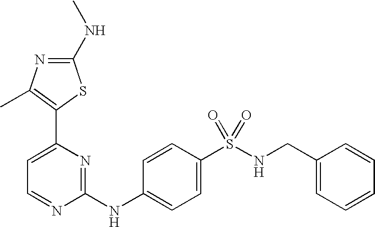 | N-Benzyl-4-[4-(4-methyl-2- methylamino-thiazol-5-yl)-pyrimidin- 2-ylamino]-benzenesulfonamide |
| 73 | 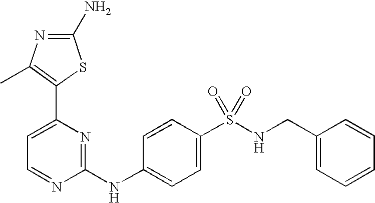 | 4-[4-(2-Amino-4-methyl-thiazol-5- yl)-pyrimidin-2-ylamino]-N-benzyl- benzenesulfonamide |
| 74 | 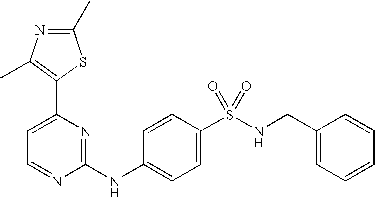 | N-Benzyl-4-[4-(2,4-dimethyl-thiazol- 5-yl)-pyrimidin-2-ylamino]- benzenesulfonamide |
| 75 | 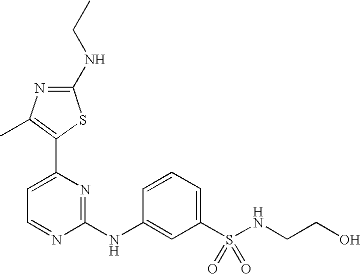 | 3-[4-(2-Ethylamino-4-methyl-thiazol- 5-yl)-pyrimidin-2-ylamino]-N-(2- hydroxy-ethyl)-benzenesulfonamide |
| 76 | 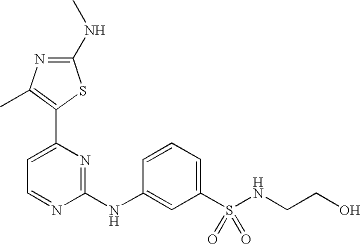 | N-(2-Hydroxy-ethyl)-3-[4-(4-methyl- 2-methylamino-thiazol-5-yl)- pyrimidin-2-ylamino]- benzenesulfonamide |
| 77 | 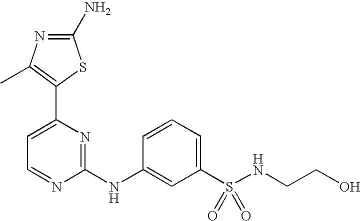 | 3-[4-(2-Amino-4-methyl-thiazol-5- yl)-pyrimidin-2-ylamino]-N-(2- hydroxy-ethyl)-benzenesulfonamide |
| 78 | 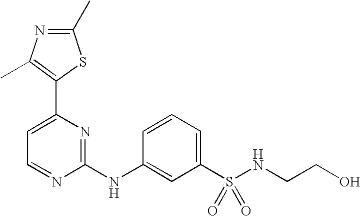 | 3-[4-(2,4-Dimethyl-thiazol-5-yl)- pyrimidin-2-ylamino]-N-(2-hydroxy- ethyl)-benzenesulfonamide |
| 79 | 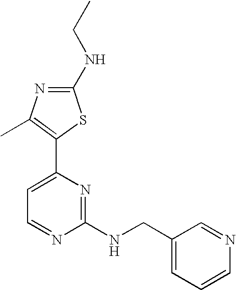 | [4-(2-Ethylamino-4-methyl-thiazol-5- yl)-pyrimidin-2-yl]-pyridin-3- ylmethyl-amine |
| 80 | 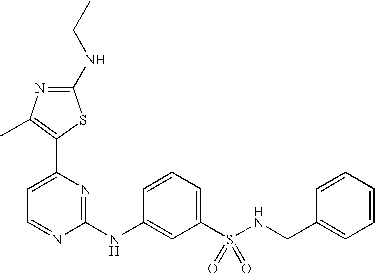 | N-Benzyl-3-[4-(2-ethylamino-4- methyl-thiazol-5-yl)-pyrimidin-2- ylamino]-benzenesulfonamide |
| 81 | 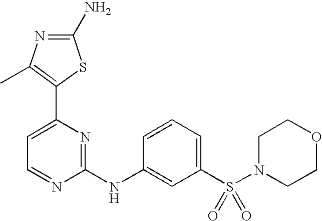 | [4-(2-Amino-4-methyl-thiazol-5-yl)- pyrimidin-2-yl]-[3-(morpholine-4- sulfonyl)-phenyl]-amine |
| 82 | 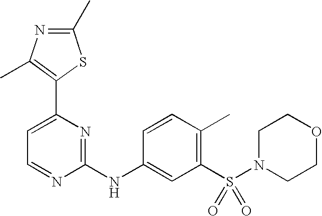 | [4-(2,4-Dimethyl-thiazol-5-yl)- pyrimidin-2-yl]-[4-methyl-3- (morpholine-4-sulfonyl)-phenyl]- amine |
| 83 | 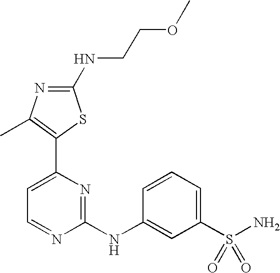 | 3-{4-[2-(2-Methoxy-ethylamino)-4- methyl-thiazol-5-yl]-pyrimidin-2- ylamino}-benzenesulfonamide |
| 84 | 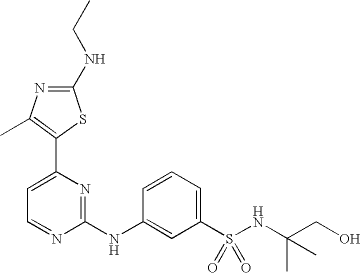 | 3-[4-(2-Ethylamino-4-methyl-thiazol- 5-yl)-pyrimidin-2-ylamino]-N-(2- hydroxy-1,1-dimethyl-ethyl)- benzenesulfonamide |
| 85 | 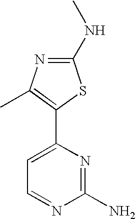 | 4-(4-Methyl-2-methylamino-thiazol- 5-yl)-pyrimidin-2-ylamine |
| 86 | 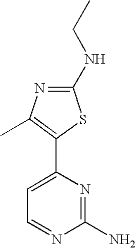 | 4-(2-Ethylamino-4-methyl-thiazol-5- yl)-pyrimidin-2-ylamine |
| 87 | 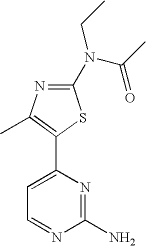 | N-[5-(2-Amino-pyrimidin-4-yl)-4- methyl-thiazol-2-yl]-N-ethyl- acetamide |
| 88 | 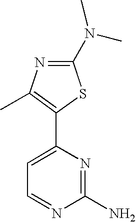 | 4-(2-Dimethylamino-4-methyl- thiazol-5-yl)-pyrimidin-2-ylamine |
| 89 | 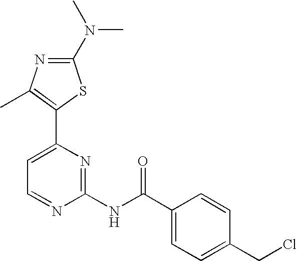 | 4-Chloromethyl-N-[4-(2- dimethylamino-4-methyl-thiazol-5- yl)-pyrimidin-2-yl]-benzamide |
| 90 | 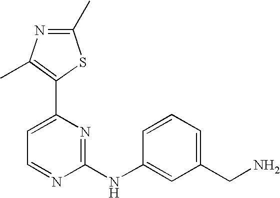 | (3-Aminomethyl-phenyl)-[4-(2,4- dimethyl-thiazol-5-yl)-pyrimidin-2- yl]-amine |
| 91 | 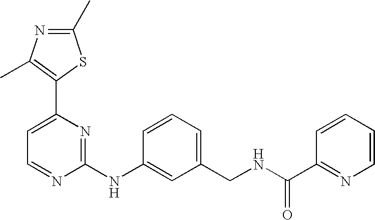 | Pyridine-2-carboxylic acid 3-[4-(2,4- dimethyl-thiazol-5-yl)-pyrimidin-2- ylamino]-benzylamide |
| 92 | 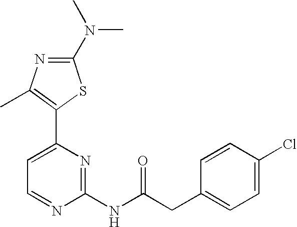 | 2-(4-Chloro-phenyl)-N-[4-(2- dimethylamino-4-methyl-thiazol-5- yl)-pyrimidin-2-yl]-acetamide |
| 93 | 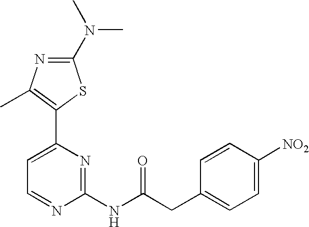 | N-[4-(2-Dimethylamino-4-methyl- thiazol-5-yl)-pyrimidin-2-yl]-2-(4- nitro-phenyl)-acetamide |
| 94 | 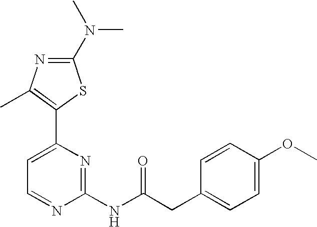 | N-[4-(2-Dimethylamino-4-methyl- thiazol-5-yl)-pyrimidin-2-yl]-2-(4- methoxy-phenyl)-acetamide |
| 95 | 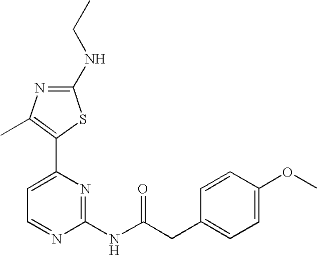 | N-[4-(2-Ethylamino-4-methyl-thiazol- 5-yl)-pyrimidin-2-yl]-2-(4-methoxy- phenyl)-acetamide |
| 96 | 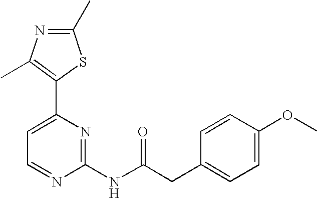 | N-[4-(2,4-Dimethyl-thiazol-5-yl)- pyrimidin-2-yl]-2-(4-methoxy- phenyl)-acetamide |
| 97 | 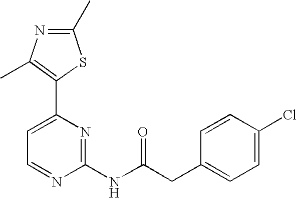 | 2-(4-Chloro-phenyl)-N-[4-(2,4- dimethyl-thiazol-5-yl)-pyrimidin-2- yl]-acetamide |
| 98 | 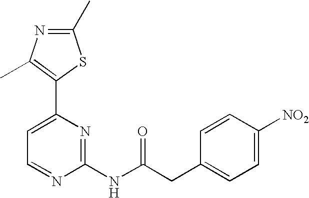 | N-[4-(2,4-Dimethyl-thiazol-5-yl)- pyrimidin-2-yl]-2-(4-nitro-phenyl)- acetamide |
| 99 | 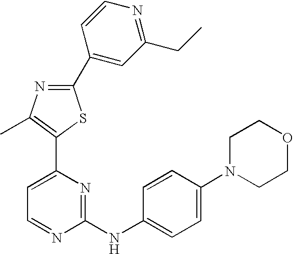 | {4-[2-(2-Ethyl-pyridin-4-yl)-4- methyl-thiazol-5-yl]-pyrimidin-2-yl}- (4-morpholin-4-yl-phenyl)-amine |
| 100 | 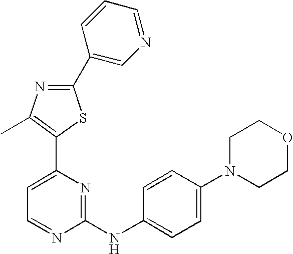 | [4-(4-Methyl-2-pyridin-3-yl-thiazol- 5-yl)-pyrimidin-2-yl]-(4-morpholin-4- yl-phenyl)-amine |
| 101 | 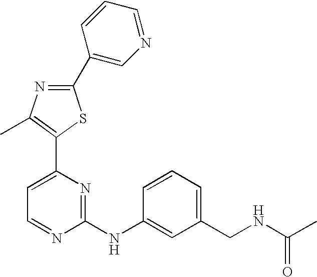 | N-{3-[4-(4-Methyl-2-pyridin-3-yl- thiazol-5-yl)-pyrimidin-2-ylamino]- benzyl}-acetamide |
| 102 | 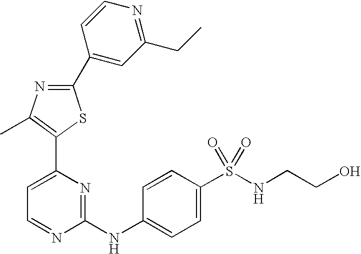 | 4-{4-[2-(2-Ethyl-pyridin-4-yl)-4- methyl-thiazol-5-yl]-pyrimidin-2- ylamino}-N-(2-hydroxy-ethyl)- benzenesulfonamide |
| 103 | 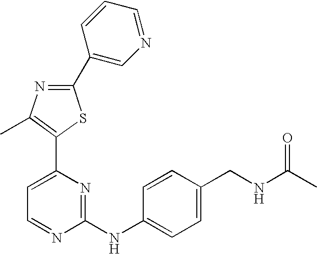 | N-{4-[4-(4-Methyl-2-pyridin-3-yl- thiazol-5-yl)-pyrimidin-2-ylamino]- benzyl}-acetamide |
| 104 | 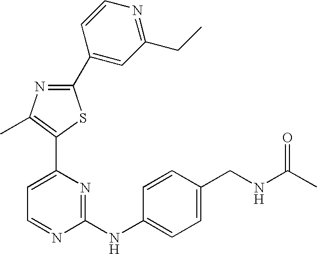 | N-(4-{4-[2-(2-Ethyl-pyridin-4-yl)-4- methyl-thiazol-5-yl]-pyrimidin-2- ylamino}-benzyl)-acetamide |
| 105 | 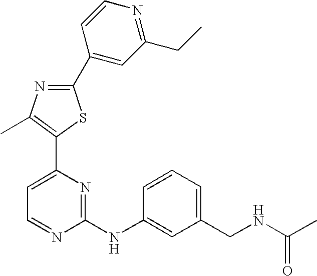 | N-(3-{4-[2-(2-Ethyl-pyridin-4-yl)-4- methyl-thiazol-5-yl]-pyrimidin-2- ylamino}-benzyl)-acetamide |
| 106 | 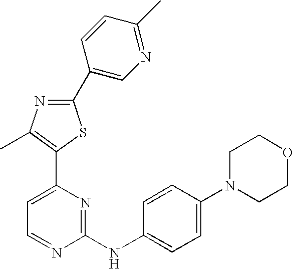 | {4-[4-Methyl-2-(6-methyl-pyridin-3- yl)-thiazol-5-yl]-pyrimidin-2-yl}-(4- morpholin-4-yl-phenyl)-amine |
| 107 | 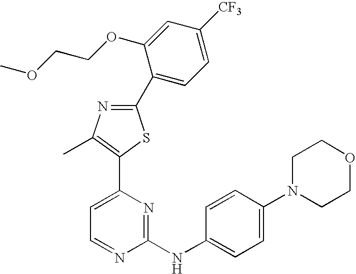 | (4-{2-[3-(2-Methoxy-ethoxy)-5- trifluoromethyl-pyridin-2-yl]-4- methyl-thiazol-5-yl}-pyrimidin-2-yl)- (4-morpholin-4-yl-phenyl)-amine |
| 108 | 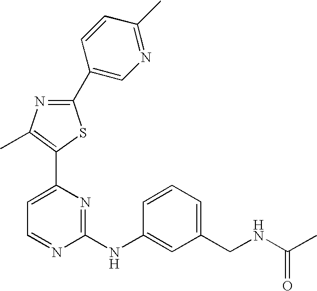 | N-(3-{4-[4-Methyl-2-(6-methyl- pyridin-3-yl)-thiazol-5-yl]-pyrimidin- 2-ylamino}-benzyl)-acetamide |
| 109 | 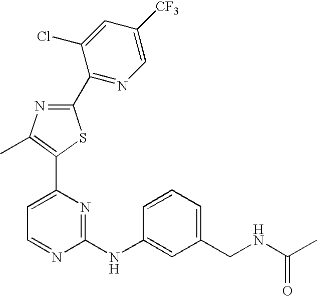 | N-(3-{4-[2-(3-Chloro-5- trifluoromethyl-pyridin-2-yl)-4- methyl-thiazol-5-yl]-pyrimidin-2- ylamino}-benzyl)-acetamide |
| 110 | 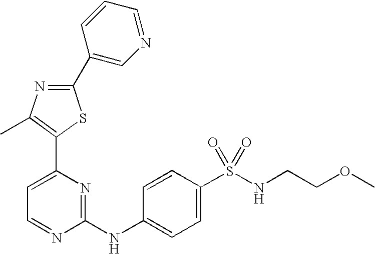 | N-(2-Methoxy-ethyl)-4-[4-(4-methyl- 2-pyridin-3-yl-thiazol-5-yl)- pyrimidin-2-ylamino]- benzenesulfonamide |
| 111 | 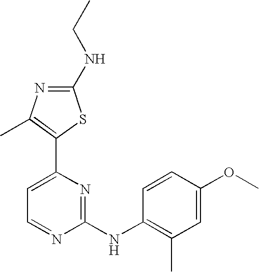 | [4-(2-Ethylamino-4-methyl-thiazol-5- yl)-pyrimidin-2-yl]-(4-methoxy-2- methyl-phenyl)-amine |
| 112 | 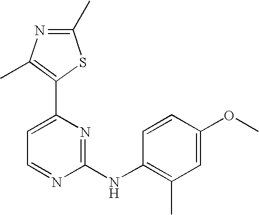 | [4-(2,4-Dimethyl-thiazol-5-yl)- pyrimidin-2-yl]-(4-methoxy-2- methyl-phenyl)-amine |
| 113 | 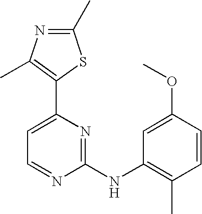 | [4-(2,4-Dimethyl-thiazol-5-yl)- pyrimidin-2-yl]-(5-methoxy-2- methyl-phenyl)-amine |
| 114 | 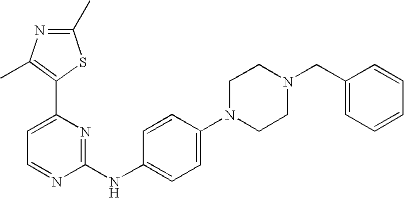 | [4-(4-Benzyl-piperazin-1-yl)-phenyl]- [4-(2,4-dimethyl-thiazol-5-yl)- pyrimidin-2-yl}-amine |
| 115 | 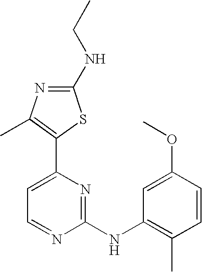 | [4-(2-Ethylamino-4-methyl-thiazol-5- yl)-pyrimidin-2-yl]-(5-methoxy-2- methyl-phenyl)-amine |
| 116 | 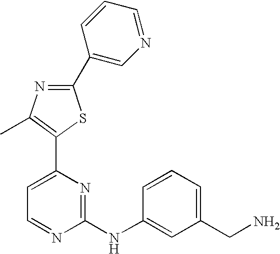 | (3-Aminomethyl-phenyl)-[4-(4- methyl-2-pyridin-3-yl-thiazol-5-yl)- pyrimidin-2-yl]amine |
| 117 | 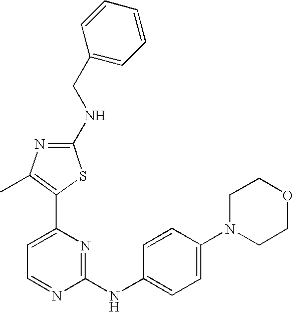 | [4-(2-Benzylamino-4-methyl-thiazol- 5-yl)-pyrimidin-2-yl]-(4-morpholin-4- yl-phenyl)-amine |
| 118 | 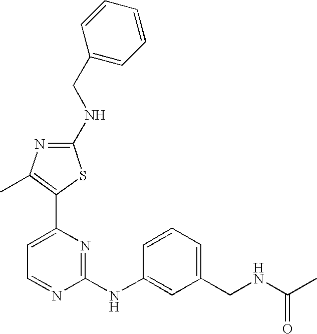 | N-{3-[4-(2-Benzylamino-4-methyl- thiazol-5-yl)-pyrimidin-2-ylamino]- benzyl}-acetamide |
| 119 | 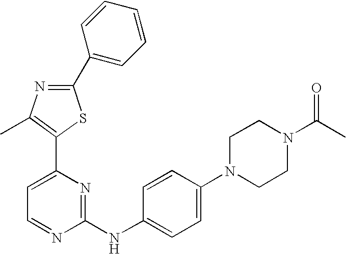 | 1-(4-{4-[4-(4-Methyl-2-pyridin-3-yl- thiazol-5-yl)-pyrimidin-2-ylamino]- phenyl}-piperazin-1-yl)-ethanone |
| 120 | 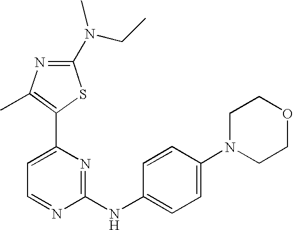 | {4-[2-(Ethyl-methyl-amino)-4- methyl-thiazol-5-yl]-pyrimidin-2-yl}- (4-morpholin-4-yl-phenyl)-amine |
| 121 | 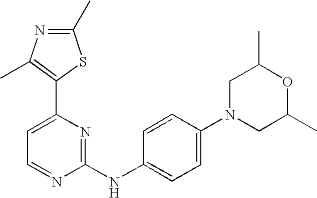 | [4-(2,6-Dimethyl-morpholin-4-yl)- phenyl]-[4-(2,4-dimethyl-thiazol-5- yl)-pyrimidin-2-yl]-amine |
| 122 | 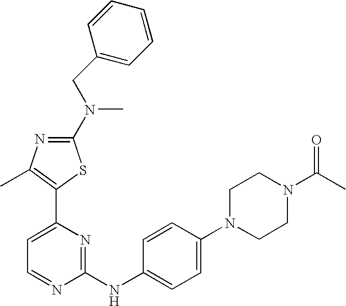 | 1-[4-(4-{4-[2-(Benzyl-methyl- amino)-4-methyl-thiazol-5-yl]- pyrimidin-2-ylamino}-phenyl)- piperazin-1-yl]-ethanone |
| 123 | 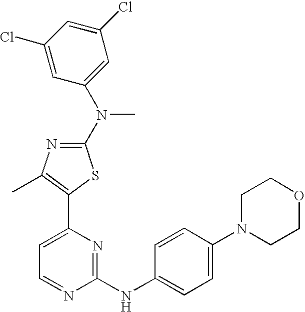 | (4-{2-[(3,5-Dichloro-phenyl)-methyl- amino]-4-methyl-thiazol-5-yl}- pyrimidin-2-yl)-(4-morpholin-4-yl- phenyl)-amine |
| 124 | 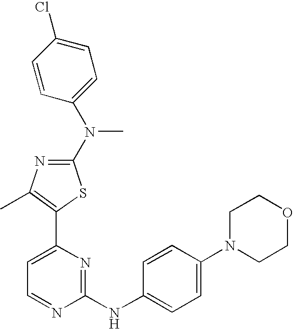 | (4-{2-[(4-Chloro-phenyl)-methyl- amino]-4-methyl-thiazol-5-yl}- pyrimidin-2-yl)-(4-morpholin-4-yl- phenyl)-amine |
| 125 | 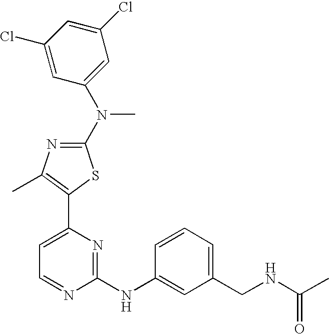 | N-[3-(4-{2-[(3,5-Dichloro-phenyl)- methyl-amino]-4-methyl-thiazol-5- yl}-pyrimidin-2-ylamino)-benzyl]- acetamide |
| 126 | 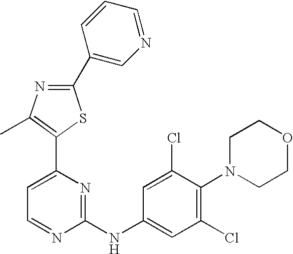 | (3,5-Dichloro-4-morpholin-4-yl- phenyl)-[4-(4-methyl-2-pyridin-3-yl- thiazol-5-yl)-pyrimidin-2-yl]-amine |
| 127 | 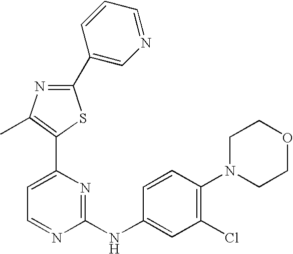 | (3-Chloro-4-morpholin-4-yl-phenyl)- [4-(4-methyl-2-pyridin-3-yl-thiazol-5- yl)-pyrimidin-2-yl]-amine |
| 128 | 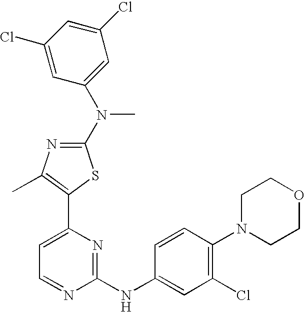 | (3-Chloro-4-morpholin-4-yl-phenyl)- (4-{2-[(3,5-dichloro-phenyl)-methyl- amino]-4-methyl-thiazol-5-yl}- pyrimidin-2-yl)-amine |
| 129 | 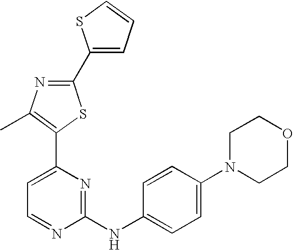 | [4-(4-Methyl-2-thiophen-2-yl-thiazol- 5-yl)-pyrimidin-2-yl]-(4-morpholin-4- yl-phenyl)-amine |
| 130 | 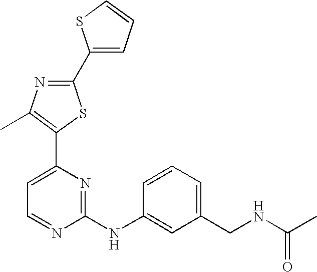 | N-{3-[4-(4-Methyl-2-thiophen-2-yl- thiazol-5-yl)-pyrimidin-2-ylamino]- benzyl}-acetamide |
| 131 | 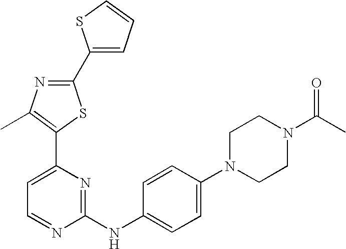 | 1-(4-{4-[4-(4-Methyl-2-thiophen-2- yl-thiazol-5-yl)-pyrimidin-2- ylamino]-phenyl}-piperazin-1-yl)- ethanone |
| 132 | 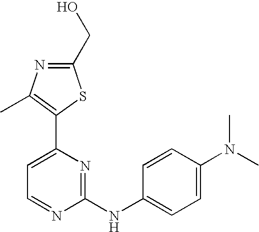 | {5-[2-(4-Dimethylamino- phenylamino)-pyrimidin-4-yl]-4- methyl-thiazol-2-yl}-methanol |
| 133 | 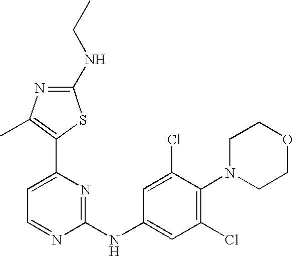 | (3,5-Dichloro-4-morpholin-4-yl- phenyl)-[4-(2-ethylamino-4-methyl- thiazol-5-yl)-pyrimidin-2-yl]-amine |
| 134 | 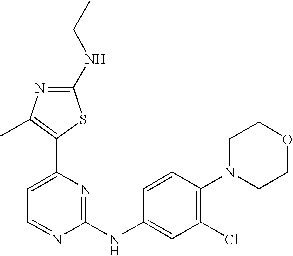 | (3-Chloro-4-morpholin-4-yl-phenyl)- [4-(2-ethylamino-4-methyl-thiazol-5- yl)-pyrimidin-2-yl]-amine |
| 135 | 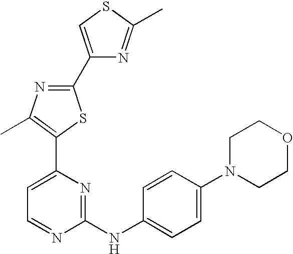 | [4-(4,2′-Dimethyl-[2,4′]bithiazolyl-5- yl)-pyrimidin-2-yl]-(4-morpholin-4- yl-phenyl)-amine |
| 136 | 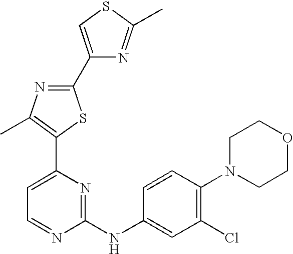 | (3-Chloro-4-morpholin-4-yl-phenyl)- [4-(4,2′-dimethyl-[2,4′]bithiazolyl-5- yl)-pyrimidin-2-yl]-amine |
| 137 | 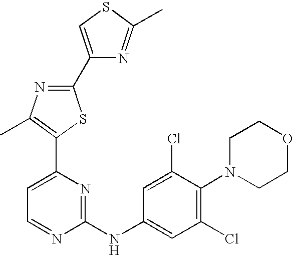 | (3,5-Dichloro-4-morpholin-4-yl- phenyl)-[4-(4,2′-dimethyl- [2,4′]bithiazolyl-5-yl)-pyrimidin-2- yl]-amine |
| 138 | 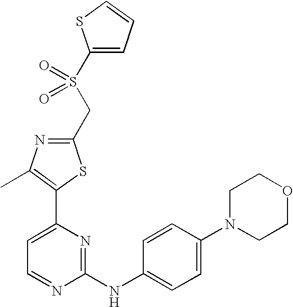 | {4-[4-Methyl-2-(thiophene-2- sulfonylmethyl)-thiazol-5-yl]- pyrimidin-2-yl}-(4-morpholin-4-yl- phenyl)-amine |
| 139 | 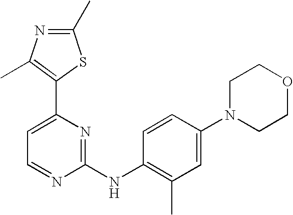 | [4-(2,4-Dimethyl-thiazol-5-yl)- pyrimidin-2-yl]-(2-methyl-4- morpholin-4-yl-phenyl)-amine |
| 140 | 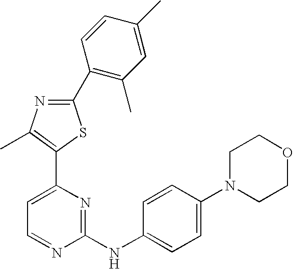 | {4-[2-(2,4-Dimethyl-phenyl)-4- methyl-thiazol-5-yl]-pyrimidin-2-yl}- (4-morpholin-4-yl-phenyl)-amine |
| 141 | 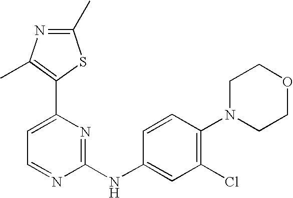 | (3-Chloro-4-morpholin-4-yl-phenyl)- [4-(2,4-dimethyl-thiazol-5-yl)- pyrimidin-2-yl]-amine |
| 142 | 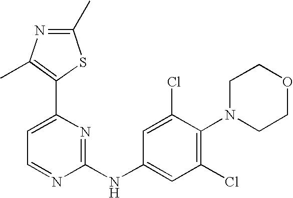 | (3,5-Dichloro-4-morpholin-4-yl- phenyl)-[4-(2,4-dimethyl-thiazol-5- yl)-pyrimidin-2-yl]-amine |
| 143 | 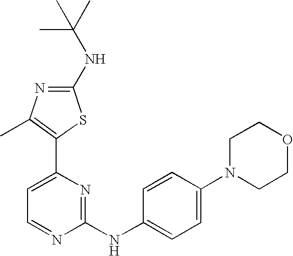 | [4-{2-tert-Butylamino-4-methyl- thiazol-5-yl)-pyrimidin-2-yl]-(4- morpholin-4-yl-phenyl)-amine |
| 144 | 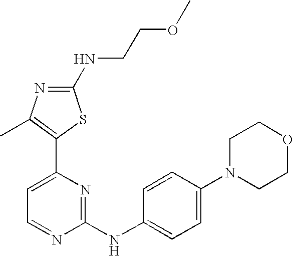 | {4-[2-(2-Methoxy-ethylamino)-4- methyl-thiazol-5-yl]-pyrimidin-2-yl}- (4-morpholin-4-yl-phenyl)-amine |
| 145 | 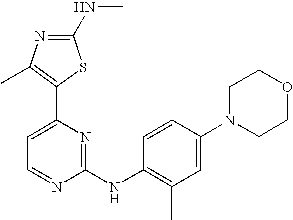 | [4-(4-Methyl-2-methylamino-thiazol- 5-yl)-pyrimidin-2-yl]-(2-methyl-4- morpholin-4-yl-phenyl)-amine |
| 146 | 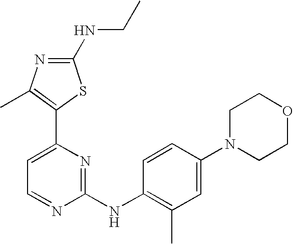 | [4-(2-Ethylamino-4-methyl-thiazol-5- yl)-pyrimidin-2-yl]-(2-methyl-4- morpholin-4-yl-phenyl)-amine |
| 147 | 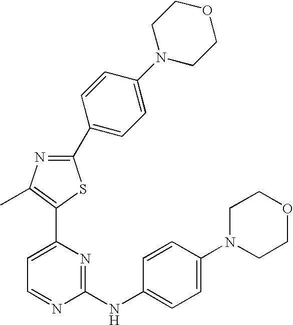 | {4-[4-Methyl-2-(4-morpholin-4-yl- phenyl)-thiazol-5-yl]-pyrimidin-2- yl}-(4-morpholin-4-yl-phenyl)-amine |
| 148 | 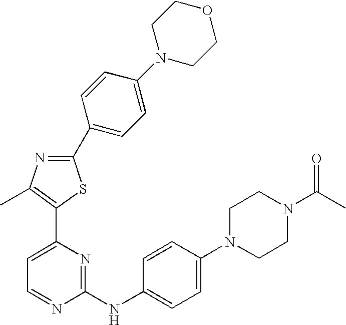 | 1-[4-(4-{4-[4-Methyl-2-(4-morpholin- 4-yl-phenyl)-thiazol-5-yl]-pyrimidin- 2-ylamino}-phenyl)-piperazin-1-yl]- ethanone |
| 149 | 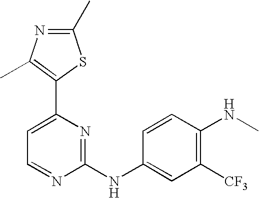 | N⁴-[4-(2,4-Dimethyl-thiazol-5-yl)- pyrimidin-2-yl]-N¹-methyl-2- trifluoromethyl-benzene-1,4-diamine |
| 150 | 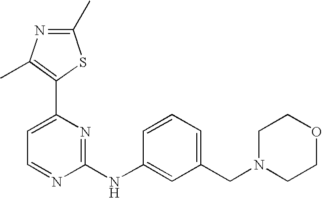 | [4-(2,4-Dimethyl-thiazol-5-yl)- pyrimidin-2-yl]-(3-morpholin-4- ylmethyl-phenyl)-amine |
| 151 | 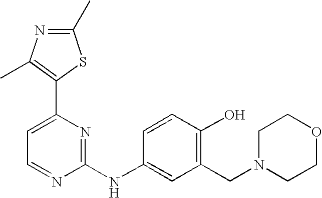 | 4-[4-(2,4-Dimethyl-thiazol-5-yl)- pyrimidin-2-ylamino]-2-morpholin-4- ylmethyl-phenol |
| 152 | 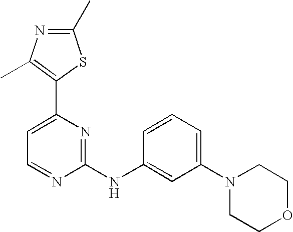 | [4-(2,4-Dimethyl-thiazol-5-yl)- pyrimidin-2-yl]-(3-morpholin-4-yl- phenyl)-amine |
| 153 | 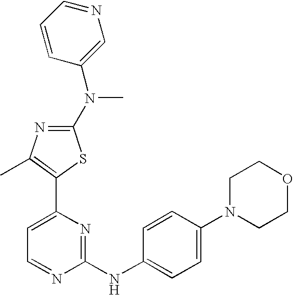 | {4-[4-Methyl-2-(methyl-pyridin-3-yl- amino)-thiazol-5-yl]-pyrimidin-2-yl}- (4-morpholin-4-yl-phenyl)-amine |
| 154 | 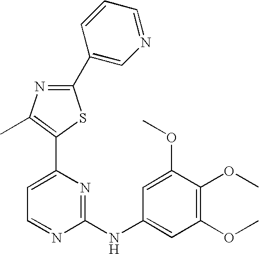 | [4-(4-Methyl-2-pyridin-3-yl-thiazol- 5-yl)-pyrimidin-2-yl]-(3,4,5- trimethoxy-phenyl)-amine |
| 155 | 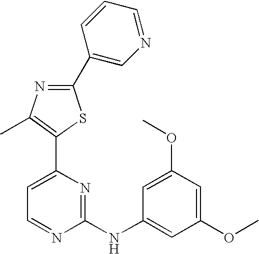 | (3,5-Dimethoxy-phenyl)-[4-(4- methyl-2-pyridin-3-yl-thiazol-5-yl)- pyrimidin-2-yl]-amine |
| 156 | 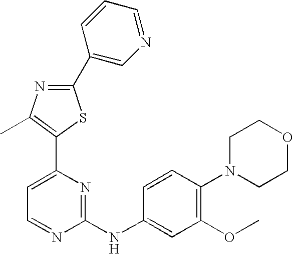 | (3-Methoxy-4-morpholin-4-yl- phenyl)-[4-(4-methyl-2-pyridin-3-yl- thiazol-5-yl)-pyrimidin-2-yl]-amine |
| 157 | 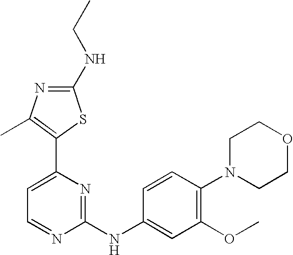 | [4-(2-Ethylanlino-4-methyl-thiazol-5- yl)-pyrimidin-2-yl]-(3-methoxy-4- morpholin-4-yl-phenyl)-amine |
| 158 | 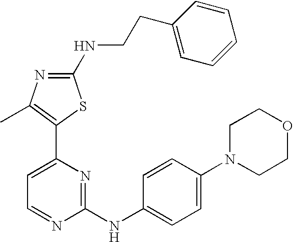 | [4-(4-Methyl-2-phenethylamino- thiazol-5-yl)-pyrimidin-2-yl]-(4- morpholin-4-yl-phenyl)-amine |
| 159 | 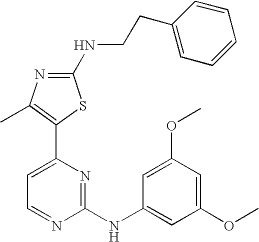 | (3,5-Dimethoxy-phenyl)-[4-(4- methyl-2-phenethylamino-thiazol-5- yl)-pyrimidin-2-yl]-amine |
| 160 | 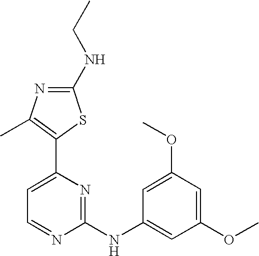 | (3,5-Dimethoxy-phenyl)-[4-(2- ethylamino-4-methyl-thiazol-5-yl)- pyrimidin-2-yl]-amine |
| 161 | 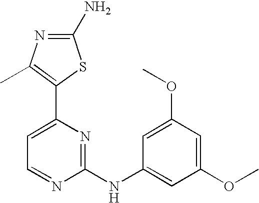 | [4-(2-Amino-4-methyl-thiazol-5-yl)- pyrimidin-2-yl]-(3,5-dimethoxy- phenyl)-amine |
| 162 | 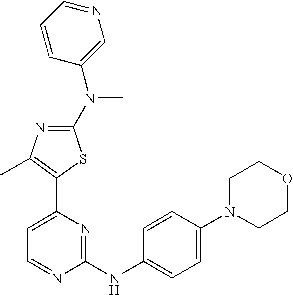 | {4-[4-Methyl-2-(methyl-pyridin-3-yl- amino)-thiazol-5-yl]-pyrimidin-2-yl}- (4-morpholin-4-yl-phenyl)-amine |
| 163 | 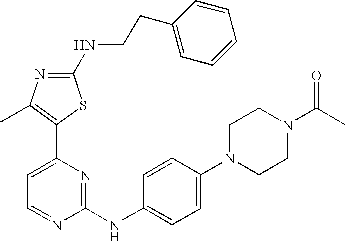 | 1-(4-{4-[4-(4-Methyl-2- phenethylamino-thiazol-5-yl)- pyrimidin-2-ylamino]-phenyl}- piperazin-1-yl)-ethanone |
| 164 | 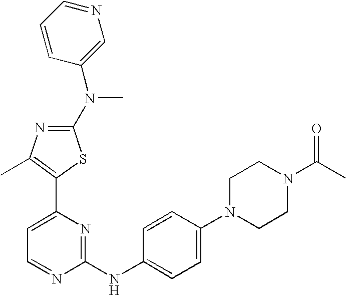 | 1-[4-(4-{4-[4-Methyl-2-(methyl- pyridin-2-yl-amino)-thiazol-5-yl]- pyrimidin-2-ylamino}-phenyl)- piperazin-1-yl]-ethanone |
| 165 | 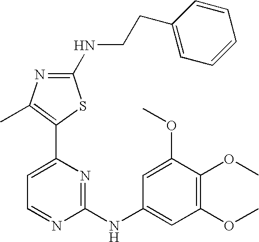 | [4-(4-Methyl-2-phenethylamino- thiazol-5-yl)-pyrimidin-2-yl]-(3,4,5- trimethoxy-phenyl)-amine |
| 166 | 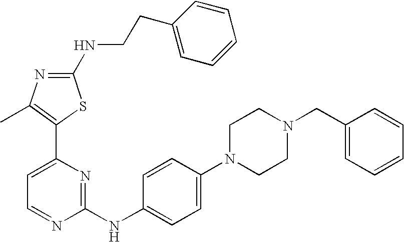 | [4-(4-Benzyl-piperazin-1-yl)-phenyl]- [4-(4-Methyl-2-phenylamino-thiazol- thiazol-5-yl)-pyrimidin-2-yl]-amine |
| 167 | 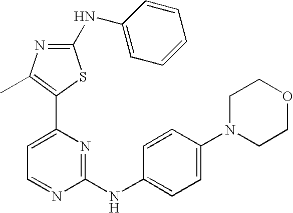 | [4-(4-Methyl-2-phenylamino-thiazol- 5-yl)-pyrimidin-2-yl]-(4-morpholin-4- yl-phenyl)-amine |
| 168 | 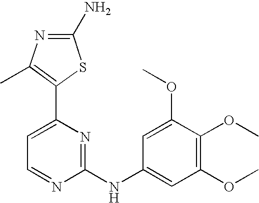 | [4-(2-Amino-4-methyl-thiazol-5-yl)- pyrimidin-2-yl]-(3,4,5-trimethoxy- phenyl)-amine |
| 169 | 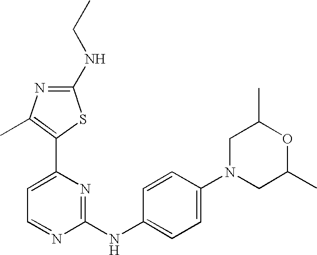 | [4-(2,6-Dimethyl-morpholin-4-yl)- phenyl]-[4-(2-ethylamino-4-methyl- thiazol-5-yl)-pyrimidin-2-yl]-amine |
| 170 | 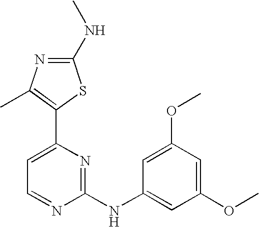 | (3,5-Dimethoxy-phenyl)-[4-(4- methyl-2-methylamino-thiazol-5-yl)- pyrimidin-2-yl]-amine |
| 171 | 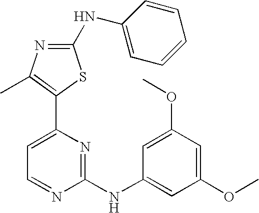 | (3,5-Dimethoxy-phenyl)-[4-(4- methyl-2-phenylamino-thiazol-5-yl)- pyrimidin-2-yl]-amine |
| 172 | 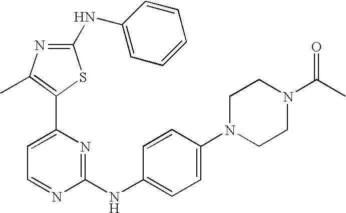 | 1-(4-{4[4-(4-Methyl-2-phenylamino- thiazol-5-yl)-pyrimidin-2-ylamino]- phenyl}-piperazin-1-yl)-ethanone |
| 173 | 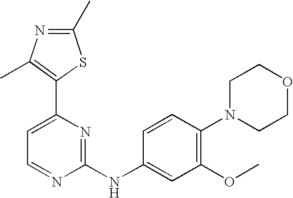 | [4-(2,4-Dimethyl-thiazol-5-yl)- pyrimidin-2-yl]-(3-methoxy-4- morpholin-4-yl-phenyl)-amine |
| 174 | 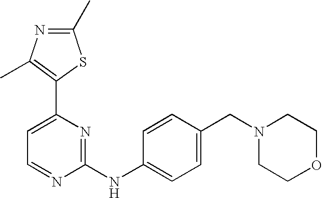 | [4-(2,4-Dimethyl-thiazol-5-yl)- pyrimidin-2-yl]-(4-morpholin-4- ylmethyl-phenyl)-amine |
| 175 | 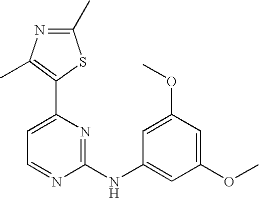 | (3,5-Dimethoxy-phenyl)-[4-(2,4- dimethyl-thiazol-5-yl)-pyrimidin-2- yl]-amine |
| 176 | 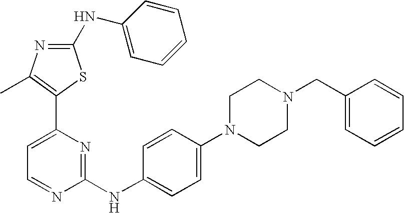 | [4-(4-Benzyl-piperazin-1-yl)-phenyl]- [4-(4-methyl-2-phenylamino-thiazol- 5-yl)-pyrimidin-2-yl]-amine |
| 177 | 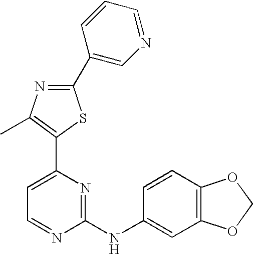 | Benzo[1,3]dioxol-5-yl-[4-(4-methyl- 2-pyridin-3-yl-thiazol-5-yl)- pyrimidin-2-yl]-amine |
| 178 | 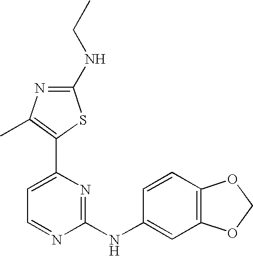 | Benzo[1,3]dioxol-5-yl-[4-(2- ethylamino-4-methyl-thiazol-5-yl)- pyrimidin-2-yl]-amine |
| 179 | 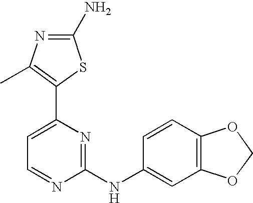 | [4-(2-Amino-4-methyl-thiazol-5-yl)- pyrimidin-2-yl]-benzo[1,3]dioxol-5- yl-amine |
| 180 | 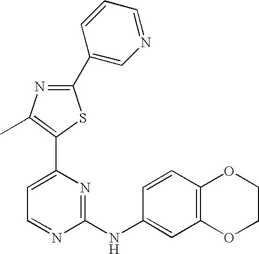 | (2,3-Dihydro-benzo[1,4]dioxin-6-yl)- [4-(4-methyl-2-pyridin-3-yl-thiazol-5- yl)-pyrimidin-2-yl]-amine |
| 181 | 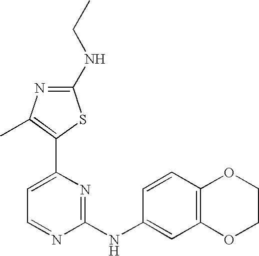 | (2,3-Dihydro-benzo[1,4]dioxin-6-yl)- [4-(2-ethylamino-4-methyl-thiazol-5- yl)-pyrimidin-2-yl]-amine |
| 182 | 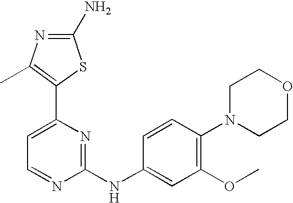 | [4-(2-Amino-4-methyl-thiazol-5-yl)- pyrimidin-2-yl]-(3-methoxy-4- morpholin-4-yl-phenyl)-amine |
| 183 | 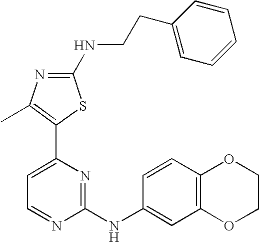 | (2,3-Dihydro-benzo[1,4]dioxin-6-yl)- [4-(4-methyl-2-phenethylamino- thiazol-5-yl)-pyrimidin-2-yl]-amine |
| 184 | 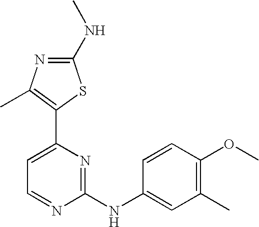 | (4-Methoxy-3-methyl-phenyl)-[4-(4- methyl-2-methylamino-thiazol-5-yl)- pyrimidin-2-yl]-amine |
| 185 | 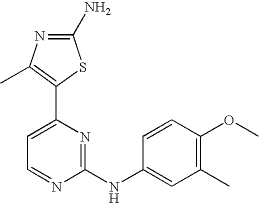 | [4-(2-Amino-4-methyl-thiazol-5-yl)- pyrimidin-2-yl]-(4-methoxy-3- methyl-phenyl)-amine |
| 186 | 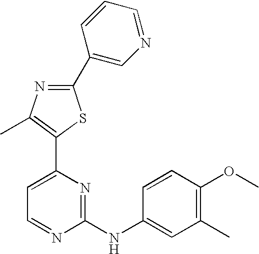 | (4-Methoxy-3-methyl-phenyl)-[4-(4- methyl-2-pyridin-3-yl-thiazol-5-yl)- pyrimidin-2-yl]-amine |
| 187 | 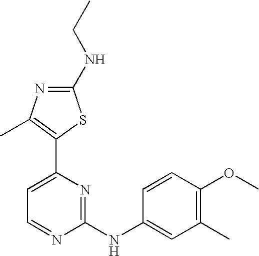 | [4-(2-Ethylamino-4-methyl-thiazol-5- yl)-pyrimidin-2-yl]-(4-methoxy-3- methyl-phenyl)-amine |
| 188 | 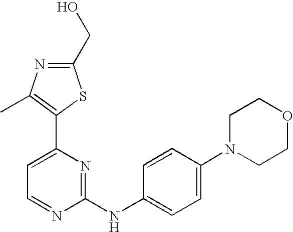 | {4-Methyl-5-[2-(4-morpholin-4-yl- phenylamino)-pyrimidin-4-yl]- thiazol-2-yl}-methanol |
| 189 | 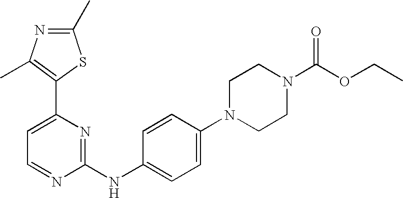 | 4-{4-[4-(2,4-Dimethyl-thiazol-5-yl)- pyrimidin-2-ylamino]-phenyl}- piperazine-1-carboxylic acid ethyl ester |
| 190 | 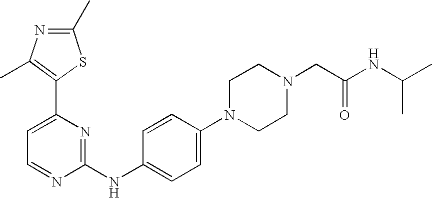 | 2-(4-{4-[4-(2,4-Dimethyl-thiazol-5- yl)-pyrimidin-2-ylamino]-phenyl}- piperazin-1-yl)-N-isopropyl- acetamide |
| 191 | 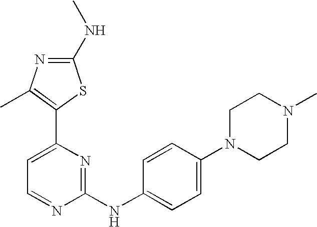 | [4-(4-Methyl-2-methylamino-thiazol- 5-yl)-pyrimidin-2-yl]-[4-(4-methyl- piperazin-1-yl)-phenyl]-amine |
| 192 | 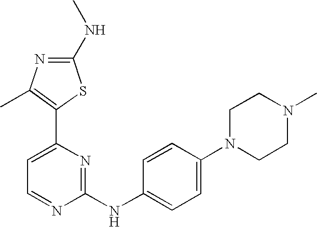 | [4-(4-Methyl-2-methylamino-thiazol- 5-yl)-pyrimidin-2-yl]-[4-(4-methyl- piperazin-1-yl)-phenyl]-amine |
| 193 | 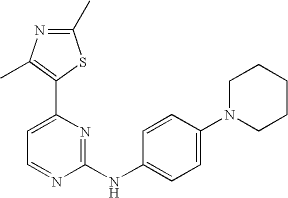 | [4-(2,4-Dimethyl-thiazol-5-yl)- pyrimidin-2-yl]-(4-piperidin-1-yl- phenyl)-amine |
| 194 | 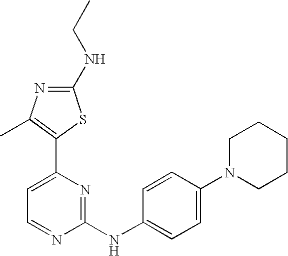 | [4-(2-Ethylamino-4-methyl-thiazol-5- yl)-pyrimidin-2-yl]-(4-piperidin-1-yl- phenyl)-amine |
| 195 | 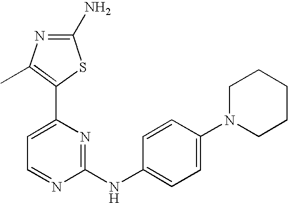 | [4-(2-Amino-4-methyl-thiazol-5-yl)- pyrimidin-2-yl]-(4-piperidin-1-yl- phenyl)-amine |
| 196 | 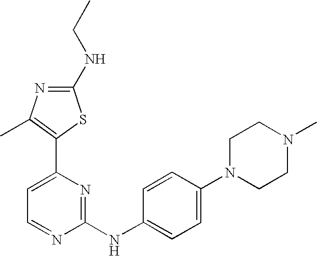 | [4-(2-Ethylamino-4-methyl-thiazol-5- yl)-pyrimidin-2-yl]-[4-(4-methyl- piperazin-1-yl)-phenyl]-amine |
| 197 | 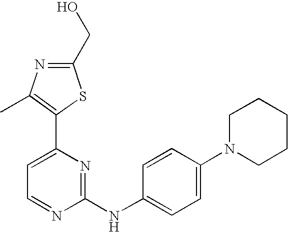 | {4-Methyl-5-[2-(4-piperidin-1-yl- phenylamino)-pyrimidin-4-yl]- thiazol-2-yl}-methanol |
| 198 | 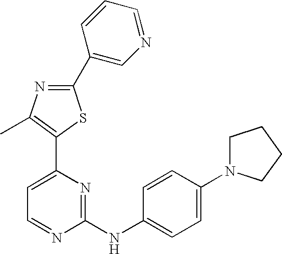 | [4-(4-Methyl-2-pyridin-3-yl-thiazol- 5-yl)-pyrimidin-2-yl]-(4-pyrrolidin-1- yl-phenyl)-amine |
| 199 | 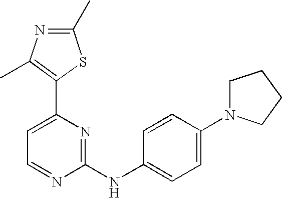 | [4-(2,4-Dimethyl-thiazol-5-yl)- pyrimidin-2-yl]-(4-pyrrolidin-1-yl- phenyl)-amine |
| 200 | 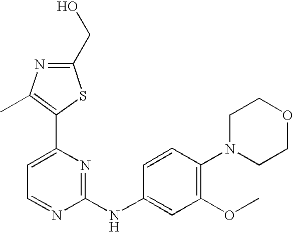 | {5-[2-(3-Methoxy-4-morpholin-4-yl- phenylamino)-pyrimidin-4-yl]-4- methyl-thiazol-2-yl}-methanol |
| 201 | 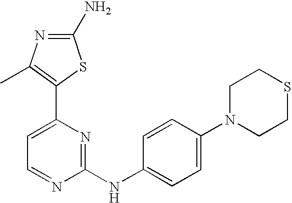 | [4-(2-Amino-4-methyl-thiazol-5-yl)- pyrimidin-2-yl]-(4-thiomorpholin-4- yl-phenyl)-amine |
| 202 | 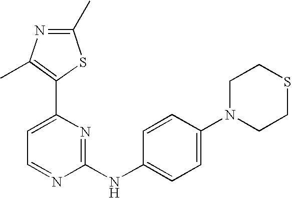 | [4-(2,4-Dimethyl-thiazol-5-yl)- pyrimidin-2-yl]-(4-thiomorpholin-4- yl-phenyl)-amine |
| 203 | 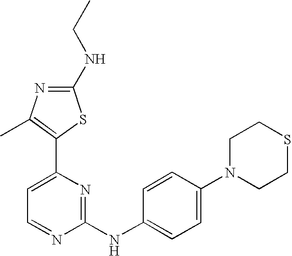 | [4-(2-Ethylamino-4-methyl-thiazol-5- yl)-pyrimidin-2-yl]-(4-thiomorpholin- 4-yl-phenyl)-amine |
| 204 | 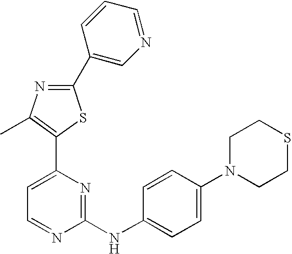 | [4-(4-Methyl-2-pyridin-3-yl-thiazol- 5-yl)-pyrimidin-2-yl]-(4- thiomorpholin-4-yl-phenyl)-amine |
| 205 | 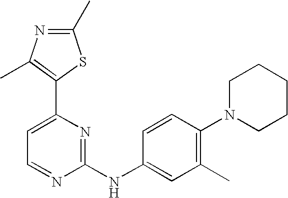 | [4-(2,4-Dimethyl-thiazol-5-yl)- pyrimidin-2-yl]-(3-methyl-4- piperidin-1-yl-phenyl)-amine |
| 206 | 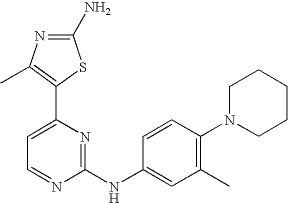 | [4-(2-Amino-4-methyl-thiazol-5-yl)- pyrimidin-2-yl]-(3-methyl-4- piperidin-1-yl-phenyl)-amine |
| 207 | 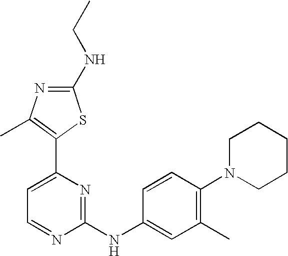 | [4-(2-Ethylamino-4-methyl-thiazol-5- yl)-pyrimidin-2-yl]-(3-methyl-4- piperidin-1-yl-phenyl)-amine |
| 208 | 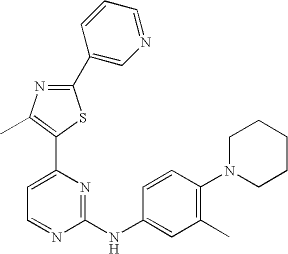 | (3-Methyl-4-piperidin-1-yl-phenyl)- [4-(4-methyl-2-pyridin-3-yl-thiazol-5- yl)-pyrimidin-2-yl]-amine |
| 209 | 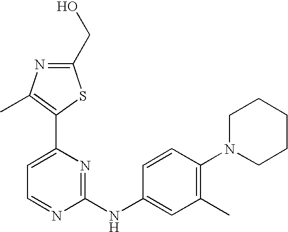 | {4-Methyl-5-[2-(3-methyl-4- piperidin-1-yl-phenylamino)- pyrimidin-4-yl]-thiazol-2-yl}- methanol |
| 210 | 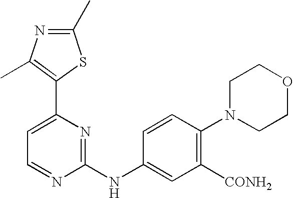 | 5-[4-(2-Amino-4-methyl-thiazol-5-yl)- pyrimidin-2-ylamino]-2-morpholin-4- yl-benzamide |
| 211 | 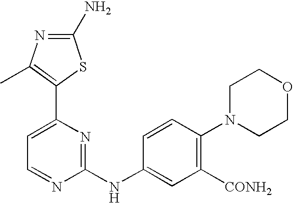 | 5-[4-(2-Amino-4-methyl-thiazol-5- yl)-pyrimidin-2-ylamino]-2- morpholin-4-yl-benzamide |
| 212 | 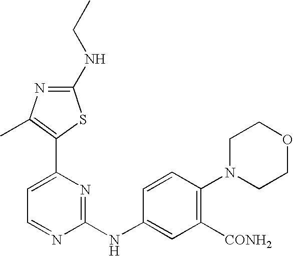 | 5-[4-(2-Ethylamino-4-methyl-thiazol- 5-yl)-pyrimidin-2-ylamino]-2- morpholin-4-yl-benzamide |
| 213 | 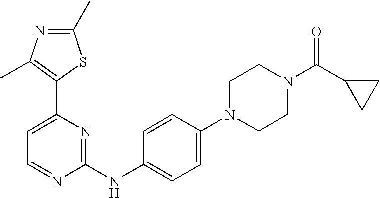 | Cyclopropyl-(4-{4-[4-(2,4-dimethyl- thiazol-5-yl)-pyrimidin-2-ylamino]- phenyl}-piperazin-1-yl)-methanone |
| 214 | 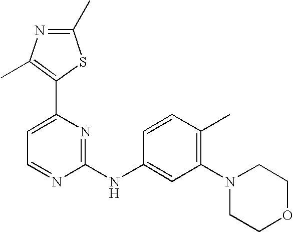 | [4-(2,4-Dimethyl-thiazol-5-yl)- pyrimidin-2-yl]-(4-methyl-3- morpholin-4-yl-phenyl)-amine |
| 215 | 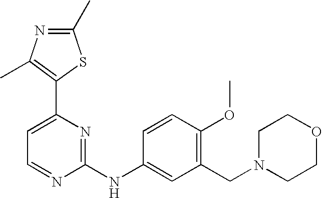 | [4-(2,4-Dimethyl-thiazol-5-yl)- pyrimidin-2-yl]-(4-methoxy-3- morpholin-4-ylmethyl-phenyl)-amine |
| 216 | 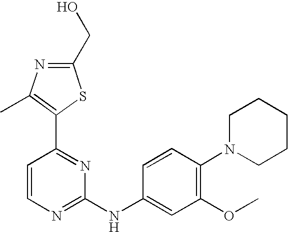 | {5-[2-(3-Methoxy-4-piperidin-1-yl- phenylamino)-pyrimidin-4-yl]-4- methyl-thiazol-2-yl}-methanol |
| 217 | 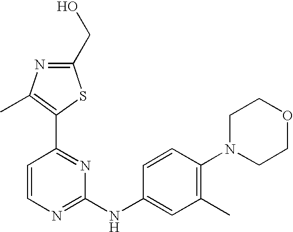 | {4-Methyl-5-[2-(3-methyl-4- morpholin-4-yl-phenylamino)- pyrimidin-4-yl]-thiazol-2-yl}- methanol |
| 218 | 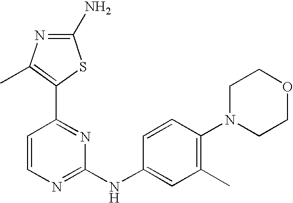 | [4-(2-Amino-4-methyl-thiazol-5-yl)- pyrimidin-2-yl]-(3-methyl-4- morpholin-4-yl-phenyl)-amine |
| 219 | 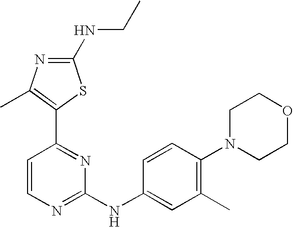 | [4-(2-Ethylamino-4-methyl-thiazol-5- yl)-pyrimidin-2-yl]-(3-methyl-4- morpholin-4-yl-phenyl)-amine |
| 220 | 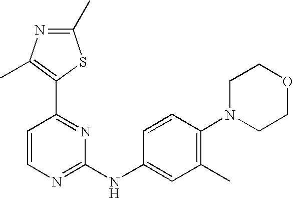 | [4-(2,4-Dimethyl-thiazol-5-yl)- pyrimidin-2-yl]-(3-methyl-4- morpholin-4-yl-phenyl)-amine |

TABLE 2
Inhibition of protein kinases by example compounds (pIC₅₀ is - log(IC₅₀, M)).

| | Kinase Inhibition pIC₅₀ | | | | | | | | |
|---|---|---|---|---|---|---|---|---|---|
| | CDK1- | CDK2- | CDK2- | CDK4- | CDK7- | CDK9- | | | Aurora A |
| No. | cyclin B | cyclin A | cycline E | cyclin D1 | cyclin H | cyclin T1 | GSK-3b | Flt3^{a} | kinase |
| 1 | | | 6.0 | | 5.5 | | | 6.9 | |
| 2 | 5.9 | | 5.8 | 6.5 | 6.1 | 6.5 | 6.2 | | |
| 3 | | | | 5.5 | 5.7 | | 5.3 | 7.7 | 7.7 |
| 4 | 6.3 | 6.0 | 6.5 | 5.9 | 6.0 | 6.6 | 6.4 | | 6.6 |
| 5 | 5.9 | 6.7 | 6.8 | 6.8 | 6.6 | 7.9 | 6.7 | 7.3 | 7.5 |
| 6 | 6.0 | 6.4 | 7.0 | 6.7 | 6.9 | 8.1 | | 7.3 | 6.9 |
| 7 | 5.5 | 5.6 | 6.4 | 6.0 | 6.2 | 6.7 | | 7.9 | 6.7 |
| 8 | 5.3 | 5.3 | 5.7 | 5.6 | 5.7 | 6.3 | 4.9 | 7.3 | 4.4 |
| 9 | 6.2 | 6.0 | 6.8 | 6.6 | 5.3 | 6.3 | 5.7 | | 6.8 |
| 10 | | 5.2 | 5.8 | | 5.2 | 6.5 | 5.2 | 6.8 | 5.7 |
| 11 | 5.8 | 5.8 | 6.8 | 5.9 | 6.2 | 5.9 | 5.8 | | 6.7 |
| 12 | 6.2 | 6.3 | 7.2 | 6.2 | 6.7 | 7.0 | 5.7 | 6.1 | 6.6 |
| 13 | 6.2 | 6.9 | 7.4 | 7.2 | 6.7 | 7.6 | | 7.5 | 7.8 |
| 14 | 6.4 | 6.2 | 7.4 | 6.9 | 6.1 | 7.1 | 6.2 | 7.1 | |
| 15 | 6.3 | 6.0 | 6.7 | 6.2 | 5.8 | 6.1 | 6.0 | 6.3 | 7.4 |
| 16 | 6.0 | 5.9 | 6.8 | 6.3 | 5.7 | 6.6 | 6.0 | 6.8 | 7.1 |
| 17 | 6.0 | 7.4 | 7.3 | 5.8 | 5.6 | 6.7 | 5.7 | 6.6 | 7.4 |
| 18 | 5.7 | 5.8 | 6.1 | 6.6 | 6.9 | 7.8 | 5.6 | 6.8 | |
| 19 | 6.7 | 6.8 | 8.3 | 7.8 | 7.3 | 8.6 | 6.8 | 7.5 | 7.4 |
| 20 | 6.8 | 7.8 | 8.6 | 5.6 | 5.4 | 7.2 | 6.5 | 6.5 | |
| 21 | 6.0 | 6.1 | 7.2 | 7.4 | 6.6 | 7.9 | 6.6 | 6.8 | 7.1 |
| 22 | 6.0 | 6.1 | 7.0 | 6.7 | 7.0 | 7.4 | | 7.1 | 7.2 |
| 23 | 6.0 | 5.9 | 6.3 | 5.7 | 6.1 | 7.7 | 6.1 | 7.9 | 8.4 |
| 24 | 5.7 | 5.9 | 6.3 | 5.9 | 6.3 | 7.5 | | 8.1 | 8.6 |
| 25 | 5.4 | 5.4 | 6.0 | | 5.5 | 5.7 | | 7.2 | 7.7 |
| 26 | 5.9 | 6.0 | 6.6 | 5.6 | 6.1 | 6.9 | 6.5 | 6.0 | 7.1 |
| 27 | 6.6 | 8.2 | 8.5 | 6.8 | 6.7 | 9.0 | 7.0 | 7.2 | 6.5 |
| 28 | | 6.1 | 7.8 | 6.6 | 7.0 | 8.5 | 6.2 | 7.1 | |
| 29 | | | 6.5 | 6.4 | 6.1 | 8.0 | | 6.0 | 7.3 |
| 30 | 5.7 | 6.0 | 7.0 | 6.1 | 6.4 | 7.7 | 6.5 | 6.7 | 7.8 |
| 31 | 6.3 | 5.9 | 6.6 | 7.3 | 6.0 | 7.7 | | 7.2 | 7.2 |
| 32 | | | | | 5.3 | 5.6 | | | 7.9 |
| 33 | 6.3 | 6.7 | 7.3 | 6.7 | 6.9 | 8.0 | 6.7 | | 5.9 |
| 34 | 7.4 | 7.9 | 8.4 | 7.0 | 6.0 | 7.5 | 5.9 | 6.5 | 7.5 |
| 35 | 7.5 | 8.2 | 8.4 | 7.7 | 7.1 | 8.0 | 6.2 | 7.2 | 7.2 |
| 36 | | | | | 5.9 | 7.4 | 5.4 | | 7.9 |
| 37 | | | 5.5 | | 5.4 | | 5.9 | 6.8 | 6.0 |
| 38 | | | | | 6.2 | 7.6 | 5.7 | 6.7 | 6.3 |
| 39 | 7.3 | 7.9 | 7.8 | 6.3 | 6.2 | 6.7 | 5.3 | | 6.9 |
| 40 | 7.1 | 7.8 | 8.5 | | | 6.8 | 6.3 | | 8.3 |
| 41 | 7.3 | 7.8 | 8.1 | 6.3 | 5.5 | | 6.1 | | 7.3 |
| 42 | | | 6.5 | | 5.2 | 7.4 | 6.0 | 7.1 | 7.6 |
| 43 | | | 8.0 | | | | | | 6.9 |
| 44 | 5.7 | 5.7 | | 5.7 | 5.5 | 6.6 | | 7.2 | |
| 45 | 5.3 | 5.5 | 5.6 | 5.5 | 5.4 | 6.4 | | 7.0 | 7.8 |
| 46 | 4.9 | | | | 5.3 | 6.0 | 5.5 | 6.7 | |
| 47 | | | | | | | | 6.7 | |
| 48 | 6.2 | 6.2 | 6.6 | 6.2 | | | | 7.2 | 7.3 |
| 49 | 6.7 | 6.8 | 7.6 | 6.0 | | 6.5 | | 6.7 | 8.3 |
| 50 | | | | | | 6.7 | | | 7.7 |
| 51 | 5.8 | | 6.2 | 6.2 | | | 6.2 | | 7.0 |
| 52 | | | 6.0 | | | | | | 7.0 |
| 53 | 5.7 | 5.7 | 6.4 | 6.8 | 6.8 | 7.0 | 6.3 | 6.7 | 6.8 |
| 54 | | | 7.0 | 6.6 | 6.1 | 7.4 | 6.2 | 6.6 | 7.6 |
| 55 | 5.5 | 5.7 | 6.6 | 5.8 | 6.2 | 7.5 | 6.3 | 6.7 | 6.8 |
| 56 | | 5.4 | | | 5.3 | 6.3 | 5.9 | | 6.9 |
| 57 | 4.1 | | 4.9 | 5.0 | 4.8 | 5.0 | 4.9 | | 7.2 |
| 58 | 5.3 | 6.0 | 6.2 | 6.6 | 6.0 | 6.7 | 5.3 | 7.6 | 7.7 |
| 59 | 6.0 | 6.3 | 6.8 | 7.4 | 6.9 | 7.0 | | 7.8 | 7.1 |
| 60 | 5.2 | 5.5 | 6.2 | 6.8 | 6.7 | 6.5 | | 7.4 | 7.3 |
| 61 | 5.2 | 5.7 | 6.4 | 6.6 | 6.4 | 6.8 | | 7.2 | 7.7 |
| 62 | | 5.5 | 7.1 | | | 5.2 | | | |
| 63 | 5.2 | 5.5 | 6.2 | 6.3 | 5.6 | 6.4 | 5.8 | | 5.8 |
| 64 | 5.2 | 5.6 | 6.4 | 6.4 | 5.8 | 6.6 | 5.8 | | 6.7 |
| 65 | 7.3 | 7.7 | 7.8 | 7.9 | 6.7 | | 6.5 | 6.7 | 6.6 |
| 66 | | 7.3 | 7.2 | | | | | | 6.5 |
| 67 | 7.3 | 7.8 | 8.2 | 6.3 | 5.4 | | 5.9 | 6.5 | 6.5 |
| 68 | 7.7 | 7.7 | 8.0 | 5.8 | 5.7 | 6.7 | 5.9 | 6.7 | 7.2 |
| 69 | 8.0 | 7.7 | 8.0 | 6.0 | 5.7 | 6.4 | 5.8 | | 7.1 |
| 70 | | | | 6.8 | | 7.0 | 5.3 | | 6.0 |
| 71 | | 5.5 | 6.2 | 5.1 | | 6.4 | | | |
| 72 | 6.1 | 5.9 | 6.8 | | | 5.2 | | | 5.6 |
| 73 | 7.0 | 7.4 | 7.9 | | 6.0 | 6.8 | | | 6.5 |
| 74 | | 5.7 | 6.4 | | | | | | 6.9 |
| 75 | 6.0 | 5.9 | 6.7 | 6.9 | 6.4 | 7.2 | 6.1 | 7.2 | |
| 76 | 5.9 | 5.8 | 6.5 | 7.0 | 6.1 | 7.2 | 6.3 | 7.0 | 7.4 |
| 77 | 6.0 | 6.0 | 6.9 | 6.9 | 6.4 | 7.6 | 6.5 | 6.8 | 7.0 |
| 78 | 5.7 | 5.7 | 6.5 | 6.3 | 5.9 | 6.8 | 6.0 | | 6.2 |
| 79 | | | 5.2 | 5.5 | 5.2 | 5.8 | | | |
| 80 | | | | | | 6.6 | 5.6 | | 6.4 |
| 81 | | | 5.5 | | 5.2 | | 7.6 | | 5.5 |
| 82 | | | 5.4 | | 5.4 | 6.4 | 5.5 | | 5.5 |
| 83 | | | 7.6 | | 7.3 | 7.8 | 6.1 | 7.4 | |
| 84 | | | 6.2 | | 5.8 | 6.8 | 5.6 | | 7.6 |
| 85 | | | | | | | | | 6.4 |
| 86 | | | | | | | | | 6.0 |
| 87 | | | | | | | | | 6.8 |
| 88 | | | | | | | | | 5.8 |
| 90 | | | 6.4 | | | | | | |
| 91 | | | 6.4 | | | | 5.4 | 6.6 | 6.1 |
| 100 | | | | | | | | 6.5 | 6.6 |
| 101 | 5.5 | | 5.9 | 5.4 | 5.4 | 5.3 | 5.1 | 6.5 | 6.9 |
| 102 | | | | | | | | | 7.2 |
| 103 | | | 5.6 | | 5.6 | 5.3 | | | |
| 104 | | | | | | | | | 7.2 |
| 105 | | | | | | | | | 6.5 |
| 107 | | | | | | | | | 6.5 |
| 108 | | | | | | | | | 5.6 |
| 109 | | | | | | | | | 6.2 |
| 110 | | | | | | | | | 6.0 |
| 111 | | | | | | | | | 5.5 |
| 112 | | | | | | | | | 5.6 |
| 113 | | | | | | | | | 5.3 |
| 114 | | | 5.9 | | 6.9 | 5.8 | | | 6.5 |
| 118 | | | | | | | | | 6.3 |
| 119 | | | | | | | | | 7.3 |
| 120 | | | 6.1 | | 5.5 | 5.7 | | | 7.5 |
| 121 | | | 5.3 | | | 5.4 | | 6.4 | 7.4 |
| 122 | | | 5.7 | | 5.8 | 5.9 | | | 6.7 |
| 123 | | | | | | | | | 7.3 |
| 124 | | | | | | | | | 6.7 |
| 125 | | | | | | | | | 5.6 |
| 126 | | | | | | | | | 6.6 |
| 127 | | | | | | | | | |
| 128 | | | | | | | | | 7.0 |
| 129 | | | | | | | | | |
| 130 | | | 6.1 | | | | | | 6.8 |
| 131 | | | 5.6 | | 5.8 | 5.6 | | | 6.6 |
| 132 | | | 6.2 | | | | | | 7.0 |
| 134 | | | 6.7 | | | | | | 6.9 |
| 135 | | | | | | | | | 5.9 |
| 138 | | | 5.4 | | 5.5 | | | | 7.3 |
| 139 | | | | | | | | | 5.8 |
| 140 | | | | | | | | | 5.8 |
| 141 | | | 6.1 | | | | | | 6.8 |
| 143 | | | 6.9 | | | | | | 7.3 |
| 144 | | | 6.6 | | | | | | 7.0 |
| 145 | | | | | | | | | 5.4 |
| 146 | | | | | | | | | 5.6 |
| 147 | | | | | | | | | 6.3 |
| 148 | | | | | | | | | 6.5 |
| 149 | | | 6.6 | | | | | | 6.4 |
| 150 | | | 5.9 | | | | | 6.9 | 6.8 |
| 151 | | | 6.7 | | | | | 7.2 | 7.2 |
| 152 | | | 6.5 | | | | | 7.5 | 7.3 |
| 153 | | | | | | | | | 6.1 |
| 154 | | | | | | | | 7.0 | 7.1 |
| 156 | | | | | | | | 7.0 | 7.0 |
| 157 | | | 6.6 | | | | | | 7.6 |
| 158 | | | 6.0 | | | | | | 6.7 |
| 159 | | | | | | | | | 6.1 |
| 160 | | | | | | | | | 6.1 |
| 161 | | | 6.8 | | | | | 8.3 | 7.0 |
| 162 | | | 5.5 | | | 5.5 | | 7.2 | 6.9 |
| 163 | | | 5.9 | | 5.6 | 6.2 | | 6.9 | 7.2 |
| 164 | | | 6.4 | | 5.4 | 5.7 | | 7.6 | 7.5 |
| 165 | | | 5.6 | | | | | 7.7 | 7.2 |
| 168 | | | 6.4 | | 5.9 | 6.5 | | 8.3 | 7.5 |
| 169 | | | 5.8 | | | 5.5 | | 7.4 | 7.3 |
| 170 | | | 6.5 | | | | | 8.0 | 7.2 |
| 172 | | | | | | | | | 6.6 |
| 173 | | | 6.1 | | 5.5 | 5.5 | | | 7.4 |
| 174 | | | 5.9 | | 6.6 | 6.0 | | 6.8 | 7.0 |
| 175 | | | 5.9 | | 5.3 | 5.8 | | 7.4 | 7.1 |
| 176 | | | | | | | | | 5.2 |
| 177 | | | | | | | | | 5.7 |
| 178 | | | 6.7 | | | | | | 6.8 |
| 179 | | | 7.0 | | | | | | 7.1 |
| 180 | | | | | | | | | 5.8 |
| 181 | | | 6.3 | | | | | | 6.7 |
| 182 | | | 6.1 | | 5.6 | 6.0 | | | 7.4 |
| 183 | | | | | | | | | 5.9 |
| 184 | | | 6.9 | | | | | | 7.2 |
| 185 | | | 6.6 | | | | | | 7.4 |
| 186 | | | | | | | | | 6.3 |
| 187 | | | 6.1 | | | | | | 7.0 |
| 188 | | | 5.8 | | | 5.4 | | | 7.1 |
| 189 | | | 5.7 | | | 5.7 | | | 7.0 |
| 190 | | | 6.0 | | 5.1 | 5.6 | | | 7.2 |
| 191 | | | | | | | | | 6.5 |
| 192 | | | | | | | | | 6.0 |
| 193 | | | | | | 5.4 | | | 6.7 |
| 194 | | | 6.1 | | | | | | 7.1 |
| 195 | | | 6.1 | | 5.8 | 6.4 | | | 7.4 |
| 196 | | | 6.2 | | | | | | 6.7 |
| 197 | | | 5.6 | | 5.1 | 5.5 | | | 7.0 |
| 198 | | | | | | | | | 5.7 |
| 199 | | | | | | | | | 6.2 |
| 200 | | | 5.7 | | | | | | 7.4 |
| 201 | | | 5.8 | | | 5.8 | | | 7.2 |
| 202 | | | 5.3 | | | | | | 6.8 |
| 203 | | | 5.7 | | | 5.5 | | | 6.9 |
| 204 | | | | | | | | | 6.3 |
| 205 | | | | | | | | | 6.4 |
| 206 | | | 5.9 | | | | | | 6.8 |
| 207 | | | | | | | | | 6.3 |
| 208 | | | | | | | | | 6.0 |
| 209 | | | | | | | | | 6.6 |
| 210 | | | | | | | | | 6.6 |
| 211 | | | 5.6 | | 5.2 | 5.7 | | | 6.8 |
| 212 | | | 6.0 | | | 5.9 | | | 7.2 |
| 213 | | | 5.8 | | | 5.6 | | | 7.4 |
| 214 | | | | | | | | | 6.6 |
| 215 | | | 5.8 | | | | | | 6.7 |
| 216 | | | 5.8 | | 5.8 | 6.2 | | | 7.2 |
| 217 | | | 5.9 | | 5.1 | 5.4 | | | 7.3 |
| 218 | | | 5.9 | | | 5.8 | | | 7.2 |
| 219 | | | 6.0 | | | 5.5 | | | 7.2 |
| 220 | | | 5.9 | | | 5.2 | | | 7.4 |

^{a}FMS-like tyrosine kinase-3.

TABLE 3
Kinase selectivity profile for selected example compounds.

| | Test compound no. | | | |
|---|---|---|---|---|
| | 59 | | 138 | |
| | Remaining | | Remaining | |
| Protein kinase | activity (%) | SD (%) | activity (%) | SD (%) |
| MKK1 | 25 | 0 | 21 | 3 |
| MAPK2/ERK2 | 59 | 9 | 39 | 2 |
| JNK/SAPK1c | 2 | 1 | 10 | 4 |
| SAPK2a/p38 | 39 | 4 | 33 | 9 |
| SAPK2b/p38β2 | 36 | 8 | 42 | 4 |
| SAPK3/p38g | 69 | 7 | 74 | 8 |
| SAPK4/p38d | 66 | 3 | 85 | 4 |
| MAPKAP-K1a | 14 | 2 | 34 | 2 |
| MAPKAP-K2 | 84 | 8 | 84 | 6 |
| MSK1 | 45 | 3 | 69 | 2 |
| PRAK | 44 | 1 | 59 | 6 |
| PKA | 22 | 1 | 29 | 4 |
| PKCa | 33 | 1 | 41 | 2 |
| PDK1 | 20 | 1 | 55 | 5 |
| PKB | 82 | 0 | 91 | 7 |
| SGK | 0 | 0 | 5 | 0 |
| p70 S6K | 13 | 2 | 41 | 2 |
| GSK3b | 37 | 6 | 21 | 1 |
| ROCK-II | 5 | 2 | 14 | 3 |
| AMPK | 2 | 1 | 28 | 6 |
| CHK1 | 10 | 0 | 55 | 8 |
| CK2 | 2 | 0 | 5 | 0 |
| PHK | 3 | 3 | 15 | 4 |
| Lck | 1 | 1 | 7 | 3 |
| CSK | 14 | 1 | 30 | 1 |
| CDK2/cyclin A | 1 | 1 | 9 | 3 |
| DYRK1a | 4 | 3 | 27 | 0 |
| CK1 | 3 | 1 | 27 | 1 |
| NEK6 | 47 | 0 | 80 | 6 |
| NEK2a | 9 | 5 | 6 | 0 |
| MAPKAP-K1b | 17 | 1 | 48 | 7 |
| IKKb | 7 | 3 | 34 | 3 |
| smMLCK | 10 | 6 | 44 | 6 |
| PRK2 | 8 | 2 | 78 | 6 |
| MNK2 | 20 | 0 | 27 | 0 |
| CAMK-1 | 30 | 4 | 91 | 8 |
| PIM2 | 80 | 2 | 93 | 2 |
| NEK7 | 50 | 1 | 79 | 6 |
| JNK3 | 13 | 0 | 15 | 10 |
| MAPKAP-K3 | 87 | 2 | 104 | 7 |
| ERK8 | 4 | 3 | 11 | 0 |
| MNK1 | 3 | 1 | 18 | 0 |
| SRPK1 | 88 | 2 | 110 | 4 |
| PKBb | 75 | 2 | 92 | 8 |
| Aurora B | 4 | 2 | 5 | 3 |

Results are expressed as percentage remaining kinase activity in the presence of 5 μM test compound compared to control (no test compound); SD: standard deviation.

TABLE 4
Kinase selectivity profile for selected example compounds.

| | Kinase inhibition IC₅₀ (μM) | | | | | | |
|---|---|---|---|---|---|---|---|
| | Test compound no. | | | | | | |
| Kinase | 19 | 27 | 34 | 37 | 38 | 55 | 59 |
| Ab1 | 0.37 | 2.1 | 7.6 | 3.8 | 2.3 | 2.1 | 0.47 |
| Akt/PKB | >10 | >10 | >10 | >10 | >10 | >10 | >10 |
| Aurora A | 0.04 | 0.32 | 0.03 | 1.1 | 0.45 | 0.16 | 0.09 |
| Aurora B | 0.02 | 0.26 | 0.005 | 0.24 | 0.14 | 0.11 | 0.016 |
| CaMKII | 4.3 | >10 | >10 | >10 | 6.2 | >10 | 1.0 |
| CDK1B | 0.20 | 0.26 | 0.04 | >10 | >10 | 2.9 | 1.2 |
| CDK2A | 0.16 | 0.007 | 0.012 | >10 | >10 | 2.1 | 0.63 |
| CDK2E | 0.005 | 0.003 | 0.004 | 3.0 | >10 | 0.27 | 0.15 |
| CDK4D1 | 0.017 | 0.18 | 0.11 | >10 | >10 | 1.6 | 0.046 |
| CDK6D3 | 0.039 | 0.33 | 0.098 | >10 | 4.8 | 2.1 | 0.029 |
| CDK7H | 0.054 | 0.20 | 0.90 | 5.9 | 0.65 | 0.62 | 0.16 |
| CDK9T1 | 0.003 | 0.001 | 0.029 | 0.022 | 0.028 | 0.03 | 0.11 |
| CK2 | 2.0 | 1.0 | 2.0 | >10 | 5.9 | 1.5 | 1.5 |
| ERK2 | >10 | >10 | >10 | >10 | >10 | >10 | >10 |
| Flt3 | 0.03 | 0.065 | 0.32 | 0.17 | 0.20 | 0.18 | 0.017 |
| GSK3b | 0.17 | 0.094 | 1.3 | 1.8 | 2.2 | 0.45 | >10 |
| GSK3a | 0.16 | 0.012 | 0.22 | 0.65 | 0.96 | 0.29 | >10 |
| Lck | 0.49 | 0.50 | 6.1 | 4.9 | 2.6 | 1.8 | 0.12 |
| PDGFb | 0.49 | 0.52 | 0.85 | 6.6 | 4.8 | 1.9 | 0.21 |
| PKA | >10 | >10 | >10 | >10 | >10 | >10 | 6.6 |
| PKC | 5.5 | >10 | >10 | 6.4 | 2.5 | 5.8 | >10 |
| Plk1 | >10 | >10 | >10 | >10 | >10 | >10 | >10 |
| S6 | 1.4 | >10 | >10 | >10 | 5.9 | 4.0 | 0.59 |
| SAPK2a | 0.58 | >10 | >10 | >10 | >10 | >10 | 3.3 |
| Src | 1.2 | | | | | | |
| VEGFR2 | 0.036 | 0.044 | 0.11 | 0.39 | | 0.24 | 0.046 |

1. A compound of formula I, or a pharmaceutically acceptable salt thereof, 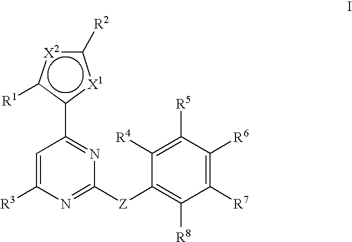
wherein:
one of X¹ and X² is S, and the other of X¹ and X² is N;
Z is NH, NHCO, NHCOCH₂, NHSO₂, NHCH₂, CH₂, CH₂CH₂, CH═CH, O, S, SO₂, or SO;
R¹, R², R³, R⁴, R⁵, R⁶, R⁷ and R⁸ are each independently H, alkyl, alkyl-R⁹, aryl, aryl-R⁹, aralkyl, aralkyl-R⁹, halogeno, NO₂, CN, OH, O-alkyl, COR⁹, COOR⁹, O-aryl, O—R⁹, NH₂, NH-alkyl, NH-aryl, NH(aralkyl), N-(alkyl)₂, N-(aryl)₂, N-(alkyl)(aryl), NH—R⁹, N—(R⁹)(R¹⁰), N-(alkyl)(R⁹), N-(aryl)(R⁹), COOH, CONH₂, CONH-alkyl, CONH-aryl, CON-(alkyl)(R⁹), CON(aryl)(R⁹), CONH—R⁹, CON—(R⁹)(R¹⁰), SO₃H, SO₂-alkyl, SO₂-alkyl-R⁹, SO₂-aryl, SO₂-aryl-R⁹, SO₂NH₂, SO₂NH—R⁹, SO₂N—(R⁹)(R¹⁰), CF₃, CO-alkyl, CO-alkyl-R⁹, CO-aryl, CO-aryl-R⁹ or R¹¹, wherein alkyl, aryl, aralkyl groups may be further substituted with one or more groups selected from halogeno, NO₂, OH, O-methyl, NH₂, COOH, CONH₂ and CF₃;
or two of R⁴-R⁸ are linked to form a cyclic ether containing one or more oxygens;
R⁹ and R¹⁰ are each independently solubilising groups selected from:
(i)
a mono-, di- or polyhydroxylated alicyclic group;
a di- or polyhydroxylated aliphatic or aromatic group;
a carbohydrate derivative;
an O- and/or S-containing heterocyclic group optionally substituted by one or more hydroxyl groups;
an aliphatic or aromatic group containing a carboxamide, sulfoxide, sulfone, or sulfonamide function; or
a halogenated alkylcarbonyl group;
(ii) COOH, SO₃H, OSO₃H, PO₃H₂, or OPO₃H₂;
(iii) Y, where Y is selected from an alicyclic, aromatic, or heterocyclic group comprising one or more of the functions ═N—, —O—, —N—, —NH₂, —NH—, a quarternary amine salt, guanidine, and amidine, where Y is optionally substituted by one or more substituents selected from:
halogen:
SO₂-alkyl;
alkyl optionally substituted by one or more OH or halogen groups;
CO-alkyl;
aralkyl;
COO-alkyl; and
an ether group optionally substituted by one or more OH groups;
(iv) a natural or unnatural amino acid, a peptide or a peptide derivative;
each R¹¹ is a solubilising group as defined for R⁹ and R¹⁰ in (i) or (iv) above; or is selected from:
(v) OSO₃H, PO₃H₂, or OPO₃H₂;
(vi) Y as defined above, but excluding guanidine and quarternary amine salts;
(vii) NHCO(CH₂)ₘ[NHCO(CH₂)_{m′}]ₚ[NHCO(CH₂)_{m″}]_{q}Y or NHCO(CH₂)ₜNH(CH₂)_{t′}Y where p and q are each 0 or 1, and m, m′, m″, t and t′ are each independently an integer from 1 to 10; and
(viii) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are optionally substituted by one or more substituents selected from OH, NH₂, halogen and NO₂, R¹⁴ and R¹⁵ are each independently H or alkyl, and n and n′ are each independently 0, 1, 2, or 3;
(ix) an ether or polyether optionally substituted by one or more hydroxyl groups or one or more Y groups;
(x) (CH₂)ᵣNH₂; where r is 0, 1, 2, or 3;
(xi) (CH₂)ᵣ—OH; where r′ is 0, 1, 2, or 3;
(xii) (CH₂)_{n″}NR¹⁷COR¹⁸ where R¹⁷ is H or alkyl, n″ is 0, 1, 2 or 3 and R¹⁸ is an aryl or heteroaryl group, each of which may be optionally substituted by one or more substituents selected from halogeno, NO₂, OH, alkoxy, NH₂, COOH, CONH₂ and CF₃;
(xiii) SO₂NR¹⁹R²⁰ where R¹⁹ and R²⁰ are each independently H, alkyl, aralkyl, CO-alkyl or aryl, with the proviso that at least one of R¹⁹ and R²⁰ is other than H, or R¹⁹ and R²⁰ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CH₂CO₂-alkyl, CONH₂ and CF₃;
(xiv) N-piperidinyl, N-pyrrolidinyl or N-thiomorpholinyl, each of which may be optionally substituted by one or more alkyl, alkoxy or CO-alkyl groups;
with the proviso that when Z is —NH— at least one of R⁴-R⁸ is selected from: (CH₂)_{n″}NR¹⁷COR¹⁸;
SO₂NR¹⁹R²⁰; and
N-piperidinyl, N-pyrrolidinyl and N-thiomorpholinyl, each of which may be optionally substituted by one or more alkyl, alkoxy or CO-alkyl groups;
or two of R⁴-R⁸ are linked to form a cyclic ether containing one or more oxygens.

2. A compound according to claim 1 wherein Z is —NH— and at least one of R⁴-R⁸ is selected from (CH₂)_{n″}NR¹⁷COR¹⁸ and SO₂NR¹⁹R²⁰.

3. A compound according to claim 1 wherein Z is —NH— and at least one of R⁴-R⁸ is N-piperidinyl, N-pyrrolidinyl or N-thiomorpholinyl, each of which may be optionally substituted by one or more alkyl, alkoxy or CO-alkyl groups.

4. A compound according to claim 1 wherein at least one of R⁴-R⁸ is selected from

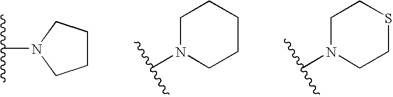

5. A compound according to claim 1 wherein Z is —NH—, one of R⁶ and R⁷ is selected from: (CH₂)_{n″}NR¹⁷COR¹⁸; SO₂NR¹⁹R²⁰; N-piperidinyl, N-pyrrolidinyl or N-thiomorpholinyl, each of which may be optionally substituted by one or more alkyl, alkoxy or CO-alkyl groups; and the other of R⁶ and R⁷ is H, alkyl or alkoxy.

6. A compound according to claim 1 wherein Z is —NH— and two of R⁴-R⁸ are linked to form a cyclic ether containing one or more oxygens.

7. A compound according to claim 1 wherein R⁶ and R⁷ are linked to form a cyclic ether containing one or more oxygens.

8. A compound according to claim 1 wherein R⁶ and R⁷ are linked to form a cyclic ether as shown below

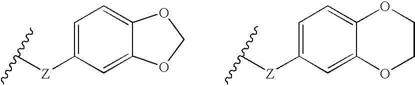

9. A compound according to claim 1 wherein Z is NHCOCH₂.

10. A compound according to claim 1 wherein at least one of R⁶ and R⁷ is (CH₂)_{n″}NR¹⁷COR¹⁸ or SO₂NR¹⁹R²⁰.

11. A compound according to claim 1 wherein at least one of R⁴-R⁸ is (CH₂)_{n″}NR¹⁷COR¹⁸.

12. A compound according to claim 11 where n″ is 1, R¹⁷ is H and R¹⁸ is phenyl or pyridinyl.

13. A compound according to claim 1 wherein at least one of R⁴-R⁸ is SO₂NR¹⁹R²⁰.

14. A compound according to claim 13 wherein
(i) one of R¹⁹ and R²⁰ is H and the other is an alkyl, aralkyl, aryl or heteroaryl group, each of which is optionally substituted by one or more alkoxy, alkyl, OH or CH₂CO₂-alkyl groups;
(ii) R¹⁹ and R²⁰ are each independently alkyl; or
(i) R¹⁹ and R²⁰ together with the nitrogen to which they are attached are linked to form a morpholine group.

15. A compound according to claim 1 wherein at least one of R⁴-R⁸ is selected from 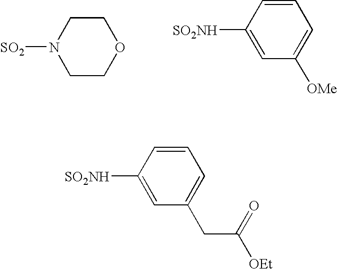
CH₂NHCOPh, CH₂NHCO-pyridinyl, SO₂NHCOMe, SO₂NHCH₂Ph, SO₂NHC(Me)₂CH₂OH, SO₂NHMe, SO₂NHⁱPr, SO₂NHEt, SO₂NEt₂, SO₂NHCH₂CH₂OH and SO₂NHCH₂CH₂OMe.

16. A compound according to claim 1 wherein R⁴, R⁵ and R⁸ are all H, one of R⁶ and R⁷ is selected from the following: 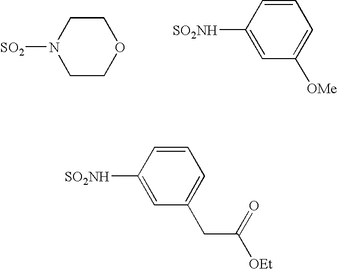
CH₂NHCOPh, CH₂NHCO-pyridinyl, SO₂NHCOMe, SO₂NHCH₂Ph, SO₂NHC(Me)₂CH₂OH, SO₂NHMe, SO₂NHⁱPr, SO₂NHEt, SO₂NEt₂, SO₂NHCH₂CH₂OH and SO₂NHCH₂CH₂OMe; and the other of R⁶ and R⁷ is H, alkyl or alkoxy.

17. A compound according to claim 1 wherein R² is selected from aryl, aryl-R⁹, NH₂, NH(alkyl), alkyl, N(alkyl)₂, N(alkyl)CO-alkyl, N(alkyl)(aryl), NH(aryl), CH₂OH, wherein said alkyl and aryl groups are optionally substituted by one or more alkoxy, halo, R¹¹ or CF₃ groups.

18. A compound according to claim 1 wherein R² is selected from NH₂, NHMe, N(Me(Et), NHEt, NH^{t}Bu, Me, NHCH₂CH₂OMe, NMe₂, CH₂OH, NHPh,

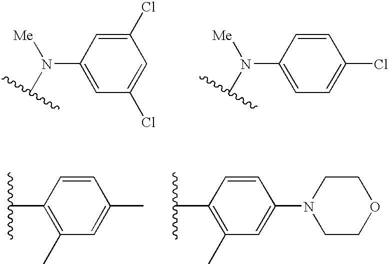

19. A compound of formula II, or a pharmaceutically acceptable salt thereof, 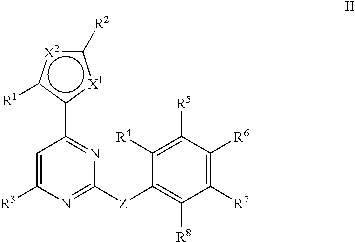
wherein:
one of X¹ and X² is S, and the other of X¹ and X² is N;
Z is NH, NHCO, NHCOCH₂, NHSO₂, NHCH₂, CH₂, CH₂CH₂, CH═CH, O, S, SO₂, or SO;
R¹, R³, R⁴, R⁵, R⁶, R⁷ and R⁸ are each independently H, alkyl, alkyl-R⁹, aryl, aryl-R⁹, aralkyl, aralkyl-R⁹, halogeno, NO₂, CN, OH, O-alkyl, COR⁹, COOR⁹, O-aryl, O—R⁹, NH₂₂, NH-alkyl, NH-aryl, NH(aralkyl), N-(alkyl)₂, N-(aryl)₂, N-(alkyl)(aryl), NH—R⁹, N—(R⁹)(R¹⁰), N-(alkyl)(R⁹), N-(aryl)(R⁹), COOH, CONH₂, CONH-alkyl, CONH-aryl, CON-(alkyl)(R⁹), CON(aryl)(R⁹), CONH—R⁹, CON—(R⁹)(R¹⁰), SO₃H, SO₂-alkyl, SO₂-alkyl-R⁹, SO₂-aryl, SO₂-aryl-R⁹, SO₂NH₂, SO₂NH—R⁹, SO₂N—(R⁹)(R¹⁰), CF₃, CO-alkyl, CO-alkyl-R⁹, CO-aryl, CO-aryl-R⁹ or R¹¹, wherein alkyl, aryl, aralkyl groups may be further substituted with one or more groups selected from halogeno, NO₂, OH, O-methyl, NH₂, COOH, CONH₂ and CF₃;
R² is selected from pyridinyl, N(alkyl)pyridinyl, NH(aralkyl) and N(alkyl)(aralkyl), wherein said pyridinyl, alkyl or aralkyl groups may be optionally substituted by one or more alkyl, CF₃ or ether groups;
R⁹ and R¹⁰ are each independently solubilising groups selected from:
(i)
a mono-, di- or polyhydroxylated alicyclic group;
a di- or polyhydroxylated aliphatic or aromatic group;
a carbohydrate derivative;
an O- and/or S-containing heterocyclic group optionally substituted by one or more hydroxyl groups;
an aliphatic or aromatic group containing a carboxamide, sulfoxide, sulfone, or sulfonamide function; or
a halogenated alkylcarbonyl group;
(ii) COOH, SO₃H, OSO₃H, PO₃H₂, or OPO₃H₂;
(iii) Y, where Y is selected from an alicyclic, aromatic, or heterocyclic group comprising one or more of the functions ═N—, —O—, —NH₂, —N—, —NH—, a quarternary amine salt, guanidine, and amidine, where Y is optionally substituted by one or more substituents selected from:
halogen:
SO₂-alkyl;
alkyl optionally substituted by one or more OH or halogen groups;
CO-alkyl;
aralkyl;
COO-alkyl; and
an ether group optionally substituted by one or more OH groups;
(iv) a natural or unnatural amino acid, a peptide or a peptide derivative;
each R¹¹ is a solubilising group as defined for R⁹ and R¹⁰ in (i) or (iv) above; or is selected from:
(v) OSO₃H, PO₃H₂, or OPO₃H₂;
(vi) Y as defined above, but excluding guanidine and quarternary amine salts;
(vii) NHCO(CH₂)ₘ[NHCO(CH₂)_{m′}]ₚ[NHCO(CH₂)_{m″}]_{q}Y or NHCO(CH₂)ₜNH(CH₂)_{t′}Y where p and q are each 0 or 1, and m, m′, m″, t and t′ are each independently an integer from 1 to 10; and
(viii) (CH₂)ₙNR¹⁴COR¹², (CH₂)_{n′}NR¹⁵SO₂R¹³, or SO₂R¹⁶, where R¹², R¹³ and R¹⁶ are each alkyl groups optionally comprising one or more heteroatoms, and which are optionally substituted by one or more substituents selected from OH, NH₂, halogen and NO₂, R¹⁴ and R¹⁵ are each independently H or alkyl, and n and n′ are each independently 0, 1, 2, or 3;
(ix) an ether or polyether optionally substituted by one or more hydroxyl groups or one or more Y groups;
(x) (CH₂)ᵣNH₂; where r is 0, 1, 2, or 3;
(xi) (CH₂)_{r′}OH; where r′ is 0, 1, 2, or 3;
(xii) (CH₂)_{n″}NR¹⁷COR¹⁸ where R¹⁷ is H or alkyl, n″ is 0, 1, 2 or 3 and R¹⁸ is an aryl or heteroaryl group, each of which may be optionally substituted by one or more substituents selected from halogeno, NO₂, OH, alkoxy, NH₂, COOH, CONH₂ and CF₃;
(xiii) SO₂NR¹⁹R²⁰ where R¹⁹ and R²⁰ are each independently H, alkyl, aralkyl, CO-alkyl or aryl, with the proviso that at least one of R¹⁹ and R²⁰ is other than H, or R¹⁹ and R²⁰ are linked to form a cyclic group optionally containing one or more heteroatoms selected from N, O and S, and wherein said alkyl, aryl or cyclic group is optionally substituted by one or more substituents selected from halogeno, NO₂, OH, alkoxy, NH₂, COOH, CH₂CO₂-alkyl, CONH₂ and CF₃;
(xiv) N-piperidinyl, N-pyrrolidinyl or N-thiomorpholinyl, each of which may be optionally substituted by one or more alkyl, alkoxy or CO-alkyl groups;
wherein at least one of R⁶ and R⁷ is a (CH₂)ₙNR¹⁴COR¹² group or an alicyclic group containing at least one —N— wherein said alicyclic group is optionally substituted by one or more alkyl, alkoxy, CO-alkyl or aralkyl groups.

20. A compound according to claim 19 wherein R² is selected from pyridinyl, N(methyl)pyridinyl, NH(aralkyl) and N(methyl)(aralkyl), wherein said pyridinyl or aralkyl groups may be optionally substituted by one or more alkyl, CF₃ or ether groups.

21. A compound according to claim 20 wherein R² is selected from N(Me)CH₂Ph, NHCH₂CH₂Ph, NHCH₂Ph,

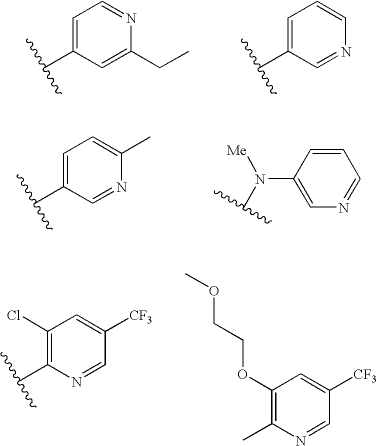

22. A compound according to claim 19 wherein R⁶ is an alicyclic group selected from

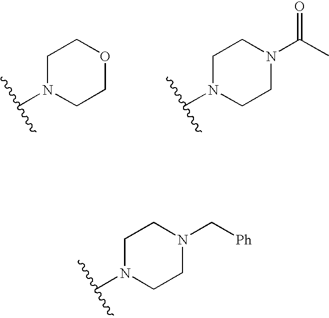

23. A compound according to claim 19 wherein R⁶ or R⁷ is CH₂NHCOMe.

24. A compound according to claim 1 or claim 19 wherein X¹ is S and X² is N.

25. A compound according to claim 1 or claim 19 wherein R³ is H and R¹ is Me.

26. A compound according to claim 1 or claim 19 wherein Z is NH.

27. A compound according to claim 1 or claim 19 wherein Z is NHCOCH₂.

28. A compound according to claim 1 which is selected from compounds [9], [21], [22], [26], [29], [30]-[33], [36]-[41], [43], [52]-[56], [62]-[78], [80]-[82], [84], [91]-[98], [102], [110], [177]-[181], [183], [193]-[195], [197]-[199], [201]-[209] and [216].

29. A compound according to claim 19 which is selected from compounds [99], [100], [101], [103], [104]-[109], [117]-[119], [122], [126], [127], [153], [156], [158] and [162]-[165].

30. A compound selected from the following:
{3-[4-(2-Amino-4-methyl-thiazol-5-yl)-pyrimidin-2-ylamino]-phenyl}-acetic acid 2-methoxy-ethyl ester [1];
[4-(2-tert-Butylamino-4-methyl-thiazol-5-yl)-pyrimidin-2-yl]-(4-methyl-3-nitro-phenyl)-amine [2];
1-(4-{3-[4-(4-Methyl-2-methylamino-thiazol-5-yl)-pyrimidin-2-ylamino]-phenyl}-piperazin-1-yl)-ethanone [3];
[4-(2,4-Dimethyl-thiazol-5-yl)-pyrimidin-2-yl]-(3-methanesulfonyl-phenyl)-amine [4];
N-{3-[4-(2-Ethylamino-4-methyl-thiazol-5-yl)-pyrimidin-2-ylamino]-benzyl}-methanesulfonamide [5];
N-{3-[4-(2-Amino-4-methyl-thiazol-5-yl)-pyrimidin-2-ylamino]-benzyl}-methanesulfonamide [6];
[4-(4-Methyl-2-methylamino-thiazol-5-yl)-pyrimidin-2-yl]-(3-piperazin-1-yl-phenyl)-amine [7];
[4-(2,4-Dimethyl-thiazol-5-yl)-pyrimidin-2-yl]-(3-piperazin-1-yl-phenyl)-amine [8];
N-{3-[4-(2,4-Dimethyl-thiazol-5-yl)-pyrimidin-2-ylamino]-benzyl}-benzamide [9];
N-{3-[4-(2-Ethylamino-4-methyl-thiazol-5-yl)-pyrimidin-2-ylamino]-benzyl}-C,C,C-trifluoro-methanesulfonamide [10];
N-{3-[4-(2,4-Dimethyl-thiazol-5-yl)-pyrimidin-2-ylamino]-benzyl}-C,C,C-trifluoro-methanesulfonamide [11];
N-{3-[4-(2-Amino-4-methyl-thiazol-5-yl)-pyrimidin-2-ylamino]-benzyl}-C,C,C-trifluoro-methanesulfonamide [12];
N-{4-[4-(2-Ethylamino-4-methyl-thiazol-5-yl)-pyrimidin-2-ylamino]-benzyl}-acetamide [13];
N-{4-[4-(4-Methyl-2-methylamino-thiazol-5-yl)-pyrimidin-2-ylamino]-benzyl}-acetamide [14];
N-{4-[4-(2,4-Dimethyl-thiazol-5-yl)-pyrimidin-2-ylamino]-benzyl}-acetamide [15];
N-{4-[4-(2-Amino-4-methyl-thiazol-5-yl)-pyrimidin-2-ylamino]-benzyl}-acetamide [16];
[4-(2-Ethylamino-4-methyl-thiazol-5-yl)-pyrimidin-2-yl]-(4-methanesulfonyl-phenyl)-amine [17];
3-[4-(2-Ethylamino-4-methyl-thiazol-5-yl)-pyrimidin-2-ylamino]-benzenesulfonamide [18];
3-[4-(4-Methyl-2-methylamino-thiazol-5-yl)-pyrimidin-2-ylamino]-benzenesulfonamide [19];
(4-Methanesulfonyl-phenyl)-[4-(4-methyl-2-methylamino-thiazol-5-yl)-pyrimidin-2-yl]-amine [20];
N-Methyl-3-[4-(4-methyl-2-methylamino-thiazol-5-yl)-pyrimidin-2-ylamino]-benzenesulfonamide [21];
3-[4-(2-Ethylamino-4-methyl-thiazol-5-yl)-pyrimidin-2-ylamino]-N-methyl-benzenesulfonamide [22];
[4-(4-Methyl-2-methylamino-thiazol-5-yl)-pyrimidin-2-yl]-(3,4,5-trimethoxy-phenyl)-amine [23];
[4-(2-Ethylamino-4-methyl-thiazol-5-yl)-pyrimidin-2-yl]-(3,4,5-trimethoxy-phenyl)-amine [24];
[4-(2,4-Dimethyl-thiazol-5-yl)-pyrimidin-2-yl]-(3,4,5-trimethoxy-phenyl)-amine [25];
3-[4-(2-Amino-4-methyl-thiazol-5-yl)-pyrimidin-2-ylamino]-N-methyl-benzenesulfonamide [26];
(3-Methanesulfonyl-phenyl)-[4-(4-methyl-2-methylamino-thiazol-5-yl)-pyrimidin-2-yl]-amine [27];
[4-(2-Ethylamino-4-methyl-thiazol-5-yl)-pyrimidin-2-yl]-(3-methanesulfonyl-phenyl)-amine [28];
N-Ethyl-3-[4-(2-ethylamino-4-methyl-thiazol-5-yl)-pyrimidin-2-ylamino]-benzenesulfonamide [29];
3-[4-(2-Amino-4-methyl-thiazol-5-yl)-pyrimidin-2-ylamino]-N-ethyl-benzenesulfonamide [30];
N-Ethyl-3-[4-(4-methyl-2-methylamino-thiazol-5-yl)-pyrimidin-2-ylamino]-benzenesulfonamide [31];
N-(3-Methoxy-phenyl)-3-[4-(4-methyl-2-methylamino-thiazol-5-yl)-pyrimidin-2-ylamino]-benzenesulfonamide [32];
3-[4-(2,4-Dimethyl-thiazol-5-yl)-pyrimidin-2-ylamino]-N-methyl-benzenesulfonamide [33];
4-[4-(4-Methyl-2-methylamino-thiazol-5-yl)-pyrimidin-2-ylamino]-benzenesulfonamide [34];
4-[4-(2-Ethylamino-4-methyl-thiazol-5-yl)-pyrimidin-2-ylamino]-benzenesulfonamide [35];
[4-(2-Ethylamino-4-methyl-thiazol-5-yl)-pyrimidin-2-yl]-[4-methyl-3-(morpholine-4-sulfonyl)-phenyl]-amine [36];
[4-(4-Methyl-2-methylamino-thiazol-5-yl)-pyrimidin-2-yl]-[4-methyl-3-(morpholine-4-sulfonyl)-phenyl]-amine [37];
[4-(2-Amino-4-methyl-thiazol-5-yl)-pyrimidin-2-yl]-[4-methyl-3-(morpholine-4-sulfonyl)-phenyl]-amine [38];
4-[4-(2-Ethylamino-4-methyl-thiazol-5-yl)-pyrimidin-2-ylamino]-N-(2-methoxy-ethyl)-benzenesulfonamide [39];
N-(2-Methoxy-ethyl)-4-[4-(4-methyl-2-methylamino-thiazol-5-yl)-pyrimidin-2-ylamino]-benzenesulfonamide [40];
4-[4-(2-Amino-4-methyl-thiazol-5-yl)-pyrimidin-2-ylamino]-N-(2-methoxy-ethyl)-benzenesulfonamide [41];
(3-Bromo-4-methyl-phenyl)-[4-(4-methyl-2-methylamino-thiazol-5-yl)-pyrimidin-2-yl]-amine [42];
4-[4-(2,4-Dimethyl-thiazol-5-yl)-pyrimidin-2-ylamino]-N-(2-methoxy-ethyl)-benzenesulfonamide [43];
{3-[4-(4-Methyl-2-methylamino-thiazol-5-yl)-pyrimidin-2-ylamino]-phenyl}-acetic acid 2-methoxy-ethyl ester [44];
{3-[4-(2-Ethylamino-4-methyl-thiazol-5-yl)-pyrimidin-2-ylamino]-phenyl}-acetic acid 2-methoxy-ethyl ester [45];
1-(4-{3-[4-(2,4-Dimethyl-thiazol-5-yl)-pyrimidin-2-ylamino]-phenyl}-piperazin-1-yl)-ethanone [46];
{3-[4-(2,4-Dimethyl-thiazol-5-yl)-pyrimidin-2-ylamino]-5-hydroxymethyl-phenyl}-methanol [47];
{3-Hydroxymethyl-5-[4-(4-methyl-2-methylamino-thiazol-5-yl)-pyrimidin-2-ylamino]-phenyl}-methanol [48];
N-{3-[4-(2,4-Dimethyl-thiazol-5-yl)-pyrimidin-2-ylamino]-benzyl}-methanesulfonamide [49];
(3-Bromo-phenyl)-[4-(2-ethylamino-4-methyl-thiazol-5-yl)-pyrimidin-2-yl]-amine [50];
[4-(2-tert-Butylamino-4-methyl-thiazol-5-yl)-pyrimidin-2-yl]-(3-nitro-phenyl)-amine [51];
N,N-Diethyl-4-[4-(4-methyl-2-methylamino-thiazol-5-yl)-pyrimidin-2-ylamino]-benzenesulfonamide [52];
3-[4-(2-Ethylamino-4-methyl-thiazol-5-yl)-pyrimidin-2-ylamino]-N-(1-methoxy-ethyl)-benzenesulfonamide [53];
N-(2-Methoxy-ethyl)-3-[4-(4-methyl-2-methylamino-thiazol-5-yl)-pyrimidin-2-ylamino]-benzenesulfonamide [54];
3-[4-(2-Amino-4-methyl-thiazol-5-yl)-pyrimidin-2-ylamino]-N-(2-methoxy-ethyl)-benzenesulfonamide [55];
3-[4-(2,4-Dimethyl-thiazol-5-yl)-pyrimidin-2-ylamino]-N-(2-methoxy-ethyl)-benzenesulfonamide [56];
1-(4-{4-[4-(2-Amino-4-methyl-thiazol-5-yl)-pyrimidin-2-ylamino]-phenyl}-piperazin-1-yl)-ethanone [57];
1-(4-{4-[4-(2-Ethylamino-4-methyl-thiazol-5-yl)-pyrimidin-2-ylamino]-phenyl}-piperazin-1-yl)-ethanone [58];
[4-(2-Ethylamino-4-methyl-thiazol-5-yl)-pyrimidin-2-yl]-(4-piperazin-1-yl-phenyl)-amine [59];
[4-(4-Benzyl-piperazin-1-yl)-phenyl]-[4-(2-ethylamino-4-methyl-thiazol-5-yl)-pyrimidin-2-yl]-amine [60];
[4-(2-Amino-4-methyl-thiazol-5-yl)-pyrimidin-2-yl]-(4-piperazin-1-yl-phenyl)-amine [61];
(3-{4-[4-(4-Methyl-2-methylamino-thiazol-5-yl)-pyrimidin-2-ylamino]-benzenesulfonylamino}-phenyl)-acetic acid ethyl ester [62];
N-Acetyl-3-[4-(4-methyl-2-methylamino-thiazol-5-yl)-pyrimidin-2-ylamino]-benzenesulfonamide [63];
N-Acetyl-3-[4-(2-amino-4-methyl-thiazol-5-yl)-pyrimidin-2-ylamino]-benzenesulfonamide [64];
4-[4-(2-Ethylamino-4-methyl-thiazol-5-yl)-pyrimidin-2-ylamino]-N-(2-hydroxy-ethyl)-benzenesulfonamide [65];
4-[4-(2,4-Dimethyl-thiazol-5-yl)-pyrimidin-2-ylamino]-N-ethyl-benzenesulfonamide [66];
N-(2-Hydroxy-ethyl)-4-[4-(4-methyl-2-methylamino-thiazol-5-yl)-pyrimidin-2-ylamino]-benzenesulfonamide [67];
4-[4-(2-Amino-4-methyl-thiazol-5-yl)-pyrimidin-2-ylamino]-N-(2-hydroxy-ethyl)-benzenesulfonamide [68];
4-[4-(2,4-Dimethyl-thiazol-5-yl)-pyrimidin-2-ylamino]-N-(2-hydroxy-ethyl)-benzenesulfonamide [69];
3-[4-(2-Ethylamino-4-methyl-thiazol-5-yl)-pyrimidin-2-ylamino]-N-isopropyl-benzenesulfonamide [70];
N-Benzyl-4-[4-(2-ethylamino-4-methyl-thiazol-5-yl)-pyrimidin-2-ylamino]-benzenesulfonamide [71]
N-Benzyl-4-[4-(4-methyl-2-methylamino-thiazol-5-yl)-pyrimidin-2-ylamino]-benzenesulfonamide [72];
4-[4-(2-Amino-4-methyl-thiazol-5-yl)-pyrimidin-2-ylamino]-N-benzyl-benzenesulfonamide [73];
N-Benzyl-4-[4-(2,4-dimethyl-thiazol-5-yl)-pyrimidin-2-ylamino]-benzenesulfonamide [74];
3-[4-(2-Ethylamino-4-methyl-thiazol-5-yl)-pyrimidin-2-ylamino]-N-(2-hydroxy-ethyl)-benzenesulfonamide [75];
N-(2-Hydroxy-ethyl)-3-[4-(4-methyl-2-methylamino-thiazol-5-yl)-pyrimidin-2-ylamino]-benzenesulfonamide [76];
3-[4-(2-Amino-4-methyl-thiazol-5-yl)-pyrimidin-2-ylamino]-N-(2-hydroxy-ethyl)-benzenesulfonamide [77];
3-[4-(2,4-Dimethyl-thiazol-5-yl)-pyrimidin-2-ylamino]-N-(2-hydroxy-ethyl)-benzenesulfonamide [78];
[4-(2-Ethylamino-4-methyl-thiazol-5-yl)-pyrimidin-2-yl]-pyridin-3-ylmethyl-amine [79];
N-Benzyl-3-[4-(2-ethylamino-4-methyl-thiazol-5-yl)-pyrimidin-2-ylamino]-benzenesulfonamide [80]
[4-(2-Amino-4-methyl-thiazol-5-yl)-pyrimidin-2-yl]-[3-(morpholine-4-sulfonyl)-phenyl]-amine [81];
[4-(2,4-Dimethyl-thiazol-5-yl)-pyrimidin-2-yl]-[4-methyl-3-(morpholine-4-sulfonyl)-phenyl]-amine [82];
3-{4-[2-(2-Methoxy-ethylamino)-4-methyl-thiazol-5-yl]-pyrimidin-2-ylamino}-benzenesulfonamide [83];
3-[4-(2-Ethylamino-4-methyl-thiazol-5-yl)-pyrimidin-2-ylamino]-N-(2-hydroxy-1,1-dimethyl-ethyl)-benzenesulfonamide [84];
4-(4-Methyl-2-methylamino-thiazol-5-yl)-pyrimidin-2-ylamine [85];
4-(2-Ethylamino-4-methyl-thiazol-5-yl)-pyrimidin-2-ylamine [86];
N-[5-(2-Amino-pyrimidin-4-yl)-4-methyl-thiazol-2-yl]-N-ethyl-acetamide [87];
4-(2-Dimethylamino-4-methyl-thiazol-5-yl)-pyrimidin-2-ylamine [88];
4-Chloromethyl-N-[4-(2-dimethylamino-4-methyl-thiazol-5-yl)-pyrimidin-2-yl]-benzamide [89];
(3-Aminomethyl-phenyl)-[4-(2,4-dimethyl-thiazol-5-yl)-pyrimidin-2-yl]-amine [90];
Pyridine-2-carboxylic acid 3-[4-(2,4-dimethyl-thiazol-5-yl)-pyrimidin-2-ylamino]-benzylamide [91];
2-(4-Chloro-phenyl)-N-[4-(2-dimethylamino-4-methyl-thiazol-5-yl)-pyrimidin-2-yl]-acetamide [92];
N-[4-(2-Dimethylamino-4-methyl-thiazol-5-yl)-pyrimidin-2-yl]-2-(4-nitro-phenyl)-acetamide [93];
N-[4-(2-Dimethylamino-4-methyl-thiazol-5-yl)-pyrimidin-2-yl]-2-(4-methoxy-phenyl)-acetamide [94];
N-[4-(2-Ethylamino-4-methyl-thiazol-5-yl)-pyrimidin-2-yl]-2-(4-methoxy-phenyl)-acetamide [95];
N-[4-(2,4-Dimethyl-thiazol-5-yl)-pyrimidin-2-yl]-2-(4-methoxy-phenyl)-acetamide [96];
2-(4-Chloro-phenyl)-N-[4-(2,4-dimethyl-thiazol-5-yl)-pyrimidin-2-yl]-acetamide [97];
N-[4-(2,4-Dimethyl-thiazol-5-yl)-pyrimidin-2-yl]-2-(4-nitro-phenyl)-acetamide [98];
{4-[2-(2-Ethyl-pyridin-4-yl)-4-methyl-thiazol-5-yl]-pyrimidin-2-yl}-(4-morpholin-4-yl-phenyl)-amine [99];
[4-(4-Methyl-2-pyridin-3-yl-thiazol-5-yl)-pyrimidin-2-yl]-(4-morpholin-4-yl-phenyl)-amine [100];
N-{3-[4-(4-Methyl-2-pyridin-3-yl-thiazol-5-yl)-pyrimidin-2-ylamino]-benzyl}-acetamide [101];
4-{4-[2-(2-Ethyl-pyridin-4-yl)-4-methyl-thiazol-5-yl]-pyrimidin-2-ylamino}-N-(2-hydroxy-ethyl)-benzenesulfonamide [102];
N-{4-[4-(4-Methyl-2-pyridin-3-yl-thiazol-5-yl)-pyrimidin-2-ylamino]-benzyl}-acetamide [103];
N-(4-{4-[2-(2-Ethyl-pyridin-4-yl)-4-methyl-thiazol-5-yl]-pyrimidin-2-ylamino}-benzyl)-acetamide [104];
N-(3-{4-[2-(2-Ethyl-pyridin-4-yl)-4-methyl-thiazol-5-yl]-pyrimidin-2-ylamino}-benzyl)-acetamide [105];
{4-[4-Methyl-2-(6-methyl-pyridin-3-yl)-thiazol-5-yl]-pyrimidin-2-yl}-(4-morpholin-4-yl-phenyl-amine [106];
(4-{2-[3-(2-Methoxy-ethoxy)-5-trifluoromethyl-pyridin-2-yl]-4-methyl-thiazol-5-yl}-pyrimidin-2-yl)-(4-morpholin-4-yl-phenyl)-amine [107];
N-(3-{4-[4-Methyl-2-(6-methyl-pyridin-3-yl)-thiazol-5-yl]-pyrimidin-2-ylamino}-benzyl)-acetamide [108];
N-(3-{4-[2-(3-Chloro-5-trifluoromethyl-pyridin-2-yl)-4-methyl-thiazol-5-yl]-pyrimidin-2-ylamino}-benzyl)-acetamide [109];
N-(2-Methoxy-ethyl)-4-[4-(4-methyl-2-pyridin-3-yl-thiazol-5-yl)-pyrimidin-2-ylamino]-benzenesulfonamide [110];
[4-(2-Ethylamino-4-methyl-thiazol-5-yl)-pyrimidin-2-yl]-(4-methoxy-2-methyl-phenyl)-amine [111];
[4-(2,4-Dimethyl-thiazol-5-yl)-pyrimidin-2-yl]-(4-methoxy-2-methyl-phenyl)-amine [112];
[4-(2,4-Dimethyl-thiazol-5-yl)-pyrimidin-2-yl]-(5-methoxy-2-methyl-phenyl)-amine [113];
[4-(4-Benzyl-piperazin-1-yl)-phenyl]-[4-(2,4-dimethyl-thiazol-5-yl)-pyrimidin-2-yl]-amine [114];
[4-(2-Ethylamino-4-methyl-thiazol-5-yl)-pyrimidin-2-yl]-(5-methoxy-2-methyl-phenyl)-amine [115];
(3-Aminomethyl-phenyl)-[4-(4-methyl-2-pyridin-3-yl-thiazol-5-yl)-pyrimidin-2-yl]-amine [116];
[4-(2-Benzylamino-4-methyl-thiazol-5-yl)-pyrimidin-2-yl]-(4-morpholin-4-yl-phenyl)-amine [117];
N-{3-[4-(2-Benzylamino-4-methyl-thiazol-5-yl)-pyrimidin-2-ylamino]-benzyl}-acetamide [118];
1-(4-{4-[4-(4-Methyl-2-pyridin-3-yl-thiazol-5-yl)-pyrimidin-2-ylamino]-phenyl}-piperazin-1-yl)-ethanone [119];
{4-[2-(Ethyl-methyl-amino)-4-methyl-thiazol-5-yl]-pyrimidin-2-yl}-(4-morpholin-4-yl-phenyl)-amine [120];
[4-(2,6-Dimethyl-morpholin-4-yl)-phenyl]-[4-(2,4-dimethyl-thiazol-5-yl)-pyrimidin-2-yl]-amine [121];
1-[4-(4-{4-[2-(Benzyl-methyl-amino)-4-methyl-thiazol-5-yl]-pyrimidin-2-ylamino}-phenyl)-piperazin-1-yl]-ethanone [122];
(4-{2-[(3,5-Dichloro-phenyl)-methyl-amino]-4-methyl-thiazol-5-yl}-pyrimidin-2-yl)-(4-morpholin-4-yl-phenyl)-amine [123];
(4-{2-[(4-Chloro-phenyl)-methyl-amino]-4-methyl-thiazol-5-yl}-pyrimidin-2-yl)-(4-morpholin-4-yl-phenyl)-amine [124];
N-[3-(4-{2-[(3,5-Dichloro-phenyl)-methyl-amino]-4-methyl-thiazol-5-yl}-pyrimidin-2-ylamino)-benzyl]-acetamide [125];
(3,5-Dichloro-4-morpholin-4-yl-phenyl)-[4-(4-methyl-2-pyridin-3-yl-thiazol-5-yl)-pyrimidin-2-yl]-amine [126];
(3-Chloro-4-morpholin-4-yl-phenyl)-[4-(4-methyl-2-pyridin-3-yl-thiazol-5-yl)-pyrimidin-2-yl]-amine [127];
(3-Chloro-4-morpholin-4-yl-phenyl)-(4-{2-[(3,5-dichloro-phenyl)-methyl-amino]-4-methyl-thiazol-5-yl}-pyrimidin-2-yl)-amine [128];
[4-(4-Methyl-2-thiophen-2-yl-thiazol-5-yl)-pyrimidin-2-yl]-(4-morpholin-4-yl-phenyl)-amine [129];
N-{3-[4-(4-Methyl-2-thiophen-2-yl-thiazol-5-yl)-pyrimidin-2-ylamino]-benzyl}-acetamide [130];
1-(4-{4-[4-(4-Methyl-2-thiophen-2-yl-thiazol-5-yl)-pyrimidin-2-ylamino]-phenyl}-piperazin-1-yl)-ethanone [131];
{5-[2-(4-Dimethylamino-phenylamino)-pyrimidin-4-yl]-4-methyl-thiazol-2-yl}-methanol [132];
(3,5-Dichloro-4-morpholin-4-yl-phenyl)-[4-(2-ethylamino-4-methyl-thiazol-5-yl)-pyrimidin-2-yl]-amine [133];
(3-Chloro-4-morpholin-4-yl-phenyl)-[4-(2-ethylamino-4-methyl-thiazol-5-yl)-pyrimidin-2-yl]-amine [134];
[4-(4,2′-Dimethyl-[2,4′]bithiazolyl-5-yl)-pyrimidin-2-yl]-(4-morpholin-4-yl-phenyl)-amine [135];
(3-Chloro-4-morpholin-4-yl-phenyl)-[4-(4,2′-dimethyl-[2,4′]bithiazolyl-5-yl)-pyrimidin-2-yl]-amine [136];
(3,5-Dichloro-4-morpholin-4-yl-phenyl)-[4-(4,2′-dimethyl-[2,4′]bithiazolyl-5-yl)-pyrimidin-2-yl]-amine [137];
{4-[4-Methyl-2-(thiophene-2-sulfonylmethyl)-thiazol-5-yl]-pyrimidin-2-yl}-(4-morpholin-4-yl-phenyl)-amine [138];
[4-(2,4-Dimethyl-thiazol-5-yl)-pyrimidin-2-yl]-(2-methyl-4-morpholin-4-yl-phenyl)-amine [139];
{4-[2-(2,4-Dimethyl-phenyl)-4-methyl-thiazol-5-yl]-pyrimidin-2-yl}-(4-morpholin-4-yl-phenyl)-amine [140];
(3-Chloro-4-morpholin-4-yl-phenyl)-[4-(2,4-dimethyl-thiazol-5-yl)-pyrimidin-2-yl]-amine [141];
(3,5-Dichloro-4-morpholin-4-yl-phenyl)-[4-(2,4-dimethyl-thiazol-5-yl)-pyrimidin-2-yl]-amine [142];
[4-(2-tert-Butylamino-4-methyl-thiazol-5-yl)-pyrimidin-2-yl]-(4-morpholin-4-yl-phenyl)-amine [143]
{4-[2-(2-Methoxy-ethylamino)-4-methyl-thiazol-5-yl]-pyrimidin-2-yl}-(4-morpholin-4-yl-phenyl)-amine [144];
[4-(4-Methyl-2-methylamino-thiazol-5-yl)-pyrimidin-2-yl]-(2-methyl-4-morpholin-4-yl-phenyl)-amine [145];
[4-(2-Ethylamino-4-methyl-thiazol-5-yl)-pyrimidin-2-yl]-(2-methyl-4-morpholin-4-yl-phenyl)-amine [146];
{4-[4-Methyl-2-(4-morpholin-4-yl-phenyl)-thiazol-5-yl]-pyrimidin-2-yl}-(4-morpholin-4-yl-phenyl)-amine [147];
1-[4-(4-{4-[4-Methyl-2-(4-morpholin-4-yl-phenyl)-thiazol-5-yl]-pyrimidin-2-ylamino}-phenyl)-piperazin-1-yl]-ethanone [148];
N⁴-[4-(2,4-Dimethyl-thiazol-5-yl)-pyrimidin-2-yl]-N¹-methyl-2-trifluoromethyl-benzene-1,4-diamine [149];
[4-(2,4-Dimethyl-thiazol-5-yl)-pyrimidin-2-yl]-(3-morpholin-4-ylmethyl-phenyl)-amine [150];
4-[4-(2,4-Dimethyl-thiazol-5-yl)-pyrimidin-2-ylamino]-2-morpholin-4-ylmethyl-phenol [151];
[4-(2,4-Dimethyl-thiazol-5-yl)-pyrimidin-2-yl]-(3-morpholin-4-yl-phenyl)-amine [152];
{4-[4-Methyl-2-(methyl-pyridin-3-yl-amino)-thiazol-5-yl]-pyrimidin-2-yl}-(4-morpholin-4-yl-phenyl)-amine [153];
[4-(4-Methyl-2-pyridin-3-yl-thiazol-5-yl)-pyrimidin-2-yl]-(3,4,5-trimethoxy-phenyl)-amine [154];
(3,5-Dimethoxy-phenyl)-[4-(4-methyl-2-pyridin-3-yl-thiazol-5-yl)-pyrimidin-2-yl]-amine [155];
(3-Methoxy-4-morpholin-4-yl-phenyl)-[4-(4-methyl-2-pyridin-3-yl-thiazol-5-yl)-pyrimidin-2-yl]-amine [156];
[4-(2-Ethylamino-4-methyl-thiazol-5-yl)-pyrimidin-2-yl]-(3-methoxy-4-morpholin-4-yl-phenyl)-amine [157];
[4-(4-Methyl-2-phenethylamino-thiazol-5-yl)-pyrimidin-2-yl]-(4-morpholin-4-yl-phenyl)-amine [158];
(3,5-Dimethoxy-phenyl)-[4-(4-methyl-2-phenethylamino-thiazol-5-yl)-pyrimidin-2-yl]-amine [159];
(3,5-Dimethoxy-phenyl)-[4-(2-ethylamino-4-methyl-thiazol-5-yl)-pyrimidin-2-yl]-amine [160];
[4-(2-Amino-4-methyl-thiazol-5-yl)-pyrimidin-2-yl]-(3,5-dimethoxy-phenyl)-amine [161];
{4-[4-Methyl-2-(methyl-pyridin-3-yl-amino)-thiazol-5-yl]-pyrimidin-2-yl}-(4-morpholin-4-yl-phenyl)-amine [162];
1-(4-{4-[4-(4-Methyl-2-phenethylamino-thiazol-5-yl)-pyrimidin-2-ylamino]-phenyl}-piperazin-1-yl)-ethanone [163];
1-[4-(4-{4-[4-Methyl-2-(methyl-pyridin-3-yl-amino)-thiazol-5-yl]-pyrimidin-2-ylamino}-phenyl)-piperazin-1-yl]-ethanone [164];
[4-(4-Methyl-2-phenethylamino-thiazol-5-yl)-pyrimidin-2-yl]-(3,4,5-trimethoxy-phenyl)-amine [165]
[4-(4-Benzyl-piperazin-1-yl)-phenyl]-[4-(4-methyl-2-phenethylamino-thiazol-5-yl)-pyrimidin-2-yl]-amine [166];
[4-(4-Methyl-2-phenylamino-thiazol-5-yl)-pyrimidin-2-yl]-(4-morpholin-4-yl-phenyl)-amine [167];
[4-(2-Amino-4-methyl-thiazol-5-yl)-pyrimidin-2-yl]-(3,4,5-trimethoxy-phenyl)-amine [168];
[4-(2,6-Dimethyl-morpholin-4-yl)-phenyl]-[4-(2-ethylamino-4-methyl-thiazol-5-yl)-pyrimidin-2-yl]-amine [169];
(3,5-Dimethoxy-phenyl)-[4-(4-methyl-2-methylamino-thiazol-5-yl)-pyrimidin-2-yl]-amine [170];
(3,5-Dimethoxy-phenyl)-[4-(4-methyl-2-phenylamino-thiazol-5-yl)-pyrimidin-2-yl]-amine [171];
1-(4-{4-[4-(4-Methyl-2-phenylamino-thiazol-5-yl)-pyrimidin-2-ylamino]-phenyl}-piperazin-1-yl)-ethanone [172];
[4-(2,4-Dimethyl-thiazol-5-yl)-pyrimidin-2-yl]*(3-methoxy-4-morpholin-4-yl-phenyl)-amine [173];
[4-(2,4-Dimethyl-thiazol-5-yl)-pyrimidin-2-yl]-(4-morpholin-4-ylmethyl-phenyl)-amine [174];
(3,5-Dimethoxy-phenyl)-[4-(2,4-dimethyl-thiazol-5-yl)-pyrimidin-2-yl]-amine [175];
[4-(4-Benzyl-piperazin-1-yl)-phenyl]-[4-(4-methyl-2-phenylamino-thiazol-5-yl)-pyrimidin-2-yl]-amine [176];
Benzo[1,3]dioxol-5-yl-[4-(4-methyl-2-pyridin-3-yl-thiazol-5-yl)-pyrimidin-2-yl]-amine [177];
Benzo[1,3]dioxol-5-yl-[4-(2-ethylamino-4-methyl-thiazol-5-yl)-pyrimidin-2-yl]-amine [178];
[4-(2-Amino-4-methyl-thiazol-5-yl)-pyrimidin-2-yl]-benzo[1,3]dioxol-5-yl-amine [179];
(2,3-Dihydro-benzo[1,4]dioxin-6-yl)-[4-(4-methyl-2-pyridin-3-yl-thiazol-5-yl)-pyrimidin-2-yl]-amine [180];
(2,3-Dihydro-benzo[1,4]dioxin-6-yl)-[4-(2-ethylamino-4-methyl-thiazol-5-yl)-pyrimidin-2-yl]-amine [181];
[4-(2-Amino-4-methyl-thiazol-5-yl)-pyrimidin-2-yl]-(3-methoxy-4-morpholin-4-yl-phenyl)-amine [182];
(2,3-Dihydro-benzo[1,4]dioxin-6-yl)-[4-(4-methyl-2-phenethylamino-thiazol-5-yl)-pyrimidin-2-yl]-amine [183];
(4-Methoxy-3-methyl-phenyl)-[4-(4-methyl-2-methylamino-thiazol-5-yl)-pyrimidin-2-yl]-amine [184];
[4-(2-Amino-4-methyl-thiazol-5-yl)-pyrimidin-2-yl]-(4-methoxy-3-methyl-phenyl)-amine [185];
(4-Methoxy-3-methyl-phenyl)-[4-(4-methyl-2-pyridin-3-yl-thiazol-5-yl)-pyrimidin-2-yl]-amine [186]
[4-(2-Ethylamino-4-methyl-thiazol-5-yl)-pyrimidin-2-yl]-(4-methoxy-3-methyl-phenyl)-amine [187];
{4-Methyl-5-[2-(4-morpholin-4-yl-phenylamino)-pyrimidin-4-yl]-thiazol-2-yl}-methanol [188];
4-{4-[4-(2,4-Dimethyl-thiazol-5-yl)-pyrimidin-2-ylamino]-phenyl}-piperazine-1-carboxylic acid ethyl ester [189];
2-(4-{4-[4-(2,4-Dimethyl-thiazol-5-yl)-pyrimidin-2-ylamino]-phenyl}-piperazin-1-yl)-N-isopropyl-acetamide [190];
[4-(4-Methyl-2-methylamino-thiazol-5-yl)-pyrimidin-2-yl]-[4-(4-methyl-piperazin-1-yl)-phenyl]-amine [191];
[4-(4-Methyl-2-methylamino-thiazol-5-yl)-pyrimidin-2-yl]-[4-(4-methyl-piperazin-1-yl)-phenyl]-amine [192];
[4-(2,4-Dimethyl-thiazol-5-yl)-pyrimidin-2-yl]-(4-piperidin-1-yl-phenyl)-amine [193];
[4-(2-Ethylamino-4-methyl-4-thiazol-5-yl)-pyrimidin-2-yl]-(4-piperidin-1-yl-phenyl)-amine [194];
[4-(2-Amino-4-methyl-thiazol-5-yl)-pyrimidin-2-yl]-(4-piperidin-1-yl-phenyl)-amine [195];
[4-(2-Ethylamino-4-methyl-thiazol-5-yl)-pyrimidin-2-yl]-[4-(4-methyl-piperazin-1-yl)-phenyl]-amine [196];
{4-Methyl-5-[2-(4-piperidin-1-yl-phenylamino)-pyrimidin-4-yl]-thiazol-2-yl}-methanol [197];
[4-(4-Methyl-2-pyridin-3-yl-thiazol-5-yl)-pyrimidin-2-yl]-(4-pyrrolidin-1-yl-phenyl [198];
[4-(2,4-Dimethyl-thiazol-5-yl)-pyrimidin-2-yl]-(4-pyrrolidin-1-yl-phenyl)-amine [199];
{5-[2-(3-Methoxy-4-morpholin-4-yl-phenylamino)-pyrimidin-4-yl]-4-methyl-thiazol-2-yl}-methanol [200];
[4-(2-Amino-4-methyl-thiazol-5-yl)-pyrimidin-2-yl]-(4-thiomorpholin-4-yl-phenyl)-amine [201];
[4-(2,4-Dimethyl-thiazol-5-yl)-pyrimidin-2-yl]-(4-thiomorpholin-4-yl-phenyl)-amine [202];
[4-(2-Ethylamino-4-methyl-thiazol-5-yl)-pyrimidin-2-yl]-(4-thiomorpholin-4-yl-phenyl)amine [203]
[4-(4-Methyl-2-pyridin-3-yl-thiazol-5-yl)-pyrimidin-2-yl]-(4-thiomorpholin-4-yl-phenyl)-amine [204];
[4-(2,4-Dimethyl-thiazol-5-yl)-pyrimidin-2-yl]-(3-methyl-4-piperidin-1-yl-phenyl)-amine [205];
[4-(2-Amino-4-methyl-thiazol-5-yl)-pyrimidin-2-yl]-(3-methyl-4-piperidin-1-yl-phenyl)-amine [206]
[4-(2-Ethylamino-4-methyl-thiazol-5-yl)-pyrimidin-2-yl]-(3-methyl-4-piperidin-1-yl-phenyl)-amine [207];
(3-Methyl-4-piperidin-1-yl-phenyl)-[4-(4-methyl-2-pyridin-3-yl-thiazol-5-yl)-pyrimidin-2-yl]-amine [208];
{4-Methyl-5-[2-(3-methyl-4-piperidin-1-yl-phenylamino)-pyrimidin-4-yl]-thiazol-2-yl}-methanol [209];
5-[4-(2,4-Dimethyl-thiazol-5-yl)-pyrimidin-2-ylamino]-2-morpholin-4-yl-benzamide [210];
5-[4-(2-Amino-4-methyl-thiazol-5-yl)-pyrimidin-2-ylamino]-2-morpholin-4-yl-benzamide [211];
5-[4-(2-Ethylamino-4-methyl-thiazol-5-yl)-pyrimidin-2-ylamino]-2-morpholin-4-yl-benzamide [212]
Cyclopropyl-(4-{4-[4-(2,4-dimethyl-thiazol-5-yl)-pyrimidin-2-ylamino]-phenyl}-piperazin-1-yl)-methanone [213];
[4-(2,4-Dimethyl-thiazol-5-yl)-pyrimidin-2-yl]-(4-methyl-3-morpholin-4-yl-phenyl)-amine [214];
[4-(2,4-Dimethyl-thiazol-5-yl)-pyrimidin-2-yl]-(4-methoxy-3-morpholin-4-ylmethyl-phenyl)-amine [215];
{5-[2-(3-Methoxy-4-piperidin-1-yl-phenylamino)-pyrimidin-4-yl]-4-methyl-thiazol-2-yl}-methanol [216];
{4-Methyl-5-[2-(3-methyl-4-morpholin-4-yl-phenylamino)-pyrimidin-4-yl]-thiazol-2-yl}-methanol [217];
[4-(2-Amino-4-methyl-thiazol-5-yl)-pyrimidin-2-yl]-(3-methyl-4-morpholin-4-yl-phenyl)-amine [218];
[4-(2-Ethylamino-4-methyl-thiazol-5-yl)-pyrimidin-2-yl]-(3-methyl-4-morpholin-4-yl-phenyl)-amine [219];
[4-(2,4-Dimethyl-thiazol-5-yl)-pyrimidin-2-yl]-(3-methyl-4-morpholin-4-yl-phenyl)-amine [220];
or a pharmaceutically acceptable salt thereof.

31. A compound according to claim 1 which is selected from the following:
1-(4-{3-[4-(4-Methyl-2-methylamino-thiazol-5-yl)-pyrimidin-2-ylamino]-phenyl}-piperazin-1-yl)-ethanone[3];
[4-(2,4-Dimethyl-thiazol-5-yl)-pyrimidin-2-yl]-(3-methanesulfonyl-phenyl)-amine [4];
N-{3-[4-(2-Ethylamino-4-methyl-thiazol-5-yl)-pyrimidin-2-ylamino]-benzyl}-methanesulfonamide [5];
N-{3-[4-(2-Amino-4-methyl-thiazol-5-yl)-pyrimidin-2-ylamino]-benzyl}-methanesulfonamide [6];
[4-(4-Methyl-2-methylamino-thiazol-5-yl)-pyrimidin-2-yl]-(3-piperazin-1-yl-phenyl)-amine[7];
[4-(2,4-Dimethyl-thiazol-5-yl)-pyrimidin-2-yl]-(3-piperazin-1-yl-phenyl)-amine[8];
N-{3-[4-(2-Ethylamino-4-methyl-thiazol-5-yl)-pyrimidin-2-ylamino]-benzyl}-C,C,C-trifluoro-methanesulfonamide [10];
N-{3-[4-(2,4-Dimethyl-thiazol-5-yl)-pyrimidin-2-ylamino]-benzyl}-C,C,C-trifluoro-methanesulfonamide [11];
N-{3-[4-(2-Amino-4-methyl-thiazol-5-yl)-pyrimidin-2-ylamino]-benzyl}-C,C,C-trifluoro-methanesulfonamide [12];
N-{4-[4-(2-Ethylamino-4-methyl-thiazol-5-yl)-pyrimidin-2-ylamino]-benzyl}-acetamide[13];
N-{4-[4-(4-Methyl-2-methylamino-thiazol-5-yl)-pyrimidin-2-ylamino]-benzyl}-acetamide[14];
N-{4-[4-(2,4-Dimethyl-thiazol-5-yl)-pyrimidin-2-ylamino]-benzyl}-acetamide [15];
N-{4-[4-(2-Amino-4-methyl-thiazol-5-yl)-pyrimidin-2-ylamino]-benzyl}-acetamide[16];
[4-(2-Ethylamino-4-methyl-thiazol-5-yl)-pyrimidin-2-yl]-(4-methanesulfonyl-phenyl)-amine[17];
(4-Methanesulfonyl-phenyl)-[4-(4-methyl-2-methylamino-thiazol-5-yl)-pyrimidin-2-yl]-amine[20];
(3-Methanesulfonyl-phenyl)-[4-(4-methyl-2-methylamino-thiazol-5-yl)-pyrimidin-2-yl]-amine[27];
[4-(2-Ethylamino-4-methyl-thiazol-5-yl)-pyrimidin-2-yl]-(3-methanesulfonyl-phenyl)-amine[28];
1-(4-{3-[4-(2,4-Dimethyl-thiazol-5-yl)-pyrimidin-2-ylamino]-phenyl}-piperazin-1-yl)-ethanone[46];
{3-[4-(2,4-Dimethyl-thiazol-5-yl)-pyrimidin-2-ylamino]-5-hydroxymethyl-phenyl}-methanol[47];
{3-Hydroxymethyl-5-[4-(4-methyl-2-methylamino-thiazol-5-yl)-pyrimidin-2-ylamino]-phenyl}-methanol [48];
N-{3-[4-(2,4-Dimethyl-thiazol-5-yl)-pyrimidin-2-ylamino]-benzyl}-methanesulfonamide[49];
1-(4-{4-[4-(2-Amino-4-methyl-thiazol-5-yl)-pyrimidin-2-ylamino]-phenyl}-piperazin-1-yl)-ethanone [57];
1-(4-{4-[4-(2-Ethylamino-4-methyl-thiazol-5-yl)-pyrimidin-2-ylamino]-phenyl}-piperazin-1-yl)-ethanone [58];
[4-(2-Ethylamino-4-methyl-thiazol-5-yl)-pyrimidin-2-yl]-(4-piperazin-1-yl-phenyl)-amine [59];
[4-(4-Benzyl-piperazin-1-yl)-phenyl]-[4-(2-ethylamino-4-methyl-thiazol-5-yl)-pyrimidin-2-yl]-amine [60];
[4-(2-Amino-4-methyl-thiazol-5-yl)-pyrimidin-2-yl]-(4-piperazin-1-yl-phenyl)-amine [61];
[4-(2-Benzylamino-4-methyl-thiazol-5-yl)-pyrimidin-2-yl]-(4-morpholin-4-yl-phenyl)-amine [117];
1-(4-{4-[4-(4-Methyl-2-pyridin-3-yl-thiazol-5-yl)-pyrimidin-2-ylamino]-phenyl}-piperazin-1-yl)-ethanone [119];
{4-[2-(Ethyl-methyl-amino)-4-methyl-thiazol-5-yl]-pyrimidin-2-yl}-(4-morpholin-4-yl-phenyl)-amine [120];
[4-(2,6-Dimethyl-morpholin-4-yl)-phenyl]-[4-(2,4-dimethyl-thiazol-5-yl)-pyrimidin-2-yl]-amine [121];
{5-[2-(4-Dimethylamino-phenylamino)-pyrimidin-4-yl]-4-methyl-thiazol-2-yl}-methanol [132];
{4-[4-Methyl-2-(thiophene-2-sulfonylmethyl)-thiazol-5-yl]-pyrimidin-2-yl}-(4-morpholin-4-yl-phenyl)-amine [138];
{4-[2-(2-Methoxy-ethylamino)-4-methyl-thiazol-5-yl]-pyrimidin-2-yl}-(4-morpholin-4-yl-phenyl)-amine [144];
N⁴-[4-(2,4-Dimethyl-thiazol-5-yl)-pyrimidin-2-yl]-N¹-methyl-2-trifluoromethyl-benzene-1,4-diamine [149];
[4-(2,4-Dimethyl-thiazol-5-yl)-pyrimidin-2-yl]-(3-morpholin-4-ylmethyl-phenyl)-amine [150];
4-[4-(2,4-Dimethyl-thiazol-5-yl)-pyrimidin-2-ylamino]-2-morpholin-4-ylmethyl-phenol [151];
(3-Methoxy-4-morpholin-4-yl-phenyl)-[4-(4-methyl-2-pyridin-3-yl-thiazol-5-yl)-pyrimidin-2-yl]-amine [156];
[4-(2-Ethylamino-4-methyl-thiazol-5-yl)-pyrimidin-2-yl]-(3-methoxy-4-morpholin-4-yl-phenyl)-amine [157];
[4-(2,6-Dimethyl-morpholin-4-yl)-phenyl]-[4-(2-ethylamino-4-methyl-thiazol-5-yl)-pyrimidin-2-yl]-amine [169];
[4-(2-Amino-4-methyl-thiazol-5-yl)-pyrimidin-2-yl]-(3-methoxy-4-morpholin-4-yl-phenyl)-amine [182];
[4-(2,4-Dimethyl-thiazol-5-yl)-pyrimidin-2-yl]-(4-piperidin-1-yl-phenyl)-amine [193];
[4-(2-Ethylamino-4-methyl-thiazol-5-yl)-pyrimidin-2-yl]-(4-piperidin-1-yl-phenyl)-amine [194];
[4-(2-Amino-4-methyl-thiazol-5-yl)-pyrimidin-2-yl]-(4-piperidin-1-yl-phenyl)-amine [195];
{4-Methyl-5-[2-(4-piperidin-1-yl-phenylamino)-pyrimidin-4-yl]-thiazol-2-yl}-methanol [197];
{5-[2-(3-Methoxy-4-morpholin-4-yl-phenylamino)-pyrimidin-4-yl]-4-methyl-thiazol-2-yl}-methanol [200];
[4-(2-Amino-4-methyl-thiazol-5-yl)-pyrimidin-2-yl]-(4-thiomorpholin-4-yl-phenyl)-amine [201];
[4-(2,4-Dimethyl-thiazol-5-yl)-pyrimidin-2-yl]-(4-thiomorpholin-4-yl-phenyl)-amine [202];
[4-(2-Ethylamino-4-methyl-thiazol-5-yl)-pyrimidin-2-yl]-(4-thiomorpholin-4-yl-phenyl)-amine [203];
[4-(2,4-Dimethyl-thiazol-5-yl)-pyrimidin-2-yl]-(3-methyl-4-piperidin-1-yl-phenyl)-amine [205];
[4-(2-Amino-4-methyl-thiazol-5-yl)-pyrimidin-2-yl]-(3-methyl-4-piperidin-1-yl-phenyl)-amine [206];
[4-(2-Ethylamino-4-methyl-thiazol-5-yl)-pyrimidin-2-yl]-(3-methyl-4-piperidin-1-yl-phenyl)-amine [207];
{4-Methyl-5-[2-(3-methyl-4-piperidin-1-yl-phenylamino)-pyrimidin-4-yl]-thiazol-2-yl}-methanol [209];
Cyclopropyl-(4-{4-[4-(2,4-dimethyl-thiazol-5-yl)-pyrimidin-2-ylamino]-phenyl}-piperazin-1-yl)-methanone [213];
[4-(2,4-Dimethyl-thiazol-5-yl)-pyrimidin-2-yl]-(4-methoxy-3-morpholin-4-ylmethyl-phenyl)-amine [215];
or a pharmaceutically acceptable salt thereof.

32. A pharmaceutical composition comprising a compound according to any one of claims 1, 19, or 30 admixed with a pharmaceutically acceptable diluent, excipient or carrier.

33. (canceled)

34. The method according to claim 48 wherein the proliferative disorder is cancer or leukaemia.

35. The method according to claim 48 wherein the proliferative disorder is glomerulonephritis, rheumatoid arthritis, psoriasis or chronic obstructive pulmonary disorder.

36. The method according to claim 48 wherein said compound is administered in combination with one or more other anticancer compounds.

37. (canceled)

38. The method according to claim 49 wherein the viral disorder is selected from human cytomegalovirus (HCMV), herpes simplex virus type 1 (HSV-1), human immunodeficiency virus type 1 (HIV-1), and varicella zoster virus (VZV).

39. A method of inhibiting a protein kinase, comprising contacting said protein kinase with a compound of claim 1, 19, or 30.

40. The method according to claim 39 wherein said protein kinase is a cyclin dependent kinase.

41. The method according to claim 40 wherein said cyclin dependent kinase is selected from CDK2, CDK7, CDK8 and CDK9.

42. The method according to claim 38 wherein said protein kinase is aurora kinase.

43. The method according to claim 42 wherein said aurora kinase is aurora kinase A, aurora kinase B or aurora kinase C.

44. The method according to claim 39 wherein said protein kinase is a tyrosine kinase.

45. The method according to claim 42 wherein said tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

46. The method according to claim 39 wherein said protein kinase is GSK.

47. The method according to claim 46 wherein said protein kinase is GSK-3β.

48. A method of treating a proliferative disorder or disease, said method comprising administering to a mammal a therapeutically effective amount of a compound according to any one of claims 1, 19, or 30.

49. A method of treating a viral disorder, said method comprising administering to a mammal a therapeutically effective amount of a compound according to any one of claims 1, 19, or 30.

50. An assay for identifying further candidate compounds capable of inhibiting one or more of a cyclin dependent kinase, an aurora kinase, GSK, a tyrosine kinase and a PLK enzyme, wherein said assay comprises contacting a compound according to claims 1, 19, or 30 with an enzyme selected from a cyclin dependent kinase, GSK, a tyrosine kinase and PLK.

51. The method according to claim 50 wherein said assay is a competitive binding assay.

52. The method according to claim 51 wherein said competitive binding assay comprises contacting the compound with an enzyme selected from a cyclin dependent kinase, GSK, a tyrosine kinase and PLK, and a candidate compound and detecting any change in the interaction between the compound and the enzyme.

53. A method for treating a CNS disorder, said method comprising administering to a mammal a therapeutically effective amount of a compound according to any one of claims 1, 19, or 30.

54. The method according to claim 53 wherein the CNS disorder is Alzheimer's disease or bipolar disorder.

55. A method for treating alopecia, said method comprising administering to a mammal a therapeutically effective amount of a compound according to any one of claims 1, 19, or 30.

56. A method for treating a stroke, said method comprising administering to a mammal a therapeutically effective amount of a compound according to any one of claims 1, 19, or 30.

57. The method according to claim 48 wherein the compound is administered in an amount sufficient to inhibit at least one PLK enzyme.

58. The method according to claim 57 wherein the PLK enzyme is PLK1.

59. The method according claim 48 wherein the compound is administered in an amount sufficient to inhibit at least one CDK enzyme.

60. The method according to claim 59 wherein the CDK enzyme is CDK1, CDK2, CDK3, CDK4, CDK6, CDK7, CDK8 and/or CDK9.

61. The method according claim 48 wherein the compound is administered in an amount sufficient to inhibit aurora kinase.

62. The method according to claim 61 wherein the aurora kinase is aurora kinase A, aurora kinase B or aurora kinase C.

63. The method according to claim 48 wherein the compound is administered in an amount sufficient to inhibit at least one tyrosine kinase.

64. The method according to claim 63 wherein the tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

65. A method for treating diabetes, said method comprising administering to a mammal a therapeutically effective amount of a compound according to any one of claims 1, 19, or 30.

66. The method according to claim 65 wherein the diabetes is Type II diabetes.

67. The method according to claim 65 wherein the compound is administered in an amount sufficient to inhibit GSK.

68. The method according to claim 67 wherein the compound is administered in an amount sufficient to inhibit GSK3β.
